(19) United States
(12) Patent Application Publication
(10) Pub. No.: US 2025/0255868 A1
(43) Pub. Date: Aug. 14, 2025

(54) HETEROCYCLIC COMPOUND ACTING AS KRAS G12D INHIBITOR

(71) Applicant: Hangzhou Innogate Pharma Co., Ltd., Hangzhou Zhejiang (CN)

(72) Inventors: Hancheng ZHANG, Hangzhou Zhejiang (CN); Wei JIA, Hangzhou Zhejiang (CN); Congcong CAI, Hangzhou Zhejiang (CN)

(21) Appl. No.: 18/554,582

(22) PCT Filed: Apr. 11, 2022
(86) PCT No.: PCT/CN2022/086183

§ 371 Date: May 2, 2025

(30) Foreign Application Priority Data
Apr. 9, 2021 (CN) 202110384952.8
Apr. 16, 2021 (CN) 202110414411.5
Jul. 7, 2021 (CN) 202110768789.5
Aug. 13, 2021 (CN) 202110932970.5
Oct. 13, 2021 (CN) 202111193190.X
Dec. 2, 2021 (CN) 202111460701.X

(51) Int. Cl. A61K 31/519; A61P 35/00; C07D 471/04; C07D 519/00

(52) U.S. Cl. CPC A61K 31/519; A61P 35/00; C07D 471/04; C07D 519/00

(57) ABSTRACT

Provided in the present invention is a compound acting as a KRAS G12D inhibitor; specifically provided in the present invention is a compound of the structure shown in the following formula (I), or an optical isomer, a pharmaceutically acceptable salt, a prodrug, a deuterated derivative, a hydrate, or a solvate thereof. The present compound can be used for the treatment or prevention of diseases or conditions associated with the activity or expression of KRAS G12D.

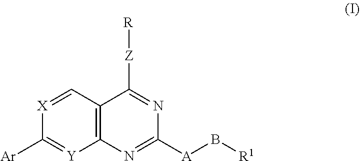

### TECHNICAL FIELD

The present invention relates to the field of pharmaceutical chemistry, specifically relates to a novel class of derivatives containing tricyclic heteroaryl, the preparation therefor and its application as a KRAS G12D inhibitor in the preparation of drugs for the treatment of a wide range of diseases such as tumors.

### BACKGROUND

KRAS (Kirsten RatSarcoma Viral Oncogene Homolog) is a small GTPase belonging to the RAS family. There are currently three RAS genes, known as HRAS, NRAS and KRAS. RAS protein plays an important role in the occurrence and development of cancer. RAS mutations exist in 20%-30% of human tumors, of which KRAS mutation is the most prevalent and more commonly found in lung cancer, pancreatic cancer and colon cancer.

KRAS protein contains 188 amino acid residues and has multiple functional motifs, including nucleotide binding domain, switch-I and switch-II domain, nucleotide exchange factor (GEF) binding domain, GTPase-activating protein (GAP) binding domain and effector protein binding domain. Under normal circumstances, KRAS activity is regulated by a variety of upstream signaling factors, such as receptor tyrosine kinases, integrins and G protein-coupled receptors. KRAS, as a molecular switch in the cell, is activated (on) or inactivated (off) through the conversion of GTP or GDP binding states, respectively, and transmits signals received upstream to effector proteins downstream. KRAS protein itself has relatively slow nucleotide exchange rate and weak GTP hydrolysis function. After receiving upstream stimulus signals, KRAS protein releases GDP and binds GTP with the assistance of guanine nucleotide exchange factors such as SOS1 (Son of Sevenless 1). GTP-bound RAS protein is in an activated state, recruits downstream effector proteins, activates Raf-MEK-ERK and PI3K-AKT-mTOR signaling pathways, and promotes cell growth, proliferation, survival, metabolism and angiogenesis. The binding of GTPase-activating protein GAP significantly promoted the GTP hydrolysis function of KRAS. GTP was hydrolyzed to GDP, and KRAS in the GDP-binding state was transformed into an inactivated state with the molecular switch off. When RAS gene is mutated, its innate or GAP-induced GTPase activity is inhibited, RAS is always in a GTP-binding state, and the molecular switch is continuously open, which leads to a variety of downstream signaling pathways in a constitutively active state, and abnormal cell proliferation and growth.

In pancreatic cancer, up to 90% of patients have KRAS mutations, and about 40% of the point mutations are KRAS G12D mutations. In addition, KRAS G12D mutations are present in approximately 13.3% of colorectal cancer, 10% of colorectal cancer, 4% of non-small cell lung cancer and 1.7% of small cell lung cancer patients.

Due to its important role in tumorigenesis and its high mutation frequency, KRAS G12D is a very attractive anti-tumor target. Currently, there are no effective KRAS G12D inhibitors on the market. Based on the above background, we developed a novel class of KRAS G12D inhibitors.

### SUMMARY

The purpose of the present invention is to provide a novel class of KRAS G12D inhibitors.

In the first aspect of the present invention, provided is a compound of the structure shown in formula(I) below, or an optical isomer, a pharmaceutically acceptable salt, a prodrug, a deuterated derivative, a hydrate, or a solvate thereof:

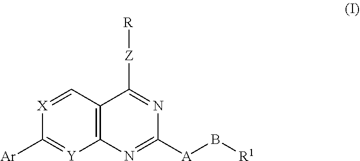

wherein:
Ar is selected from aryl or heteroaryl; the aryl or heteroaryl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄ haloalkenyl, C₂₋₄alkynyl, C₂₋₄haloalkynyl, C₃₋₆cycloalkyl, 3- to 6-membered heterocyclyl, C₃₋₆ cycloalkyl-O—, 3- to 6-membered heterocyclyl-O—, C₃₋₆cycloalkyl C₂₋₄ alkynyl, 3- to 6-membered heterocyclyl C₂₋₄alkynyl, C₁₋₄haloalkyl C₂₋₄alkynyl, NR²R², CN, SR², —OC(O)R^{k}, —O—P(O)(OR^{m})₂, and —O—CH(Rⁿ)—O—P(O)(OR^{m})₂; wherein, each R² is independently hydrogen or C₁₋₄ alkyl; R^{k} is selected from C₁₋₁₂ alkyl, wherein, the alkyl is optionally substituted by one or more groups selected from the group consisting of: hydroxyl, C₁₋₄alkoxy, NR²R², C(O)OH, C(O)OC₁₋₂ alkyl, and CN; R^{m} is selected from hydrogen and C₁₋₄ alkyl; Rⁿ is selected from hydrogen and C₁₋₄ alkyl; the cycloalkyl or heterocyclyl as described above is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂-4 alkenyl, C₂₋₄alkynyl, CN, and ═M; wherein M is selected from O and CR³R⁴; R³ and R⁴ are independently selected from the group consisting of: hydrogen, fluorine, and C₁₋₄ alkyl;
R is selected from 5 to 12 membered heterocyclyl, including partially unsaturated or saturated monocyclic or polycyclic heterocyclyl, and the polycyclic heterocyclyl includes spiral, fused, or bridged heterocyclyl; the heterocyclyl is optionally substituted by one or more groups selected from the group consisting of: halogen, C₁₋₄ alkyl, ═O, hydroxyl, CN, and —CO(O)—CH(Rⁿ)—O—C(O)—U; wherein, Rⁿ is selected from hydrogen and C₁₋₄ alkyl; U is selected from C₁₋₁₈ alkyl;
Z is selected from chemical bonds, —O—, —S—, —NR⁵—, —C(R^{a})₂—, —C═C—, —CR^{a}═CR^{a}—, —N═, and —CR^{a}═; wherein, R⁵ is selected from hydrogen or C₁₋₄ alkyl; R^{a} is selected from hydrogen, halogen, and C₁₋₄ alkyl;
A is selected from chemical bonds, —O—, —S—, and —NR⁶—; wherein, R⁶ is selected from hydrogen and C₁₋₄ alkyl;
B is selected from —(CR⁷R⁸)ₘ—, —(CR⁷R⁸)ₘ-T-(CR⁷R⁸)ₘ—; wherein, T is selected from —C═C—, —CR^{a}═CR^{a}—, C₃₋₆cycloalkyl, and 3- to 6-membered heterocyclyl; wherein, the cycloalkyl or heterocyclyl is optionally substituted by one or more groups selected from the group consisting of: halogen, C₁₋₄alkyl, C₁₋₄ haloalkyl, hydroxyl, C₁₋₄alkoxy, C₁₋₄haloalkoxy, and CN; R⁷ and R⁸ are independently selected from the group consisting of: hydrogen, halogen, and C₁₋₄ alkyl; or R⁷ and R⁸ together with the C atom to which they attached form a C₃₋₆cycloalkyl; R^{a} is selected from hydrogen, halogen, and C₁₋₄ alkyl; each m is independently selected from 0, 1, 2, and 3;
R¹ is selected from C₃₋₈cycloalkyl or 4- to 12-membered heterocyclyl, wherein the heterocyclyl includes partially unsaturated or saturated monocyclic or polycyclic heterocyclyl, and the polycyclic heterocyclyl includes spiral, fused, or bridged heterocyclyl; the cycloalkyl or heterocyclyl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, —CH₂OC(O) NR⁹R¹⁰, CN, SR², C₂₋₄ alkenyl, C₂₋₄alkynyl, C₃₋₈cycloalkyl, 3 to 8-membered heterocyclyl, NR^{h}R^{h}, —(CR⁷R⁸)ₘ—OR^{h}, —(CR⁷R⁸)ₘ—NR^{h}R^{h}, and ═M; wherein, R⁹ and R¹⁰ are independently selected from hydrogen or C₁₋₄ alkyl, or R⁹ and R¹⁰ together with the N atom to which they attached form a 4-to-8-membered heterocyclyl comprising 1 or 2 N atoms and 0 or 1 heteroatom selected from O and S; each R^{h} is independently hydrogen, C₁₋₄ alkyl, or C₁₋₄haloalkyl; M is selected from O and CR³R⁴; the definitions of R², R³, R⁴, R⁷, R⁸, and m are as described above; X and Y are independently selected from N and CR¹¹; wherein, R¹¹ is selected from hydrogen, halogen, C₁₋₄ alkyl, C₁₋₄haloalkyl, C₁₋₄ alkoxy, C₁₋₄haloalkoxy, C₂₋₄alkynyl, C₃₋₆cycloalkyl, and CN;
provided that when R¹ is not substituted by ═M, and M is selected from CR³R⁴,

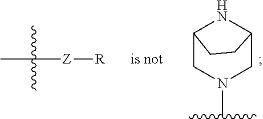

the structural fragment
wherein, each of the above-mentioned alkyl, alkenyl, alkynyl, cycloalkyl, heterocyclyl, aryl, and heteroaryl is optionally and independently substituted by 1-3 substituents independently selected from the group consisting of: halogen, C₁₋₄alkyl, C₁₋₄haloalkyl, C₂₋₄alkenyl, C₂₋₄alkynyl, C₃₋₈cycloalkyl, 3- to 8-membered heterocyclyl, aryl, heteroaryl, CN, NO₂, OR^{h}, SR^{h}, NR^{h}R^{h}, C(O) R^{t}, C(O) OR^{h}, C (O) NR^{h}R^{h}, NR^{h}C(O) R^{t}, NR^{h}S(O)₂R^{t} and S(O)₂R^{t}, provided that the formed chemical structure is stable and meaningful; wherein, R^{t} is C₁₋₄ alkyl, C₂₋₄ alkenyl, C₂₋₄ alkynyl, C₃₋₈cycloalkyl, 4- to 8-membered heterocyclyl, aryl, or heteroaryl; each R^{h} is independently hydrogen, C₁₋₄ alkyl, or C₁₋₄ haloalkyl; or two R^{h} together with the N atom to which they attached form a 3-to-8-membered heterocyclyl comprising 1 or 2 N atoms and 0 or 1 heteroatom selected from O and S; and
unless otherwise specified, the above-mentioned aryl is an aromatic group containing 6-12 carbon atoms; the heteroaryl is a 5- to 15-membered (preferably 5- to 12-membered) heteroaromatic group.

In another preferred embodiment, when R¹ is not substituted by ═M, and M is selected from CR³R⁴, the structural fragment

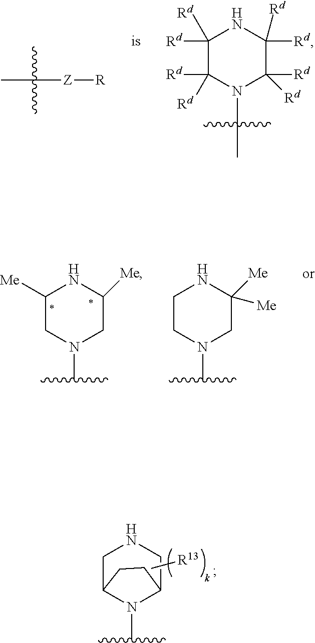

wherein, each R^{d} is independently selected from hydrogen, C₁₋₄ alkyl, and C₃₋₆cycloalkyl; or two R^{d} together with the same C atom to which they attached form a C₃₋₆ cycloalkyl.

In another preferred embodiment, when R¹ is not substituted by ═M, and M is selected from CR³R⁴, the structural fragment

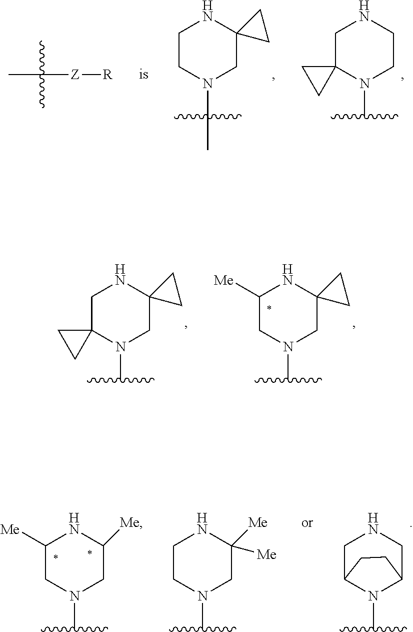

In another preferred embodiment, when R is not substituted by ═M, and M is selected from CR³R⁴, the structural fragment

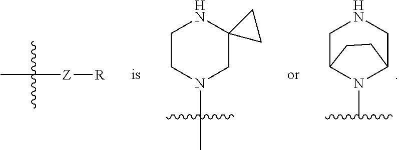

In another preferred embodiment, formula (I) is formula (II):

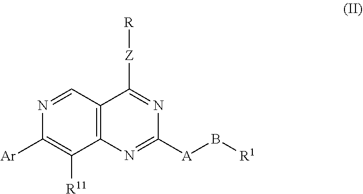

wherein:
Ar is selected from aryl or heteroaryl; the aryl or heteroaryl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄ haloalkenyl, C₂₋₄alkynyl, C₂₋₄haloalkynyl, C₃₋₆cycloalkyl, 3- to 6-membered heterocyclyl, C₃₋₆ cycloalkyl-O—, 3- to 6-membered heterocyclyl-O—, C₃₋₆cycloalkyl C₂₋₄ alkynyl, 3- to 6-membered heterocyclyl C₂₋₄alkynyl, C₁₋₄haloalkyl C₂₋₄alkynyl, NR²R², CN, SR², —OC(O)R^{k}, —O—P(O)(OR^{m})₂, and —O—CH (Rⁿ)—O—P(O)(OR^{m})₂; wherein, each R² is independently hydrogen or C₁₋₄ alkyl; R^{k} is selected from C₁-12 alkyl, wherein, the alkyl is optionally substituted by one or more groups selected from the group consisting of: hydroxyl, C₁₋₄alkoxy, NR²R², C(O)OH, C(O)OC₁₋₂ alkyl, and CN; R^{m} is selected from hydrogen, and C₁₋₄ alkyl; Rⁿ is selected from hydrogen and C₁₋₄ alkyl; the cycloalkyl or heterocyclyl as described above is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄alkynyl, CN, and ═M; wherein, M is selected from 0 and CR³R⁴; R³ and R⁴ are independently selected from the group consisting of: hydrogen, fluorine, and C₁₋₄ alkyl;
R is selected from 5 to 12 membered heterocyclyl, including partially unsaturated or saturated monocyclic or polycyclic heterocyclyl, and the polycyclic heterocyclyl includes spiral, fused, or bridged heterocyclyl; the heterocyclyl is optionally substituted by one or more groups selected from the group consisting of: halogen, C₁₋₄ alkyl, ═O, hydroxyl, CN, and —CO(O)—CH (Rⁿ)—O—C(O)—U; wherein, Rⁿ is selected from hydrogen and C₁₋₄ alkyl; U is selected from C₁₋₁₈ alkyl;
Z is selected from chemical bonds, —O—, —S—, —NR⁵—, —C(R^{a})₂—, —C═C—, —CR^{a}═CR^{a}—, —N═, and —CR^{a}═; wherein, R⁵ is selected from hydrogen and C₁₋₄ alkyl; R^{a} is selected from hydrogen, halogen, and C₁₋₄ alkyl;
A is selected from chemical bonds, —O—, —S—, and —NR⁶—; wherein, R⁶ is selected from hydrogen and C₁₋₄ alkyl;
B is selected from —(CR⁷R⁸)ₘ—, and —(CR⁷R⁸)ₘ-T-(CR⁷R⁸)ₘ—; wherein, T is selected from —C═C—, —CR^{a}═CR^{a} —, C₃₋₆cycloalkyl, and 3- to 6-membered heterocyclyl; wherein, the cycloalkyl or heterocyclyl is optionally substituted by one or more groups selected from the group consisting of: halogen, C₁₋₄alkyl, C₁₋₄ haloalkyl, hydroxyl, C₁₋₄alkoxy, C₁₋₄haloalkoxy, and CN; R⁷ and R⁸ are independently selected from the group consisting of: hydrogen, halogen, and C₁₋₄ alkyl; or R⁷ and R⁸ together with the C atom to which they attached form a C₃₋₆cycloalkyl; R^{a} is selected from hydrogen, halogen, and C₁₋₄ alkyl; each m is independently selected from 0, 1, 2, and 3;
R¹ is selected from C₃₋₈cycloalkyl or 4- to 12-membered heterocyclyl, wherein the heterocyclyl includes partially unsaturated or saturated monocyclic or polycyclic heterocyclyl, and the polycyclic heterocyclyl includes spiral, fused, or bridged heterocyclyl; the cycloalkyl or heterocyclyl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, —CH₂OC (O) NR⁹R¹⁰, CN, SR², C₂₋₄ alkenyl, C₂₋₄alkynyl, C₃₋₈cycloalkyl, 3 to 8-membered heterocyclyl, NR^{h}R^{h}, —(CR⁷R⁸)ₘ—OR^{h}, —(CR⁷R⁸)ₘ—NR^{h}R^{h}, and ═M; wherein, R⁹ and R¹⁰ are each independently selected from hydrogen and C₁₋₄ alkyl, or R⁹ and R¹⁰ together with the N atom to which they attached form a 4-to-8-membered heterocyclyl comprising 1 or 2 N atoms and 0 or 1 heteroatom selected from O and S; each R^{h} is independently hydrogen, C₁₋₄ alkyl, or C₁₋₄haloalkyl; M is selected from O and CR³R⁴;
R², R³, R⁴, R⁷, R⁸, and m are as defined above;
R¹¹ is selected from hydrogen, halogen, C₁₋₄ alkyl, C₁₋₄haloalkyl, C₁₋₄ alkoxy, C₁₋₄haloalkoxy, C₂₋₄alkynyl, C₃₋₆cycloalkyl, and CN.

In another preferred embodiment, the structural fragments

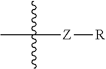

in formula (I) or (II) is selected from the group consisting of:

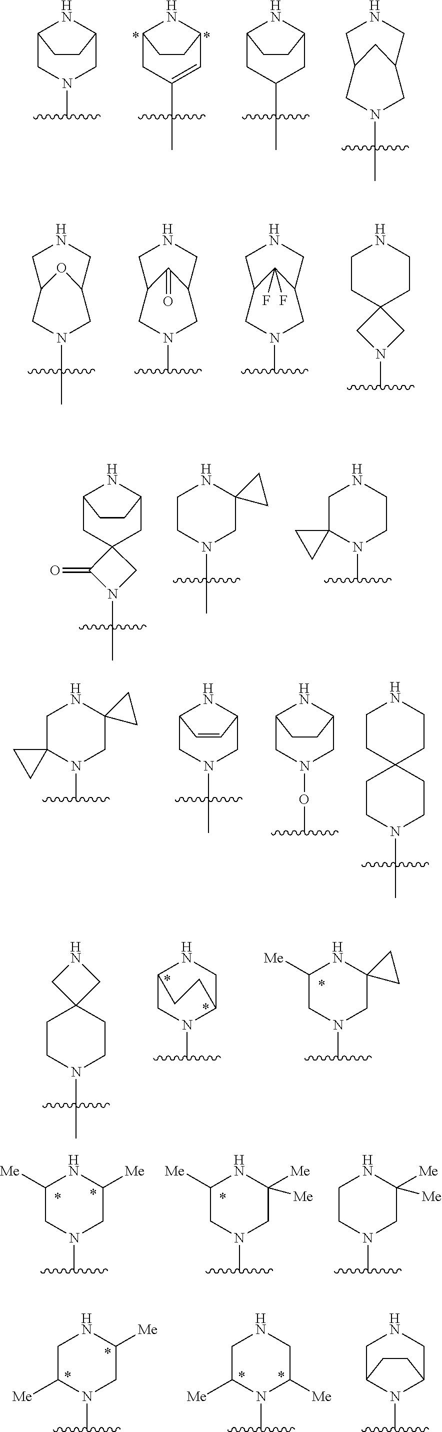

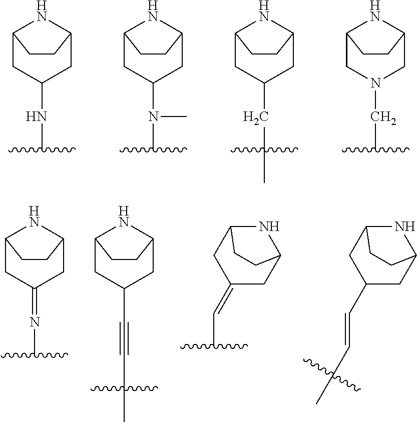

[symbol] represents the connection site between the above structural fragment and the rest of the structure of formula (I) or formula (II);
“*” represents a chiral center;
the above-mentioned group is optionally substituted by 0, 1, or 2 R¹³, wherein R¹³ is selected from halogen, C₁₋₄ alkyl, ═O, hydroxyl, and CN.

In another preferred embodiment, Ar is selected from the group consisting of: phenyl, and naphthyl.

In another preferred embodiment, Ar is selected from the group consisting of:

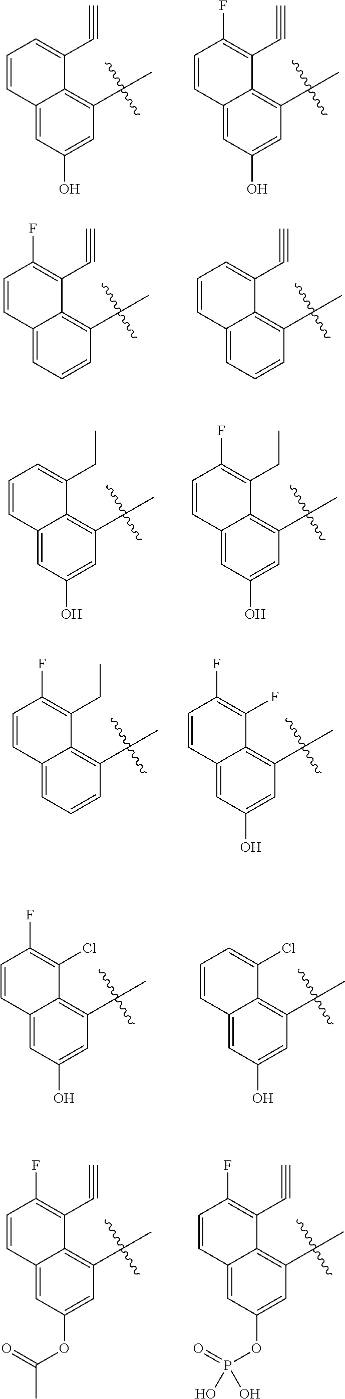

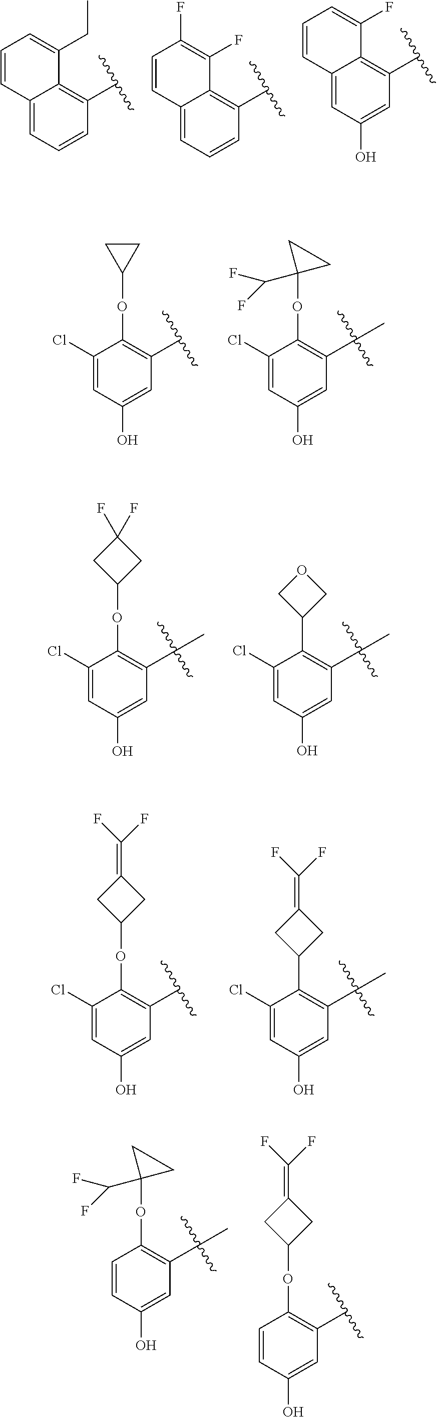

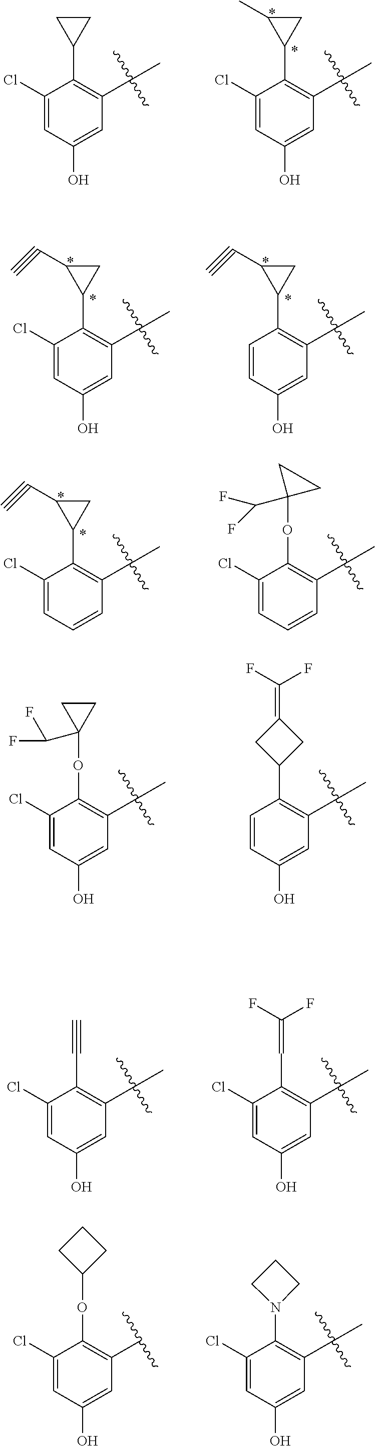

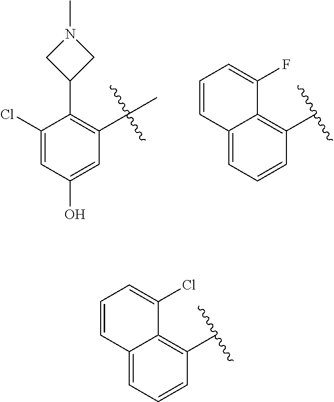 [SMILES: *c1cc(O)cc(Cl)c1C1CN(C)C1 ; *c1cccc2cccc(F)c12 ; *c1cccc2cccc(Cl)c12]

“ [symbol] ” represents the connection site between the above-mentioned structural fragment and the rest of the structure of formula (I) or formula (II);
“*” represents a chiral center.

In another preferred embodiment, the structural fragment

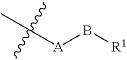

of formula (I) or formula (II) is selected from the group consisting of:

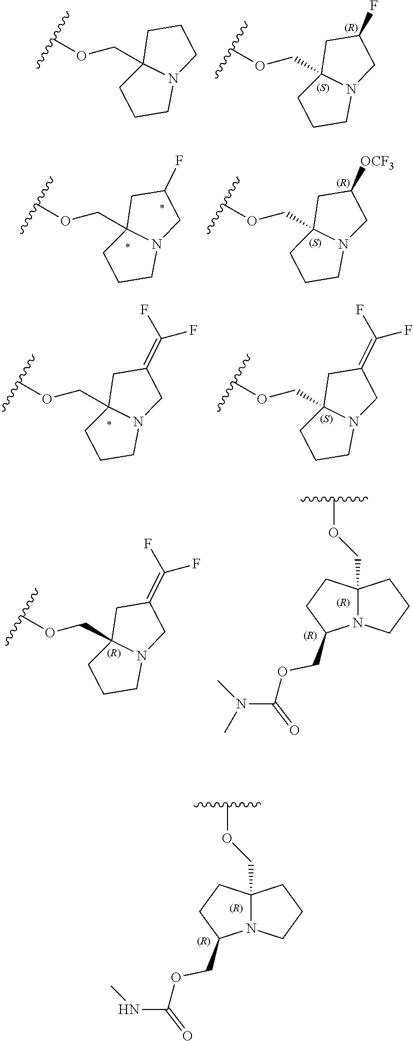

“*” represents a chiral center;
“ [symbol] ” represents the connection site between the above-mentioned structural fragment and other structures in formula (I) or formula (II).

In another preferred embodiment, formula (I) is formula (IIIa) or formula (IIIb),

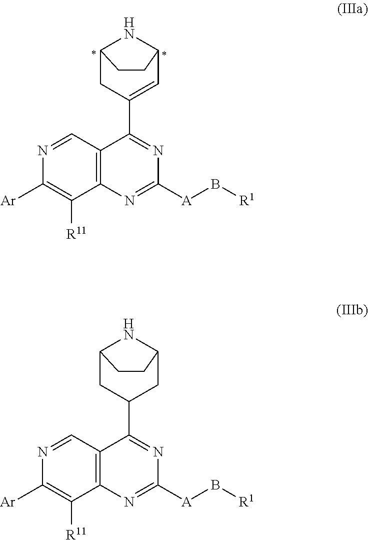

“*” represents a chiral center;
wherein, Ar, A, B, R¹, and R¹¹ are as defined above.

In another preferred embodiment, formula (I) is formula (IV),

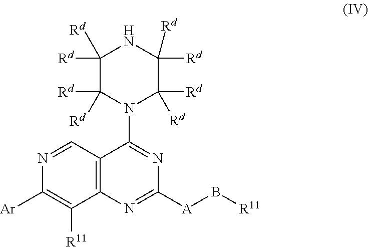

each R^{d} is independently selected from hydrogen, C₁₋₄ alkyl, and C₃₋₆cycloalkyl; or two R^{d} together with the same C atom they attached form a C₃₋₆cycloalkyl; and at least two R^{d} together with the same C atom they attached form a C₃₋₆cycloalkyl;
Ar, A, B, R¹, R¹¹ are as defined above.

In another preferred embodiment, formula (I) is formula_(V):

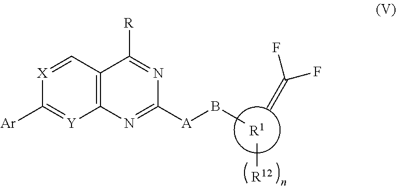

R¹is selected from C₃₋₈cycloalkyl and 4- to 12-membered heterocyclyl;
R¹² is selected from hydrogen, halogen, C₁₋₄ alkyl, C₁₋₄haloalkyl, —CH₂OC (O) NR⁹R¹⁰, C₂₋₄ alkenyl, C₂₋₄ alkynyl, C₃₋₈cycloalkyl, 3 to 8-membered heterocyclyl, CN, OR^{h}, SR^{h}, NR^{h}R^{h}, —(CR⁷R⁸)ₘ—OR^{h}, and —(CR⁷R⁸)ₘ—NR^{h}R^{h};
n is selected from 0, 1, and 2;
Ar, X, Y, R, A, B, R⁷, R⁸, R⁹, R¹⁰, R^{h}, and m are as defined above.

In another preferred embodiment, formula (I) has the structure of formula (VI),

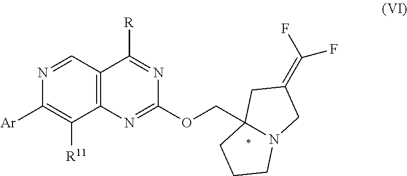

R is a group selected from the group consisting of:

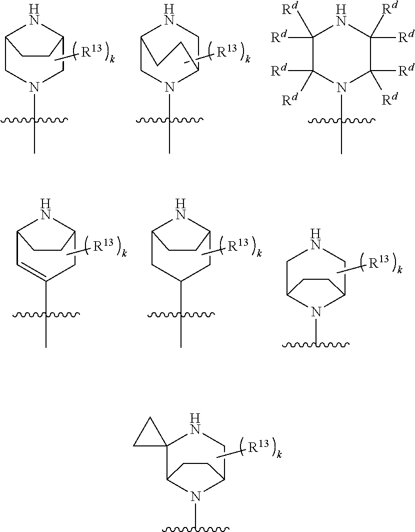

Ar is selected from the group consisting of:

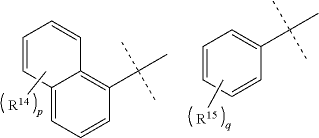

“*” represents a chiral center;
“ [symbol] ” represents the connection site between R and other structures of the compound of formula (VI);
“---” represents the connection site between Ar and other structures of the compound of formula (VI);
R¹³ is selected from halogen, C₁₋₄ alkyl, ═O, hydroxyl, and CN;
R¹⁴ and R¹⁵ are independently selected from halogen, C₁₋₄ alkyl, C₁₋₄haloalkyl, hydroxyl, C₁₋₄alkoxy, C₁₋₄haloalkoxy, C₂₋₄ alkenyl, C₂₋₄ haloalkenyl, C₂₋₄ alkynyl, C₂₋₄haloalkynyl, C₃₋₆ cycloalkyl, 3 to 6-membered heterocyclyl, C₃₋₆cycloalkyl-O—, 3 to 6-membered heterocyclyl-O—, NR²R², CN, SR², —OC(O)R^{k}, —O—P(O) (OR^{m}) ₂, and —O—CH (Rⁿ)—O—P(O) (OR^{m})₂; wherein, each R² is independently hydrogen or C₁₋₄ alkyl; R^{k} is selected from C₁₋₁₂ alkyl, wherein the alkyl is optionally substituted by one or more groups selected from the group consisting of: hydroxyl, C₁₋₄alkoxy, NR²R², C(O)OH, C(O)OC₁₋₂ alkyl, and CN; R^{m} is selected from hydrogen and C₁₋₄ alkyl; Rⁿ is selected from hydrogen and C₁₋₄ alkyl group; the cycloalkyl or heterocyclyl mentioned above is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄alkynyl, CN, and ═M; wherein, M is selected from 0 and CR³R⁴; R³ and R⁴ are independently selected from the group consisting of: hydrogen, fluorine, and C₁₋₄ alkyl;
k is selected from 0, 1, and 2;
p is selected from 0, 1, 2, 3, 4, and 5;
q is selected from 0, 1, 2, 3 and 4;
R¹¹ is as defined above; R^{d} is as defined above.

In another preferred embodiment, formula (I) is formula (VII),

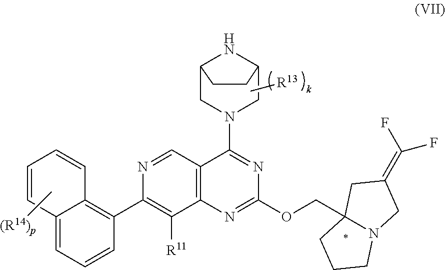

“*” represents a chiral center;
R¹³, R¹⁴, R¹¹, k, and p are as defined above.

In another preferred embodiment, formula (I) is formula (VIII),

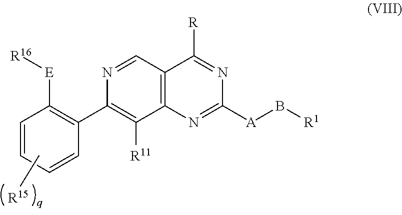

E is selected from chemical bonds, —O—, —S—, —NR⁵—, —C═C—, and —CR^{a}═CR^{a}—;
R¹⁶ is selected from C₃₋₆cycloalkyl and 3- to 6-membered heterocyclyl; the cycloalkyl or heterocyclyl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄alkynyl, CN, and ═M; wherein, M is selected from O and CR³R⁴; R³ and R⁴ are independently selected from the group consisting of: hydrogen, fluorine, and C₁₋₄ alkyl; and
R, A, B, R¹, R¹¹, R⁵, and R^{a} are as defined above; R¹⁵ and q are as defined above.

In another preferred emboiment, formula (I) is formula (IX),

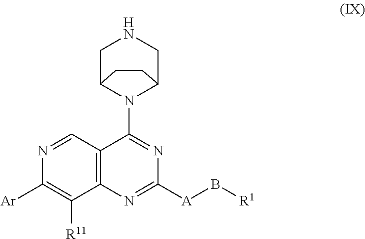

wherein, Ar, A, B, R¹, and R¹¹ are as defined above.

In another preferred embodiment, formula (I) is formula (X),

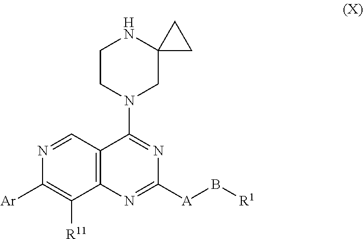

wherein, Ar, A, B, R¹, R¹¹ are as defined above.

In another preferred embodiment, in formula (IX) or formula (X), the structural fragment

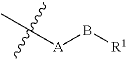

is selected form the group consisting of:

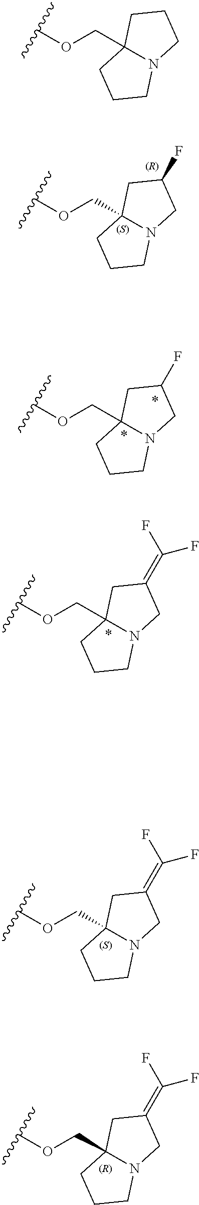

Ar is selected from the group consisting of:

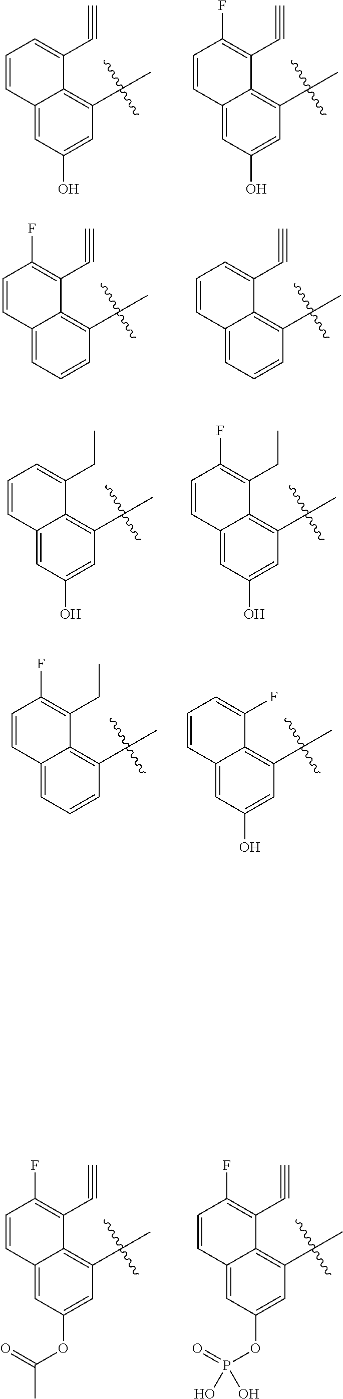

“*” represents a chiral center;
“ [symbol] ” represents the connection site between the structural fragment

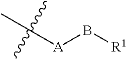

or Ar and other structures in formula (IX) or formula (X).

In another preferred embodiment, formula (I) is formula (XI),

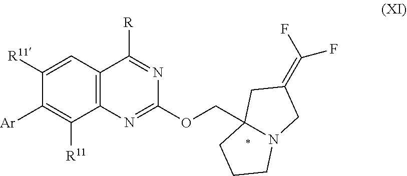

“*” represents a chiral center;
R¹¹ is selected from hydrogen, halogen, and C₁₋₄ alkyl;
R^{11′} is selected from hydrogen, halogen, C₁₋₄ alkyl, C₁₋₄alkoxy, C₁₋₄ haloalkoxy, and CN;
Ar and R are as defined above.

In another preferred embodiment, formula (I) is formula (XII),

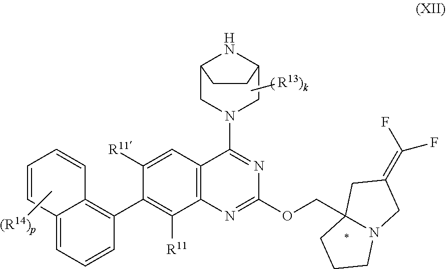

“*” represents a chiral center;
R¹¹ and R^{11′} are as defined above; R¹³, R¹⁴, k, and p are as defined above.

In another preferred embodiment, formula (I) is formula (XIII),

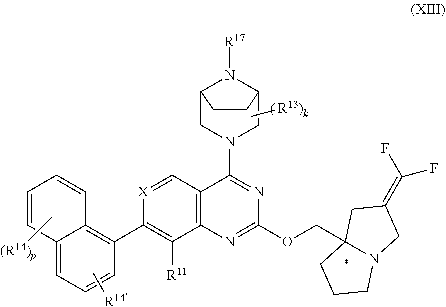

“*” represents a chiral center;
R¹¹ is selected from hydrogen, halogen, and C₁₋₄ alkyl;
X is selected from N and CR^{11′}; wherein, R^{11′} is selected from hydrogen, halogen, C₁₋₄ alkyl, C₁₋₄alkoxy, C₁₋₄ halogenated alkoxy, and CN;
R¹³ is selected from halogen, C₁₋₄ alkyl, ═O, hydroxyl, and CN;
R¹⁴ is selected from halogen, C₁₋₄ alkyl, C₁₋₄haloalkyl, hydroxyl, C₁₋₄alkoxy, C₁₋₄haloalkoxy, C₂₋₄ alkenyl, C₂₋₄haloalkenyl, C₂₋₄alkynyl, C₂₋₄haloalkynyl, C₃₋₆ cycloalkyl, 3 to 6-membered heterocyclyl, C₃₋₆cycloalkyl-O—, 3 to 6-membered heterocyclyl-O—, NR²R², CN, and SR²; wherein, each R² is independently hydrogen or C₁₋₄ alkyl;
R¹⁴ is selected from hydrogen, —OC(O)R^{k}, —O—P(O)(OR^{m})₂, and —O—CH (^{Rn})—O—P(O)(OR^{m})₂; wherein, R^{k} is selected from C₁₋₁₂ alkyl, wherein the alkyl is optionally substituted by one or more groups selected from the group consisting of: hydroxyl, C₁₋₄alkoxy, NR²R², C(O)OH, C(O)OC₁₋₂ alkyl, and CN; R^{m} is selected from hydrogen and C₁₋₄ alkyl; Rⁿ is selected from hydrogen and C₁₋₄ alkyl;
R¹⁷ is selected from hydrogen and —CO(O)—CH(Rⁿ)—O—C(O)—U; wherein, Rⁿ is selected from hydrogen and C₁₋₄ alkyl; U is selected from C₁₋₁₈ alkyl;
k is selected from 0, 1, or 2;
p is selected from 0, 1, 2, 3, or 4.

In another aspect of the present invention, provided is a compound of the structure shown in formula (XIV), or an optical isomer, a pharmaceutically acceptable salt, a prodrug, a deuterated derivative, a hydrate, or a solvate thereof:

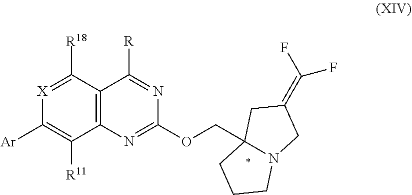

wherein:
“*” represents a chiral center;
R¹¹ is selected from hydrogen, halogen, and C₁₋₄ alkyl;
X is selected from N and CR^{11′}; wherein, R^{11′} is selected from hydrogen, halogen, C₁₋₄ alkyl, C₁₋₄alkoxy, C₁₋₄ haloalkoxy, and CN;
R¹⁸ is selected from halogen, C₁₋₄ alkyl, and C₁₋₄ alkoxy;
Ar and R are as defined above.

In another preferred example, the compound of formula (I) is selected from the group consisting of:

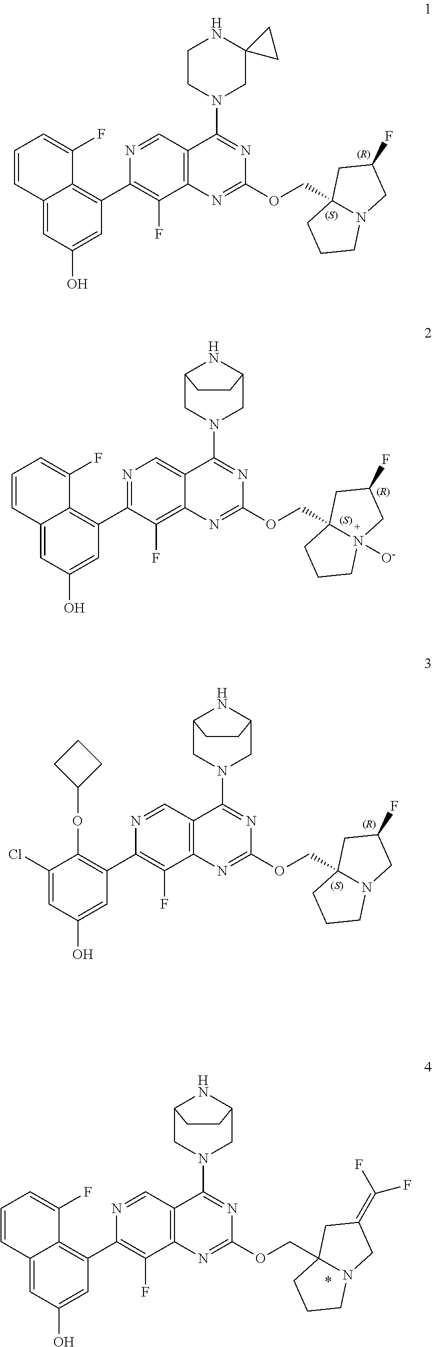

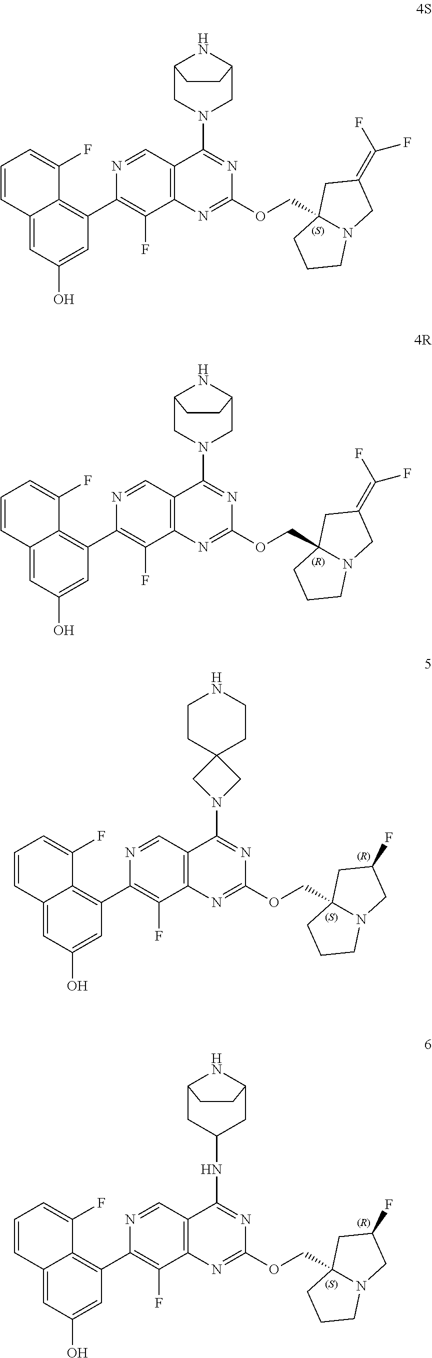

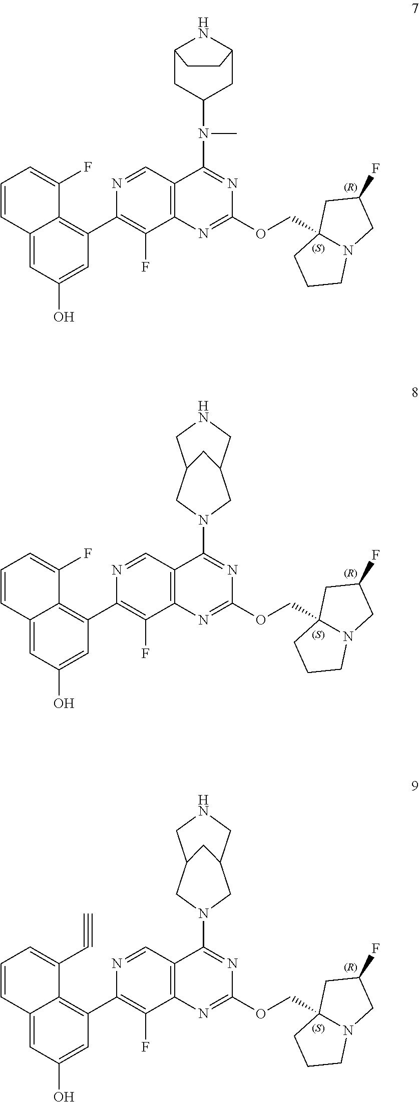

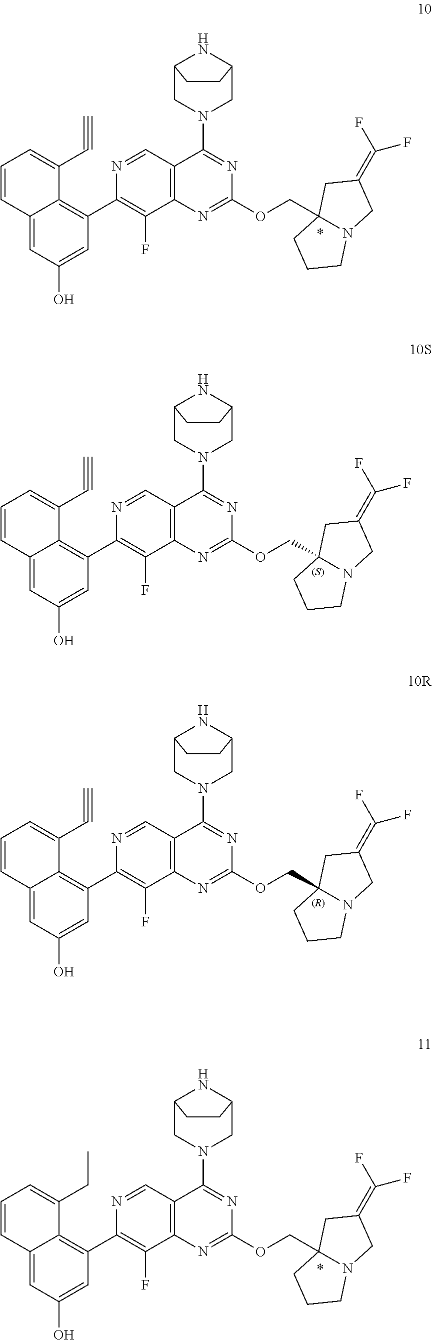

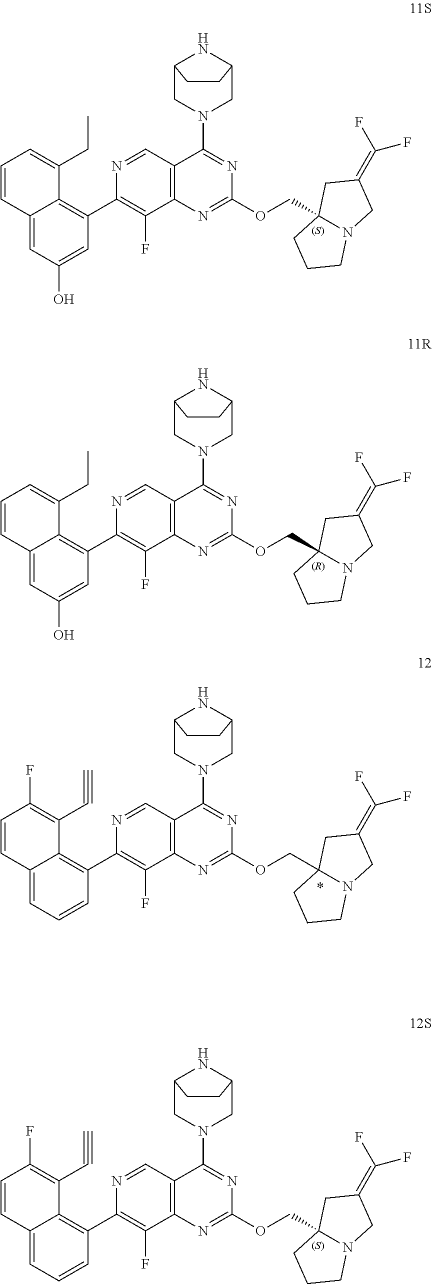

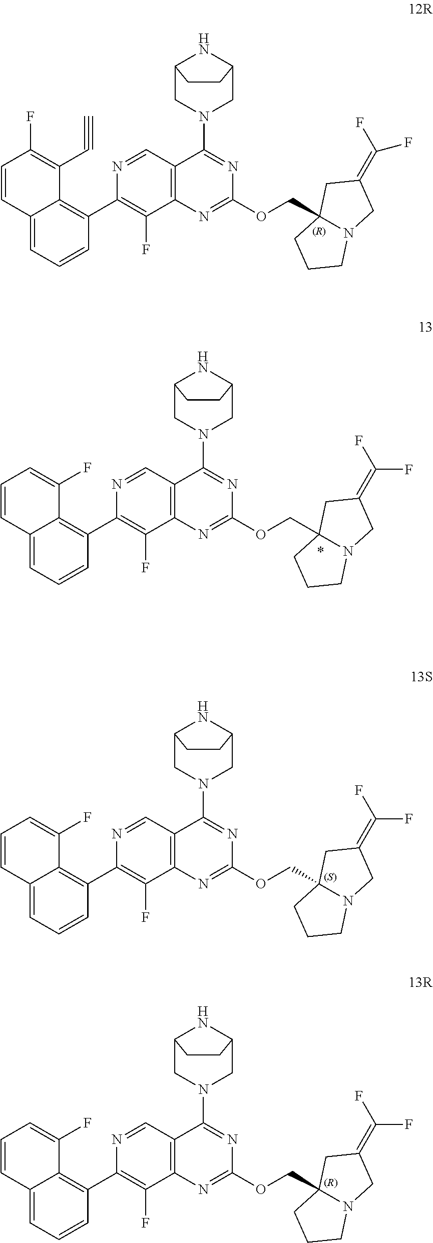

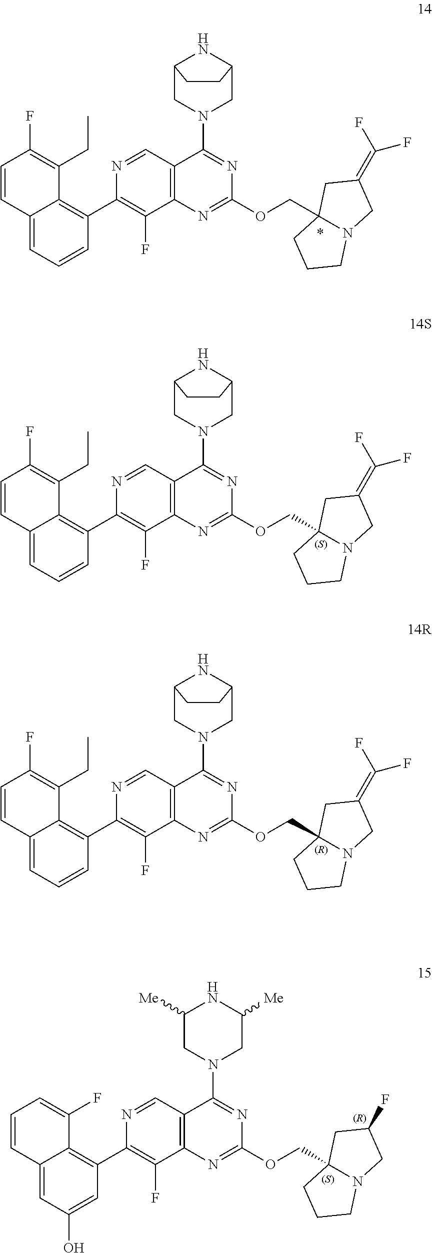

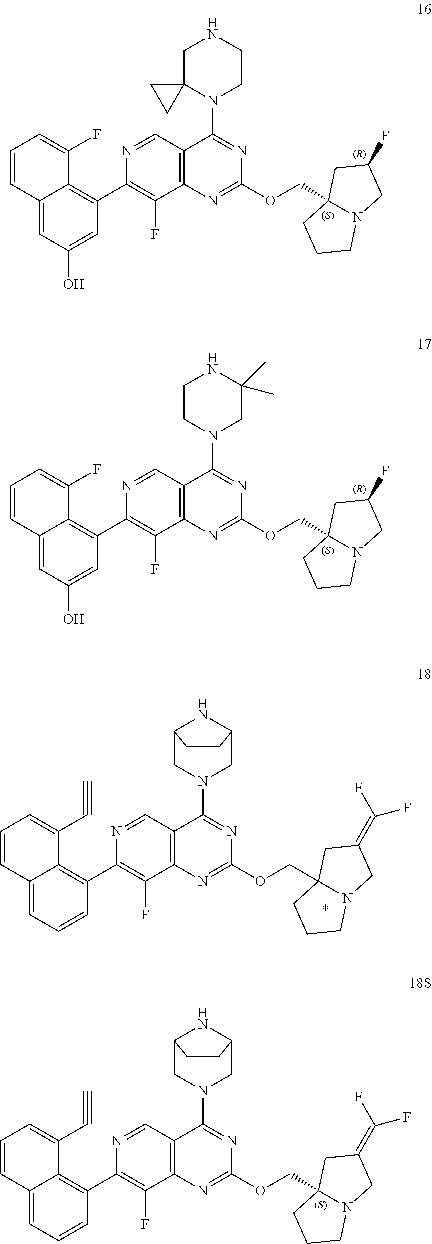

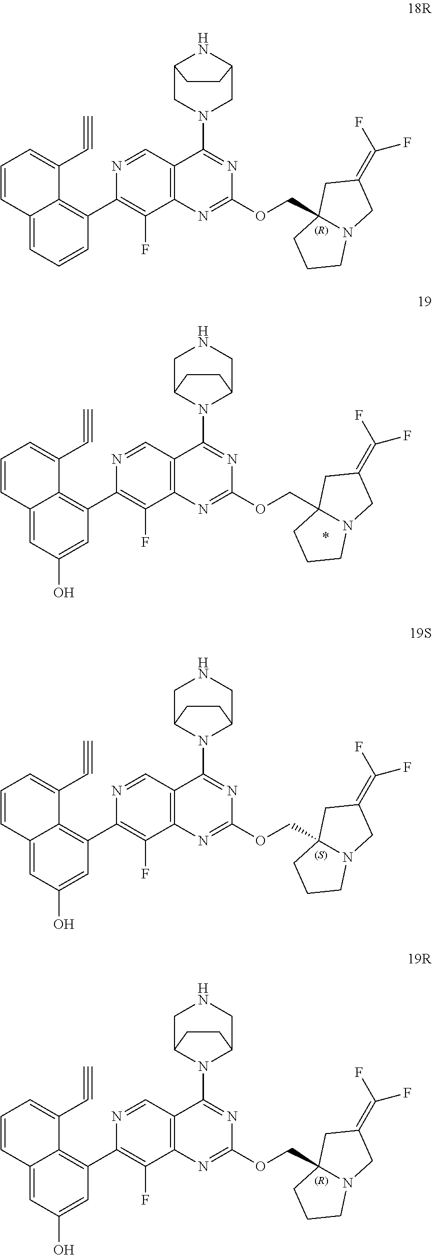

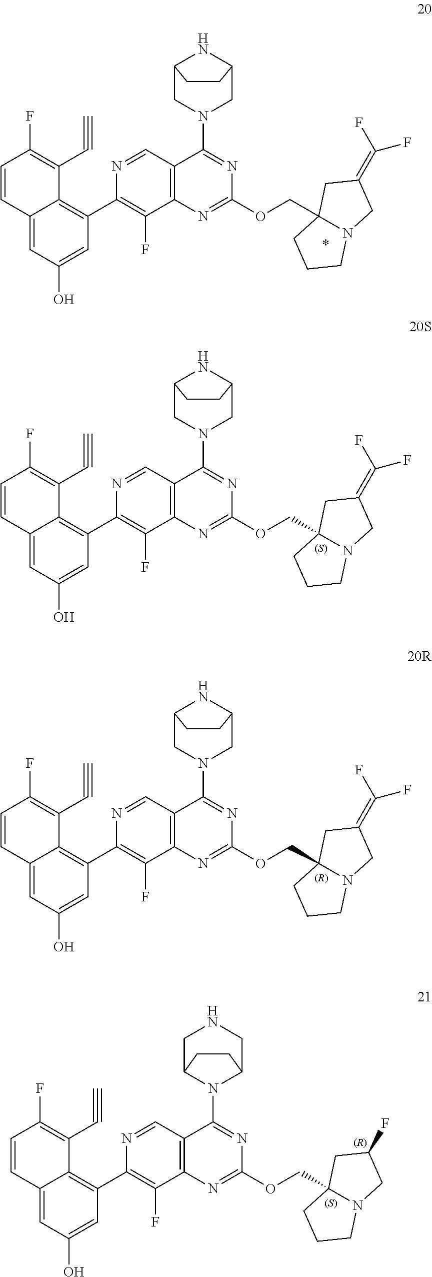

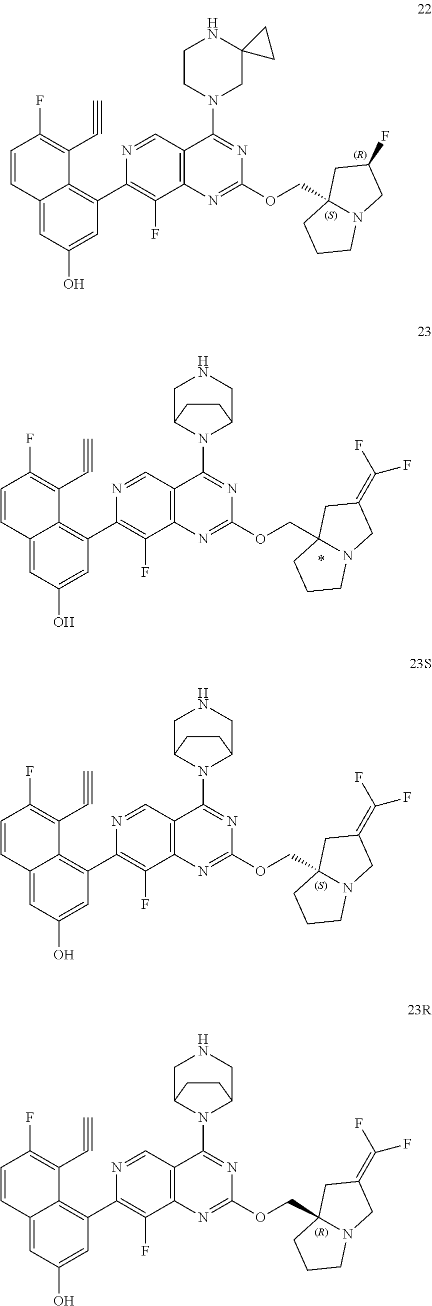

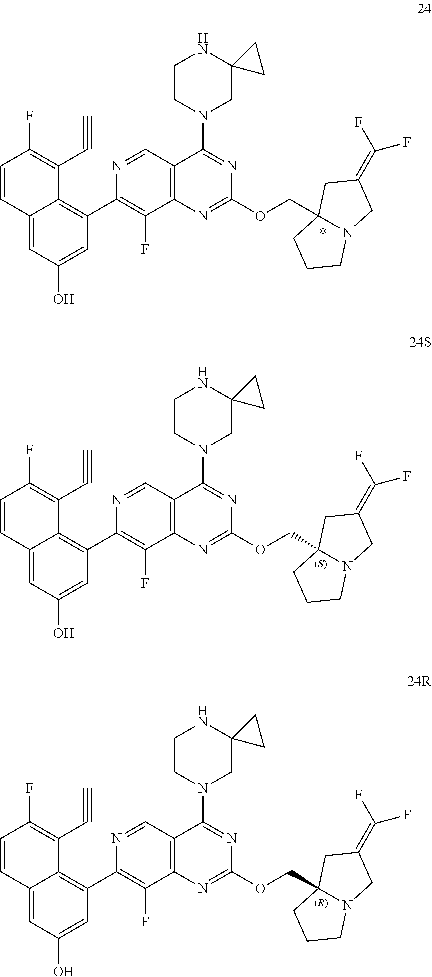

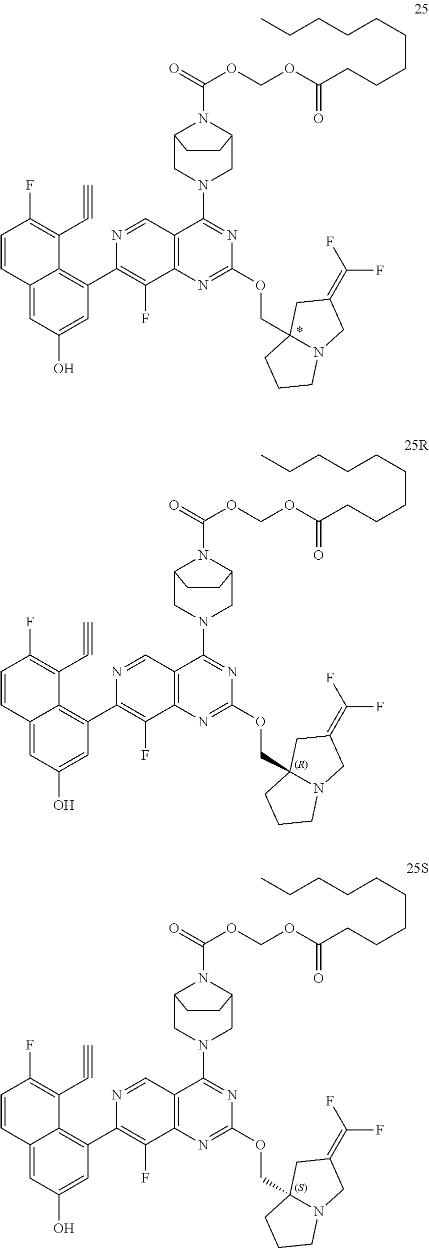

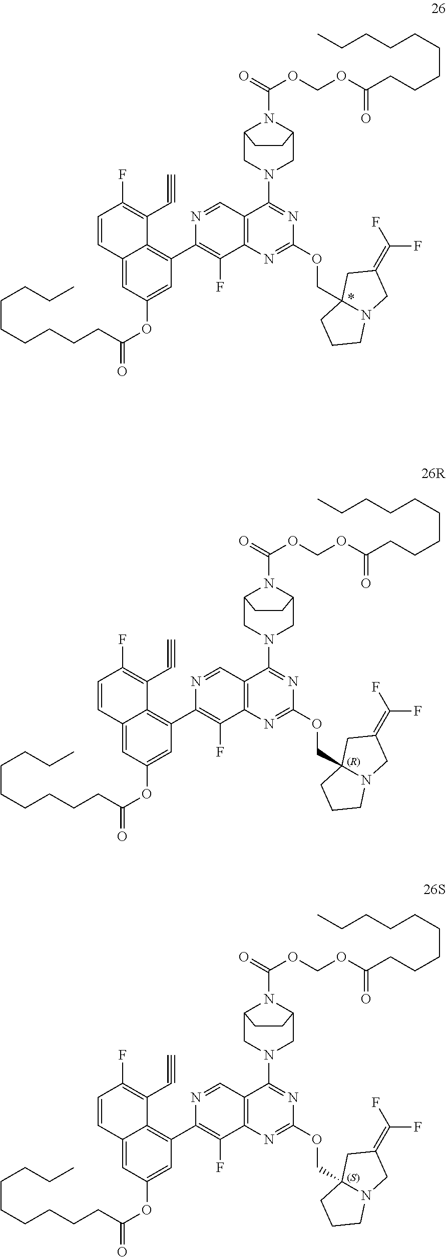

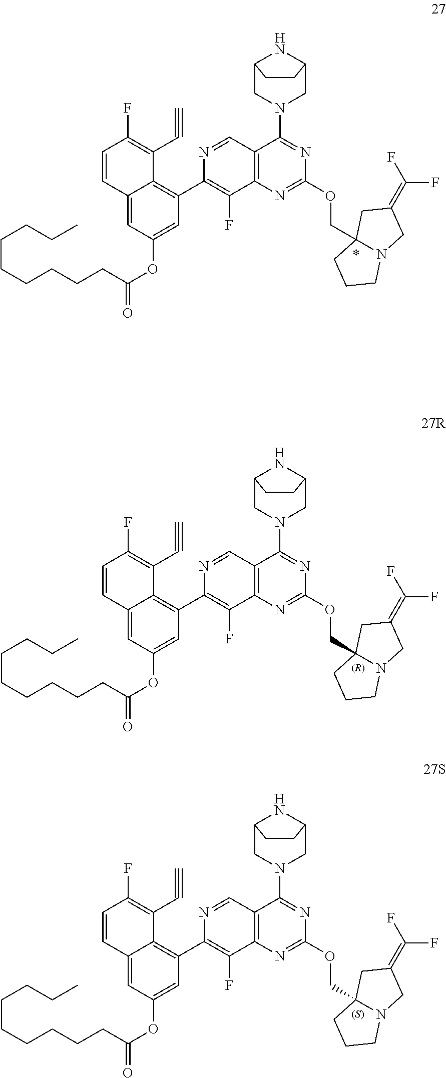

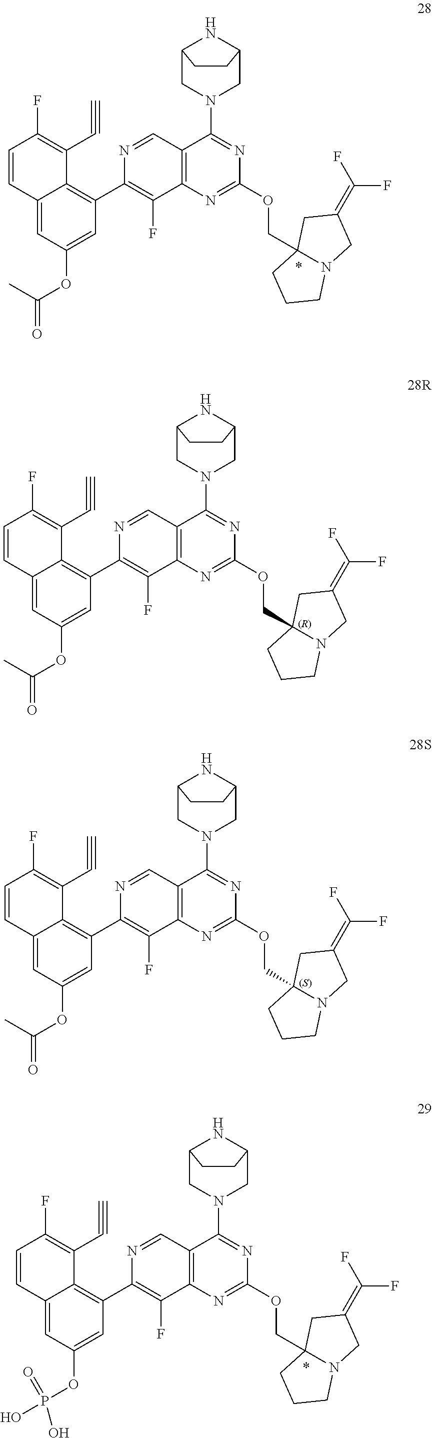

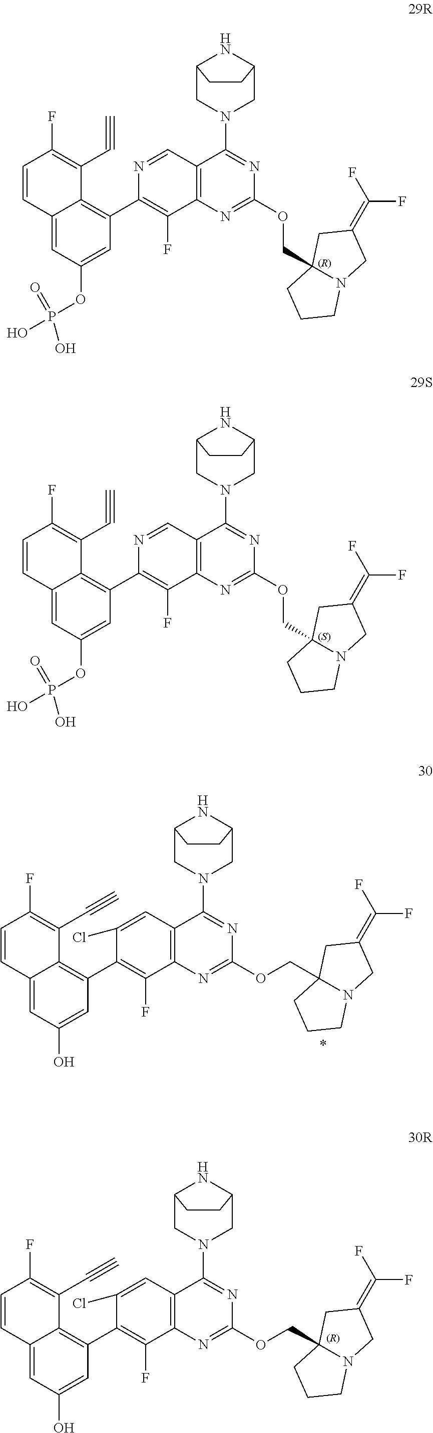

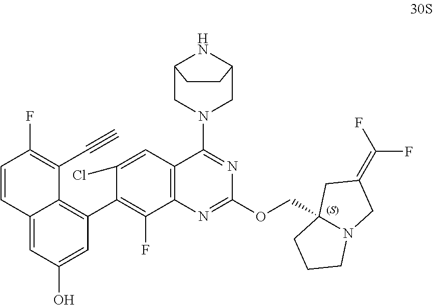

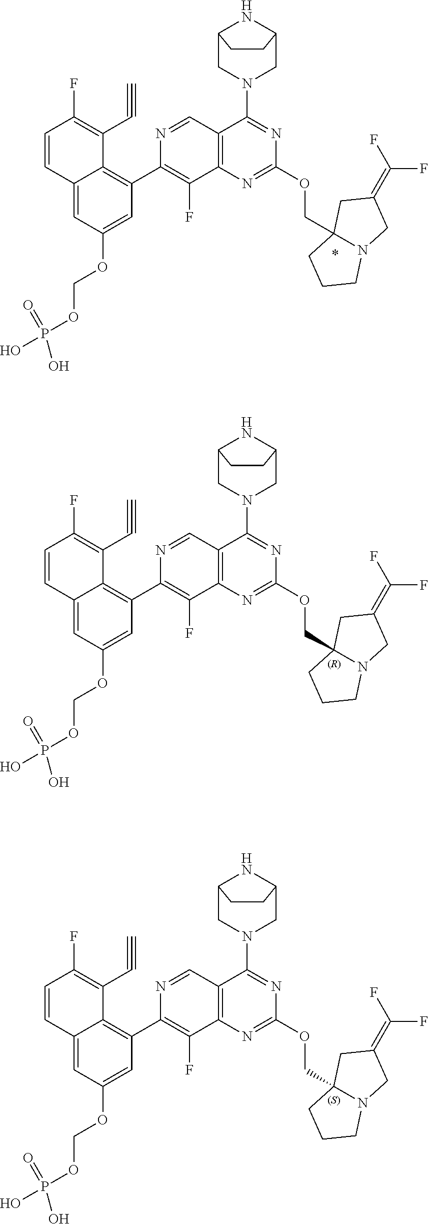

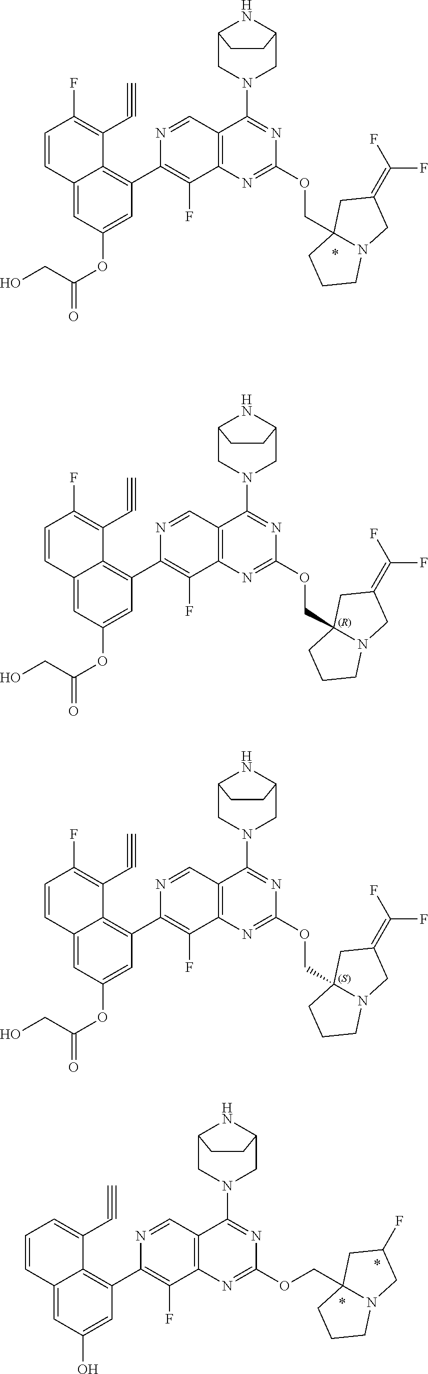

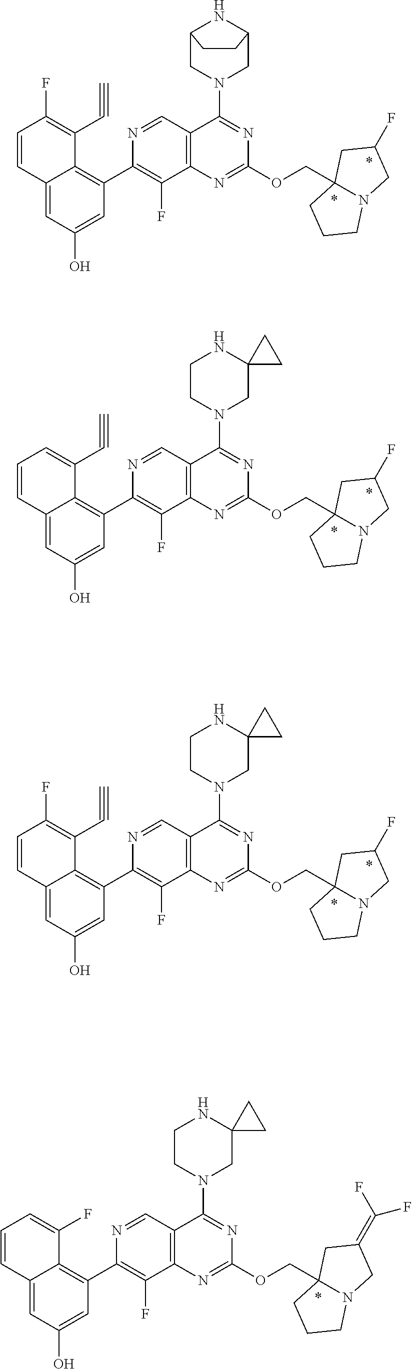 [SMILES: C#Cc1c(F)ccc2cc(O)cc(-c3ncc4c(N5CC6CCC(C5)N6)nc(OCC56CCCN5CC(F)C6)nc4c3F)c12 ; C#Cc1cccc2cc(O)cc(-c3ncc4c(N5CCNC6(CC6)C5)nc(OCC56CCCN5CC(F)C6)nc4c3F)c12 ; C#Cc1c(F)ccc2cc(O)cc(-c3ncc4c(N5CCNC6(CC6)C5)nc(OCC56CCCN5CC(F)C6)nc4c3F)c12 ; Oc1cc(-c2ncc3c(N4CCNC5(CC5)C4)nc(OCC45CCCN4CC(=C(F)F)C5)nc3c2F)c2c(F)cccc2c1]

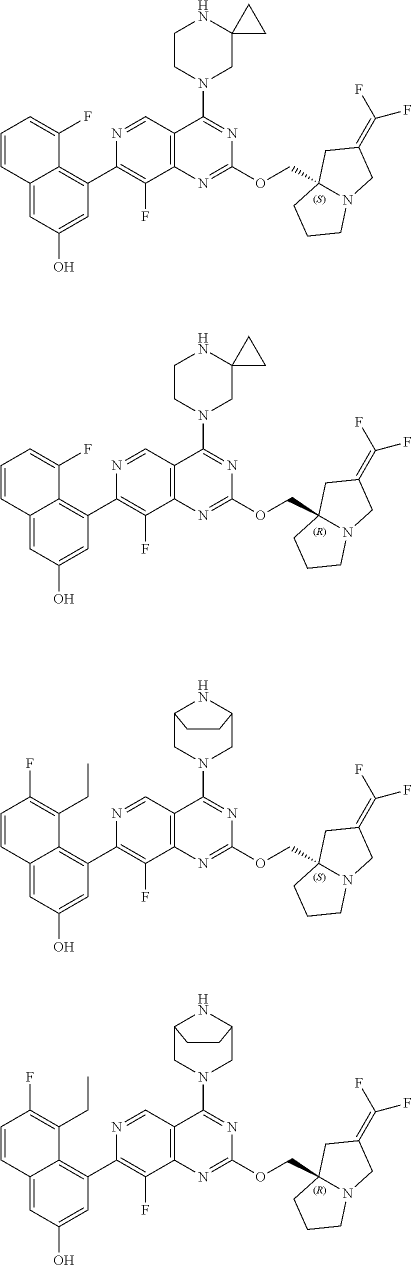

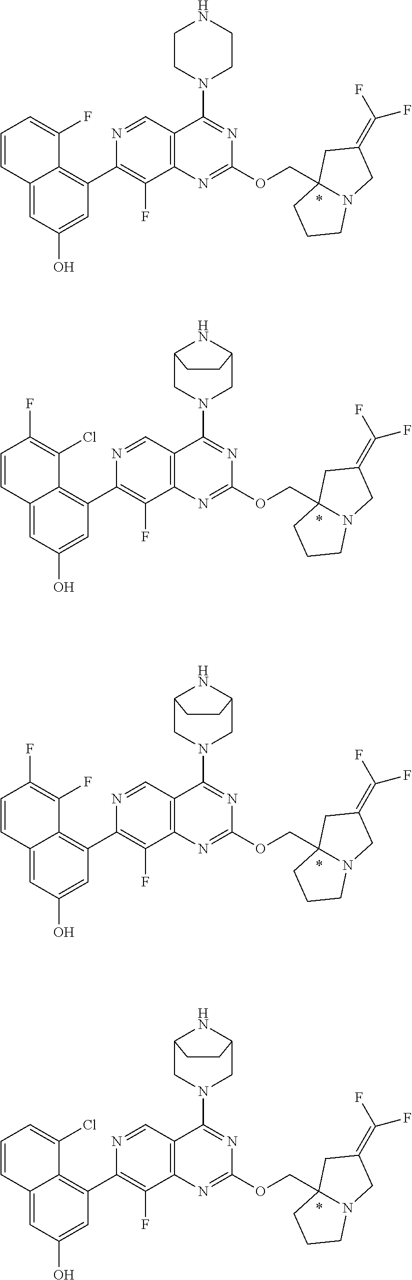 [SMILES: Oc1cc(-c2ncc3c(N4CCNCC4)nc(OCC45CCCN4CC(=C(F)F)C5)nc3c2F)c2c(F)cccc2c1 ; Oc1cc(-c2ncc3c(N4CC5CCC(C4)N5)nc(OCC45CCCN4CC(=C(F)F)C5)nc3c2F)c2c(Cl)c(F)ccc2c1 ; Oc1cc(-c2ncc3c(N4CC5CCC(C4)N5)nc(OCC45CCCN4CC(=C(F)F)C5)nc3c2F)c2c(F)c(F)ccc2c1 ; Oc1cc(-c2ncc3c(N4CC5CCC(C4)N5)nc(OCC45CCCN4CC(=C(F)F)C5)nc3c2F)c2c(Cl)cccc2c1]

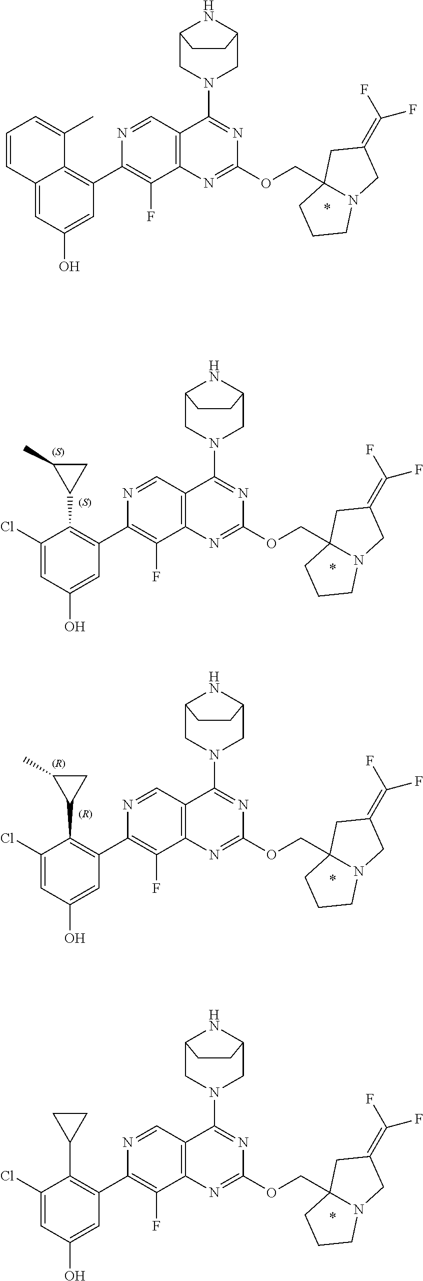

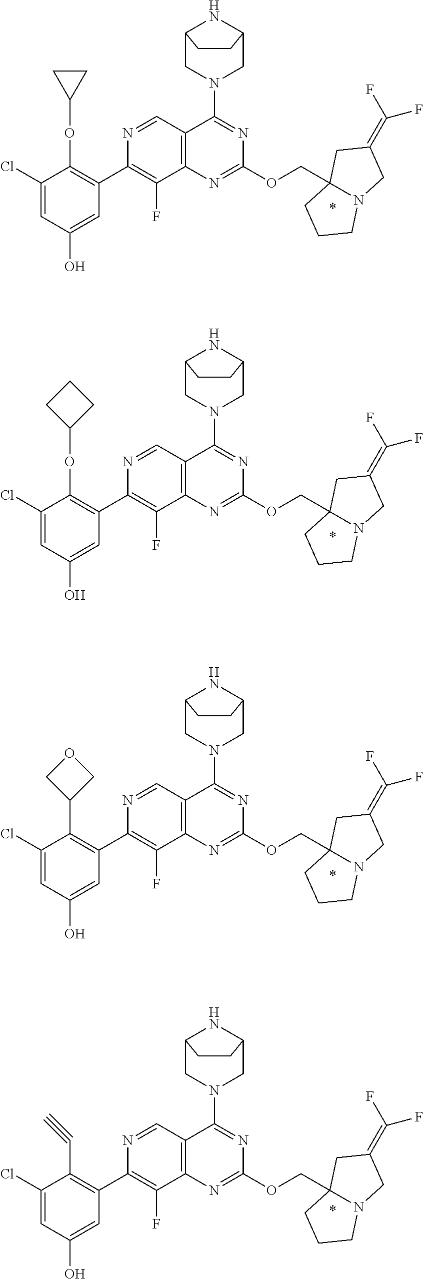 [SMILES: Oc1cc(Cl)c(OC2CC2)c(-c2ncc3c(N4CC5CCC(C4)N5)nc(OCC45CCCN4CC(=C(F)F)C5)nc3c2F)c1 ; Oc1cc(Cl)c(OC2CCC2)c(-c2ncc3c(N4CC5CCC(C4)N5)nc(OCC45CCCN4CC(=C(F)F)C5)nc3c2F)c1 ; Oc1cc(Cl)c(C2COC2)c(-c2ncc3c(N4CC5CCC(C4)N5)nc(OCC45CCCN4CC(=C(F)F)C5)nc3c2F)c1 ; C#Cc1c(Cl)cc(O)cc1-c1ncc2c(N3CC4CCC(C3)N4)nc(OCC34CCCN3CC(=C(F)F)C4)nc2c1F]

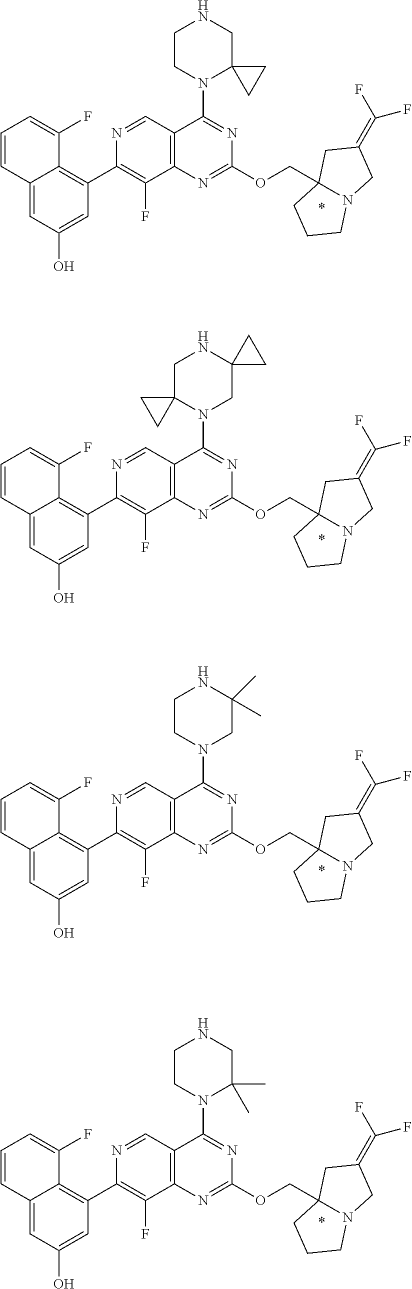 [SMILES: Oc1cc(-c2ncc3c(N4CCNCC45CC5)nc(OCC45CCCN4CC(=C(F)F)C5)nc3c2F)c2c(F)cccc2c1 ; Oc1cc(-c2ncc3c(N4CC5(CC5)NCC45CC5)nc(OCC45CCCN4CC(=C(F)F)C5)nc3c2F)c2c(F)cccc2c1 ; CC1(C)CN(c2nc(OCC34CCCN3CC(=C(F)F)C4)nc3c(F)c(-c4cc(O)cc5cccc(F)c45)ncc23)CCN1 ; CC1(C)CNCCN1c1nc(OCC23CCCN2CC(=C(F)F)C3)nc2c(F)c(-c3cc(O)cc4cccc(F)c34)ncc12]

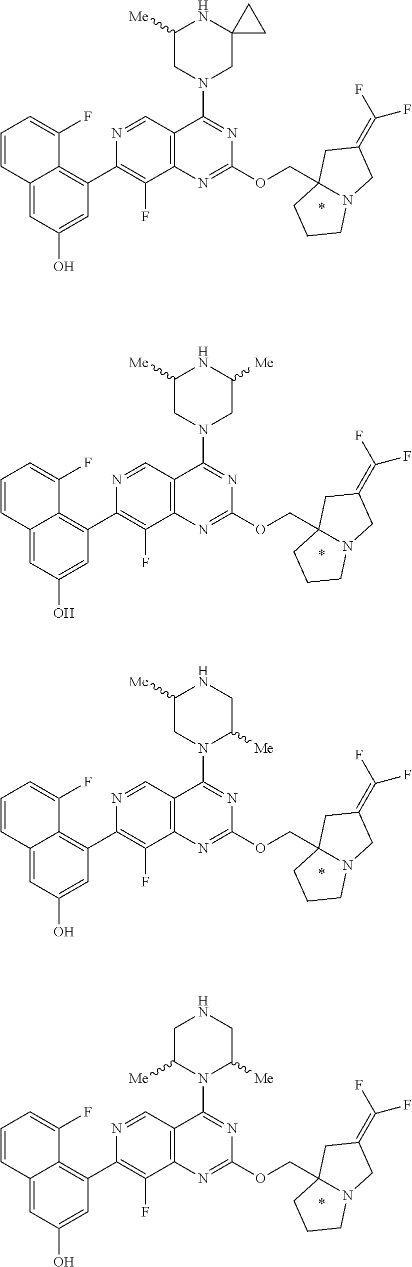

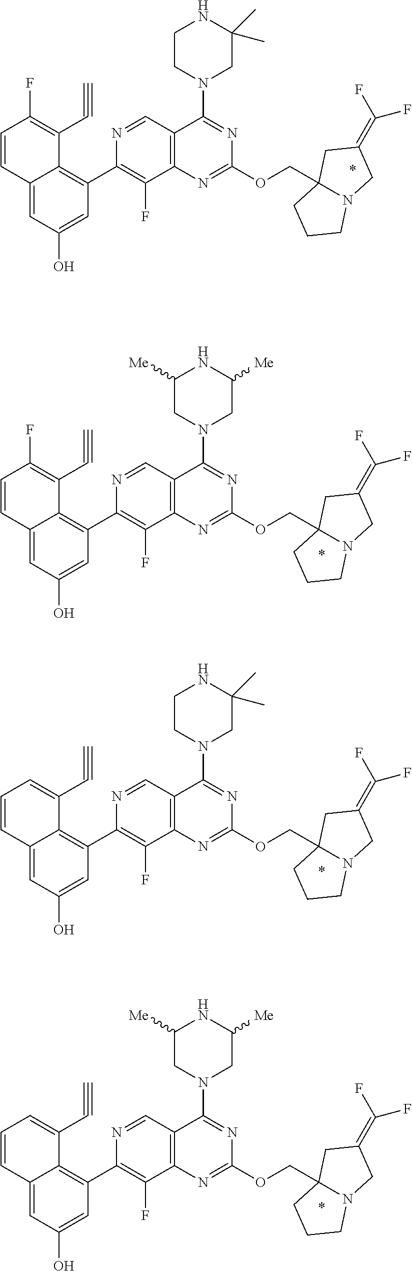

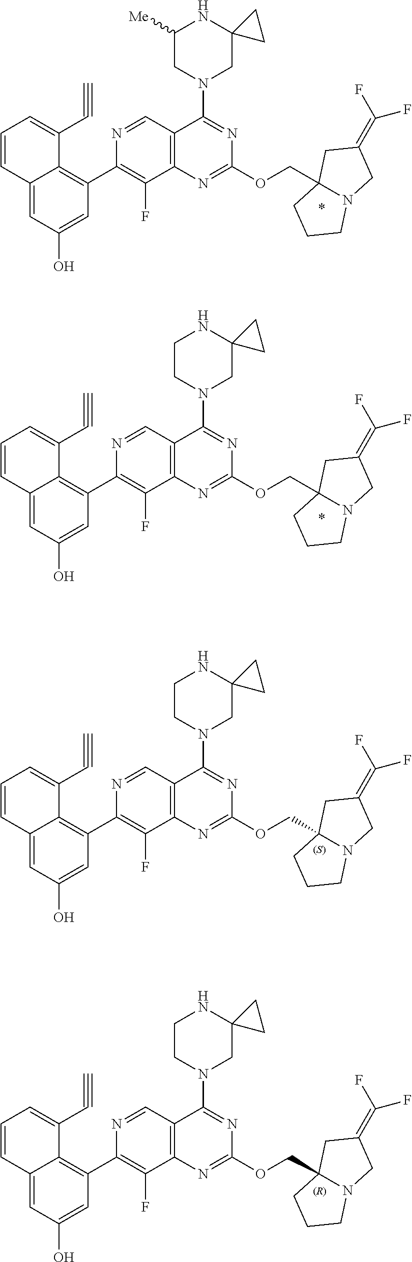

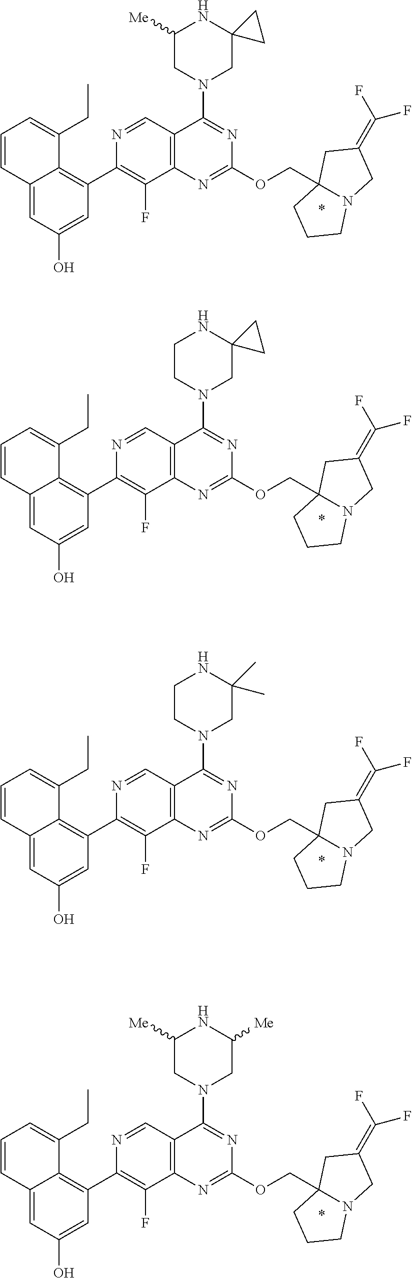

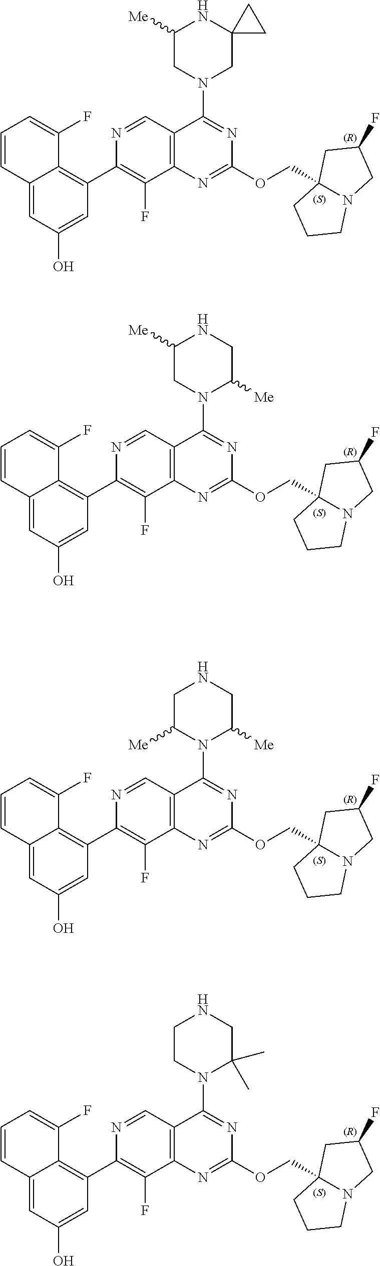

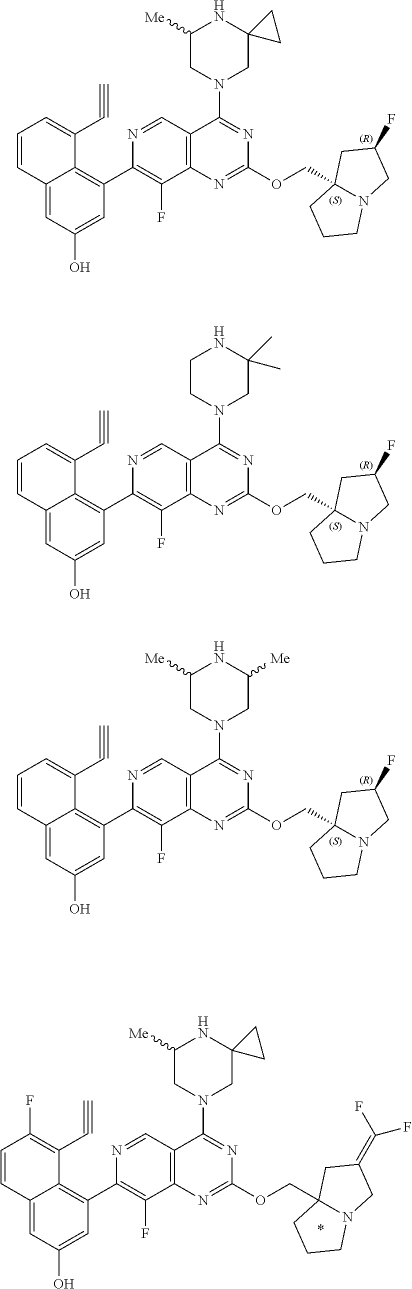

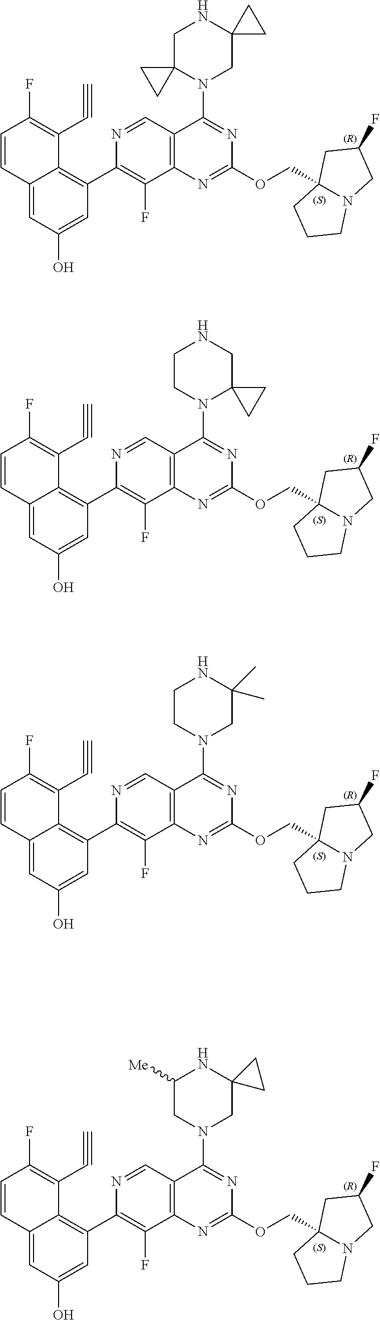

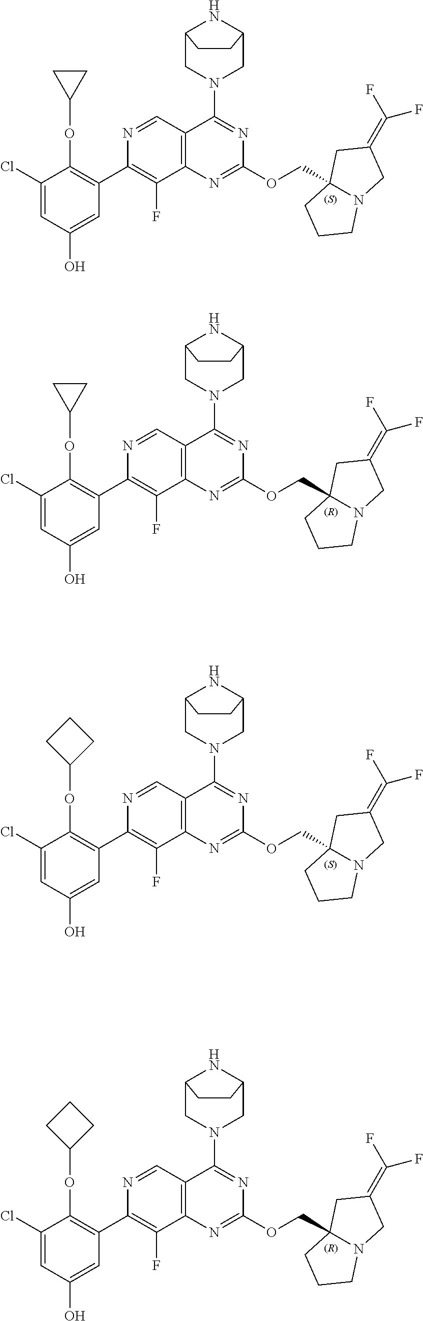

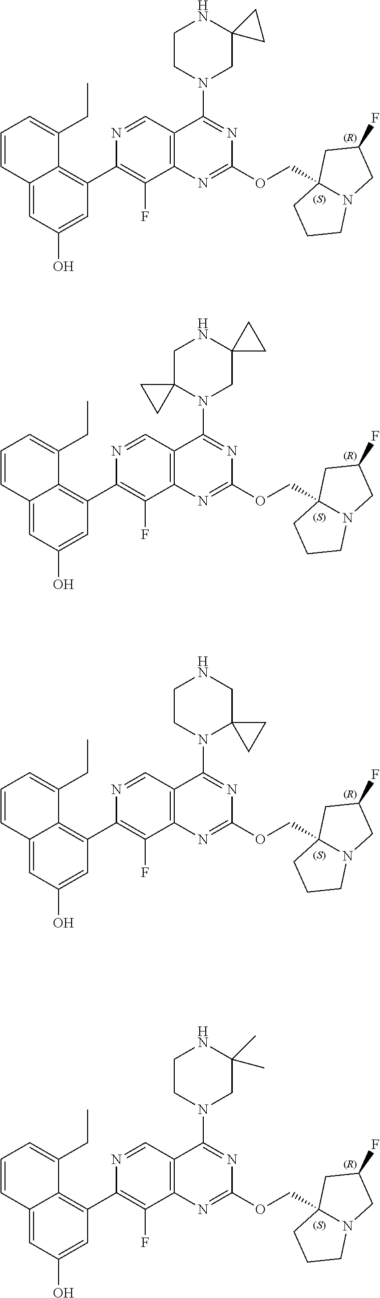

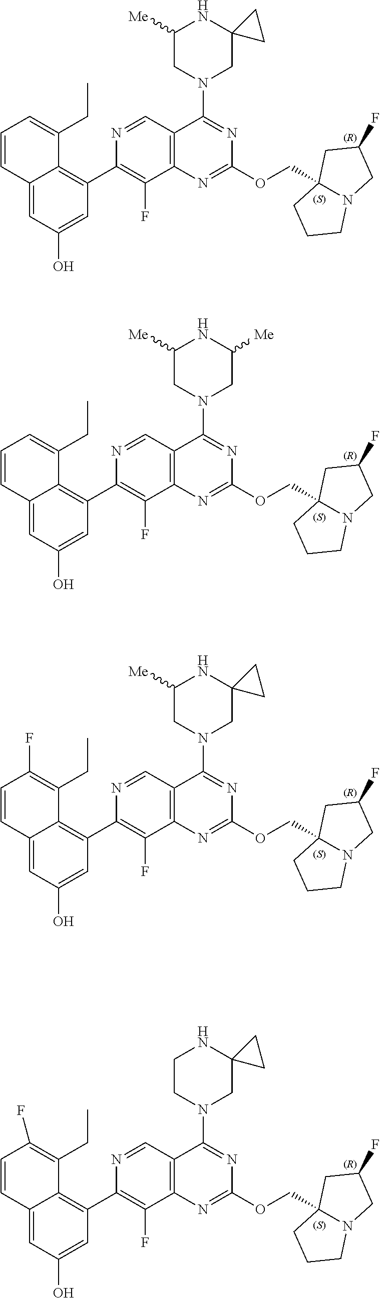

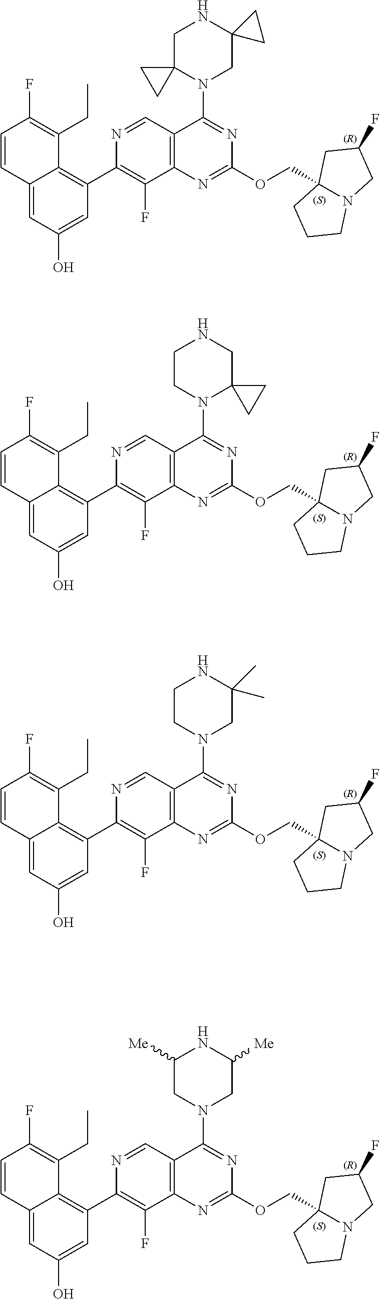

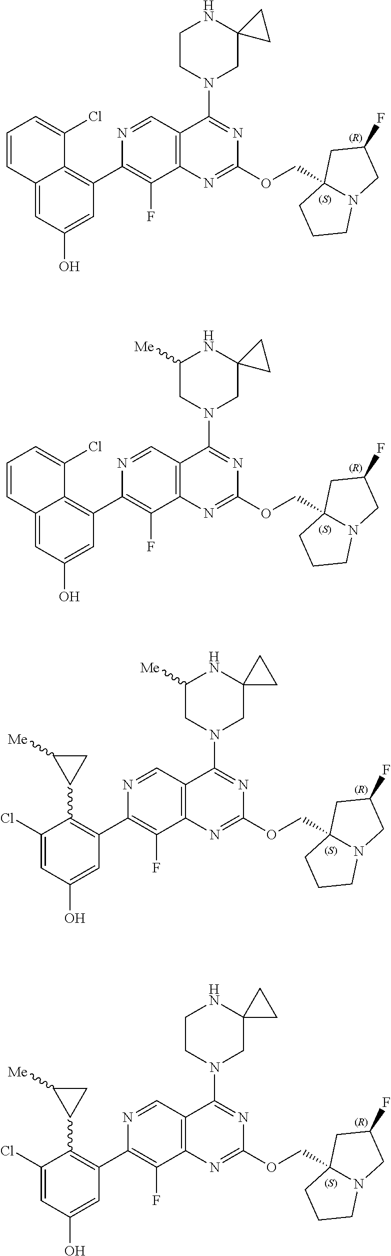

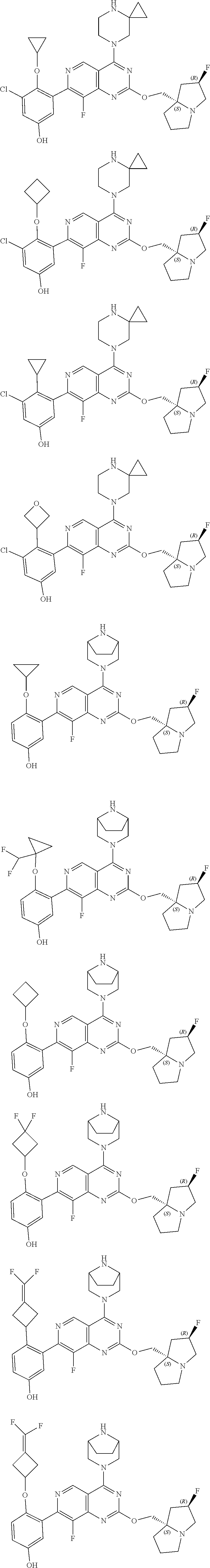

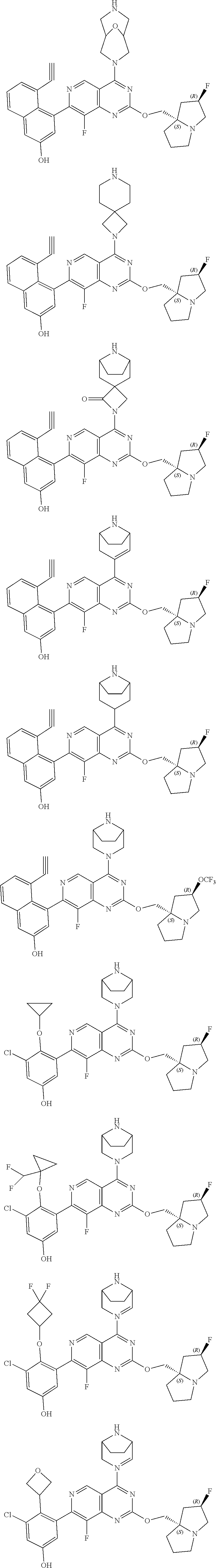

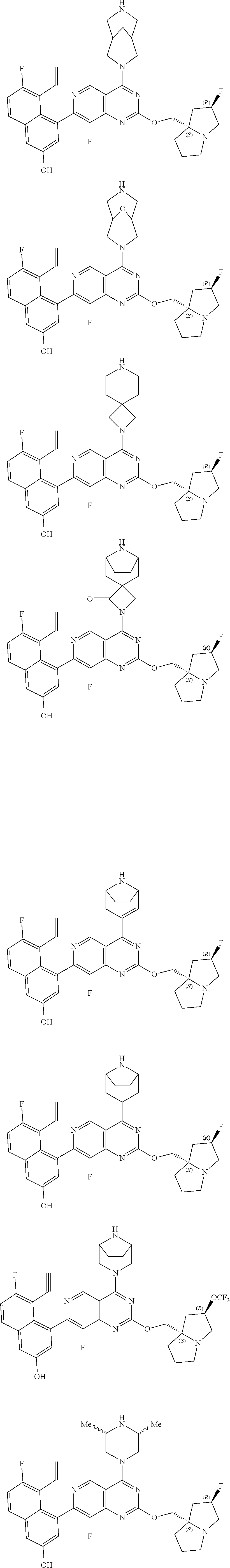

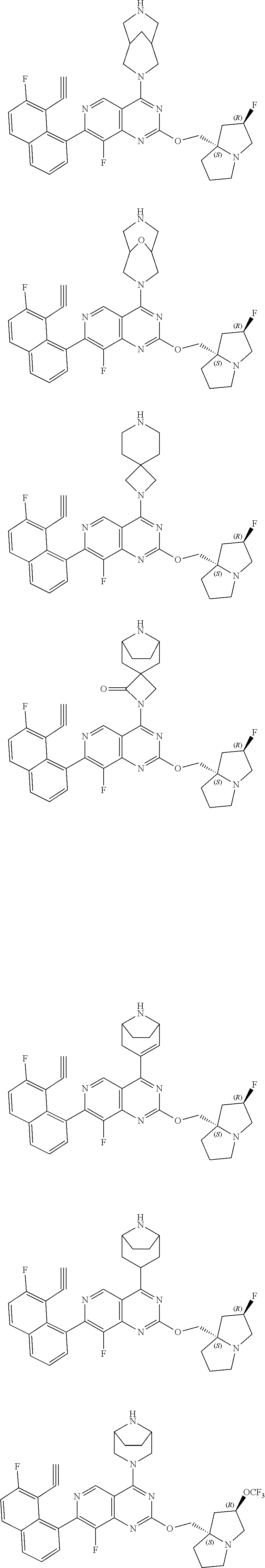

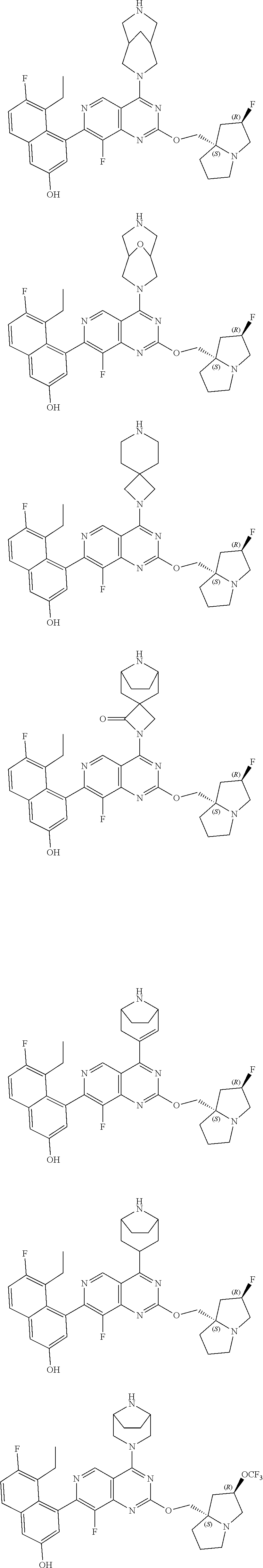

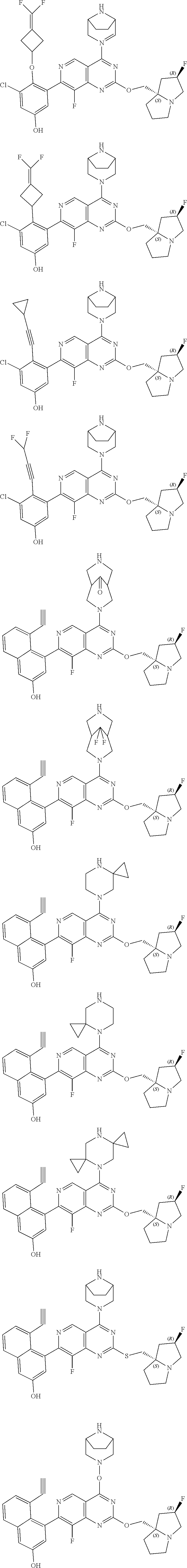

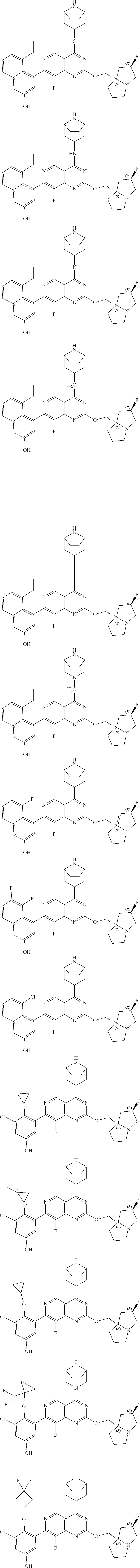

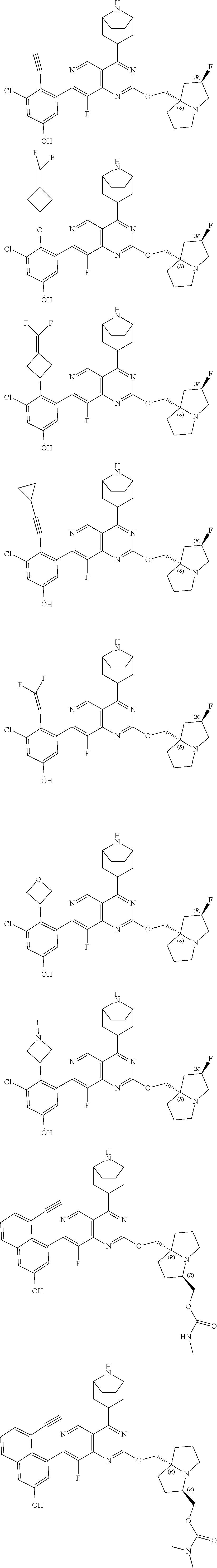

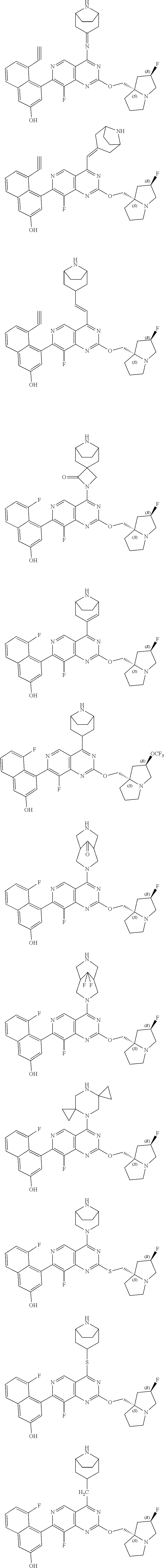

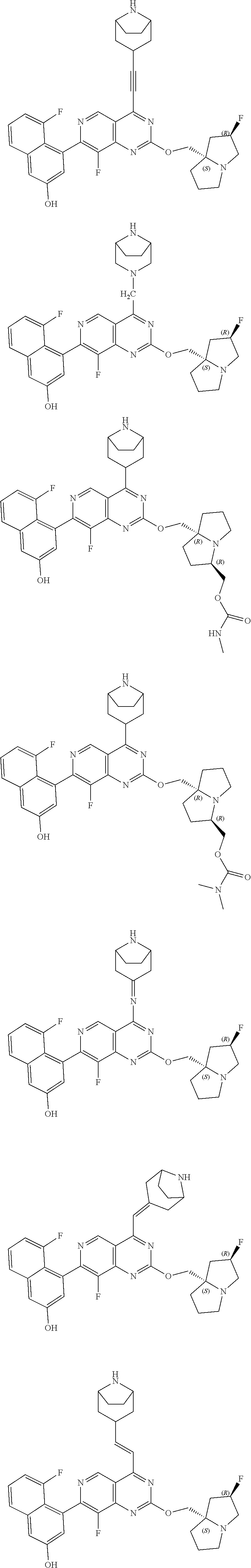

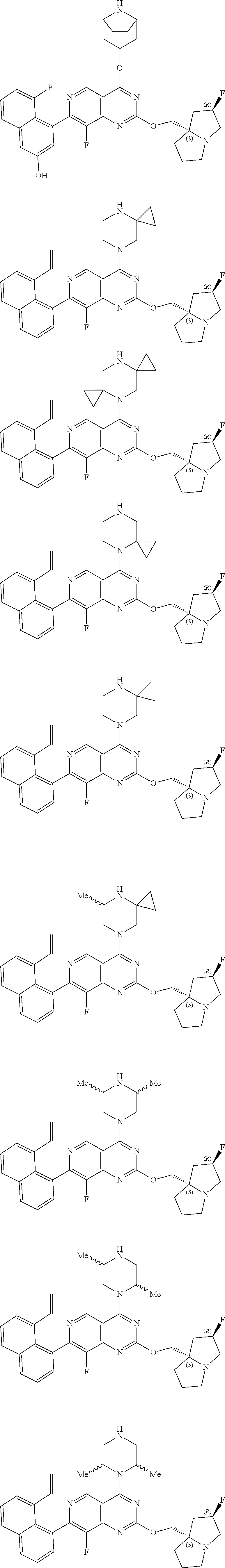

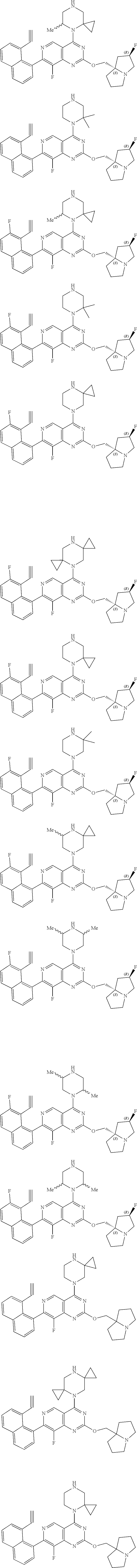

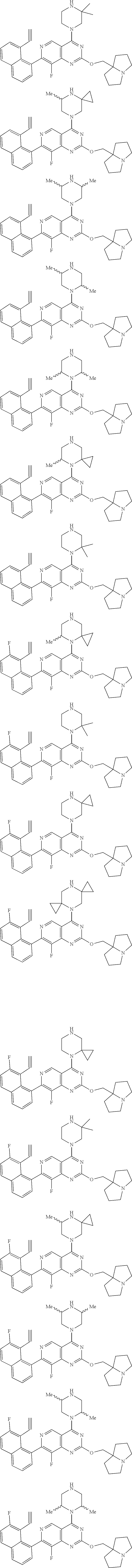

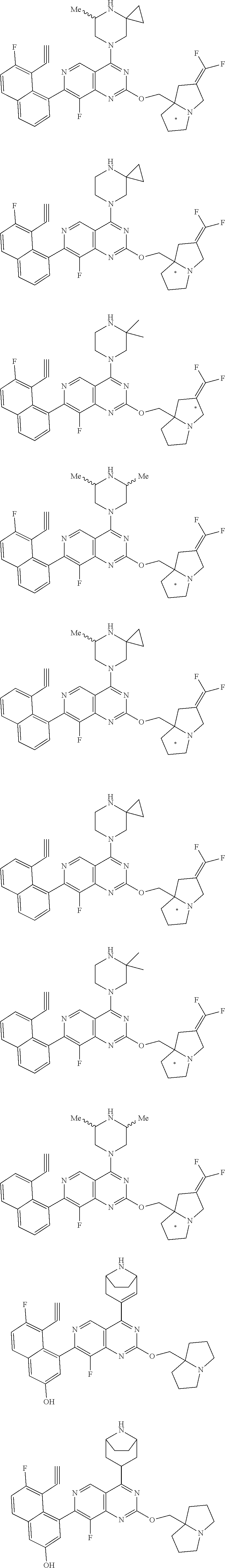

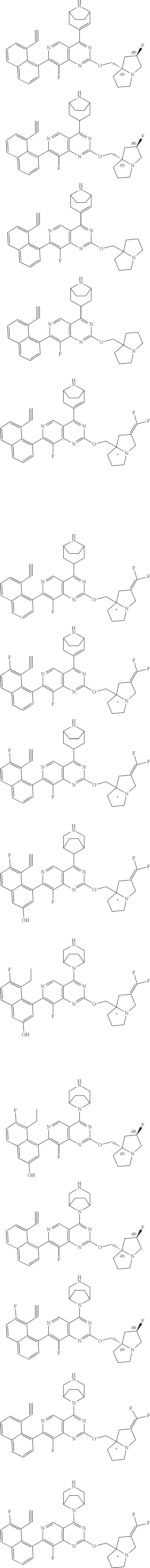

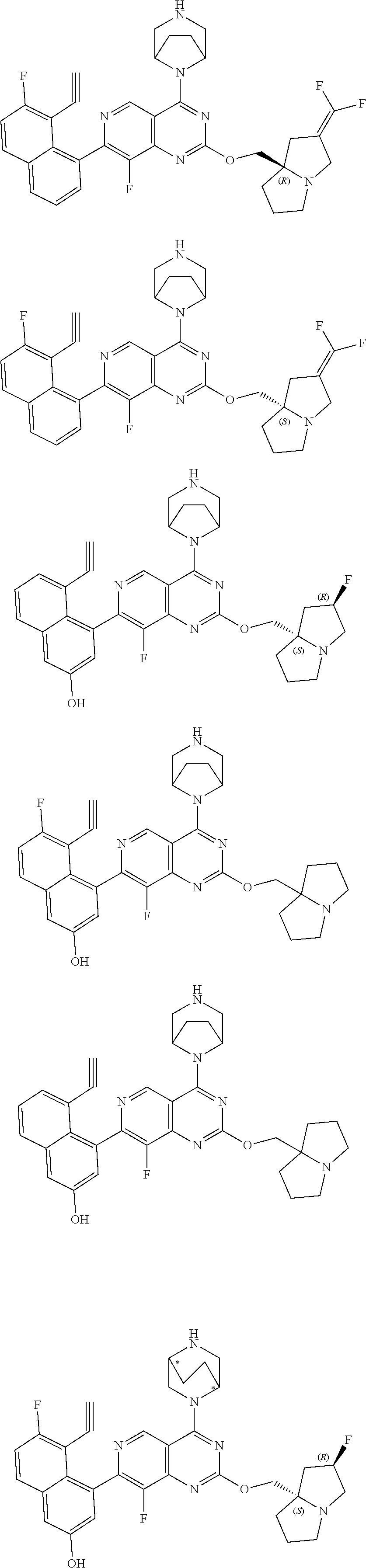

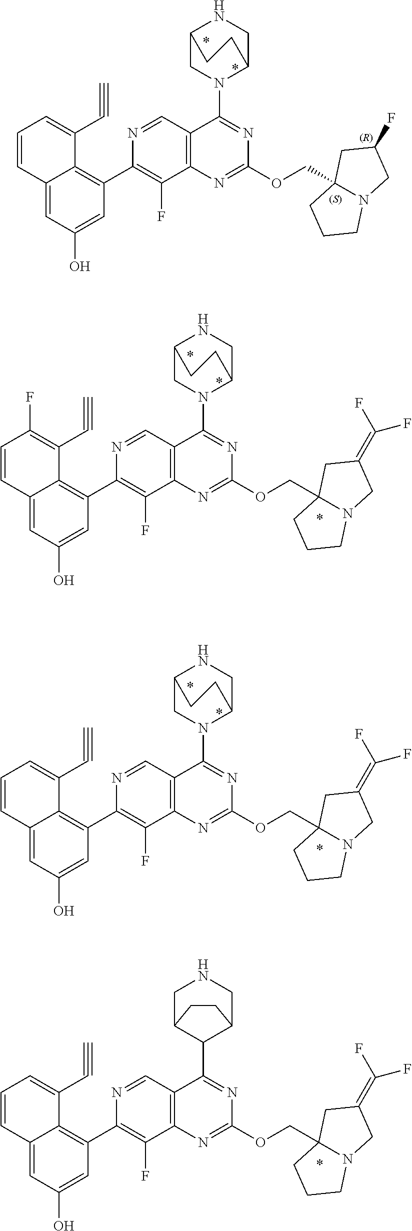

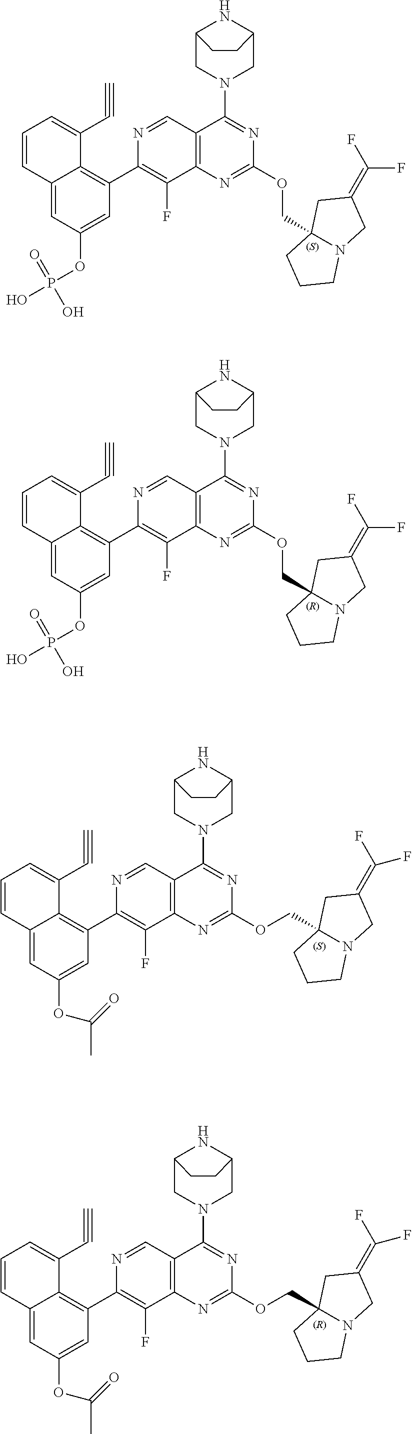

in the above structural formula, “*” represents a chiral center, which can be optionally in the R or S configuration, or a mixture of R and S configurations; the carbon atoms connected by the bond “ [symbol] ” can be optionally in R or S configuration, or optionally in the cis- or trans- configuration.

In the second aspect of the present invention, provided is a pharmaceutical composition, wherein, comprising the compound according to the first aspect of the present invention, or an optical isomer, a pharmaceutically acceptable salt, a prodrug, a deuterated derivative, a hydrate, or a solvate thereof, and pharmaceutically acceptable carriers.

In the third aspect of the present invention, provided is a use of the compound according to the first aspect of the present invention, or an optical isomer, a pharmaceutically acceptable salt, a prodrug, a deuterated derivative, a hydrate, or a solvate thereof, in the preparation of a pharmaceutical composition for treating diseases, disorders or conditions related to KRAS G12D activity or expression level.

In another preferred embodiment, the disease, disorder or condition is selected from the solid tumor and blood tumor consisting of: pancreatic cancer, non-small cell lung cancer, small cell lung cancer, lung adenocarcinoma, lung squamous carcinoma, colon cancer, colorectal cancer, thyroid cancer, embryonal rhabdomyosarcoma, skin granular cell tumor, melanoma, liver cancer, rectal cancer, bladder cancer, throat cancer, breast cancer, prostate cancer, glioma, ovarian cancer, head and neck squamous cell cancer, cervical cancer, esophageal cancer, kidney cancer, skin cancer, lymphoma, stomach cancer, acute myeloid leukemia, myelofibrosis, B-cell lymphoma, monocytic leukemia, polycythemia megalosplenica, eosinophilic leukocytosis syndrome, myeloma, etc.

### DETAILED DESCRIPTION OF THE INVENTION

After long-term and intensive research, the present inventors have unexpectedly discovered a class of structurally novel KRAS G12D inhibitors, as well as preparation methods therefor and applications thereof. The compound of the present invention can be applied to treat various diseases related to the activity of the small GTP enzyme. On this basis, the present invention is completed.

### Terms

Unless otherwise specified, the term “or” mentioned in this article has the same meaning as “and/or” (referring to “or” and “and”).

Unless otherwise specified, among all compounds of the present invention, each chiral carbon atom (chiral center) can be optionally in the R configuration or S configuration, or a mixture of R configuration and S configuration.

As used herein, when used alone or as a part of other substituents, the term “alkyl” refers to a straight (i.e. unbranched) or branched chain containing only carbon atoms saturated hydrocarbon group, or a group with a combination of straight and branched chain. When alkyl is preceded by a limitation on carbon atom number (e.g., C₁₋₁₀), it means that said alkyl contains 1-10 carbon atoms. For example, C₁₋₈ alkyl refers to an alkyl group containing 1-8 carbon atoms, including methyl, ethyl, propyl, isopropyl, butyl, isobutyl, sec-butyl, tert-butyl, or similar groups.

As used herein, the term “alkenyl”, when used alone or as part of other substituents, refers to a straight or branched carbon chain group having at least one carbon-carbon double bond. Alkenyl may be substituted or unsubstituted. When alkenyl is preceded by a limitation on carbon atom number (e.g., C₂₋₈), it means that said alkenyl contains 2-8 carbon atoms. For example, C₂₋₈ alkenyl refers to an alkenyl group containing 2-8 carbon atoms, including vinyl, propenyl, 1,2-butenyl, 2,3-butenyl, butadienyl, or the like.

As used herein, the term “alkynyl”, when used alone or as part of other substituents, refers to an aliphatic hydrocarbon group with at least one triple bond. Alkynyl can be linear or branched, or a combination thereof. When alkynyl is preceded by a limitation on carbon atom number (e.g., C₂₋₈alkynyl), it means that said alkynyl contains 2-8 carbon atoms. For example, the term “C₂₋₈alkynyl” refers to straight or branched alkynyl groups with 2-8 carbon atoms, including ethynyl, propynyl, isopropynyl, butynyl, isobutynyl, sec-butynyl, tertbutynyl, or similar groups.

As used herein, when used alone or as part of other substituents, the term “cycloalkyl” refers to a saturated or partial saturated monocyclic, bicyclic or polycyclic (fused ring, bridge ring, or spiral ring) ring system. When a certain cycloalkyl is preceded by a limitation on carbon atom number (e.g., C₃-10), it means that said cycloalkyl contains 3-10 carbon atoms. In some preferred embodiments, the term “C₃₋₈cycloalkyl” refers to a saturated or partially unsaturated monocyclic or bicyclic alkyl with 3-8 carbon atoms, including cyclopropyl, cyclobutyl, cyclopentyl, cycloheptyl, or similar groups. The term “spirocycloalky” refers to a bicyclic or polycyclic group that shares one carbon atom (referred to as spiro atom) between a single ring, which may contains one or more double bonds, but none of the rings has a fully conjugated 7L electron system. The term “fused cycloalkyl” refers to an all-carbon bicyclic or polycyclic group in which each ring in the system shares an adjoining pair of carbon atoms with the other rings in the system, wherein one or more of the rings may contain one or more double bonds, but none of the rings has a fully conjugated 7L electron system. “Bridged cycloalkyl” means an all-carbon polycyclic group in which any two rings share two carbon atoms that are not directly connected, and these may contain one or more double bonds, but none of the rings has a fully conjugated 7r-electron system. The atoms contained in cycloalkyl are all carbon atoms. The following are some examples of cycloalkyl, but the present invention is not limited to the following cycloalkyl groups:

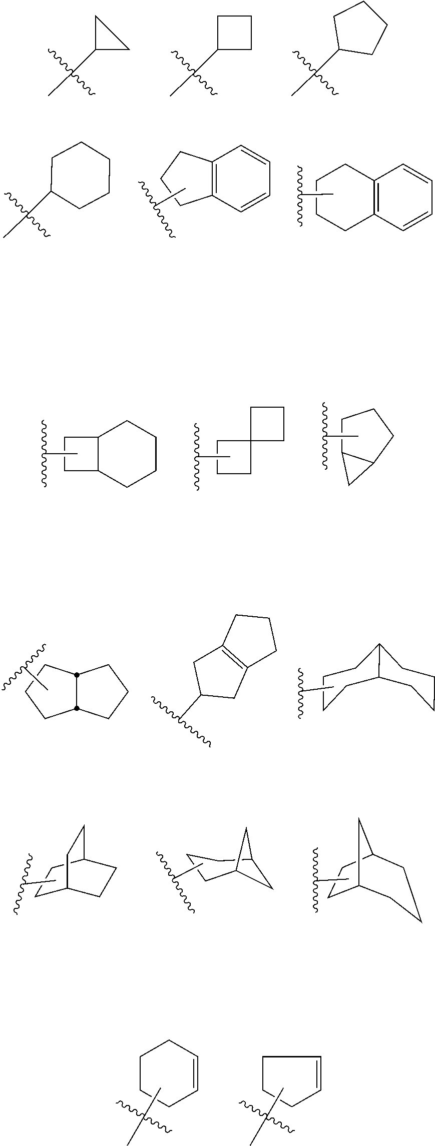

Unless otherwise stated, the following terms used in the specification and claims have the following meanings. “Aryl” refers to an all-carbon monocyclic group or fused polycyclic group (i.e. Rings that share two adjacent carbon atom pairs) with conjugated 7L electron systems, such as phenyl and naphthyl. The aromatic ring can be fused with other cyclic groups (including saturated and unsaturated rings), but cannot contain heteroatoms such as nitrogen, oxygen, or sulfur. Meanwhile, the point connecting to the parent structure must be on the carbon atom of the ring with a conjugated 7L electron system. Aryl can be either substituted or unsubstituted. The following are some examples of aryl, but the present invention is not limited to the following aryls:

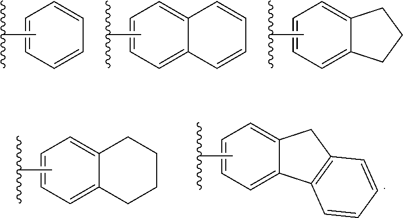

“Heteroaryl” refers to an aromatic monocyclic or polycyclic group containing one to more heteroatoms (optionally selected from nitrogen, oxygen, and sulfur), or a polycyclic group formed by heterocyclyl (containing one or more heteroatoms optionally selected from nitrogen, oxygen, and sulfur) fusing with aryl, with the connection site located on aryl. Heteroaryl can be either substituted or unsubstituted. The following are some examples of heteroaryl, but the present invention is not limited to the following heteroaryl:

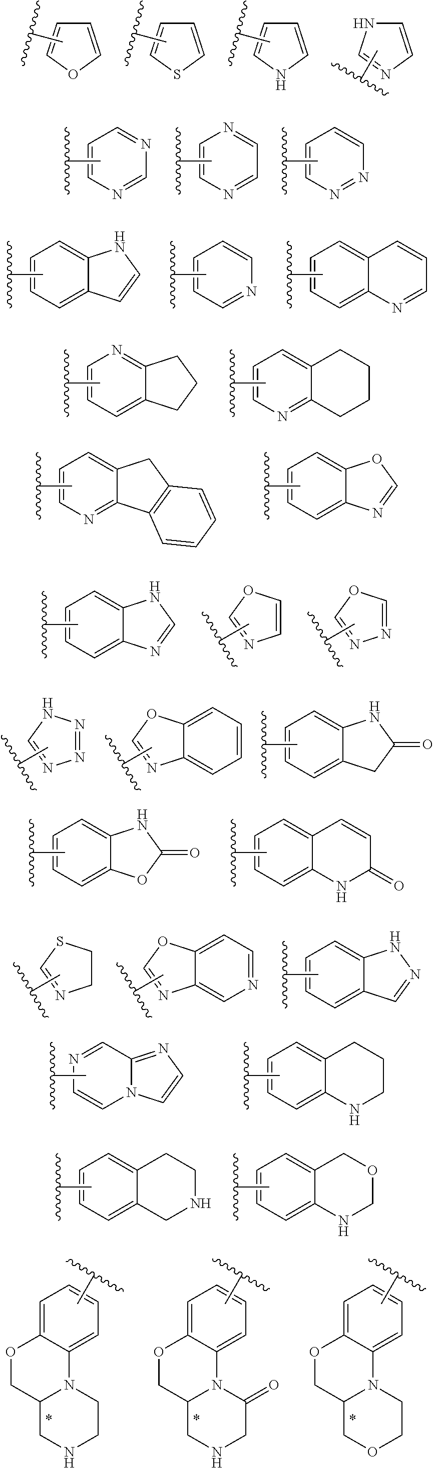

“Heterocyclyl” refers to a saturated or partially unsaturated monocyclic or polycyclic hydrocarbon substituent, where one or more ring atoms are selected from nitrogen, oxygen, or sulfur, and the remaining ring atoms are carbon. Non limiting examples of monocyclic heterocyclyl include pyrrolidinyl, piperidinyl, piperazinyl, morpholinyl, thiomorpholinyl, and homopiperazinyl. Polycyclic heterocyclyl refers to spiral, fused, and bridged heterocyclyl. “Spiral heterocyclyl” refers to a polycyclic heterocyclic group in which each ring in the system shares one atom (called a spiral atom) with other rings in the system, and one or more ring atoms are selected from nitrogen, oxygen, or sulfur, and the remaining ring atoms are carbon. The term “fused heterocyclyl” refers to a polycyclic heterocyclic group in which each ring in the system shares an adjacent pair of atoms with other rings in the system, wherein one or more of the rings may contain one or more double bonds, but none of the rings has a fully conjugated 7L electron system, and one or more ring atoms are selected from nitrogen, oxygen, or sulfur, with the remaining ring atoms being carbon. “Bridged heterocyclyl” refers to a polycyclic heterocyclic group where any two rings share two atoms that are not directly connected, which can contain one or more double bonds, but none of the rings has a fully conjugated R electron system, and one or more ring atoms are selected from nitrogen, oxygen, or sulfur, with the remaining ring atoms being carbon. If both saturated ring and aromatic ring are present in the heterocyclic group (e.g. saturated ring fused with aromatic ring), the point of attachment to the parent structure must be on the saturated ring. Note: when the point of attachment to the parent structure is on the aromatic ring, it is called heteroaryl, rather than heterocyclyl. The following are some examples of heterocyclyl, but the present invention is not limited to the following heterocyclyl:

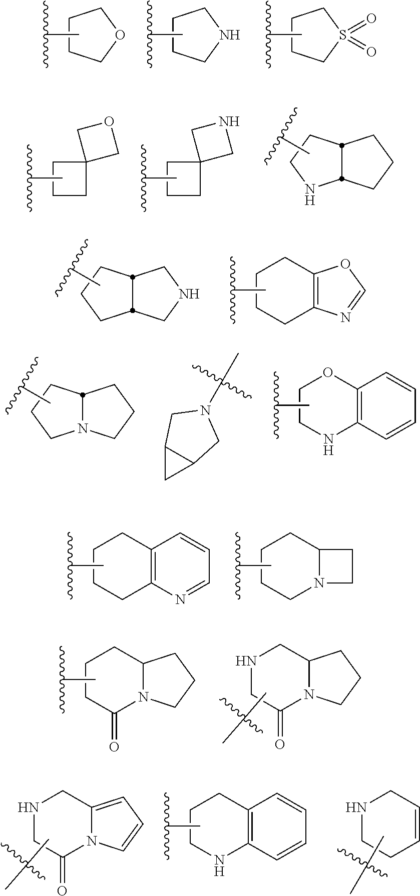

As used herein, when used alone or as part of other substituents, the term “halogen” refers to F, Cl, Br, and I.

As used herein, the term “substituted” (with or without “optionally” modification) refers to one or more hydrogen atoms on a specific group is substituted by a specific substituent. The specific substituent is the corresponding substituent described above, or the substituent appearred in each example. Unless otherwise specified, an optionally substituted group can have a substituent selected from a specific group at any substitutable site of the group, and the substituent can be the same or different at each position. A cyclic substituent, such as heterocyclyl, can be connected to another ring, such as cycloalkyl, so as to form a spirodicyclic system, where two rings share a common carbon atom. Those skilled in the art should understand that the expected combinations of substituents in the present invention are those that are stable or chemically achievable. The substituent is, such as (but not limited to) C₁₋₈ alkyl, C₂₋₈ alkenyl, C₂₋₈alkynyl, C₃₋₈cycloalkyl, 3- to 12-membered heterocyclyl, aryl, heteroaryl, halogen, hydroxyl, carboxyl (—COOH), C₁₋₈ aldehyde, C₂₋₁₀ acyl, C₂₋₁₀ ester, amino.

For convenience and conventional understanding, the terms “arbitrarily substituted” or “optionally substituted” only apply to sites that can be substituted by substituents, and do not include those substitutions that cannot be chemically achieved.

As used herein, unless otherwise specified, the term “pharmaceutically acceptable salt” refers to a salt that is suitable for contacting with the tissue of an object (such as a person) without causing undue side effects. In some embodiments, pharmaceutically acceptable salts of a certain compound of the present invention include salts of compounds of the present invention with acidic groups (such as potassium salts, sodium salts, magnesium salts, calcium salts) or salts of compounds of the present invention with alkaline groups (such as sulfate, hydrochloride, phosphate, nitrate, carbonate).

### Uses

The present invention provides a Use of a class of compounds of formula (I), or deuterated derivatives thereof, salts, isomers (enantiomers or diastereoisomers, if present), hydrates thereof, and pharmaceutically acceptable carriers, or excipients in the inhibition of KRAS G12D.

The compound of the present invention can be used as KRAS G12D inhibitor.

The compound of the present invention is a single inhibitor of KRAS G12D, which achieves the goal of preventing, alleviating, or curing diseases by regulating the activity of KRAS G12D. The disease includes various solid tumors and blood tumors such as pancreatic cancer, non-small cell lung cancer, small cell lung cancer, lung adenocarcinoma, lung squamous carcinoma, colon cancer, colorectal cancer, thyroid cancer, embryonal rhabdomyosarcoma, skin granular cell tumor, melanoma, liver cancer, rectal cancer, bladder cancer, throat cancer, breast cancer, prostate cancer, glioma, ovarian cancer, head and neck squamous cell cancer, cervical cancer, esophageal cancer, kidney cancer, skin cancer, lymphoma, stomach cancer, acute myeloid leukemia, myelofibrosis, B-cell lymphoma, monocytic leukemia, polycythemia megalosplenica, eosinophilic leukocytosis syndrome, myeloma, etc.

The compounds of the present invention, and deuterated derivatives thereof, pharmaceutically acceptable salts or isomers (if present) or hydrates thereof and/or combinations thereof, can be formulated together with pharmaceutically acceptable excipients or carriers, and the resulting compositions can be administered in vivo to mammals, such as men, women, and animals, for the treatment of conditions, disorders, and diseases. The composition can be: tablets, pills, suspensions, solutions, emulsions, capsules, aerosols, sterile injections, sterile powder, etc. In some embodiments, pharmaceutically acceptable excipients include microcrystalline cellulose, lactose, sodium citrate, calcium carbonate, calcium hydrogen phosphate, mannitol, hydroxypropyl- p-cyclodextrin, p-cyclodextrins (increased), glycine, disintegrants (such as starch, croscarmellose sodium, composite silicates, and polymer polyethylene glycol), granulating adhesives (such as polyvinyl pyrrolidone, sucrose, gelatin, and arabic gum), and lubricants (such as magnesium stearate, glycerol, and talc powder). In a preferred embodiment, the pharmaceutical composition is a dosage form suitable for oral administration, including but not limited to tablets, solutions, suspensions, capsules, granules, and powders. The amount of compound or pharmaceutical composition of the present invention administered to patients is not fixed, and is usually administered in a pharmaceutically effective amount. At the same time, the actual amount of compound administered can be determined by the physician based on actual situation, including the treatment symptoms, the chosen route of administration, the actual compound administered, and individual condition of the patient. The dosage of the compounds of the present invention depends on the specific use of the treatment, the mode of administration, the state of the patient, and the judgement of the physician. The proportion or concentration of the compounds of the present invention in a pharmaceutical composition depends on a variety of factors, including dosage, physicochemical properties, route of administration, and the like.

It should be understood that within the scope of the present invention, the above-mentioned technical features of the present invention and the technical features specifically described in the following (such as embodiments) can be combined with each other to form a new or preferred technical solution.

### General Synthetic Schemes

### Abbreviations and Chemical Reagents

Ac=Acetyl
Boc=tert-butoxycarbonyl
t-BuOK=potassium tert-butoxide
Catacxium-A-Pd-G3═Methylsulfonic acid [n-butyldi (1-adamantinyl) phosphine](2-amino-1,1′-biphenyl-2-yl) palladium (II)
m-CPBA=m-chloroperbenzoic acid
DAST=Diethylamine sulfur trifluoride
DCM=dichloromethane
DIPEA or DIEA=diisopropylethylamine
DIBAL-H=diisobutylaluminium hydride
DMAP=4-dimethylaminopyridine
DMF=dimethylform amide
DMSO=dimethyl sulfoxide
EtOAc or EA=ethyl acetate
HATU=1-[Bis(dimethylamino)methylene]-1 H-1,2,3-triazolo [4,5-b]pyridinium 3-oxid hexafluorophosphate
LiHMDS=lithium bis(trimethylsily)amide
Me═Methane
NMP=N-methylpyrrolidone
MOM═Methoxymethyl
Ph=Benzyl
Py=pyridine
i-PrOH=isopropyl alcohol
TBAF=tetrabutylammonium fluoride solution
TIPS=triisopropylsilyl group
TEA=triethylamine
Tf=trifluoromethanesulfonyl
TFA=Trifluoroacetic acid
THF=Tetrahydrofuran
TMS=Trimethylsilyl
TsOH=p-Toluenesulfonic acid

The compound of formula (I-A), (I-B), (I-C) of the present invention can be prepared by the following method:

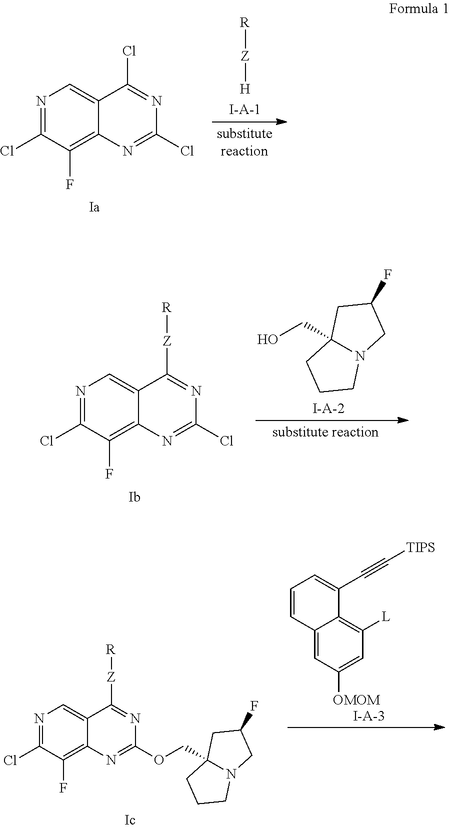

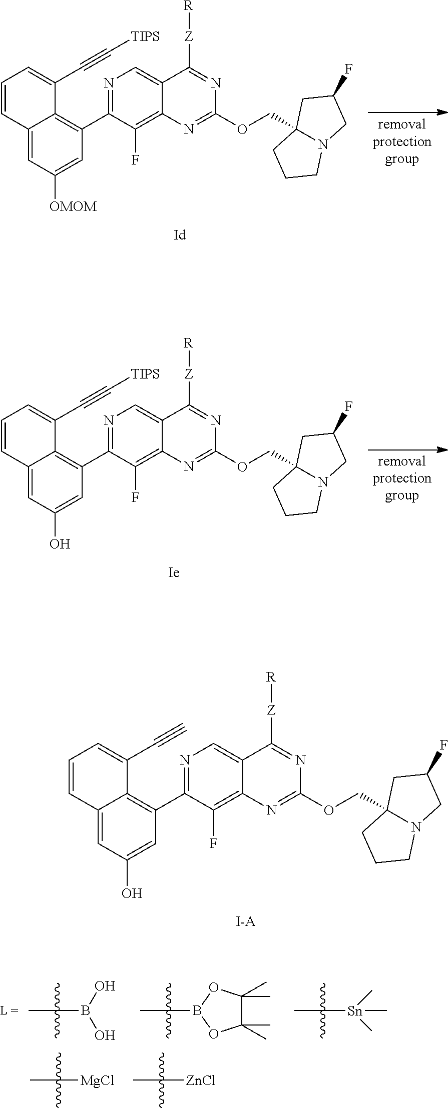

In an inert solvent and basic condition, substitution reaction takes place to obtain the intermediate (Ib) with compound (Ia). Intermediate (Ic) can be obtained through substitution reaction with (Ib) under inert solvent and basic condition. And then reacts with compound (I-A-3) to obtain the intermediate (Id). The target can be obtained after one or two deprotection reactions.

The definitions of Z and R in the above formula are the same as the expressions above.

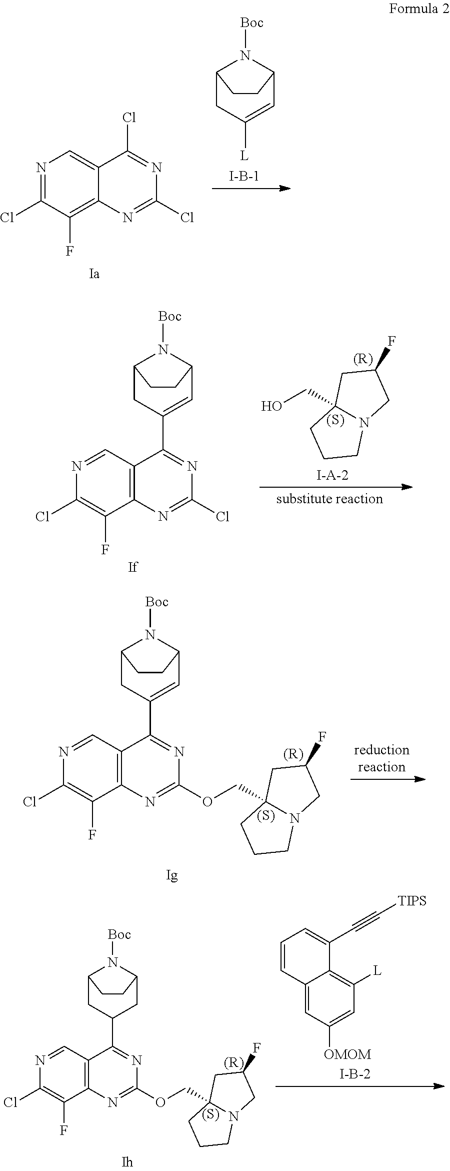

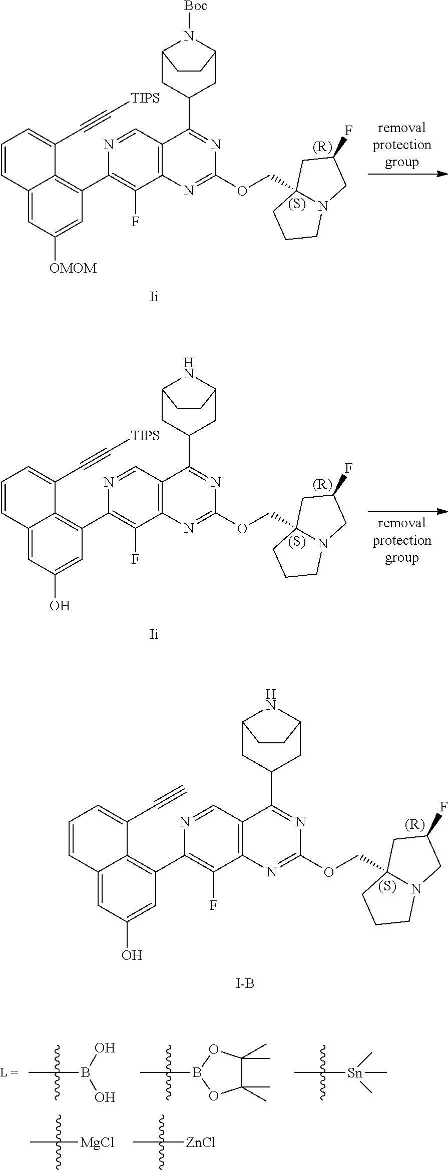

In an inert solvent, compound (Ia) reacted with (I-B-I) to produce intermediate (If). Intermediate (Ig) can be obtained through substitution reaction with (If) under inert solvent and basic condition. Intermediate (Ig) was selective reduced of extracellular double bonds to get intermediate (Ih). And then reacts with compound (I-1B-2) to obtain the intermediate (Ii) in an inert solvent. The target can be obtained after one or two deprotection reactions.

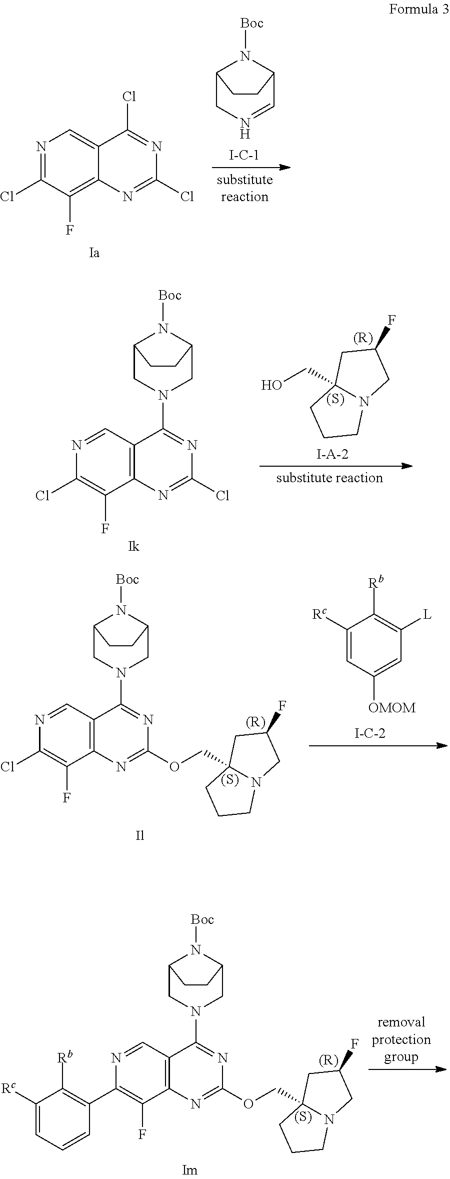

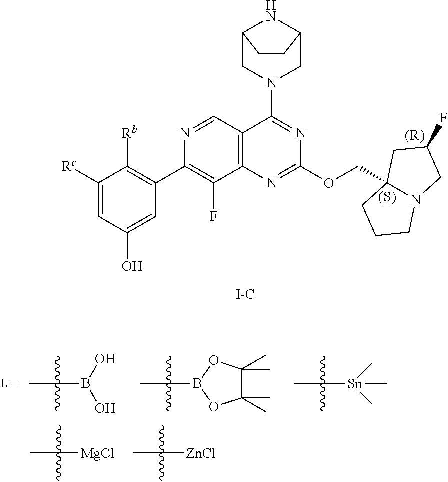

In an inert solvent and basic condition, substitution reaction takes place to obtain the intermediate (Ik) with compound (Ia). Intermediate (Il) can be obtained through substitution reaction with (Ik) under basic condition. And then reacts with compound (I-C-2) to obtain the intermediate (Im). The target can be obtained after deprotection reaction.

The definitions of R^{b} and R^{c} in the above formula are the same as the expressions of substitution groups on Ar above.

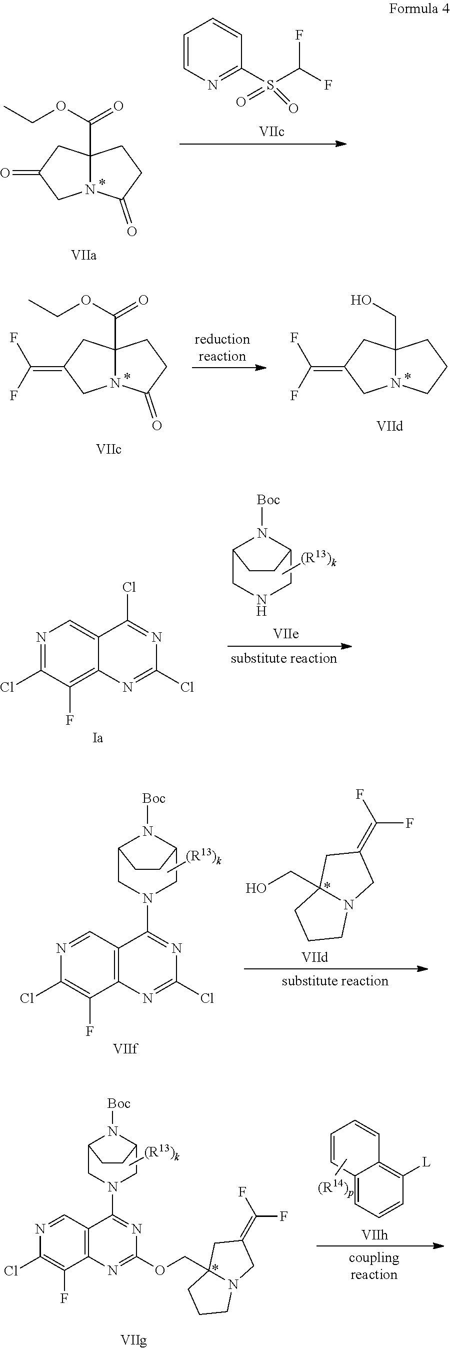

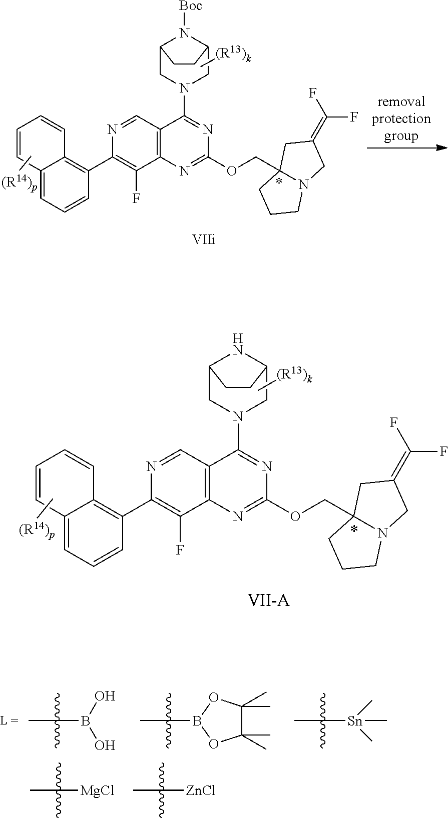

In an inert solvent, compound (VIIa) reacted with (VIIb) to produce intermediate (VIIc). (VIIc) was obtained through reduction reaction. Compound (Ia) reacted with compound (VIIe) to give intermediate (VIIf) under substitution reaction. Intermediate (VIIg) can be obtained through substitution reaction with between intermediate (VIIf) and compound (VIId) under basic condition. In an inert solvent, intermediate (VIIg) reacted with (VIIh) to produce intermediate (VIIi). The target can be obtained after deprotection reaction. The target compound with a single chiral configuration can employ the R or S isomer of compound (VIIa) in the first step of the reaction, ultimately result in the target compound with a chirality of R or S; The intermediate (VIIi) can also be separated by chiral separation and the protective group can be removed to obtain the target compound with chiral R or S; The chiral separation of the racemate of compound (VII-A) can also be used to obtain target compounds with chirality of R or S.

The definitions of R¹³, R¹⁴, k and p in the above formula are the same as the expressions of claim 9.

The definitions of each functional group in the above formulas are as mentioned above. The reagents and conditions for each step can be selected from the conventional reagents or conditions for the preparation method in this field. After the compound structure of the present invention is disclosed, the above selection can be processed by technical personnel in this field based on their knowledge in this field.

### Pharmaceutical Composition and Mode of Administration

Since the compound of the present invention has excellent activity of inhibiting KRAS G12D, the compound of the present invention and its various crystal forms, pharmaceutically acceptable inorganic or organic salts, hydrates or solvates, and pharmaceutical compositions containing the compound of the present invention as the main active ingredient can be used to treat, prevent and alleviate diseases related to KRAS G12D activity or expression level.

The pharmaceutical composition of the present invention comprises the compound of the present invention or a pharmacologically acceptable salt thereof within a safe and effective amount range, and pharmaceutically acceptable excipients or carriers. Wherein, “safe and effective amount” refers to: the amount of the compound is sufficient to obviously improve the condition without causing severe side effects. Usually, the pharmaceutical composition contains 1-2000 mg of the compound of the present invention per dose, more preferably, 5-500 mg of the compound of the present invention per dose. Preferably, the “one dose” is a capsule or tablet.

“Pharmaceutically acceptable carrier” refers to one or more compatible solid or liquid fillers or gel substances, which are suitable for human use and must have sufficient purity and low toxicity. “Compatibility” herein refers to the ability of components of a composition to blend with each other and with the compounds of the invention without significantly reducing the efficacy of the compounds. Examples of pharmaceutically acceptable carriers include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oil (such as soybean oil, sesame oil, peanut oil, olive oil, etc.), polyols (such as propylene glycol, glycerin, mannitol, sorbitol), emulsifiers (e.g. Tween)®, wetting agents (such as sodium dodecyl sulfate), colorants, flavoring agents, stabilizers, antioxidants, preservatives, pyrogen-free water, etc.

The mode of administration of the compounds or pharmaceutical compositions of the present invention are not particularly limited, and representative mode of administration include, but are not limited to, oral, intratumoral, rectal, parenteral (intravenous, intramuscular or subcutaneous), and topical administration.

Solid dosage forms for oral administration include capsules, tablets, pills, powders and granules. In these solid dosage forms, the active compound is mixed with at least one conventional inert excipient (or carrier), such as sodium citrate or dicalcium phosphate, or with the following ingredients: (a) filler or compatibilizer, such as starch, lactose, sucrose, glucose, mannitol and silicic acid; (b) adhesives, such as hydroxymethyl cellulose, alginate, gelatin, polyvinylpyrrolidone, sucrose and arabic gum; (c) moisturizing agents, such as glycerol; (d) disintegrating agents, such as agar, calcium carbonate, potato starch or cassava starch, algic acid, some complex silicates, and sodium carbonate; (e) retarding solvent, such as paraffin; (f) absorption accelerators, such as quaternary amine compounds; (g) wetting agents, such as cetyl alcohol and glycerol monostearate; (h) adsorbents, such as kaolin; and (i) lubricants, such as talc, calcium stearate, magnesium stearate, solid polyethylene glycol, sodium dodecyl sulfate, or mixtures thereof. In capsules, tablets and pills, dosage forms may also contain buffers.

Solid dosage forms such as tablets, sugar pills, capsules and granules may be prepared using coating and shell materials such as casing and other materials well known in the art. They may comprise an opacifying agent, and the release of the active compound in such a composition may be released in a delayed manner in a part of the digestive tract. Examples of embedding components that can be employed are polymeric substances and wax substances. If necessary, the active compound may also form a microcapsule form with one or more of the excipients described above.

Liquid dosage forms for oral administration include pharmaceutically acceptable emulsions, solutions, suspensions, syrups or tinctures. In addition to the active compounds, the liquid dosage form may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or mixtures thereof.

In addition to these inert diluents, the composition may also contain auxiliaries such as wetting agents, emulsifiers, suspending agents, sweeteners, flavoring agents and spices.

In addition to the active compound, the suspension may comprise suspending agents, such as ethoxylatedisooctadecanol, polyoxyethylene sorbitol and dehydrated sorbitol esters, microcrystalline cellulose, methanolic aluminum, agar, and any mixtures thereof.

The composition for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

Dosage forms of the compound of the invention for topical administration include ointments, powder, patches, propellants and inhalants. The active ingredient is mixed under sterile conditions with a physiologically acceptable carrier and any preservatives, buffers or propellants as may be required.

The compound of the present invention can be administered alone or in combination with other pharmaceutically acceptable compounds.

When using the pharmaceutical composition, a safe and effective amount of the compound of the present invention is administered to mammals in need of treatment (such as humans), where the dosage at the time of administration is the pharmaceutically considered effective dose, which is typically 1 to 2,000 mg per day, more preferably 5 to 500 mg per day for a 60 kg body weight human. Of course, the specific dosage should also consider the route of administration, the patient's health condition and other factors, which are within the skill range of skilled doctors.

### Main Advantages of the Present Invention

1. The present invention provided a compound as shown in formula I.

2. The present invention provided a novel KRAS G12D inhibitor, and the preparation therefor and application thereof, the inhibitor can inhibit the activity of KRAS G12D at extremely low concentrations.

3. The present invention provided a class of pharmaceutical compositions for treating diseases related to KRAS G12D activity.

The present invention is further described below in conjunction with specific embodiments. It is to be understood that these examples are intended to illustrate the invention only and not to limit the scope of the invention. The experimental methods in the following examples that do not specify specific conditions are usually based on conventional conditions or conditions recommended by the manufacturer. Unless otherwise specified, percentages and portions are calculated by weight.

### Example 1: Preparation of Compound 1

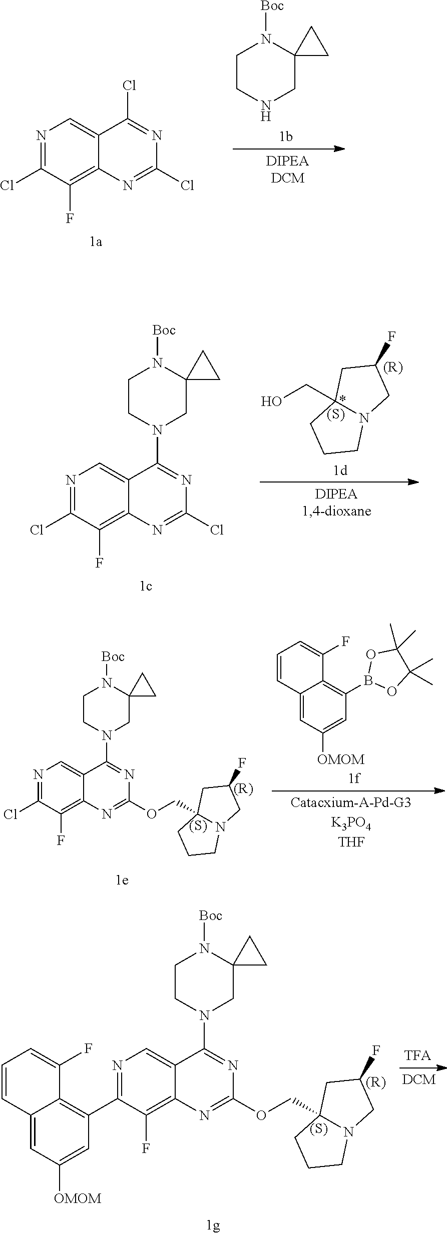

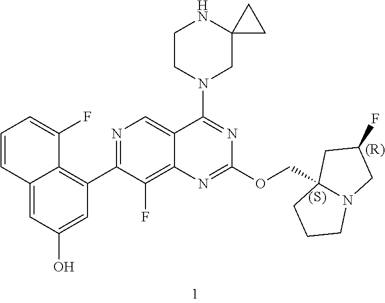

Compound 1a was synthesized according to WO2021041671. Compound 1a (60 mg, 0.24 mmol) and N,N-diisopropylethylamine(215 mg, 1.66 mmol) were dissolved in dichloromethane (3 mL). 1b (50 mg, 0.24 mmol) was added to the mixture under −40° C. Then the reaction mixture was stirred at −40° C. for 1 h. After the reaction was completed, water was added. Then the mixture was extracted with dichloromethane (3×15 mL), and the collected organic phase was washed with sat.NaCl, dried with anhydrous sodium sulfate, filterated and concentrated to give the crude product. The crude product was purified by column chromatography (dichloromethane:ethyl acetate=5:1) to afford a yellow solid 1c (90 mg, yield: 88%). MS m/z 428.3 [M+H]⁺.

Compound 1d was commercially available. The compound 1c (60 mg, 0.14 mmol), 1d (34 mg, 0.21 mmol) and N,N-diisopropylethylamine(50 mg, 0.42 mmol) were dissolved in dioxane (1.5 mL). Then the reaction mixture was stirred at 90° C. overnight. After the reaction was completed, the mixture was concentrated to give the crude product. The crude product was purified by column chromatography (dichloromethane: methanol=5:1) to afford yellow oil 1e (42 mg, yield: 54%). MS m/z 428.3 [M+H]⁺.

Compound 1f was synthesized according to WO2021041671. Compound 1e (42 mg, 0.08 mmol), if (43 mg, 0.13 mmol) and Methylsulfonic acid [n-butyldi (1-adamantinyl) phosphine](2-amino-1,1′-biphenyl-2-yl) palladium (II) (5.5 mg, 0.008 mmol) were added to the reaction bottle, purged with N₂ for 3 times. Then tetrahydrofuran (1.5 mL) and potassium phosphate aqueous solution (1.5 M, 0.15 mL) was added. Then the reaction mixture was stirred at 60° C. for 2 h under N₂. After the reaction was completed, the mixture was cooled to RT, water was added. Then the mixture was extracted with ethyl acetate (3×10 mL), and the collected organic phase was washed with sat.NaCl, dried with anhydrous sodium sulfate, filterated and concentrated. The crude product was purified by column chromatography (dichloromethane: methanol=25:1) to afford yellow oil 1g (33 mg, yield: 60%). MS m/z 721.8 [M+H]⁺.

The compound 1g (29 mg, 0.04 mmol) was dissolved in dichloromethane (3 mL). Then trifluoroacetic acid (0.5 mL) was added. The reaction mixture was stirred at room temperature for 2 h. After the reaction was completed, the mixture was concentrated and adjusted to pH=7 with ammonia. The mixture was concentrated again. The crude product was purified by column chromatography (dichloromethane: methanol=15:1, 2% ammonia) to afford a yellow solid 1 (13.6 mg, yield: 59%). ¹H NMR (500 MHz, CD₃OD) δ 9.13 (s, 1H), 7.60 (d, J=7.8 Hz, 1H), 7.42-7.37 (m, 1H), 7.34 (t, J=2.2 Hz, 1H), 7.18-7.15 (m, 1H), 6.94-6.88 (m, 1H), 5.65-5.49 (m, 1H), 4.75-4.66 (m, 2H), 4.39-4.34 (m, 2H), 4.20 (s, 2H), 4.05-3.83 (m, 3H), 3.57-3.43 (m, 3H), 2.76-2.65 (m, 1H), 2.65-2.54 (m, 1H), 2.47-2.41 (m, 1H), 2.38-2.29 (m, 2H), 2.22-2.14 (m, 1H), 1.09-1.02 (m, 4H) ppm. MS m/z 577.6 [M+H]⁺.

### Example 2: Preparation of Compound 2

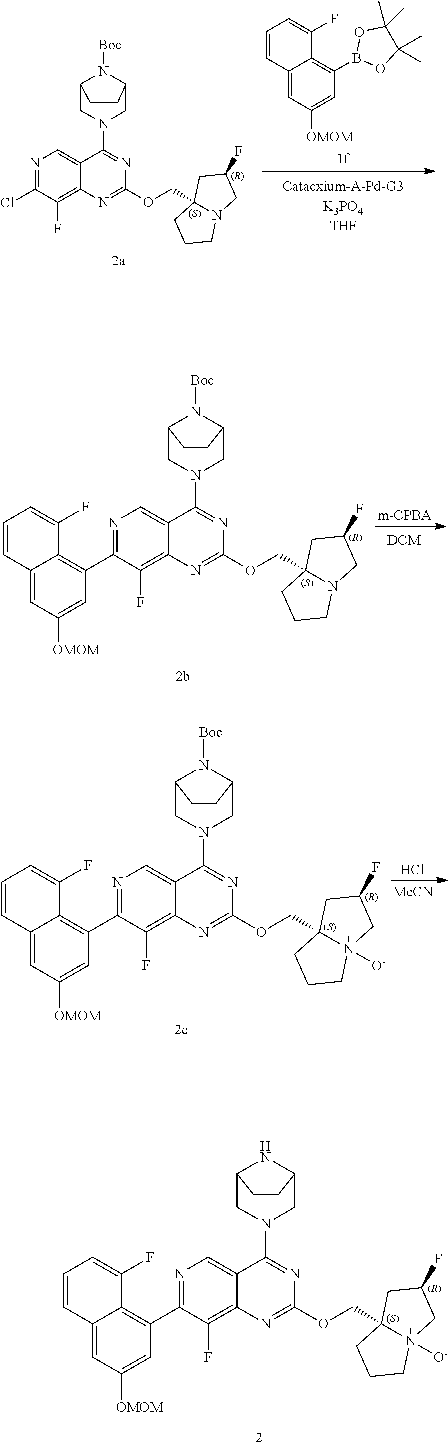

Compound if and 2a was synthesized according to WO2021041671. Compound 2a (22 mg, 0.04 mmol), if (27 mg, 0.08 mmol),Methylsulfonic acid [n-butyldi (1-adamantinyl) phosphine](2-amino-1,1′-biphenyl-2-yl) palladium (II) (3 mg, 0.004 mmol) and potassium phosphate aqueous solution (1.5 M, 0.08 mL) were added to dioxane (1 mL), purged with N₂ for 1 min. Then the reaction mixture was stirred at 100° C. for 2 h under sealed tube.

After the reaction was completed, the mixture was cooled to RT, water was added. Then the mixture was extracted with ethyl acetate (3×10 mL), and the collected organic phase was washed with sat.NaCl, dried with anhydrous sodium sulfate, filterated and concentrated. The crude product was purified by column chromatography (dichloromethane: methanol=25:1) to afford yellow solid 2b (27 mg, yield: 94%). MS m/z 721.9 [M+H]⁺. The compound 2b (25 mg, 0.03 mmol) was dissolved in dichloromethane (2 mL). Then 85% mCPBA (8 mg, 0.04 mmol) was added. The reaction mixture was stirred at room temperature for 1 h. After the reaction was completed, the mixture was concentrated. The crude product was purified by column chromatography (dichloromethane: methanol=8:1, 2% ammonia) to afford a yellow solid 2c (11 mg, yield: 43%). MS m/z 737.9 [M+H]⁺. The compound 2c (10 mg, 0.01 mmol) was dissolved in acetonitrile (1.5 mL). Then 4M HCl in dioxane (0.2 mL) was added. After the reaction was completed, the mixture was concentrated and adjusted to pH=7 with ammonia. The mixture was concentrated again. The crude product was purified by column chromatography (dichloromethane: methanol=10:1, 2% ammonia) to afford a yellow solid 2 (5.3 mg, yield: 66%). ¹H NMR (500 MHz, CD₃OD) δ 9.07 (s, 1H), 7.60 (d, J=8.0 Hz, 1H), 7.42-7.36 (m, 1H), 7.33 (t, J=2.2 Hz, 1H), 7.16 (d, J=1.5 Hz, 1H), 6.91 (dd, J=13.1, 7.6 Hz, 1H), 5.72-5.53 (m, 1H), 5.00 (d, J=11.7 Hz, 1H), 4.80-4.72 (m, 2H), 4.53 (d, J=11.8 Hz, 1H), 4.19-3.99 (m, 3H), 3.93 (s, 2H), 3.84-3.79 (m, 2H), 3.75-3.68 (m, 1H), 3.13-3.00 (m, 1H), 2.50-2.35 (m, 2H), 2.29-2.21 (m, 3H), 2.01-1.91 (m, 4H) ppm. MS m/z 593.7 [M+H]⁺.

### Example 3: Preparation of Compound 3

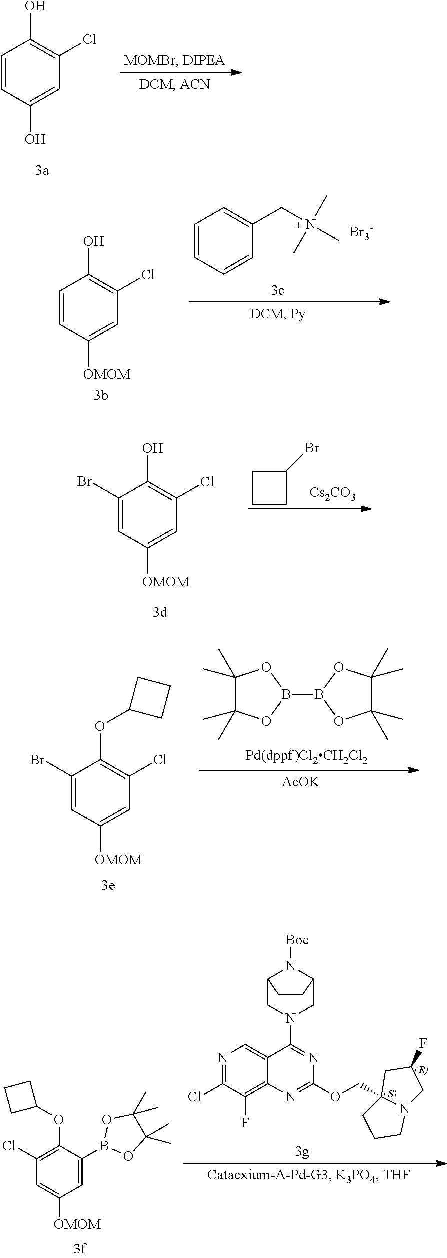

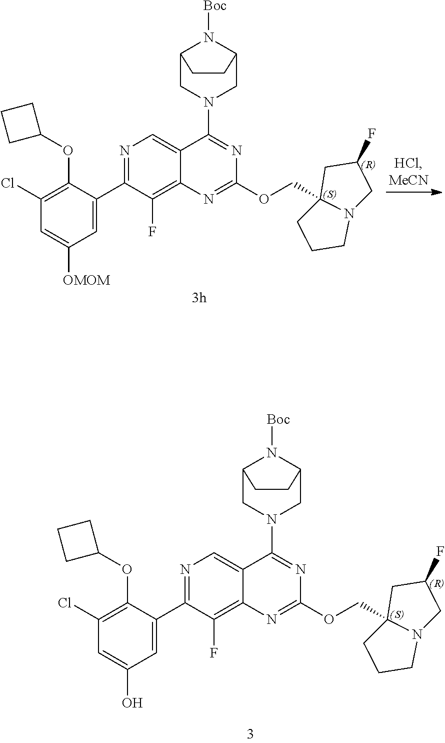

Compound 3a (10g, 70 mmol) was dissolved in dichloromethane (30 mL and acetonitrile (30 mL). Then N, N-diisopropylethylamine(9.50 g, 84 mmol) and bromomethyl ether (8.75 g, 70 mmol) were added. Then the reaction mixture was stirred for 2 h. After most of the raw materials were consumed, the mixture was concentrated and ethyl acetate was added, 1N HCl was added to adjust the system to weak acidity. The collected organic phase was washed with sat.NaCl, dried with anhydrous sodium sulfate, filterated and concentrated to give the crude product. The crude product was purified by column chromatography (petroleum ether: ethyl acetate=75:25) to afford 3b (600 mg, yield: 4.6%). ¹H NMR (500 MHz, CD₃OD) δ 6.91 (t, J=1.6 Hz, 1H), 6.72 (d, J=1.6 Hz, 2H), 4.97 (s, 2H), 3.34 (s, 3H) ppm.

Compound 3b (640 mg, 3.4 mmol) was dissolved in dichloromethane (10 mL). Then pyridine (200 mg) in dichloromethane (1 mL) and compound 3c (1.24 g, 3.4 mmol) were added. Then the reaction mixture was stirred at room temperature for 2 h. After most of the raw materials were consumed, iN HCl was added to adjust the system to weak acidity. Then the mixture was washed with a solution of sodium bisulfite, extracted with dichloromethane.The collected organic phase was washed with sat.NaCl, dried with anhydrous sodium sulfate, filterated and concentrated. The crude product was purified by column chromatography (petroleum ether: ethyl acetate=85:15) to afford 3d (420 mg, yield: 45%). ¹H NMR (500 MHz, CDCl₃) δ 7.16 (d, J=2.8 Hz, 1H), 7.06 (d, J=2.8 Hz, 1H), 5.58 (brs, 1H), 5.08 (s, 2H), 3.47 (s, 3H) ppm.

The compound 3d (250 mg, 0.93 mmol) was dissolved in DMF (2 mL). Then cesium carbonate (630 mg, 1.9 mmol) and bromocyclobutane (630 mg, 4.7 mmol) were added. The reaction mixture was stirred at 80° C. for 16 h. After the reaction was completed, the mixture was quenched with water, extracted with ethyl acetate. The collected organic phase was washed with sat.NaCl, dried with anhydrous sodium sulfate, filterated and concentrated. The crude product was purified by column chromatography (petroleum ether: ethyl acetate=90:10) to afford 3e (220 mg). ¹H NMR (500 MHz, CDCl₃) δ 7.16 (d, J=2.9 Hz, 1H), 7.04 (d, J=2.9 Hz, 1H), 5.10 (s, 2H), 4.53-4.47 (m, 1H), 3.47 (s, 3H), 2.43-2.22 (m, 4H), 1.73 (m, 1H), 1.44 (m, 1H) ppm.

Compound 3e (15 mg, 0.046 mmol), Pd(dppf)C₁₋₂—CH₂C₁₋₂(4 mg, 0.005 mmol),bis(pinacolato)diboron (24 mg, 0.093 mmol) and potassium acetate (15 mg, 0.138 mmol) were dissolved in dioxane (1 mL). Then the reaction mixture was stirred at 100° C. for 4 h under N₂. After the reaction was completed, water was added. Then the mixture was extracted with ethyl acetate for 2 times, and the collected organic phase was washed with sat.NaCl, dried with anhydrous sodium sulfate, filterated and concentrated. The crude product was purified by column chromatography (petroleum ether: ethyl acetate=95:5) to afford 3f (5 mg, yield: 45%). ¹H NMR (500 MHz, CDCl₃) δ 7.09 (d, J=3.0 Hz, 1H), 6.92 (d, J=3.0 Hz, 1H), 5.12 (s, 2H), 4.13-4.04 (m, 1H), 3.47 (s, 3H), 2.03-1.93 (m, 4H), 1.35-1.15 (m, 14H) ppm.

Compound 3g was synthesized according to WO2021041671. Compound 3f (34 mg, 0.061 mmol), Methylsulfonic acid [n-butyldi (1-adamantinyl) phosphine](2-amino-1,1′-biphenyl-2-yl) palladium (II) (5 mg, 0.0061 mmol) and 3g (32 mg, 0.086 mmol) were added to tetrahydrofuran (2 mL), then potassium phosphate aqueous solution (1.5 M, 0.5 mL) was added. Then the reaction mixture was stirred at 60° C. for 3 h under N₂. After the reaction was completed, water was added. Then the mixture was extracted with ethyl acetate for 2 times, and the collected organic phase was washed with sat.NaCl, dried with anhydrous sodium sulfate, filterated and concentrated. The crude product was purified by column chromatography (dichloromethane: methanol=95:5) to afford 3h (31 mg, yield: 37%). MS m/z 757.5 [M+H]⁺.

The compound 3h (15 mg, 0.02 mmol) was dissolved in acetonitrile (1 mL). Then HCl in dioxane (6M, 1 mL) was added. The reaction mixture was stirred at room temperature for 30 min. After the reaction was completed, the mixture was concentrated and adjusted pH to alkalinity with two drops ammonia. Then the reaction was diluted with dichloromethane, dried with anhydrous sodium sulfate. The mixture was purified by Pre-TLC to afford compound 3 (7 mg, yield: 57%). ¹H NMR (500 MHz, CD₃OD) δ 9.05 (s, 1H), 6.97 (d, J=2.9 Hz, 1H), 6.79 (d, J=2.9 Hz, 1H), 5.38-5.27 (m, 1H), 4.60 (t, J=12.0 Hz, 2H), 4.29 (dd, J=34.1, 10.6 Hz, 2H), 4.16-4.06 (m, 1H), 3.74-3.66 (m, 3H), 3.35 (d, J=14.1 Hz, 1H), 3.30-3.22 (m, 3H), 3.04 (td, J=9.7, 5.8 Hz, 1H), 2.40-2.13 (m, 3H), 2.07-2.00 (m, 2H), 1.88 (m, 4H), 1.80-1.69 (m, 3H), 1.41 (m, 2H), 1.29-1.19 (m, 2H) ppm.

### Example 4: Preparation of Compound 4, 4S, 4R

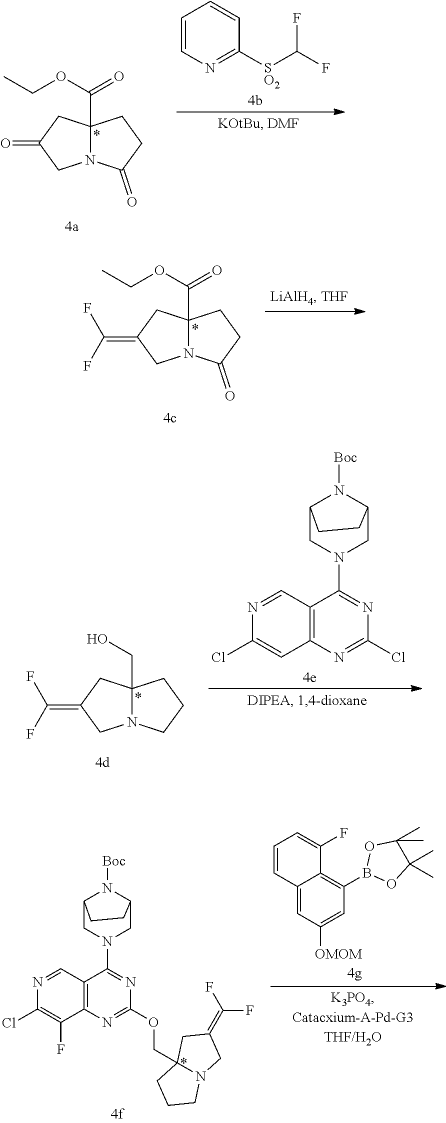

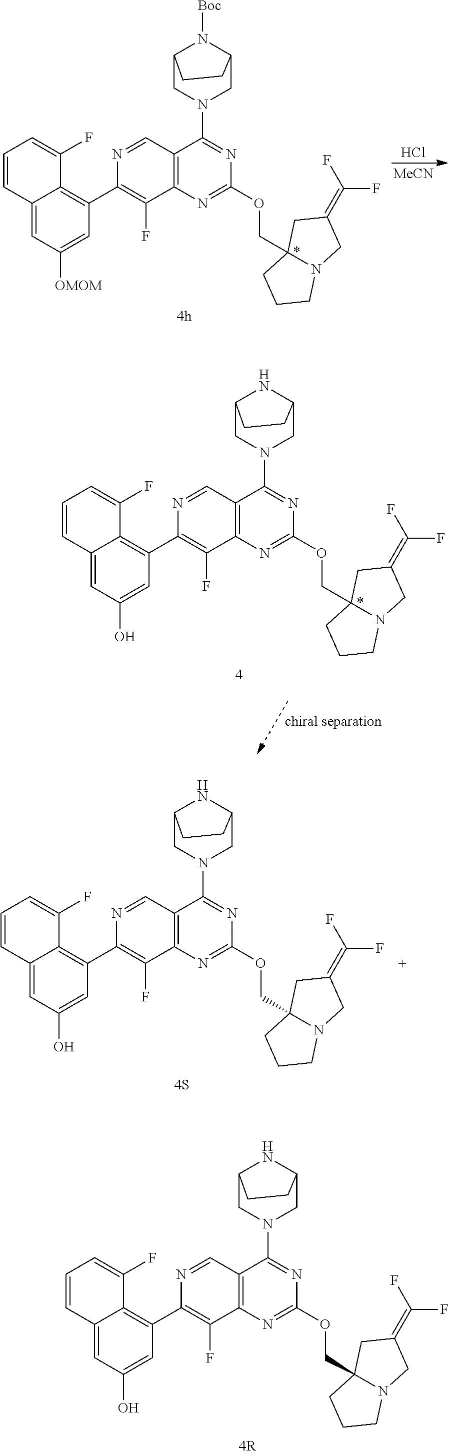

Compound 4b was synthesized according to (European Journal of Organic Chemistry, 2015, vol. 2015, #4, p. 871-875). t-BuOK(851 mg, 7.58 mmol) was dissolved in DMF (10 mL). The mixture was cooled to −50° C. under N₂. Then 4a (890 mg, 4.21 mmol, racemate) and 4b (733 mg, 3.79 mmol) in DMF (5 mL) was slowly added. The mixture was slowly warmed to room temperature and stirred for 0.5 h. After the reaction was completed, the reaction was quenched with sat.NH₄Cl and con.HCl (3 mL) was added. The mixture was was stirred for another 1 h. Then water (30 mL) was added and extracted with ethyl acetate (3×30 mL), and the collected organic phase was washed with sat.NaCl, dried with anhydrous sodium sulfate, filterated and concentrated to give the crude product. The crude product was purified by column chromatography to afford yellow oil 4c (188 mg, yield: 18%). ¹H NMR (500 MHz, CDCl₃) δ 4.39-4.33 (m, 1H), 4.23 (q, J=7.1 Hz, 2H), 3.78-3.72 (m, 1H), 3.16-3.11 (m, 1H), 2.85-2.76 (m, 1H), 2.66-2.60 (m, 1H), 2.53-2.46 (m, 1H), 2.43-2.36 (m, 1H), 2.19-2.11 (m, 1H), 1.29 (t, J=7.1 Hz, 3H). MS m/z 246.3 [M+H]⁺.

Lithium aluminum hydride(58 mg, 0.77 mmol) was dissolved in THF (1 mL), Then 4c (188 mg, 0.77 mmol) in THF (2 mL) was added under ice bath and nitrogen atmosphere. Then the reaction mixture was stirred at 65° C. for 3 h. water (0.06 mL), 15% NaOH (0.06 mL) and water (0.18 mL) were added under ice bath. The mixture was stirred at room temperature for 15 min. Then the reaction was dried with anhydrous sodium sulfate, filterated and concentrated. The crude product was purified by column chromatography to afford yellow oil 4d (38 mg, yield: 26%).

Compound 4e was synthesized according to WO2021041671. The compound 4e (80 mg, 0.19 mmol), 4d (38 mg, 0.20 mmol) and N,N-diisopropylethylamine(72 mg, 0.56 mmol) were dissolved in dioxane (1.5 mL). Then the reaction mixture was stirred at 90° C. for 24 h. After the reaction was completed, the mixture was concentrated to give the crude product. The crude product was purified by column chromatography to afford a white like solid compound 4f (33 mg, yield: 30%). MS m/z 581.5, 583.4 [M+H]⁺.

Compound 4g was synthesized according to WO2021041671. Compound 4f (29 mg, 0.05 mmol), Methylsulfonic acid [n-butyldi (1-adamantinyl) phosphine](2-amino-1,1′-biphenyl-2-yl) palladium (II) (5 mg, 0.0061 mmol), 4g (32 mg, 0.10 mmol) were added to tetrahydrofuran (2 mL) and potassium phosphate (32 mg, 0.15 mmol) were dissolved in dioxane/water (0.5 mL/0.1 mL). Then the reaction mixture was stirred at 90° C. for 2 h under N₂. After the reaction was completed, the mixture was filterated through diatomite and concentrated. The crude product was purified by column chromatography to afford 4h (27 mg, yield: 72%). MS m/z 751.5 [M+H]⁺.

The compound 4h (27 mg, 0.36 mmol) was dissolved in acetonitrile (0.5 mL). Then 4 M HCl in dioxane (0.2 mL) was added. The reaction mixture was stirred at room temperature for 1 h. After the reaction was completed, the mixture was concentrated and adjusted pH to 7-8 with ammonia. Then the reaction was concentrated again. The mixture was purified by Pre-TLC to afford white solid 4 (13 mg, yield: 60%, racemate). ¹H NMR (500 MHz, CD₃OD) δ 9.04 (s, 1H), 7.59 (d, J=8.3 Hz, 1H), 7.41-7.36 (m, 1H), 7.33 (brs, 1H), 7.16 (d, J=1.7 Hz, 1H), 6.91 (dd, J=13.0, 7.6 Hz, 1H), 4.71-4.64 (m, 2H), 4.35 (dd, J=43.8, 10.8 Hz, 2H), 3.87-3.72 (m, 5H), 3.46 (d, J=14.1 Hz, 1H), 3.20-3.14 (m, 1H), 2.81 (d, J=15.8 Hz, 1H), 2.76-2.71 (m, 1H), 2.53 (d, J=16.0 Hz, 1H), 2.18-2.09 (m, 1H), 2.04-1.97 (m, 1H), 1.96-1.83 (m, 6H) ppm. ¹⁹F NMR (471 MHz, CD₃OD) δ −92.24-−93.42 (m), −115.68-−115.86 (m), −140.74 (d, J=2.7 Hz) ppm. MS m/z 607.7 [M+H]⁺. Compound 4 (racemate) is separated by chiral column to obtain compounds 4S and 4R

### Example 5: Preparation of Compound 5

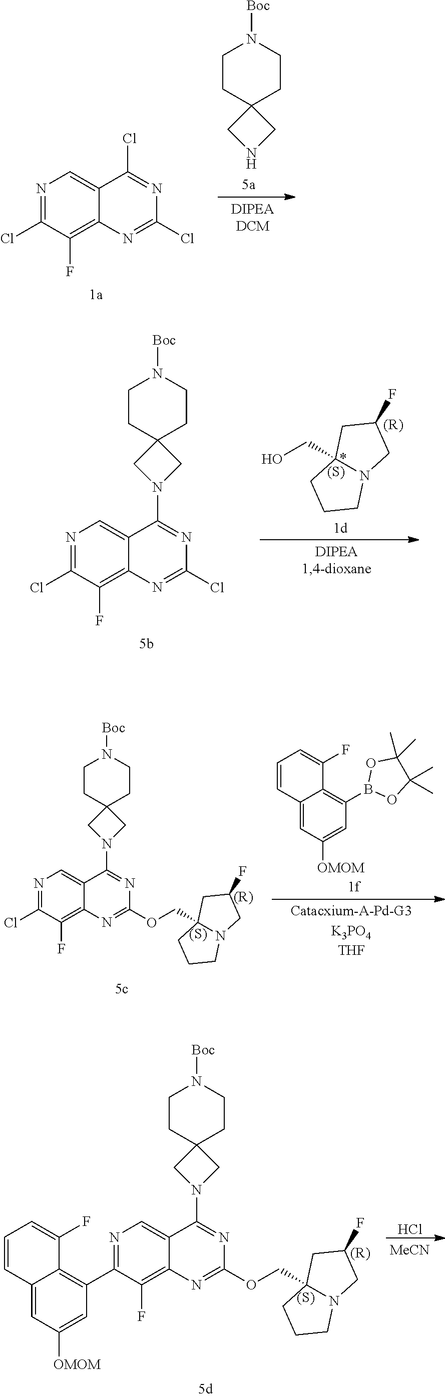

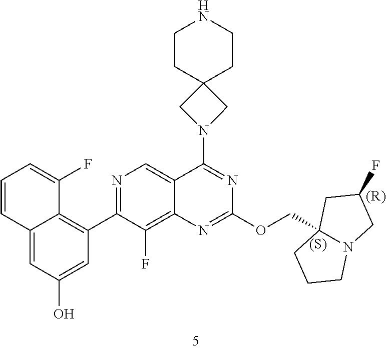

Compound 5d was synthesized according to the method of 1g.

The compound 5d (19 mg, 0.026 mmol) was dissolved in acetonitrile (2 mL). Then 4 M HCl in dioxane (0.2 mL) was added. The reaction mixture was stirred at room temperature for 1 h. After the reaction was completed, the mixture was concentrated and adjusted pH to 7 with ammonia. Then the reaction was concentrated again. The mixture was purified by column chromatography (dichloromethane: methanol=15:1, 2% ammonia) to afford yellow solid 5 (13.8 mg, yield: 90%). ¹H NMR (500 MHz, CD₃OD) δ 8.94 (s, 1H), 7.59 (d, J=8.0 Hz, 1H), 7.41-7.35 (m, 1H), 7.32 (t, J=2.1 Hz, 1H), 7.14 (d, J=2.3 Hz, 1H), 6.94-6.87 (m, 1H), 5.38-5.21 (m, 1H), 4.68-4.55 (m, 2H), 4.35-4.16 (m, 4H), 3.29-3.14 (m, 4H), 3.06-2.97 (m, 4H), 2.37-1.85 (m, 10H) ppm. MS m/z 591.5 [M+H]⁺.

### Example 6: Preparation of Compound 6

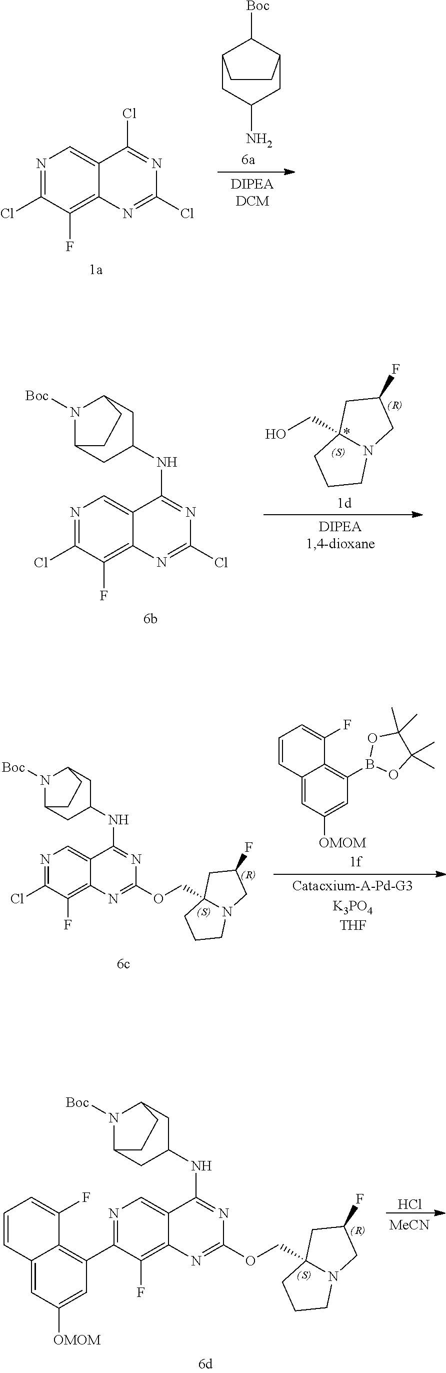

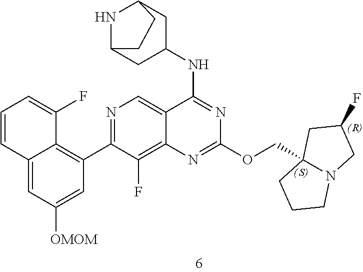

Compound 6 was synthesized according to the method of 1 to give a yellow solid 6 (9.32 mg). ¹H NMR (500 MHz, CD₃OD) δ 9.18 (s, 1H), 7.59 (d, J=7.8 Hz, 1H), 7.41-7.35 (m, 1H), 7.32 (t, J=2.2 Hz, 1H), 7.14 (d, J=2.4 Hz, 1H), 6.93-6.88 (m, 1H), 5.37-5.24 (m, 1H), 4.48-4.44 (m, 1H), 4.35-4.23 (m, 2H), 3.87-3.80 (m, 2H), 3.29-3.15 (m, 3H), 3.05-2.96 (m, 1H), 2.37-2.17 (m, 8H), 2.14-2.05 (m, 3H), 2.03-1.95 (m, 2H), 1.93-1.83 (m, 1H) ppm. MS m/z 591.7 [M+H]⁺.

### Example 7: Preparation of Compound 7

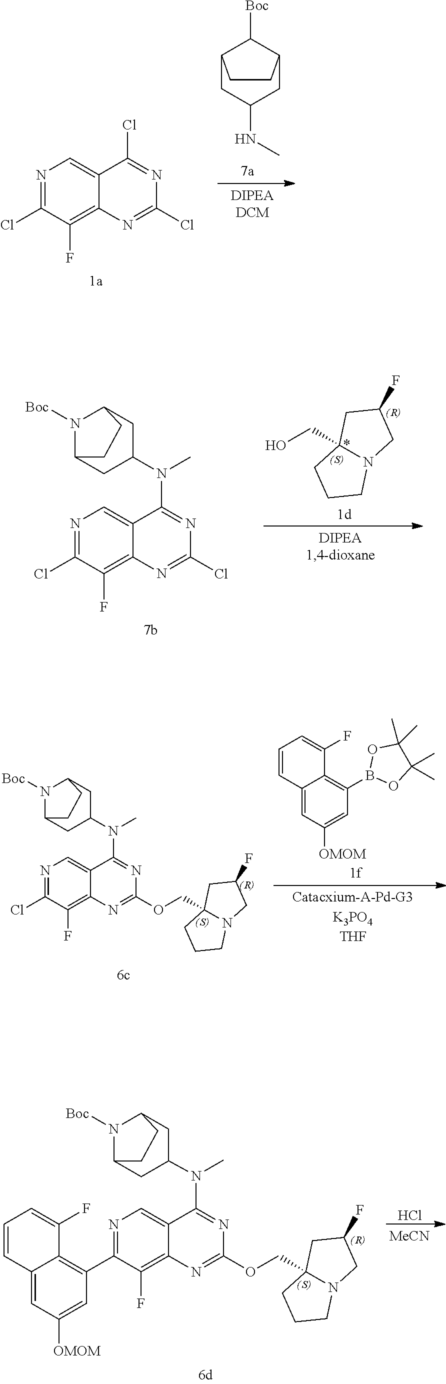

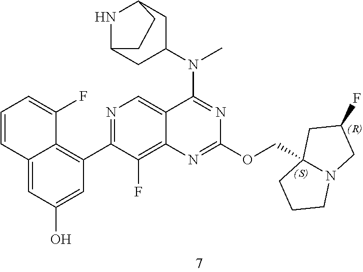

Compound 7 was synthesized according to the method of 1 to give a white solid 7 (4.19 mg). ¹H NMR (500 MHz, CD₃OD) δ 9.16 (s, 1H), 7.59 (d, J=8.3 Hz, 1H), 7.42-7.36 (m, 1H), 7.33 (t, J=2.0 Hz, 1H), 7.16 (d, J=2.3 Hz, 1H), 6.94-6.88 (m, 1H), 5.41-5.24 (m, 1H), 5.22-5.10 (m, 1H), 4.32 (dd, J=31.5, 10.6 Hz, 2H), 4.00-3.91 (m, 2H), 3.46 (s, 3H), 3.37-3.32 (m, 1H) 3.28-3.20 (m, 2H), 3.11-3.00 (m, 1H), 2.68-2.56 (m, 2H), 2.39-1.83 (m, 12H) ppm. MS m/z 605.6 [M+H]⁺.

### Example 8: Preparation of Compound 8

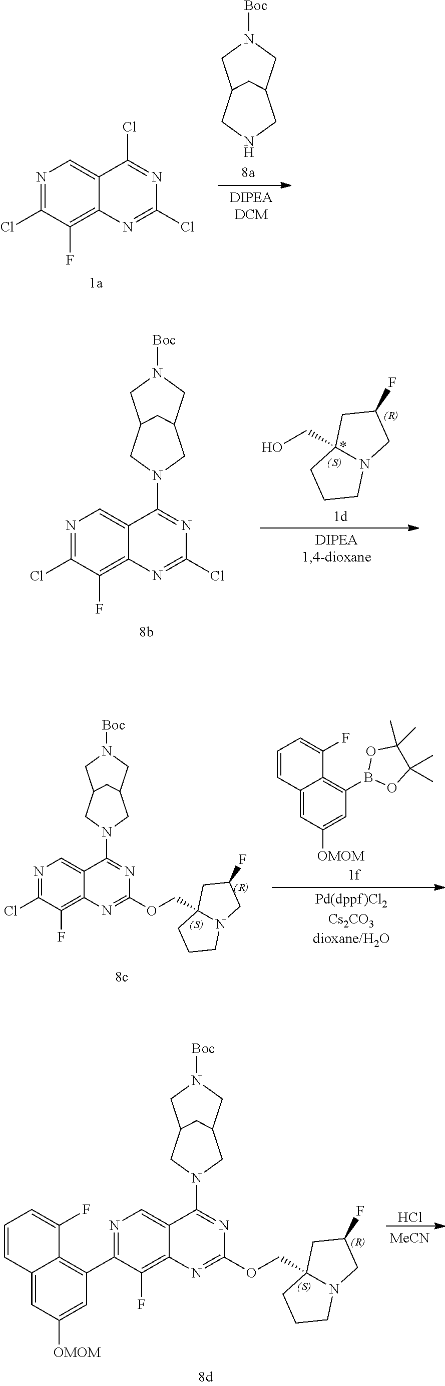

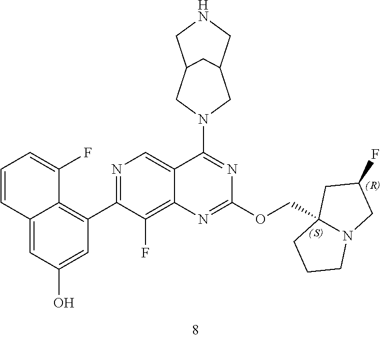

Compound 8c was synthesized according to the method of 1e

Compound 8c (30 mg, 0.05 mmol), lf (25 mg, 0.07 mmol),[1,1′-bis(diphenylphosphine) ferrocene]palladium dichloride (8 mg, 0.01 mmol) and Cs₂CO₃ (52 mg, 0.16 mmol) were dissolved in dioxane/water (1.2/0.4 mL), purged with N₂ for 1 min. Then the reaction mixture was stirred at 90° C. overnight under sealed tube. After the reaction was completed, the mixture was cooled to RT, water was added. Then the mixture was extracted with ethyl acetate (3×10 mL), and the collected organic phase was washed with sat.NaCl, dried with anhydrous sodium sulfate, filterated and concentrated. The crude product was purified by column chromatography (dichloromethane: methanol=30:1) to afford colorless oil 8d (9 mg, yield: 23%). MS m/z 735.9 [M+H]⁺.

Compound 8 was synthesized according to the method of 5 (2.91 mg). ¹H NMR (500 MHz, CD₃OD) δ 9.16 (s, 1H), 7.59 (d, J=8.3 Hz, 1H), 7.42-7.36 (m, 1H), 7.33 (t, J=2.1 Hz, 1H), 7.16 (d, J=2.4 Hz, 1H), 6.95-6.89 (m, 1H), 5.39-5.24 (m, 1H), 4.87-4.78 (m, 2H), 4.37-4.25 (m, 2H), 3.89-3.77 (m, 2H), 3.36-3.32 (m, 2H), 3.29-3.20 (m, 3H), 3.18-3.12 (m, 2H), 3.06-3.00 (m, 1H), 2.38-2.28 (m, 1H), 2.27-2.07 (m, 6H), 2.04-1.97 (m, 2H), 1.95-1.85 (m, 1H) ppm. MS m/z 591.6 [M+H]⁺.

### Example 9: Preparation of Compound 9

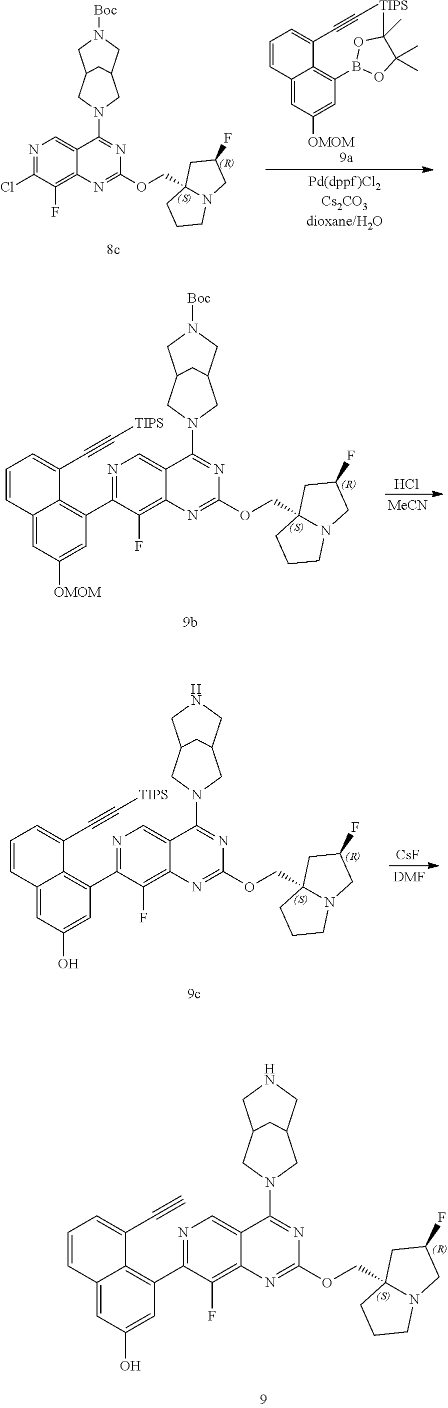

Compound 9a was synthesized according to WO2021041671.Compound 9b was synthesized according to the method of 8d

The compound 9b (14 mg, 0.015 mmol) was dissolved in acetonitrile (2 mL). Then 4 M HCl in dioxane (0.3 mL) was added. The reaction mixture was stirred at 0° C. for 1 h. After the reaction was completed, the mixture was concentrated to afford yellow solid 9c (19 mg, yield: 1000%). MS m/z 753.8 [M+H].

The compound 9c (19 mg, 0.025 mmol) was dissolved in DMF (0.5 mL). Then Cesium Fluoride (38.2 mg, 0.25 mmol) was added. Then water was added and extracted with ethyl acetate (3×5 mL), and the collected organic phase was washed with sat.NaCl, dried with anhydrous sodium sulfate, filterated and concentrated to give the crude product. The crude product was purified by Pre-TLC (dichloromethane: methanol=10:1) to afford yellow solid9 (1.02 mg). ¹H NMR (500 MHz, CD₃OD) δ 9.11 (s, 1H), 7.82 (d, J=7.6 Hz, 1H), 7.50 (d, J=6.1 Hz, 1H), 7.41-7.37 (m, 1H), 7.33 (d, J=2.5 Hz, 1H), 7.15 (d, J=2.5 Hz, 1H), 5.39-5.23 (m, 1H), 4.85-4.75 (m, 2H), 4.36-4.23 (m, 2H), 3.88-3.77 (m, 2H), 3.36-3.33 (m, 1H), 3.29-3.20 (m, 4H), 3.18-3.10 (m, 2H), 3.07-2.99 (m, 2H), 2.37-2.20 (m, 2H), 2.16-1.96 (i, 7H), 1.94-1.86 (m, 1H) ppm. MS m/z 597.6 [M+H]⁺.

### Example 10: Preparation of Compound 10, l0S, 10R

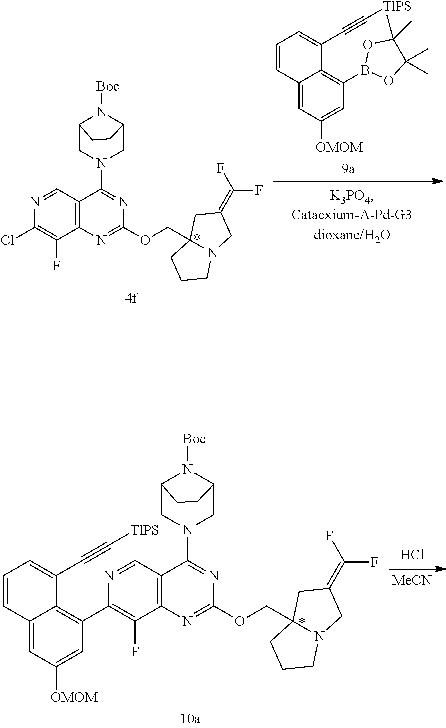

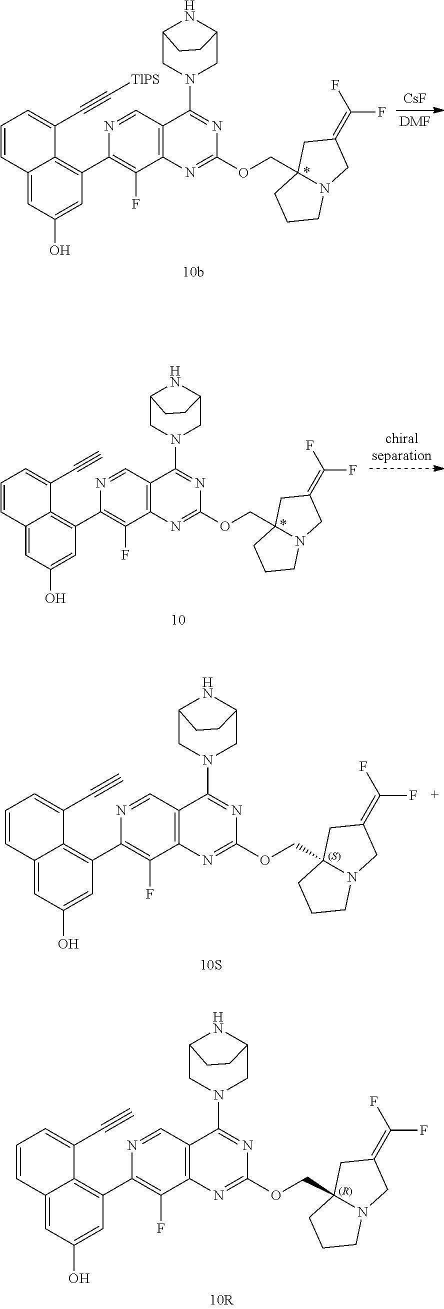

Compound 4f (18 mg, 0.03 mmol), potassium phosphate (19 mg, 0.09 mmol), Methylsulfonic acid [n-butyldi (1-adamantinyl) phosphine](2-amino-1,1′-biphenyl-2-yl) palladium (II) (2.1 mg, 0.003 mmol), 9a (30 mg, 0.06 mmol) were added to the reaction bottle, purged with N₂ for 3 times. Then dioxane/water (0.5 mL/0.1 mL) was added. Then the reaction mixture was stirred at 100° C. for 2 h under N₂. After the reaction was cooled, water was added and extracted with ethyl acetate (3×10 mL), and the collected organic phase was washed with sat.NaCl, dried with anhydrous sodium sulfate filterated and concentrated to give the crude product. The crude product was purified by column chromatography (dichloromethane: methanol=25:1) to afford yellow oil 10a (21 mg, yield: 75%). MS m/z 913.9 [M+H]⁺.

The compound 10a(21 mg, 0.02 mmol) was dissolved in acetonitrile (1 mL). Then 4 M HCl in dioxane (0.3 mL) was added. The reaction mixture was stirred at RT for 1 h. After the reaction was completed, the mixture was concentrated to afford yellow solid 10b (18 mg, yield: 100%). MS m/z 768.8 [M+H]⁺.

The compound 10b (18 mg, 0.02 mmol) was dissolved in DMF (0.5 mL). Then Cesium Fluoride (30 mg, 0.20 mmol) was added. The mixture was stirred for 2 h. Then water was added and extracted with ethyl acetate (3×5 mL), and the collected organic phase was washed with sat.NaCl, dried with anhydrous sodium sulfate, filterated and concentrated to give the crude product. The crude product was purified by Pre-TLC (dichloromethane: methanol=10:1) to afford yellow solid10 (5 mg, racemate). ¹H NMR (500 MHz, CD₃OD) δ 9.00 (s, 1H), 7.83-7.79 (m, 1H), 7.50 (dd, J=7.1, 1.1 Hz, 1H), 7.40-7.37 (m, 1H), 7.32 (d, J=2.5 Hz, 1H), 7.15 (d, J=2.5 Hz, 1H), 4.69-4.55 (m, 2H), 4.38-4.25 (m, 2H), 3.83-3.77 (m, 1H), 3.76-3.63 (m, 4H), 3.47-3.41 (m, 1H), 3.19-3.12 (m, 1H), 3.02 (s, 1H), 2.82-2.77 (m, 1H), 2.74-2.67 (m, 1H), 2.55-2.48 (m, 1H), 2.1-2.09 (m, 1H), 2.04-1.96 (m, 1H), 1.94-1.79 (m, 6H) ppm. MS m/z 613.7 [M+H]⁺. Compound 10 (racemate) is separated by chiral column to obtain compounds 10S and 10R

### Example 11: Preparation of Compound 11, 11S, 11R

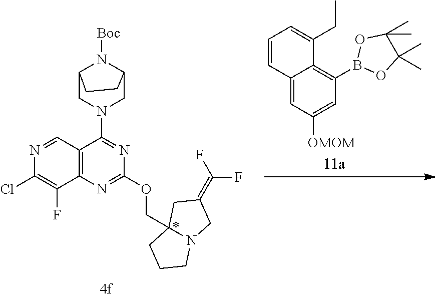

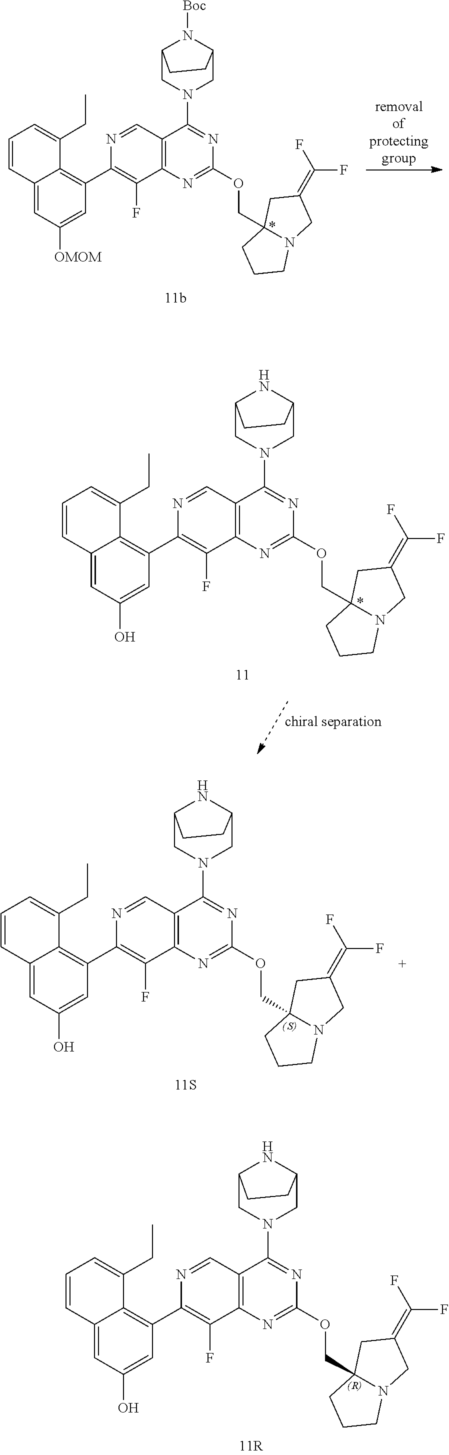

Compound 11, 11S, 11R was synthesized through compound 4f and 11a (patent WO2021041671) according to the method of 4

### Example 12: Preparation of Compound 12, 12S, 12R

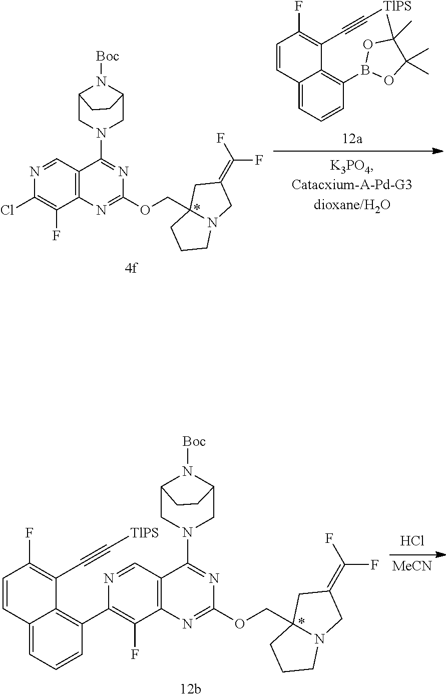

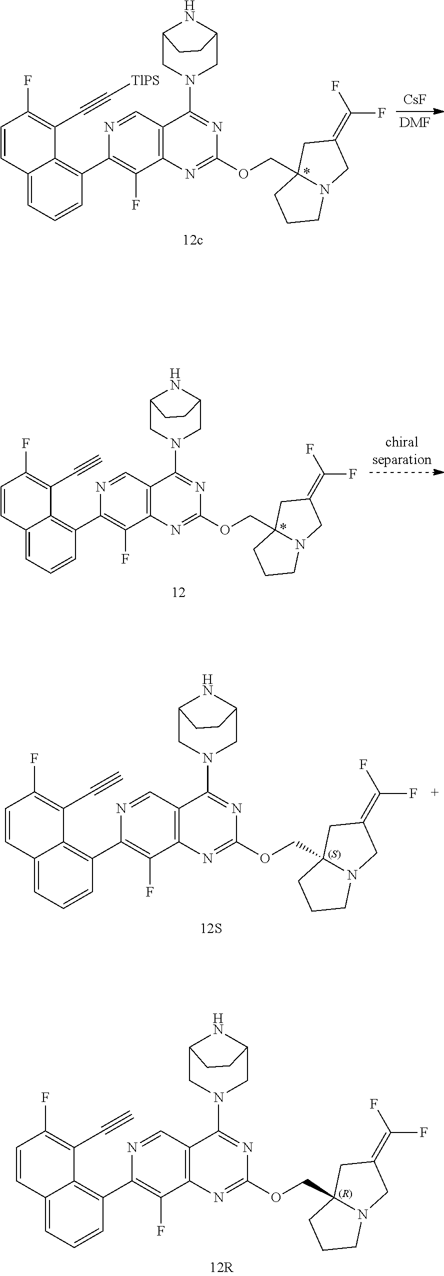

Compound 12a was synthesized according to WO2021041671.

Compound 4f (16 mg, 0.03 mmol), potassium phosphate (19 mg, 0.09 mmol), Methylsulfonic acid [n-butyldi (1-adamantinyl) phosphine](2-amino-1,1′-biphenyl-2-yl) palladium (II) (2.1 mg, 0.003 mmol), 12a (27 mg, 0.06 mmol) were added to the reaction bottle, purged with N₂ for 3 times. Then dioxane/water (0.5 mL/0.1 mL) was added. The reaction mixture was stirred at 100° C. for 2 h under N₂. After the reaction was completed, the mixture was cooled to RT. Water was added and extracted with ethyl acetate (3×10 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 12b (19 mg, yield: 70%). MS m/z 871.9 [M+H]⁺.

The compound 12b (19 mg, 0.02 mmol) was dissolved in acetonitrile (1 mL). Then 4 M HCl in dioxane (0.3 mL) was added. The reaction mixture was stirred at RT for 1 h. After the reaction was completed, the mixture was concentrated to afford yellow solid 12c (17 mg, yield: 100%).

The compound 12c (17 mg) was dissolved in DMF (0.5 mL). Then Cesium Fluoride (33 mg, 0.22 mmol) was added. The mixture was stirred for 2 h at RT. Then water was added and extracted with ethyl acetate (3×5 mL), and the collected organic phase was washed with sat.NaCl, dried with anhydrous sodium sulfate, filterated and concentrated to give the crude product. The crude product was purified by Pre-TLC (dichloromethane: methanol=10:1) to afford white solid12 (5 mg, racemate). ¹H NMR (500 MHz, CD₃OD) δ 9.02 (s, 1H), 8.12-8.09 (m, 2H), 7.68-7.62 (m, 2H), 7.44 (t, J=8.5 Hz, 1H), 4.65-4.58 (m, 2H), 4.38-4.25 (m, 2H), 3.83-3.77 (m, 1H), 3.75-3.63 (m, 4H), 3.45-3.40 (m, 2H), 3.17-3.10 (m, 1H), 2.83-2.76 (m, 1H), 2.71-2.67 (m, 1H), 2.54-2.48 (m, 1H), 2.14-2.09 (m, 1H), 2.03-1.95 (m, 1H), 1.94-1.84 (m, 4H), 1.83-1.78 (m, 2H) ppm. MS m/z 615.6 [M+H]⁺. Compound 12 (racemate) is separated by chiral column to obtain compounds 12S and 12R

### Example 13: Preparation of Compound 13, 13S, 13R

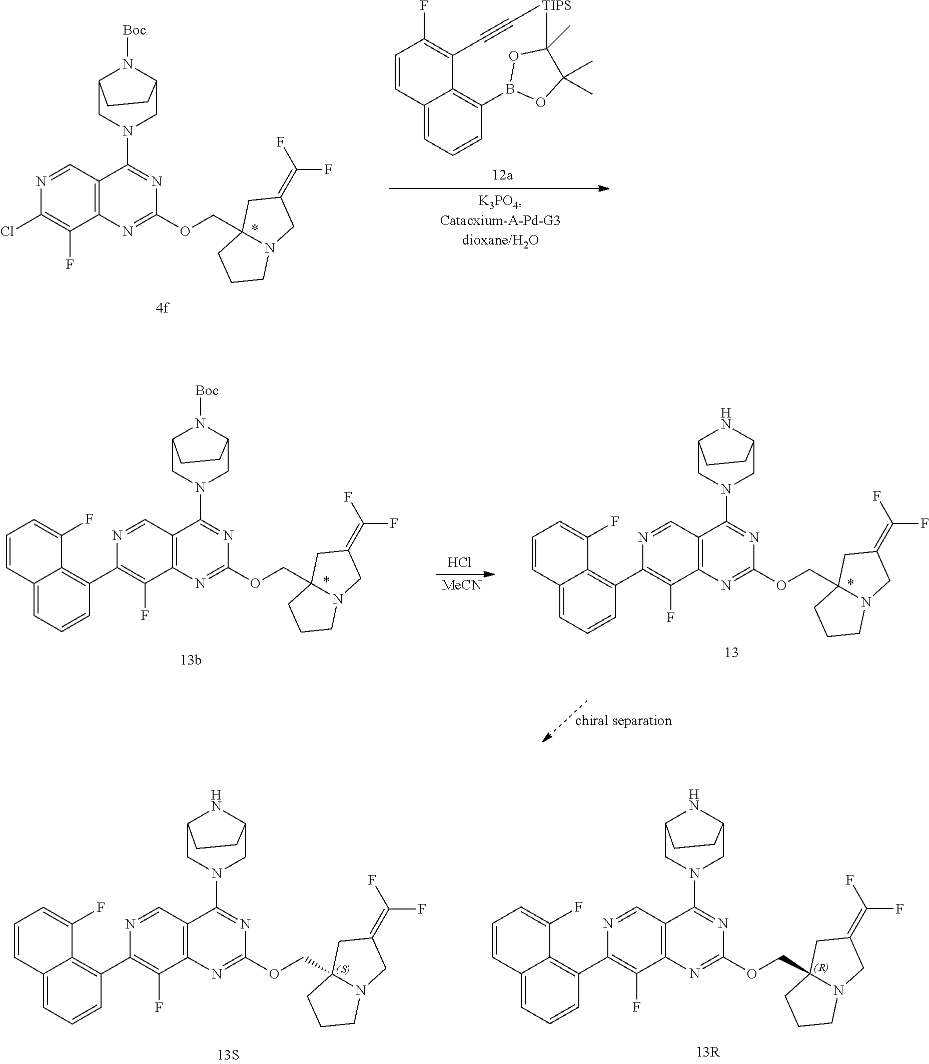

Compound 13a was synthesized according to WO2021041671.

Compound 4f (31 mg, 0.05 mmol), potassium phosphate (32 mg, 0.15 mmol), Methylsulfonic acid [n-butyldi (1-adamantinyl) phosphine](2-amino-1,1′-biphenyl-2-yl) palladium (II) (3.5 mg, 0.005 mmol), 13a (22 mg, 0.08 mmol) were added to the reaction bottle, purged with N₂ for 3 times. Then dioxane/water (0.5 mL/0.1 mL) was added. The reaction mixture was stirred at 60° C. for 1 h under N₂. After the reaction was completed, the mixture was cooled to RT. Water was added and extracted with ethyl acetate (3×10 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 13b (5.2 mg, yield: 14%). MS m/z 691.7 [M+H]⁺.

The compound 13b(5.2 mg, 0.007 mmol) was dissolved in acetonitrile (1 mL). Then 4 M HCl in dioxane (0.2 mL) was added. The reaction mixture was stirred at RT for 0.5 h. After the reaction was completed, the mixture was concentrated. The crude product was purified by column chromatography (dichloromethane: methanol=15:1) to afford off-white solid 13 (4.1 mg, yield: 92%, racemate). LCMS: MS m/z 591.6 [M+H]+, purity: 100% (254 nm). Compound 13 (racemate) is separated by chiral column to obtain compounds 13S and 13R

### Example 14: Preparation of Compound 14, 14S, 14R

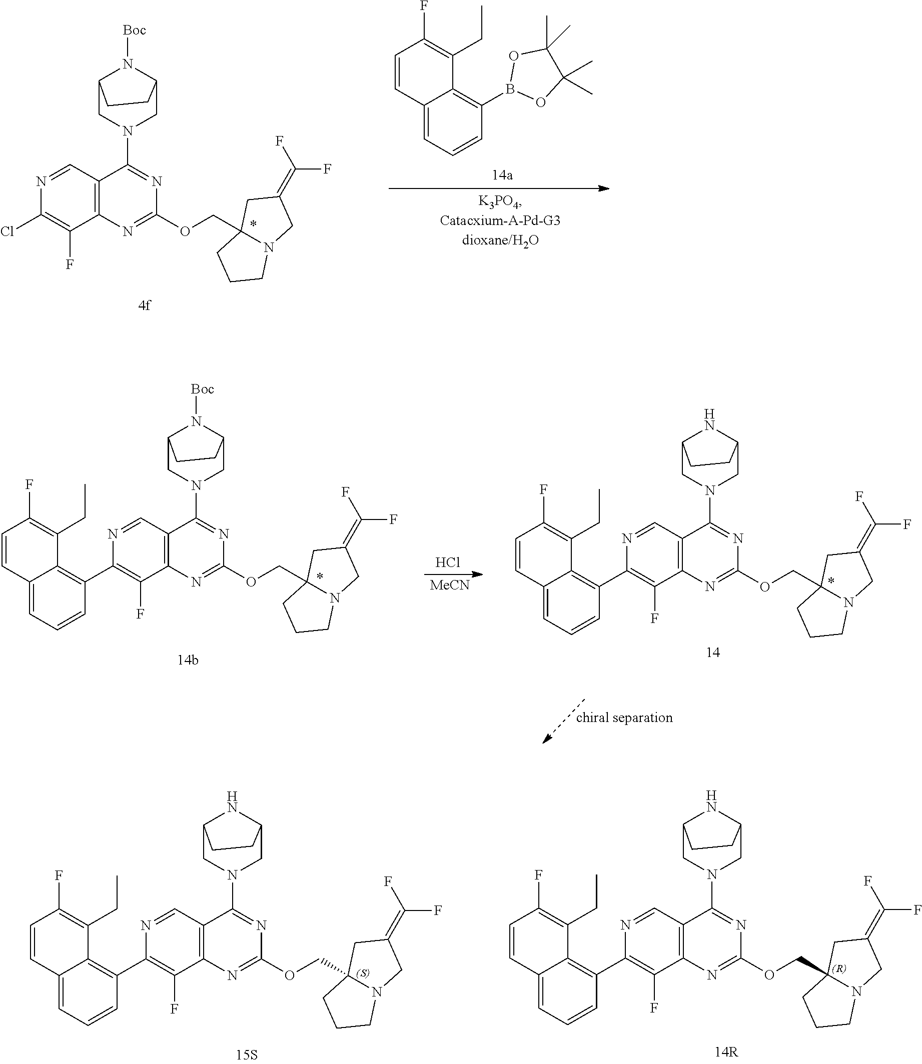

Compound 14a was synthesized according to WO2021041671.

Compound 4f (16 mg, 0.03 mmol), potassium phosphate (19 mg, 0.09 mmol), Methylsulfonic acid [n-butyldi (1-adamantinyl) phosphine](2-amino-1,1′-biphenyl-2-yl) palladium (II) (2.1 mg, 0.003 mmol), 14a (15 mg, 0.05 mmol) were added to the reaction bottle, purged with N₂ for 3 times. Then dioxane/water (0.5 mL/0.1 mL) was added. The reaction mixture was stirred at 100° C. for 2 h under N₂. After the reaction was completed, the mixture was cooled to RT. Water was added and extracted with ethyl acetate (3×10 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 14b (12 mg, yield: 60%). MS m/z 719.8 [M+H]⁺.

The compound 14b (12 mg, 0.017 mmol) was dissolved in acetonitrile (1 mL). Then 4 M HCl in dioxane (0.3 mL) was added. The reaction mixture was stirred at RT for 0.5 h. After the reaction was completed, the mixture was concentrated. The crude product was purified by column chromatography (dichloromethane: methanol=15:1) to afford off-white solid 14 (7 mg, yield: 70%, racemate). ¹H NMR (500 MHz, CD₃OD) δ 9.07 (s, 1H), 8.05 (dd, J=8.1, 1.1 Hz, 1H), 7.92 (dd, J=9.0, 6.0 Hz, 1H), 7.57-7.52 (m, 1H), 7.46 (d, J=6.9 Hz, 1H), 7.36 (t, J=9.4 Hz, 1H), 4.67-4.59 (m, 2H), 4.39-4.27 (m, 2H), 3.83-3.77 (m, 1H), 3.76-3.66 (m, 4H), 3.45-3.40 (m, 1H), 3.17-3.12 (m, 1H), 2.83-2.77 (m, 1H), 2.73-2.67 (m, 1H), 2.59-2.49 (m, 2H), 2.30-2.21 (m, 1H), 2.16-2.10 (m, 1H), 2.04-1.96 (m, 1H), 1.94-1.77 (m, 6H), 0.82 (t, J=7.4 Hz, 3H) ppm. MS m/z 619.7 [M+H]⁺. Compound 14 (racemate) is separated by chiral column to obtain compounds 14S and 14R

### Example 15: Preparation of Compound 15

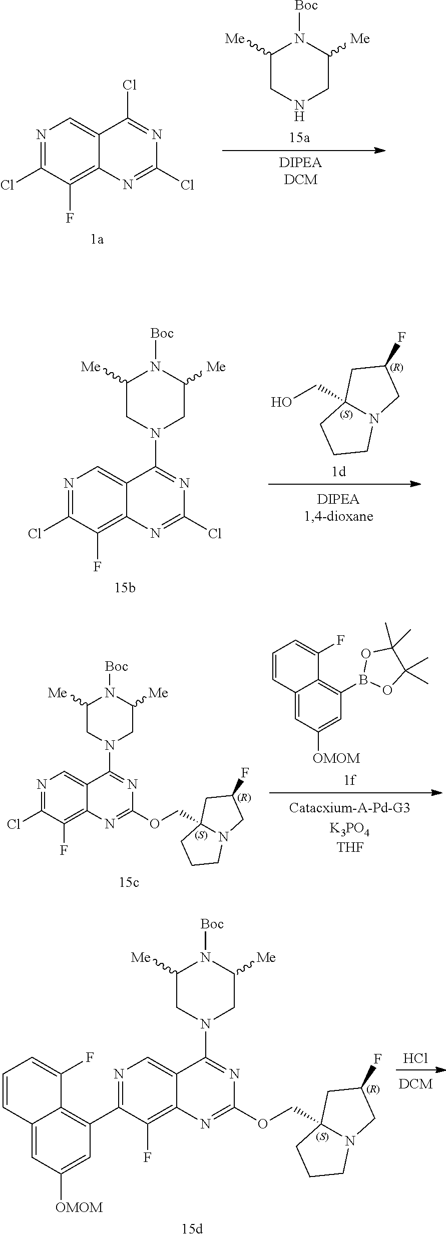

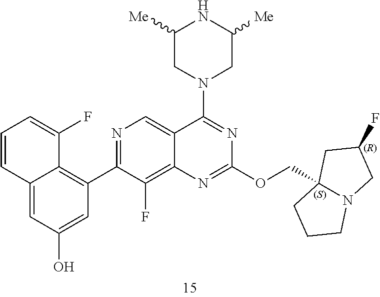

Compound 15 was synthesized according to the method of 1 to give a white solid 15 (8.5 mg). ¹H NMR (500 MHz, CD₃OD) δ 9.03 (s, 1H), 7.59 (d, J=8.4 Hz, 1H), 7.44-7.34 (m, 1H), 7.34-7.30 (m, 1H), 7.18-7.14 (m, 1H), 6.93-6.89 (m, 1H), 5.39-5.24 (m, 1H), 4.64-4.55 (m, 2H), 4.34-4.25 (m, 2H), 3.36-3.33 (m, 1H), 3.29-3.22 (m, 3H), 3.10-2.98 (m, 4H), 2.40-2.12 (m, 3H), 2.06-1.96 (m, 2H), 1.95-1.86 (m, 1H), 1.20 (d, J=5.0 Hz, 6H) ppm. MS m/z 579.7 [M+H]⁺.

### Example 16: Preparation of Compound 16

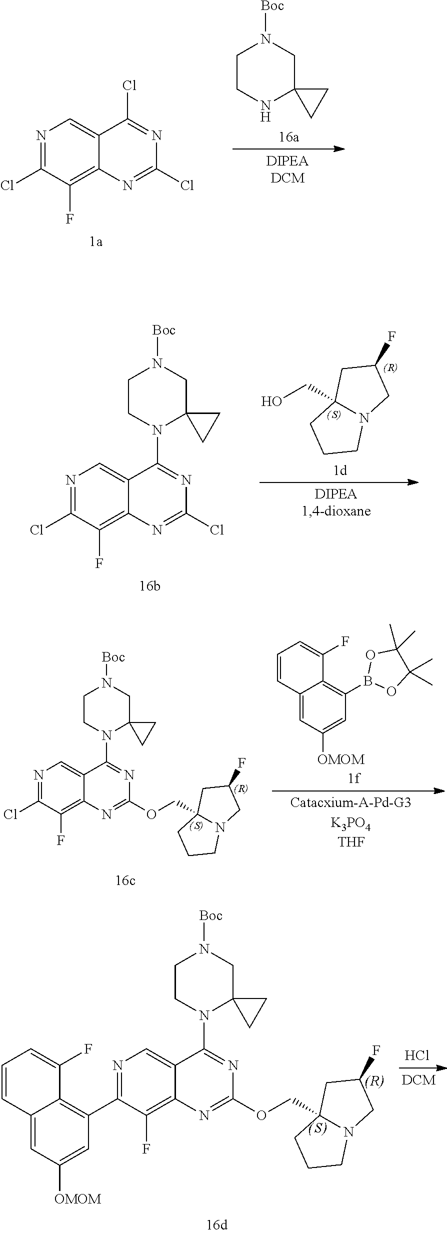

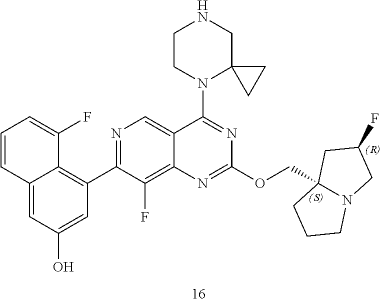

Compound 16 was synthesized according to the method of 1 to give a white solid 15 (5.86 mg). ¹H NMR (500 MHz, CD₃OD) δ 9.12 (s, 1H), 7.59 (d, J=8.2 Hz, 1H), 7.42-7.35 (m, 1H), 7.34-7.31 (in, 1H), 7.17 (s, 1H), 6.92 (dd, J=13.1, 7.6 Hz, 1H), 5.40-5.20 (in, 1H), 4.38-4.23 (m, 2H), 3.35-3.32 (m, 3H), 3.29-3.16 (m, 4H), 3.10-3.00 (m, 3H), 2.39-2.10 (m, 3H), 2.05-1.96 (m, 2H), 1.96-1.82 (m, 1H), 1.23-1.17 (m, 2H), 1.09-1.00 (in, 2H) ppm. MS m/z 577.6 [M+H]⁺.

### Example 17: Preparation of Compound 17

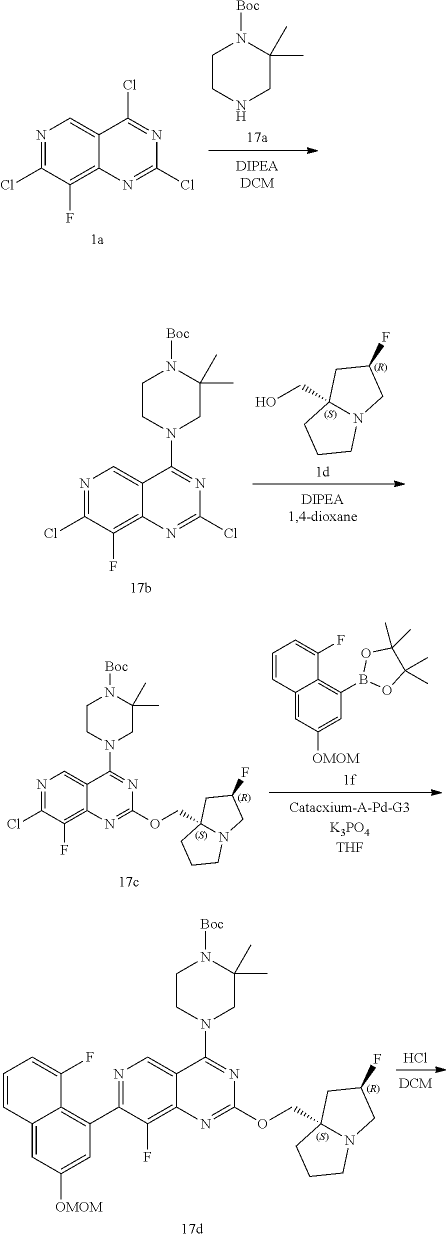

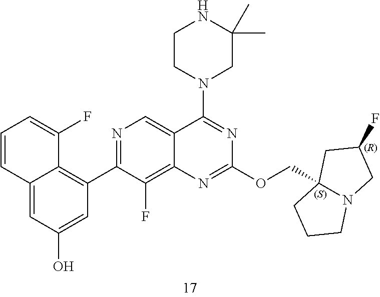

Compound 17 was synthesized according to the method of 1 to give a white solid 17 (9.96 mg). ¹H NMR (500 MHz, CD₃OD) δ 9.07 (s, 1H), 7.59 (d, J=8.2 Hz, 1H), 7.42-7.36 (m, 1H), 7.34-7.31 (m, 1H), 7.17 (d, J=2.4 Hz, 1H), 6.92 (dd, J=13.1, 7.6 Hz, 1H), 5.43-5.23 (m, 1H), 4.38-4.25 (m, 2H), 4.07-4.01 (m, 2H), 3.96-3.86 (m, 2H), 3.38-3.33 (m, 1H), 3.28-3.23 (m, 2H), 3.17-3.12 (m, 2H), 3.09-3.02 (m, 1H), 2.42-2.11 (m, 3H), 2.07-1.97 (m, 2H), 1.96-1.86 (m, 1H), 1.26 (d, J=4.3 Hz, 6H) ppm. MS m/z 579.6 [M+H]⁺.

### Example 18: Preparation of Compound 18, 18S, 18R

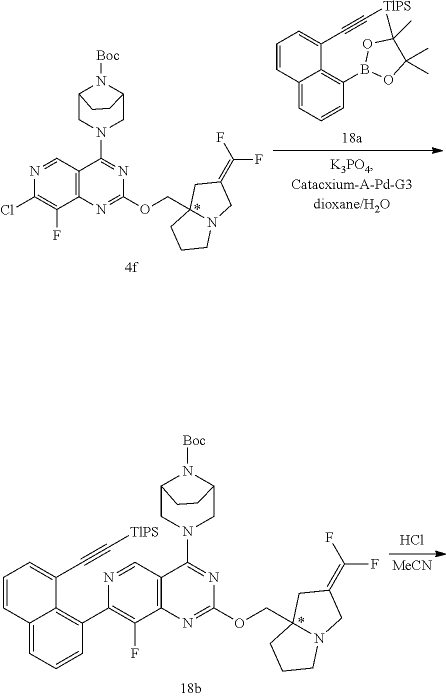

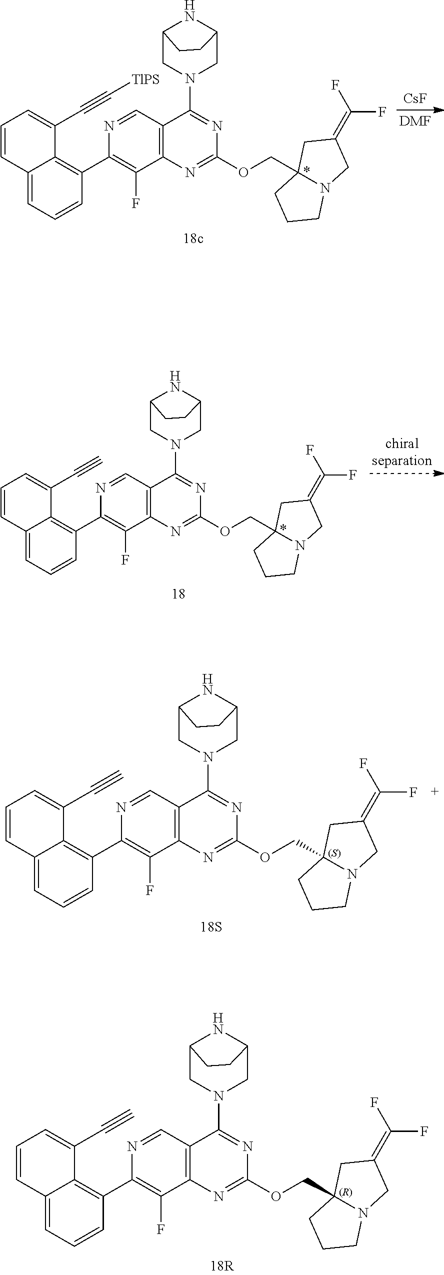

Compound 4f (16 mg, 0.03 mmol), potassium phosphate (19 mg, 0.09 mmol), Methylsulfonic acid [n-butyldi (1-adamantinyl) phosphine](2-amino-1,1′-biphenyl-2-yl) palladium (II) (2.1 mg, 0.003 mmol), 18a (24 mg, 0.06 mmol) were added to the reaction bottle, purged with N₂ for 3 times. Then dioxane/water (0.5 mL/0.1 mL) was added. The reaction mixture was stirred at 100° C. for 2 h under N₂. After the reaction was completed, the mixture was cooled to RT. Water was added and extracted with ethyl acetate (3×10 mL). The collected organic phase was washed with sat. NaCl, dried with anhydrous sodium sulfate filterated and concentrated to give the crude product. The crude product was purified by column chromatography (dichloromethane: methanol=25:1) to afford yellow oil 18b (15 mg, yield: 64%). MS m/z 853.9 [M+H]⁺.

The compound 18b (15 mg, 0.02 mmol) was dissolved in acetonitrile (1 mL). Then 4 M HCl in dioxane (0.3 mL) was added. The reaction mixture was stirred at RT for 1 h. After the reaction was completed, the mixture was concentrated to afford yellow solid 18c (13 mg, yield: 100%).

The compound 18c (13 mg) was dissolved in DMF (0.5 mL). Then Cesium Fluoride (26 mg, 0.17 mmol) was added. The mixture was stirred for 2 h at RT. Then water was added and extracted with ethyl acetate (3×5 mL), and the collected organic phase was washed with sat.NaCl, dried with anhydrous sodium sulfate, filterated and concentrated to give the crude product. The crude product was purified by Pre-TLC (dichloromethane: methanol=10:1) to afford white solid18 (5 mg, 54% yield, racemate). ¹H NMR (500 MHz, CD₃OD) δ 9.02 (s, 1H), 8.10 (dd, J=8.3, 1.2 Hz, 1H), 8.08-8.04 (dd, J=8.3, 1.2 Hz, 1H), 7.74 (dd, J=7.2, 1.2 Hz, 1H), 7.69-7.65 (m, 1H), 7.59 (dd, J=7.1, 1.2 Hz, 1H), 7.55-7.50 (m, 1H), 4.71-4.59 (m, 2H), 4.41-4.35 (m, 1H), 4.31-4.26 (m, 1H), 3.85-3.73 (m, 5H), 3.49-3.43 (m, 1H), 3.20-3.15 (m, 1H), 3.10 (s, 1H), 2.83-2.78 (m, 1H), 2.76-2.69 (m, 1H), 2.56-2.49 (m, 1H), 2.16-2.10 (m, 1H), 2.04-1.97 (m, 1H), 1.97-1.84 (m, 6H). MS m/z 597.6 [M+H]⁺. Compound 18 (racemate) is separated by chiral column to obtain compounds 18S and 18R

### Example 19: Preparation of Compound 19, 19S, 19R

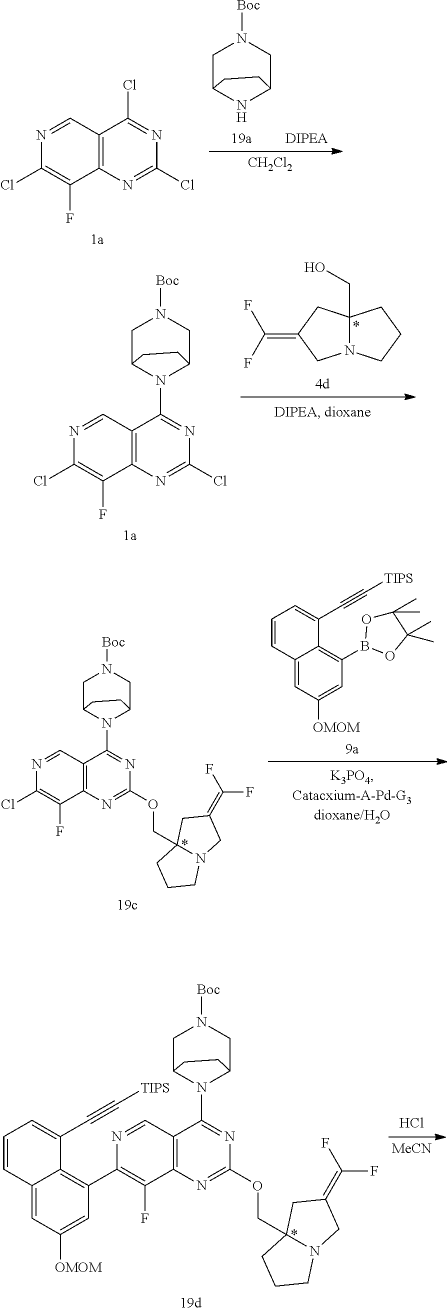

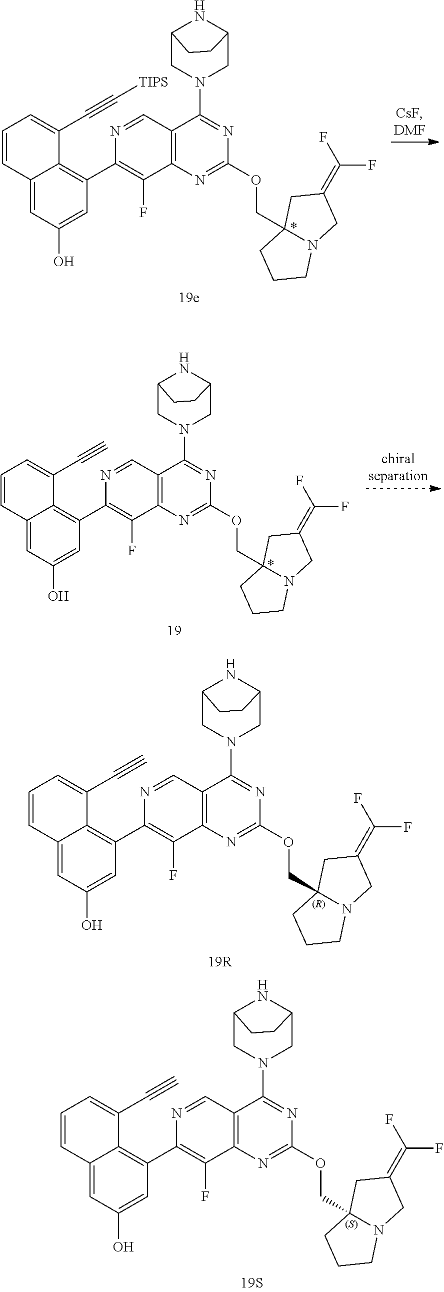

Compound 1a (150 mg, 0.59 mmol) and N,N-diisopropylethyl amine(384 mg, 2.97 mmol) were dissolved in dichloromethane (3 mL). 19a (126 mg, 0.59 mmol) in dichloromethane (1 mL) was added to the mixture under −40° C. Then the reaction mixture was stirred at −40° C. for 0.5 h. After the reaction was completed, water was added. Then the mixture was extracted with dichloromethane (3×15 mL), and the collected organic phase was washed with sat.NaCl, dried with anhydrous sodium sulfate, filterated and concentrated to give the crude product. The crude product was purified by column chromatography (dichloromethane:ethyl acetate=5:1) to afford a yellow solid 19b (142 mg, yield: 56%). MS m/z 428.3 [M+H]⁺.

The compound 19b (97 mg, 0.23 mmol), 4d (43 mg, 0.23 mmol) and N, N-diisopropylethylamine(89 mg, 0.69 mmol) were dissolved in dioxane (1 mL). Then the reaction mixture was stirred at 90° C. for 48 h under N₂. After the reaction was completed, the mixture was concentrated to give the crude product. The crude product was purified by column chromatography to afford a white solid 19c (40 mg, yield: 30%). MS m/z 4581.5, 583.4 [M+H]⁺.

Compound 19c (17 mg, 0.03 mmol), potassium phosphate (19 mg, 0.09 mmol), Methylsulfonic acid [n-butyldi (1-adamantinyl) phosphine](2-amino-1,1′-biphenyl-2-yl) palladium (II) (2.1 mg, 0.003 mmol), 9a (29 mg, 0.06 mmol) were added to the reaction bottle, purged with N₂ for 3 times. Then dioxane/water (0.5 mL/0.1 mL) was added. The reaction mixture was stirred at 100° C. for 2 h under N₂. After the reaction was completed, the mixture was cooled to RT. Water was added and extracted with ethyl acetate (3×10 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 19d (19 mg, yield: 71%). MS m/z 913.8 [M+H]⁺.

The compound 19d (19 mg, 0.02 mmol) was dissolved in acetonitrile (1 mL). Then 4 M HCl in dioxane (0.3 mL) was added. The reaction mixture was stirred at RT for 1 h. After the reaction was completed, the mixture was concentrated to afford yellow solid 19e (16 mg, yield: 100%). MS m/z 768.8 [M+H]⁺.

The compound 19e (16 mg) was dissolved in DMF (0.5 mL). Then Cesium Fluoride (30 mg, 0.20 mmol) was added. The mixture was stirred for 2 h at RT. Then water was added and extracted with ethyl acetate (3×5 mL), and the collected organic phase was washed with sat.NaCl, dried with anhydrous sodium sulfate, filterated and concentrated to give the crude product. The crude product was purified by Pre-TLC (dichloromethane: methanol=10:1) to afford yellow solid19 (5 mg, 39% yield, racemate). ¹H NMR (500 MHz, CD₃OD) δ 9.04 (s, 1H), 7.82 (dd, J=8.0, 0.8 Hz, 1H), 7.51 (dd, J=7.1, 1.1 Hz, 1H), 7.41-7.37 (m, 1H), 7.32 (d, J=2.5 Hz, 1H), 7.16 (d, J=2.5 Hz, 1H), 5.19-5.05 (m, 2H), 4.37-4.24 (m, 2H), 3.82-3.79 (m, 1H), 3.47-3.42 (m, 1H), 3.28-3.19 (m, 2H), 3.18-3.12 (m, 1H), 3.08 (s, 1H), 2.98-2.92 (m, 2H), 2.84-2.78 (m, 1H), 2.74-2.69 (m, 1H), 2.56-2.48 (m, 1H), 2.16-2.10 (m, 4H), 2.05-1.94 (m, 2H), 1.95-1.86 (m, 2H). MS m/z 613.7 [M+H]⁺. Compound 19 (racemate) is separated by chiral column to obtain compounds 19S and 19R

### Example 20: Preparation of Compound 20, 20S, 20R

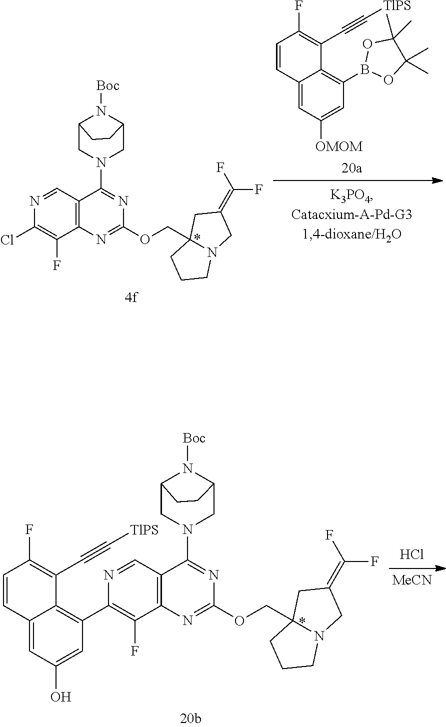

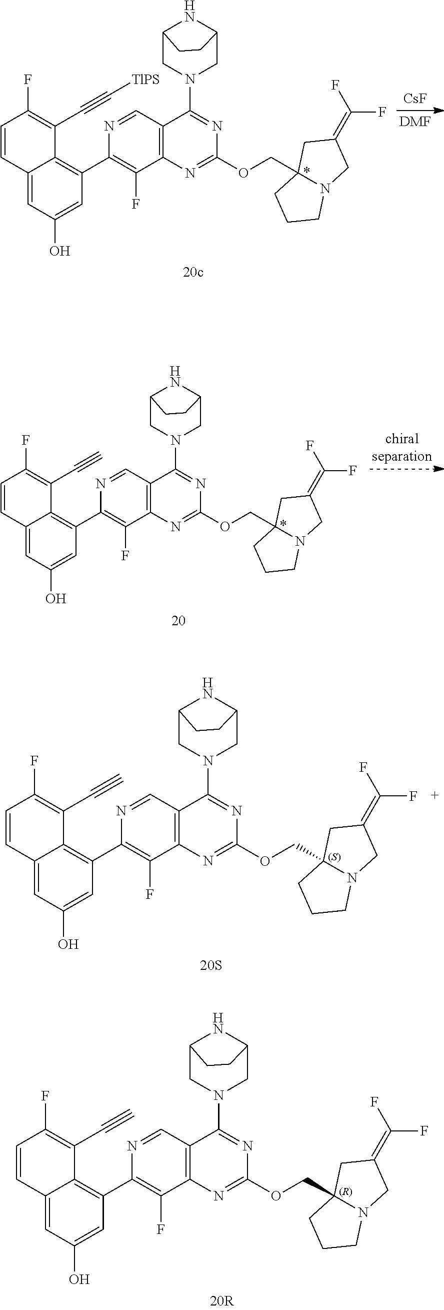

Compound 4f (307 mg, 0.53 mmol), potassium phosphate (337 mg, 1.59 mmol), Methylsulfonic acid [n-butyldi (1-adamantinyl) phosphine](2-amino-1,1′-biphenyl-2-yl) palladium (II) (38 mg, 0.05 mmol), 20a (406 mg, 0.79 mmol, synthesized according to WO2021041671 or purchased) were added to the reaction bottle, purged with N₂ for 3 times. Then dioxane/water (5 mL/1 mL) was added. The reaction mixture was stirred at 100° C. for 2 h under N₂. After the reaction was completed, the mixture was cooled to RT. Water was added and extracted with ethyl acetate (3×30 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 20b (486 mg, yield: 99%). MS m/z 931.9 [M+H]⁺.

The compound 20b (486 mg, 0.52 mmol) was dissolved in acetonitrile (10 mL). Then 4 M HCl in dioxane (3 mL) was added. The reaction mixture was stirred at RT for 1 h. After the reaction was completed, the mixture was concentrated to afford yellow solid 20c (411 mg, yield: 100%).

The compound 20c (411 mg, 0.52 mmol) was dissolved in DMF (10 mL). Then Cesium Fluoride (790 mg, 5.20 mmol) was added. The mixture was stirred overnight at RT. Then water was added and extracted with ethyl acetate, and the collected organic phase was washed with sat.NaCl, dried with anhydrous sodium sulfate, filterated and concentrated to give the crude product. The crude product was purified by column chromatography (dichloromethane: methanol=10:1) to afford white solid20 (174 mg, 53% yield). Compound 20 was subjected to HPLC using chiral columns (CHIRALPAK AD-H, ADH0CE-XG136, 0.46 cm I.D×25 cm L; Hexane/EtOH/DEA=60/40/0.1 (V/V/V); The first isomer obtained was compound 20-P1 (rt=4.93 min, 41 mg) and the second isomer was compound 20-P2 (rt=6.18 min, 43 mg)

Compound 20-P1 and 20-P2:one is 20S, the other is 20R

Compound 20-P1: ¹H NMR (500 MHz, CD₃OD) δ 9.02 (s, 1H), 7.86 (dd, J=9.1, 5.7 Hz, 1H), 7.35 (d, J=2.5 Hz, 1H), 7.32 (t, J=8.9 Hz, 1H), 7.20 (d, J=2.5 Hz, 1H), 4.73-4.61 (m, 2H), 4.42-4.25 (m, 2H), 3.90-3.75 (m, 5H), 3.47 (d, J=14.0 Hz, 1H), 3.36 (s, 1H), 3.21-3.15 (m, 1H), 2.84-2.71 (m, 2H), 2.58-2.50 (m, 1H), 2.18-2.08 (m, 1H), 2.06-1.85 (m, 7H) ppm. MS m/z 631.2 [M+H]⁺.

Compound 20-P2: ¹H NMR (500 MHz, CD₃OD) δ 9.01 (s, 1H), 7.85 (dd, J=9.1, 5.7 Hz, 1H), 7.34 (d, J=2.5 Hz, 1H), 7.32 (t, J=9.0 Hz, 1H), 7.20 (d, J=2.5 Hz, 1H), 4.71-4.59 (m, 2H), 4.43-4.23 (m, 2H), 3.87-3.72 (m, 5H), 3.45 (d, J=14.0 Hz, 1H), 3.35 (s, 1H), 3.19-3.13 (m, 1H), 2.85-2.69 (m, 2H), 2.56-2.47 (m, 1H), 2.16-2.07 (m, 1H), 2.05-1.83 (m, 7H) ppm. MS m/z 631.2 [M+H]⁺.

### Example 21: Preparation of Compound 21

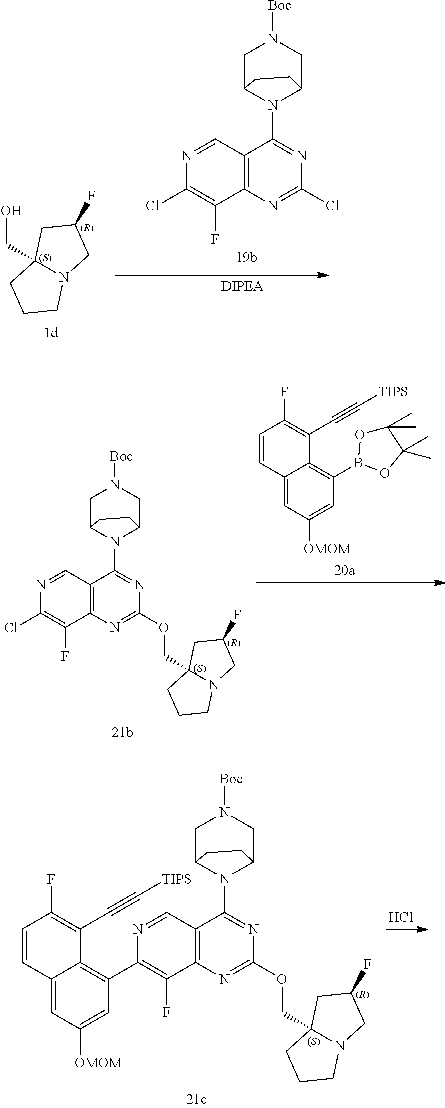

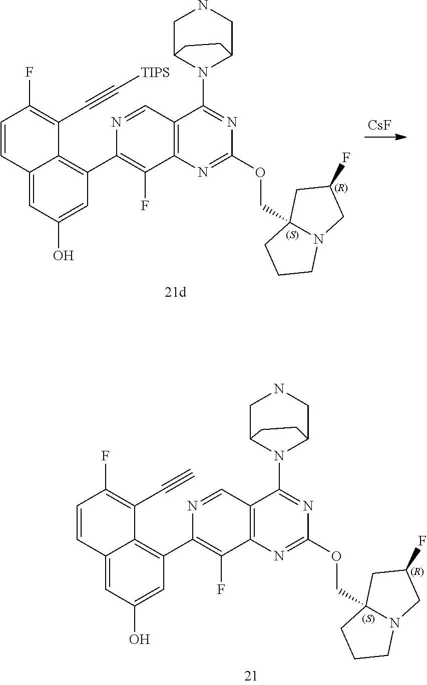

The compound 19b (150 mg, 0.35 mmol) and 1d (84 mg, 0.53 mmol) were dissolved in dioxane (2 mL). Then N, N-diisopropylethylamine(137 mg, 1.05 mmol) was added. The reaction mixture was stirred at 90° C. for 18 h under N₂. After the reaction was completed, the mixture was concentrated to give the crude product. The crude product was purified by column chromatography to afford a white solid 21b (120 mg, yield: 62%). MS m/z 551.2, 553.2 [M+H]⁺.

Compound 21b (38 mg, 0.07 mmol), potassium phosphate (44 mg, 0.21 mmol), Methylsulfonic acid [n-butyldi (1-adamantinyl) phosphine](2-amino-1,1′-biphenyl-2-yl) palladium (II) (4.2 mg, 0.007 mmol), 20a (53 mg, 0.10 mmol) were added to the reaction bottle, purged with N₂ for 3 times. Then dioxane/water (0.5 mL/0.1 mL) was added. The reaction mixture was stirred at 100° C. for 2 h under N₂. After the reaction was completed, the mixture was cooled to RT. Water was added and extracted with ethyl acetate (3×10 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 21c (60 mg, yield: 96%). MS m/z 901.5 [M+H]⁺.

The compound 21c (60 mg, 0.07 mmol) was dissolved in acetonitrile (1 mL). Then 4 M HCl in dioxane (0.3 mL) was added. The reaction mixture was stirred at RT for 1 h. After the reaction was completed, the mixture was concentrated to afford yellow solid 21d (50 mg, yield: 100%). MS m/z 757.5 [M+H]⁺.

The compound 21d (52 mg, 0.07 mmol) was dissolved in DMF (1 mL). Then Cesium Fluoride (96 mg, 0.7 mmol) was added. The mixture was stirred for 12 h at RT. Then the mixture was filterated and concentrated to give the crude product. The crude product was purified by column chromatography (dichloromethane: methanol=25:1) to afford yellow solid2l (18 mg, 48% yield). ¹H NMR (500 MHz, CD₃OD) δ 9.04 (s, 1H), 7.86 (dd, J=9.2, 5.7 Hz, 1H), 7.35 (d, J=2.5 Hz, 1H), 7.32 (t, J=8.9 Hz, 1H), 7.21 (d, J=2.5 Hz, 1H), 5.31 (d, J=54.1 Hz, 1H), 5.12 (d, J=25.4 Hz, 2H), 4.37-4.17 (m, 2H), 3.41 (d, J=7.2 Hz, 1H), 3.27-3.24 (m, 2H), 3.23-3.20 (m, 2H), 3.20-3.17 (m, 1H), 3.06-3.00 (m, 1H), 2.97-2.91 (m, 2H), 2.37-2.21 (m, 2H), 2.15-2.09 (m, 5H), 2.05-1.96 (m, 2H), 1.93-1.86 (m, 1H) ppm. MS m/z 601.1 [M+H]⁺.

### Example 22: Preparation of Compound 22

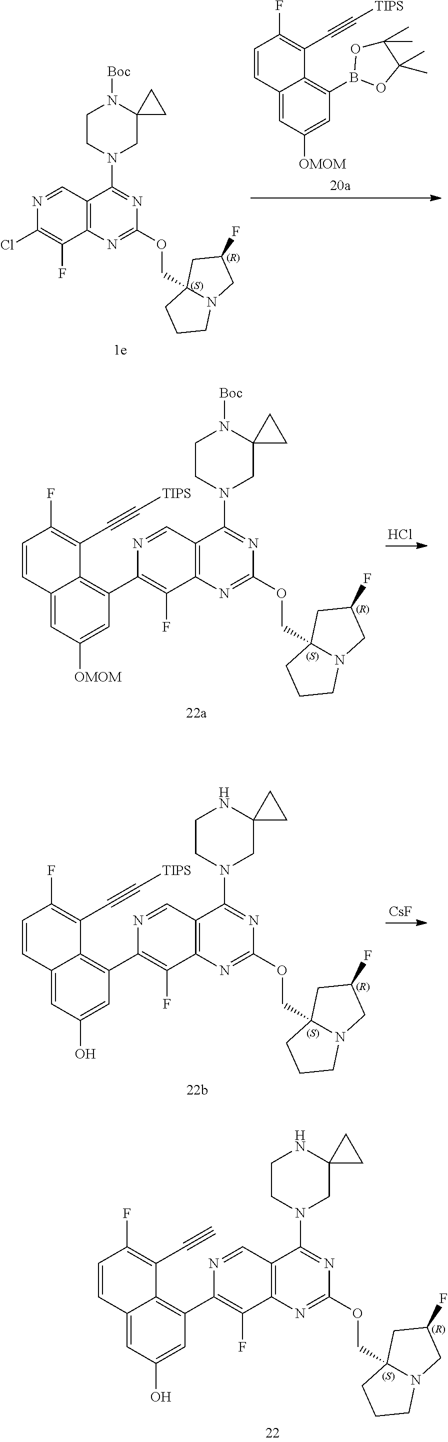

Compound 1e (25 mg, 0.05 mmol), potassium phosphate (32 mg, 0.15 mmol), Methylsulfonic acid [n-butyldi (1-adamantinyl) phosphine](2-amino-1,1′-biphenyl-2-yl) palladium (II) (3 mg, 0.005 mmol), 20a (35 mg, 0.07 mmol) were added to the reaction bottle, purged with N₂ for 3 times. Then dioxane/water (0.5 mL/0.1 mL) was added. The reaction mixture was stirred at 100° C. for 2 h under N₂. After the reaction was completed, the mixture was cooled to RT. Water was added and extracted with ethyl acetate (3×10 mL). The collected organic phase was washed with sat.NaCl, dried with anhydrous sodium sulfate filterated and concentrated to give the crude product. The crude product was purified by column chromatography (dichloromethane: methanol=25:1) to afford 2 yellow oil 2a (21 mg, yield: 51%). MS m/z 902.5 [M+H]⁺.

The compound 22a (22 mg, 0.02 mmol) was dissolved in acetonitrile (1 mL). Then 4 M HCl in dioxane (0.1 mL) was added. The reaction mixture was stirred at RT for 1 h. After the reaction was completed, the mixture was concentrated to afford yellow solid 22b (19 mg, yield: 100%). MS m/z 757.5 [M+H]⁺.

The compound 21b (20 mg, 0.02 mmol) was dissolved in DMF (1 mL). Then Cesium Fluoride (96 mg, 0.7 mmol) was added. The mixture was stirred for 12 h at RT. Then the mixture was filterated and concentrated to give the crude product. The crude product was purified by column chromatography (dichloromethane: methanol=25:1) to afford yellow solid22 (8 mg, 53% yield). MS m/z 601.2 [M+H]⁺. ¹H NMR (500 MHz, CD₃OD) δ 8.98 (s, 1H), 7.87-7.84 (m, 1H), 7.35 (d, J=2.5 Hz, 1H), 7.32 (t, J=9.0 Hz, 1H), 7.21 (d, J=2.5 Hz, 1H), 5.31 (d, J=53.9 Hz, 1H), 4.35-4.18 (m, 2H), 4.14-4.10 (m, 2H), 3.98 (s, 2H), 3.38 (d, J=7.3 Hz, 1H), 3.30-3.23 (m, 2H), 3.13 (t, J=5.1 Hz, 2H), 3.06-2.99 (m, 1H), 2.36-2.21 (m, 2H), 2.17-2.10 (m, 1H), 2.05-1.96 (m, 2H), 1.95-1.85 (m, 1H), 0.70 (s, 4H) ppm.

### Example 23: Preparation of Compound 23

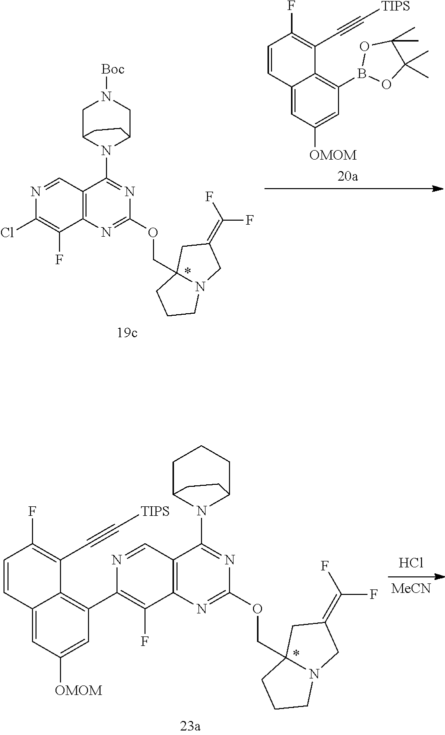

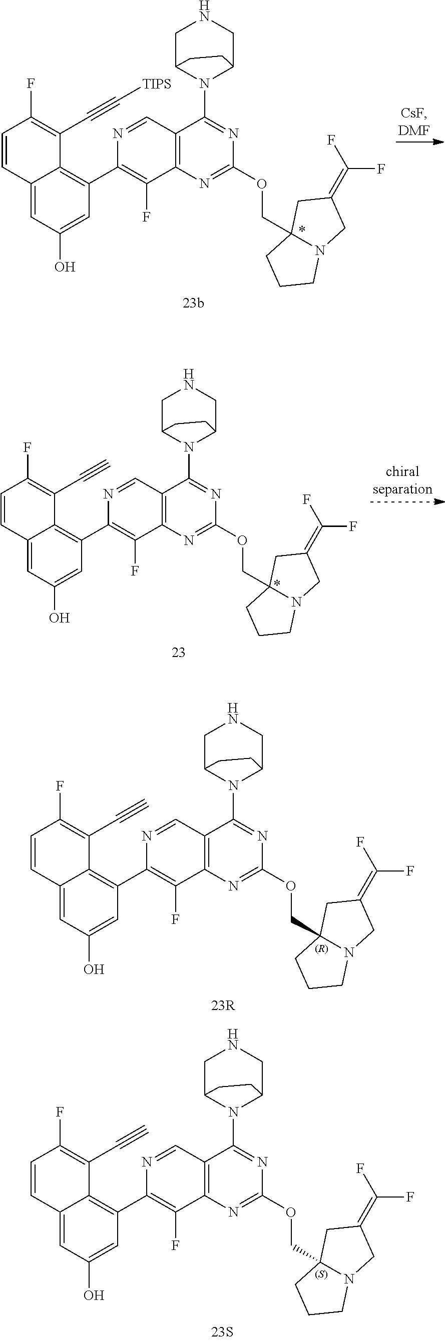

Compound 19c (135 mg, 0.23 mmol), potassium phosphate (146 mg, 0.69 mmol), Methylsulfonic acid [n-butyldi (1-adamantinyl) phosphine](2-amino-1,1′-biphenyl-2-yl) palladium (II) (17 mg, 0.02 mmol), 20a (179 mg, 0.35 mmol) were added to the reaction bottle, purged with N₂ for 3 times. Then dioxane/water (2.5 mL/0.5 mL) was added. The reaction mixture was stirred at 100° C. for 2 h under N₂. After the reaction was completed, the mixture was cooled to RT. Water was added and extracted with ethyl acetate (3×20 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 23a (120 mg, yield: 55%). MS m/z 931.4 [M+H]⁺.

The compound 23a (120 mg, 0.13 mmol) was dissolved in acetonitrile (4 mL). Then 4 M HCl in dioxane (1.0 mL) was added. The reaction mixture was stirred at RT for 1 h. After the reaction was completed, the mixture was concentrated to afford yellow solid 23b (101 mg, yield: 100%). MS m/z 787.7 [M+H]⁺.

The compound 23b (101 mg, 0.13 mmol) was dissolved in DMF (2 mL). Then Cesium Fluoride (195 mg, 1.28 mmol) was added. The mixture was stirred overnight at RT. Then water was added and extracted with ethyl acetate (3×10 mL), and the collected organic phase was washed with sat.NaCl, dried with anhydrous sodium sulfate, filterated and concentrated to give the crude product. The crude product was purified by column chromatography (dichloromethane: methanol=10:1) to afford yellow solid23 (48 mg, 59% yield, racemate). ¹H NMR (500 MHz, CD₃OD) δ 9.05 (s, 1H), 7.86 (dd, J=9.0, 5.7 Hz, 1H), 7.35 (d, J=2.5 Hz, 1H), 7.32 (t, J=9.0 Hz, 1H), 7.22 (d, J=2.5 Hz, 1H), 5.19-5.06 (m, 2H), 4.42-4.25 (m, 2H), 3.88-3.80 (m, 1H), 3.50-3.43 (m, 1H), 3.41 (s, 1H), 3.30-3.22 (m, 2H), 3.21-3.15 (m, 1H), 3.04-2.95 (m, 2H), 2.84-2.78 (m, 1H), 2.77-2.70 (m, 1H), 2.58-2.49 (m, 1H), 2.20-2.07 (m, 4H), 2.07-1.86 (m, 4H) ppm. MS m/z 631.1 [M+H]⁺. Compound 23 (racemate) is separated by chiral column to obtain compounds 23S and 23R

### Example 24: Preparation of Compound 24

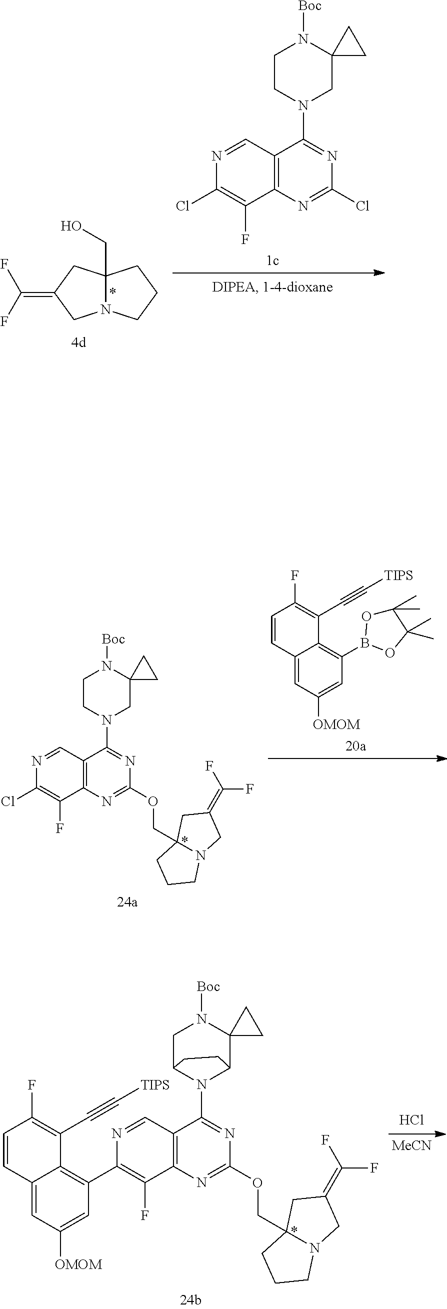

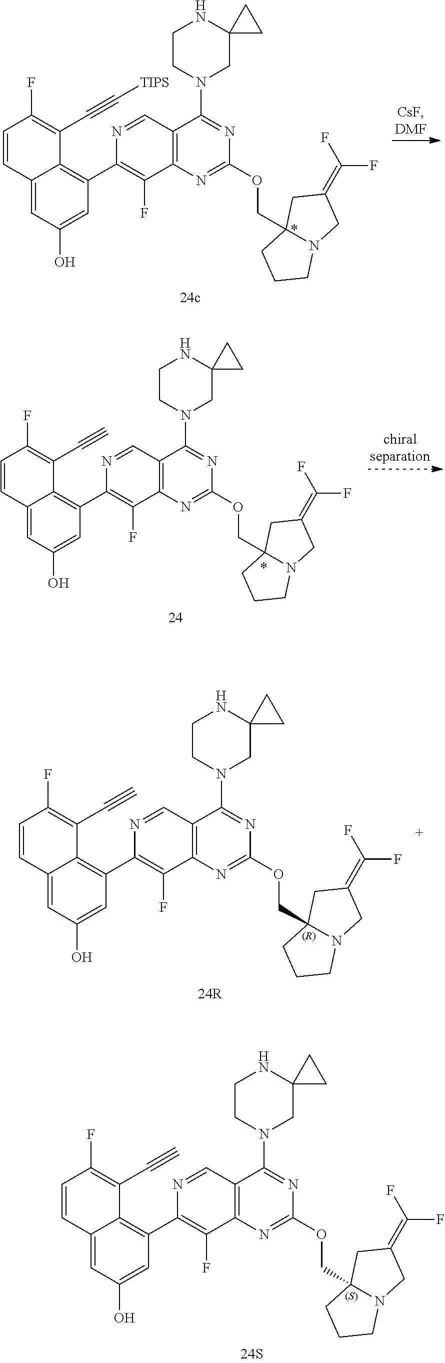

The compound 4d (100 mg, 0.53 mmol) and 1c (204 mg, 0.48 mmol) were dissolved in dioxane (1.5 mL). Then N, N-diisopropylethylamine(190 mg, 1.47 mmol) was added. The reaction mixture was stirred at 90° C. for 48 h under N₂. After the reaction was completed, the mixture was concentrated to give the crude product. The crude product was purified by column chromatography to afford a white solid 24a (127 mg, yield: 41%). ¹H NMR (500 MHz, CD₃OD) δ 8.84 (s, 1H), 4.33 (dd, J=42.2, 10.9 Hz, 2H), 4.14-4.09 (m, 2H), 3.95 (s, 2H), 3.89-3.82 (m, 1H), 3.77-3.72 (m, 2H), 3.54-3.48 (m, 1H), 3.24-3.17 (m, 1H), 2.83-2.74 (m, 2H), 2.58-2.51 (m, 1H), 2.19-2.09 (m, 1H), 2.07-1.99 (m, 1H), 1.97-1.89 (m, 2H), 1.48 (s, 9H), 1.06-1.02 (m, 2H), 0.93-0.89 (m, 2H) ppm. MS m/z 581.5, 583.4 [M+H]⁺.

Compound 24a (127 mg, 0.22 mmol), potassium phosphate (140 mg, 0.66 mmol), Methylsulfonic acid [n-butyldi (1-adamantinyl) phosphine](2-amino-1,1′-biphenyl-2-yl) palladium (II) (16 mg, 0.02 mmol), 20a (168 mg, 0.33 mmol) were added to the reaction bottle, purged with N₂ for 3 times. Then dioxane/water (2.5 mL/0.5 mL) was added. The reaction mixture was stirred at 100° C. for 2 h under N₂. After the reaction was completed, the mixture was cooled to RT. Water was added and extracted with ethyl acetate (3×20 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 24b (117 mg, yield: 57%). MS m/z 931.4 [M+H]⁺.

The compound 24b (117 mg, 0.12 mmol) was dissolved in acetonitrile (4 mL). Then 4 M HCl in dioxane (1 mL) was added. The reaction mixture was stirred at RT for 1 h. After the reaction was completed, the mixture was concentrated to afford yellow solid 24c (99 mg, yield: 100%). MS m/z 787.6 [M+H]⁺.

The compound 24c (99 mg, 0.12 mmol) was dissolved in DMF (2 mL). Then Cesium Fluoride (191 mg, 1.26 mmol) was added. The mixture was stirred overnight at RT. Then water was added and extracted with ethyl acetate (3×10 mL), and the collected organic phase was washed with sat.NaCl, dried with anhydrous sodium sulfate, filterated and concentrated to give the crude product. The crude product was purified by column chromatography (dichloromethane: methanol=10:1) to afford yellow solid24 (57 mg, 72% yield, racemate). ¹H NMR (500 MHz, CD₃OD) δ 8.98 (s, 1H), 7.85 (dd, J=9.0, 5.5 Hz, 1H), 7.35 (d, J=2.5 Hz, 1H), 7.32 (t, J=9.0 Hz, 1H), 7.22 (d, J=2.5 Hz, 1H), 4.39-4.24 (m, 2H), 4.16-4.07 (m, 2H), 3.98 (s, 2H), 3.86-3.78 (m, 1H), 3.50-3.43 (m, 1H), 3.39 (s, 1H), 3.21-3.09 (m, 3H), 2.84-2.77 (m, 1H), 2.77-2.69 (m, 1H), 2.57-2.48 (m, 1H), 2.17-2.07 (m, 1H), 2.06-1.96 (m, 1H), 1.96-1.86 (m, 2H), 0.75-0.65 (m, 4H) ppm. MS m/z 631.1 [M+H]⁺. Compound 24 (racemate) is separated by chiral column to obtain compounds 24S and 24R

### Example 25: Preparation of Compound 25

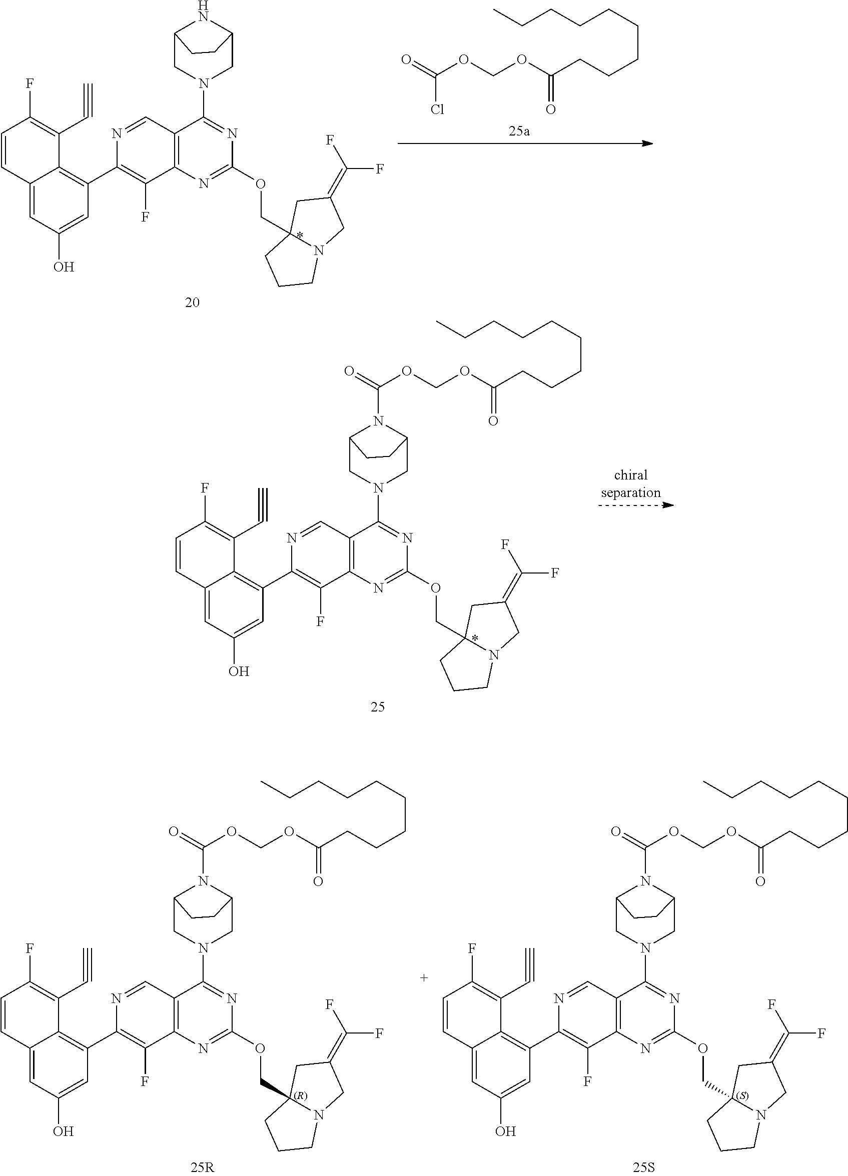

The compound 20 (34 mg, 0.05 mmol) and N, N-diisopropylethylamine(19 mg, 0.15 mmol) were dissolved in DCM (2 mL). Then 25a (13 mg, 0.05 mmol) in DCM (1 mL) was added at −40° C. The reaction mixture was stirred at RT for 0.5 h. After the reaction was completed, water was added and extracted with DCM (3×10 mL). The collected organic phase was washed with sat.NaCl, dried with anhydrous sodium sulfate filterated and concentrated to give the crude product. The crude product was purified by column chromatography (dichloromethane: methanol=20:1) to afford white solid 25 (12 mg, yield: 26%). ¹H NMR (500 MHz, CD₃OD) δ 9.05 (s, 1H), 7.86 (dd, J=9.0, 5.7 Hz, 1H), 7.36 (d, J=2.5 Hz, 1H), 7.32 (t, J=9.0 Hz, 1H), 7.20 (d, J=2.5 Hz, 1H), 5.83 (s, 2H), 4.76-4.63 (m, 2H), 4.59-4.41 (m, 4H), 4.08-4.01 (m, 1H), 3.84-3.65 (m, 3H), 3.42-3.35 (m, 1H), 3.34 (s, 1H), 3.00-2.94 (m, 1H), 2.93-2.87 (m, 1H), 2.69-2.62 (m, 1H), 2.39 (t, J=7.3 Hz, 2H), 2.26-2.19 (m, 1H), 2.14-1.96 (m, 5H), 1.89-1.83 (m, 2H), 1.67-1.59 (m, 2H), 1.38-1.26 (m, 12H), 0.91-0.85 (m, 3H). MS m/z 859.3 [M+H]⁺. Compound 25 (racemate) is separated by chiral column to obtain compounds 25S and 25R.

### Example 26: Preparation of Compound 26

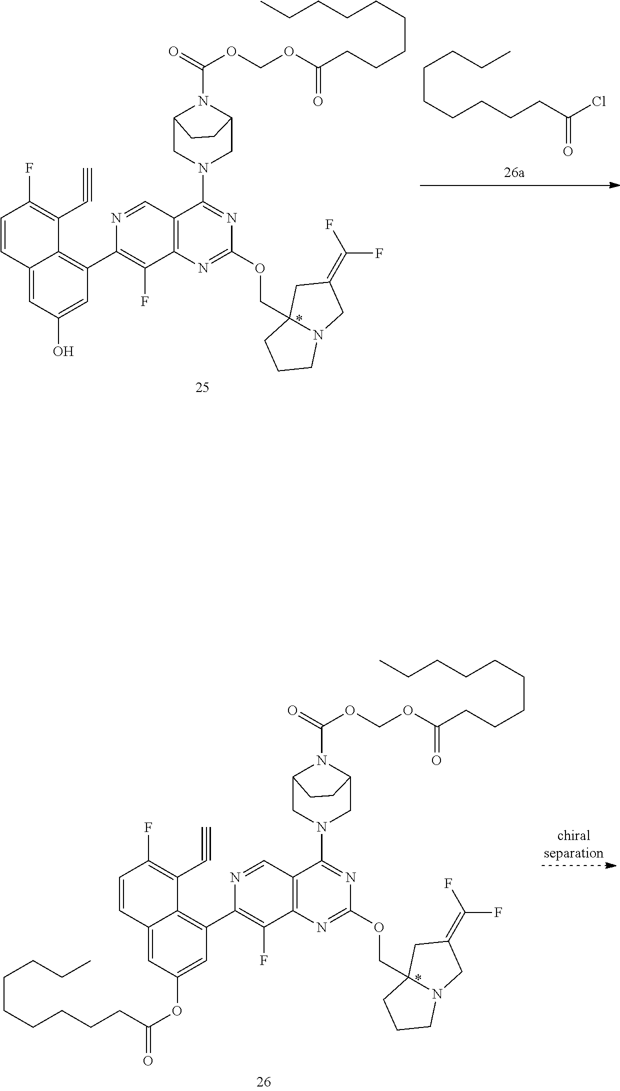

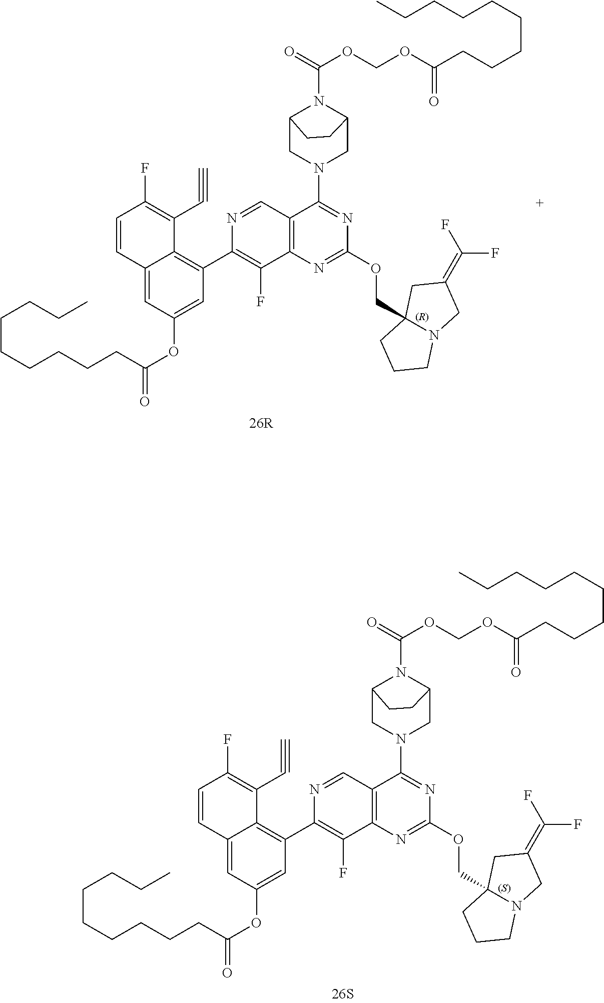

The compound 25 (6 mg, 0.007 mmol) and N, N-diisopropylethylamine(3 mg, 0.02 mmol) were dissolved in DCM (1 mL). Then Quinoyl chloride (2 mg, 0.01 mmol) in DCM (1 mL) was added at −40° C. The reaction mixture was stirred at RT for 0.5 h. After the reaction was completed, water was added and extracted with DCM (3×10 mL). The collected organic phase was washed with sat.NaCl, dried with anhydrous sodium sulfate filterated and concentrated to give the crude product. The crude product was purified by column chromatography (dichloromethane: methanol=20:1) to afford white solid 26 (4 mg, yield: 56%). ¹H NMR (500 MHz, CD₃OD) δ 9.06 (s, 1H), 8.09 (dd, J=9.0, 5.7 Hz, 1H), 7.88 (d, J=2.4 Hz, 1H), 7.48 (t, J=9.0 Hz, 1H), 7.44 (d, J=2.4 Hz, 1H), 5.83 (s, 2H), 4.74-4.63 (m, 2H), 4.58-4.47 (m, 3H), 4.47-4.41 (m, 1H), 4.09-4.01 (m, 1H), 3.84-3.73 (m, 2H), 3.72-3.65 (m, 1H), 3.45 (s, 1H), 3.42-3.37 (m, 1H), 2.99-2.93 (m, 1H), 2.93-2.86 (m, 1H), 2.69-2.62 (m, 3H), 2.39 (t, J=7.3 Hz, 2H), 2.26-2.20 (m, 1H), 2.14-2.07 (m, 1H), 2.06-1.97 (m, 4H), 1.89-1.83 (m, 2H), 1.80-1.73 (m, 2H), 1.66-1.59 (m, 2H), 1.48-1.41 (m, 2H), 1.38-1.23 (m, 22H), 0.92-0.83 (m, 6H). MS m/z 1013.4 [M+H]⁺. Compound 26 (racemate) is separated by chiral column to obtain compounds 26S and 26R.

### Example 27: Preparation of Compound 27

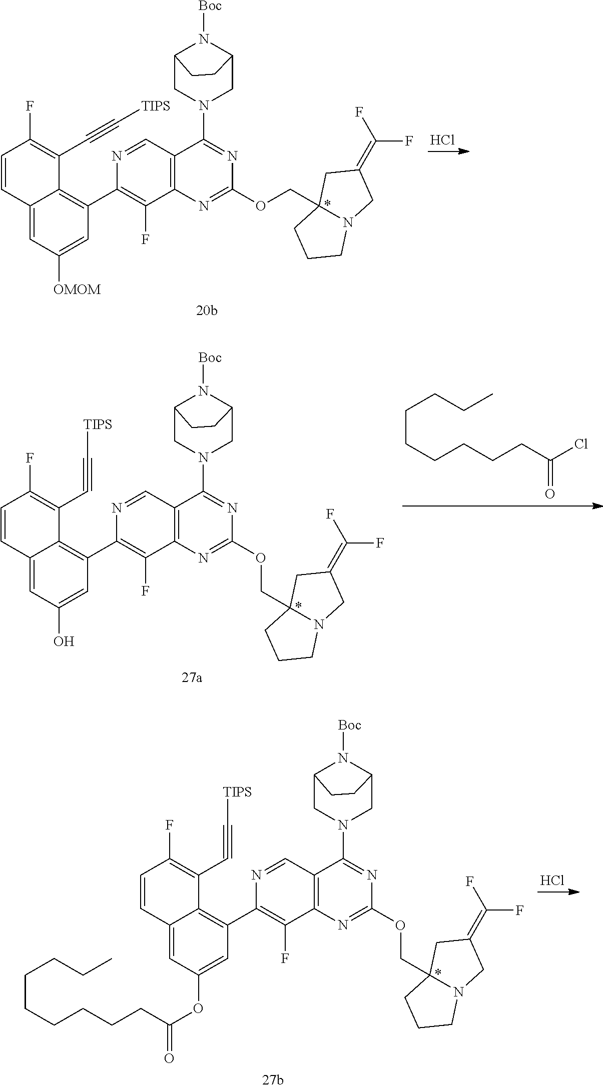

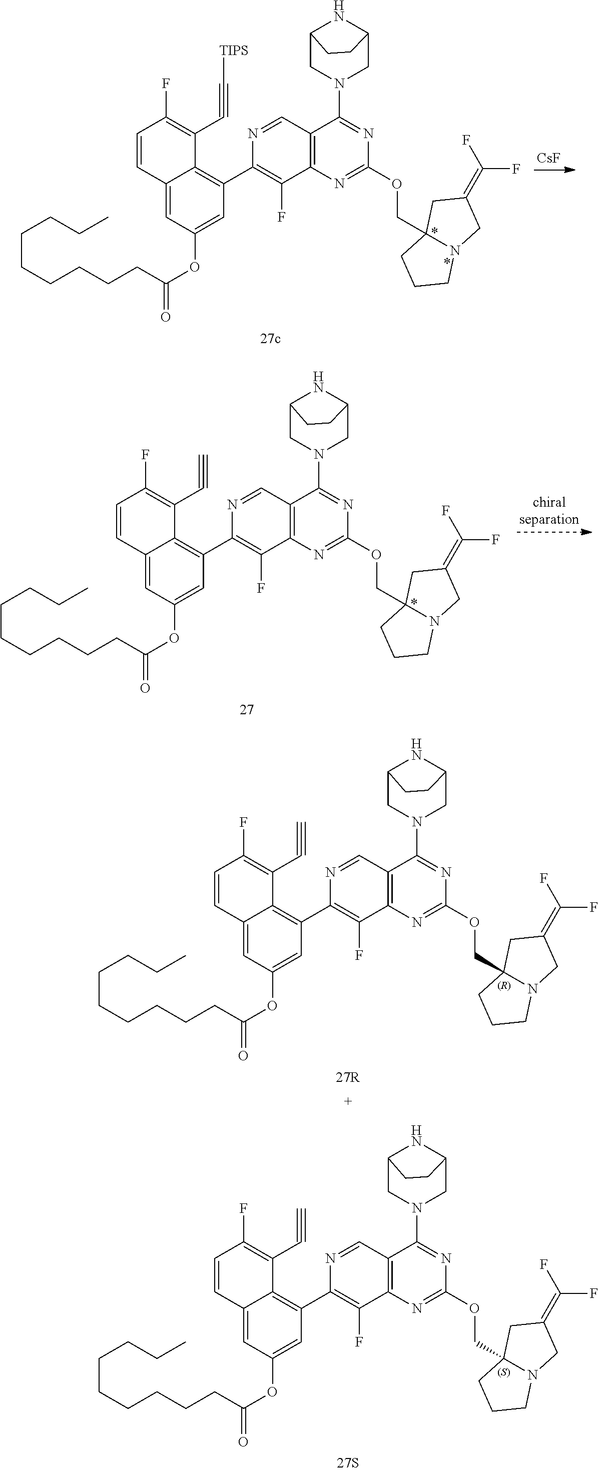

The compound 20b (100 mg, 0.11 mmol) was dissolved in DCM (3 mL). Then 4 M HCl in dioxane (0.1 mL) was added. The reaction mixture was stirred at RT for 1 h. After the reaction was completed, the mixture was concentrated. The crude product was purified by column chromatography (dichloromethane: methanol=20:1) to afford white solid 27a (67 mg, yield: 71%). MS m/z 887.1 [M+H]⁺.

The compound 27a (13 mg, 0.015 mmol) and N, N-diisopropylethylamine(6 mg, 0.05 mmol) were dissolved in DCM (2 mL). Then Quinoyl chloride(4 mg, 0.02 mmol) was added at −40° C. The reaction mixture was stirred at RT for 0.5 h. After the reaction was completed, water was added and extracted with DCM (3×10 mL). The collected organic phase was washed with sat.NaCl, dried with anhydrous sodium sulfate filterated and concentrated to afford yellow solid 27b (16 mg, yield: 100%). MS m/z 1041.4 [M+H]⁺.

The compound 27b (16 mg, 0.015 mmol) was dissolved in acetonitrile (2 mL). Then 4 M HCl in dioxane (0.5 mL) was added. The reaction mixture was stirred at RT for 3 h. After the reaction was completed, the mixture was concentrated to afford yellow solid 27c (14 mg, yield: 100%). MS m/z 941.4 [M+H]⁺.

The compound 27c(14 mg, 0.015 mmol) was dissolved in DMF (1 mL). Then Cesium Fluoride (23 mg, 0.15 mmol) was added. The mixture was stirred overnight at RT. Then water was added and extracted with ethyl acetate, and the collected organic phase was washed with sat.NaCl, dried with anhydrous sodium sulfate, filterated and concentrated to give the crude product. The crude product was purified by column chromatography (dichloromethane: methanol=10:1) to afford yellow solid27 (3 mg, 26% yield, racemate). MS m/z 785.3 [M+H]⁺. ¹H NMR (500 MHz, CD₃OD) δ 9.04 (s, 1H), 8.09 (dd, J=9.0, 5.7 Hz, 1H), 7.88 (d, J=2.4 Hz, 1H), 7.48 (t, J=9.0 Hz, 1H), 7.44 (d, J=2.4 Hz, 1H), 4.75-4.65 (m, 2H), 4.45-4.39 (m, 1H), 4.34-4.27 (m, 1H), 3.94-3.74 (m, 5H), 3.47 (t, J=12.5 Hz, 2H), 3.19 (dd, J=11.0, 6.9 Hz, 1H), 2.84-2.79 (m, 1H), 2.76 (d, J=8.4 Hz, 1H), 2.65 (t, J=7.4 Hz, 2H), 2.58-2.52 (m, 1H), 2.18-2.10 (m, 1H), 2.09-1.85 (m, 7H), 1.80-1.73 (m, 2H), 1.45-1.23 (m, 12H), 0.91-0.86 (m, 3H). Compound 27 (racemate) is separated by chiral column to obtain compounds 27S and 27R

### Example 28: Preparation of Compound 28

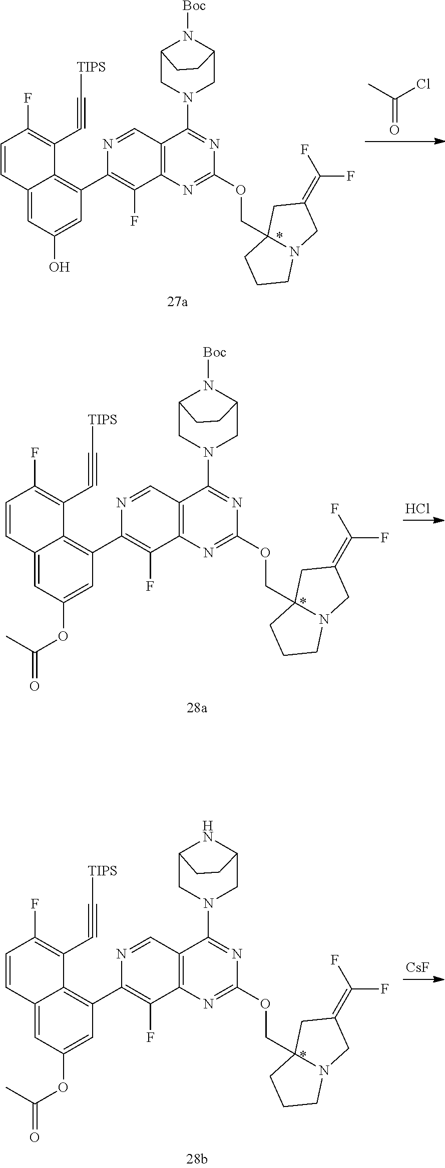

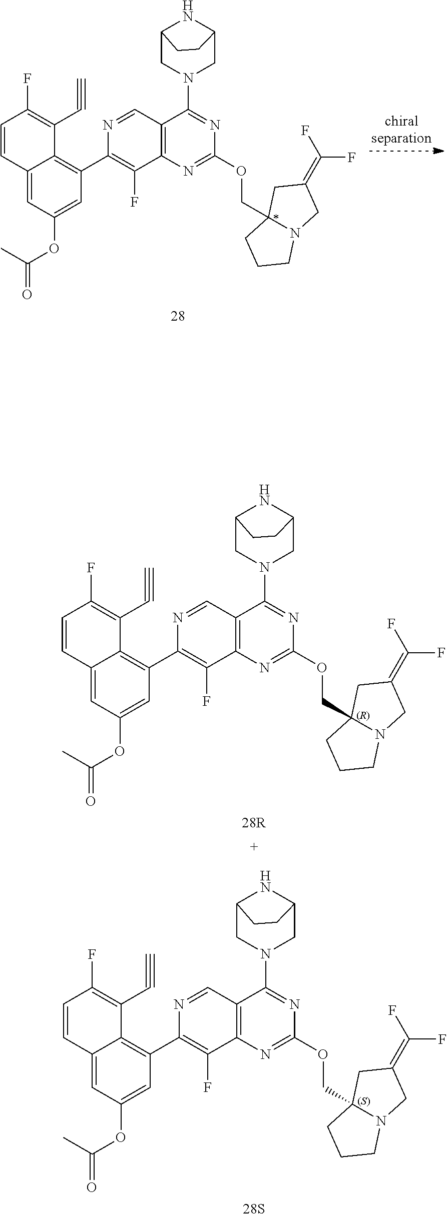

The compound 27a (11 mg, 0.012 mmol) and N, N-diisopropylethylamine(6 mg, 0.05 mmol) were dissolved in DCM (2 mL). Then acetylchloride(2 mg, 0.02 mmol) was added at −40° C. The reaction mixture was stirred at RT for 0.5 h. After the reaction was completed, water was added and extracted with DCM (3×10 mL). The collected organic phase was washed with sat.NaCl, dried with anhydrous sodium sulfate filterated and concentrated to afford yellow solid 28a (11 mg, yield: 100%). MS m/z 929.6 [M+H]⁺.

The compound 28a (11 mg, 0.012 mmol) was dissolved in acetonitrile (2 mL). Then 4 M HCl in dioxane (0.5 mL) was added. The reaction mixture was stirred at RT for 3 h. After the reaction was completed, the mixture was concentrated to afford yellow solid 28b (10 mg, yield: 100%). MS m/z 829.5 [M+H]⁺.

The compound 28b (10 mg, 0.012 mmol) was dissolved in DMF (1 mL). Then Cesium Fluoride (18 mg, 0.12 mmol) was added. The mixture was stirred overnight at RT. Then water was added and extracted with ethyl acetate, and the collected organic phase was washed with sat.NaCl, dried with anhydrous sodium sulfate, filterated and concentrated to give the crude product. The crude product was purified by column chromatography (dichloromethane: methanol=10:1) to afford yellow solid28 (2 mg, 25% yield). MS m/z 673.1 [M+H]⁺. Compound 28 (racemate) is separated by chiral column to obtain compounds 28S and 28R

### Example 29: Preparation of Compound 29

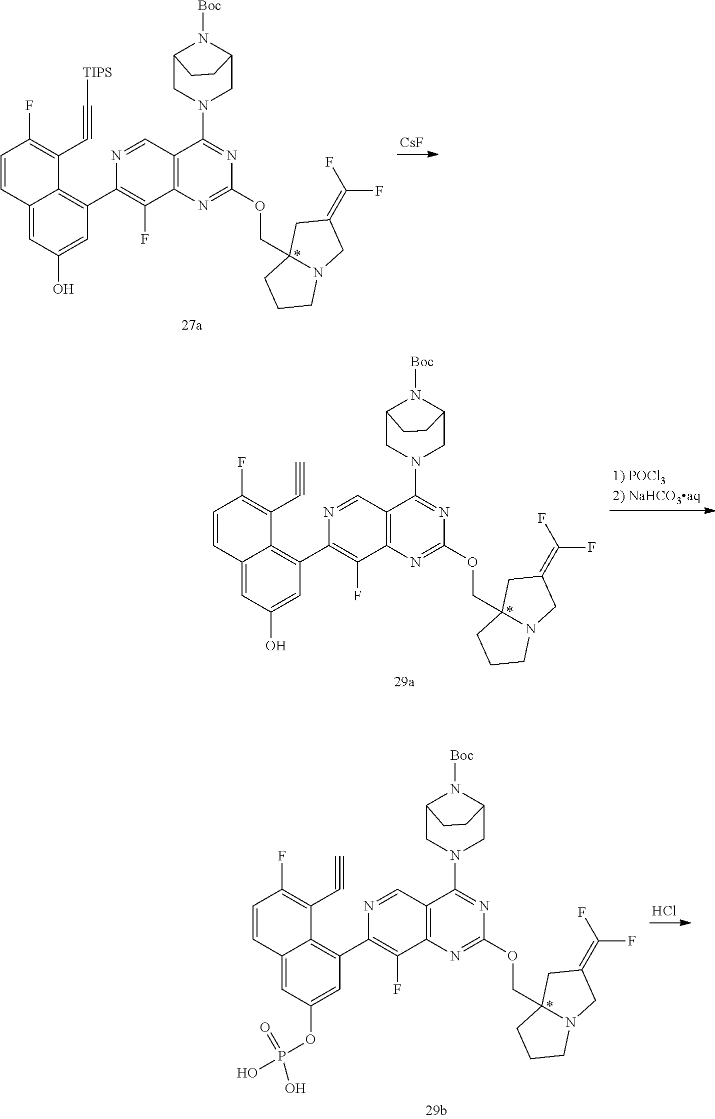

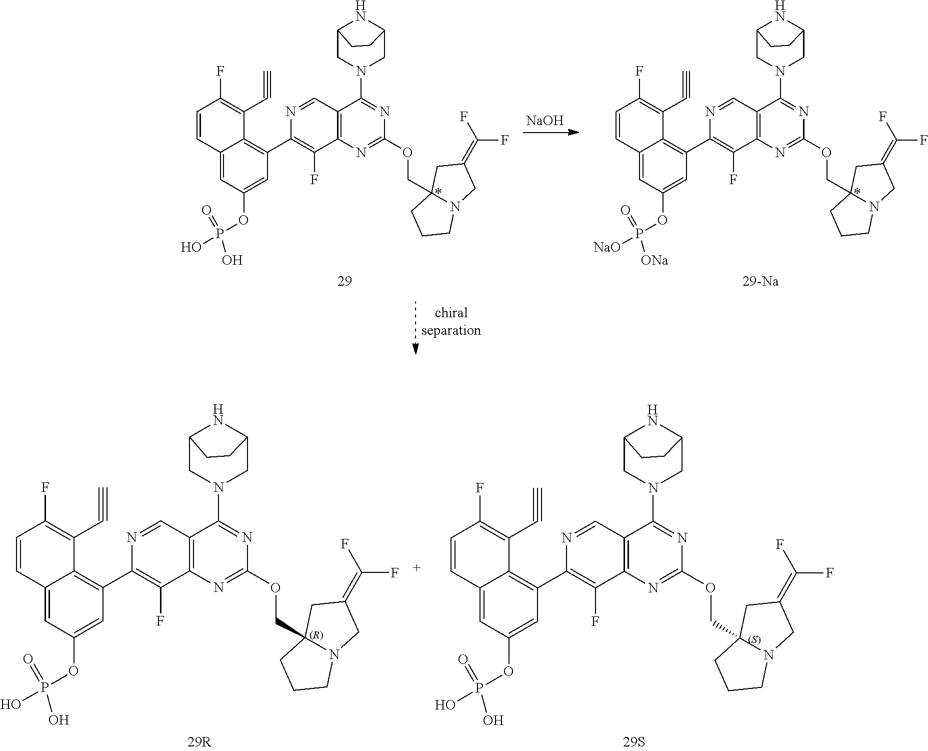

The compound 27a(39 mg, 0.04 mmol) was dissolved in DMF (1 mL). Then Cesium Fluoride (67 mg, 0.44 mmol) was added. The mixture was stirred overnight at RT. Then water was added and extracted with ethyl acetate, and the collected organic phase was washed with sat.NaCl, dried with anhydrous sodium sulfate, filterated and concentrated to give the crude product. The crude product was purified by column chromatography (dichloromethane: methanol=10:1) to afford yellow solid29a (23 mg, 73% yield). MS m/z 731.3 [M+H]⁺.

The compound 29a (10 mg, 0.014 mmol) and N, N-diisopropylethylamine(6 mg, 0.05 mmol) were dissolved in acetonitrile (2 mL). Then phosphorus oxychloride(3 mg, 0.02 mmol) was added at ice bath. The reaction mixture was stirred at ice bath for 10 min. Then sat. NaHCO3 (0.5 mL) was added. The reaction mixture was stirred at RT for 1 h. After the reaction was completed, water was added and extracted with ethyl acetate. The collected organic phase was washed with sat.NaCl, dried with anhydrous sodium sulfate filterated and concentrated to afford yellow solid 29b (10 mg, yield: 91%). MS m/z 811.3 [M+H]⁺.

The compound 29b (10 mg, 0.012 mmol) was dissolved in acetonitrile (2 mL). Then 4 M HCl in dioxane (0.5 mL) was added. The reaction mixture was stirred at RT for 3 h. After the reaction was completed, the mixture was concentrated. The crude product was purified by Pre-TLC (dichloromethane: methanol=10:1) to afford yellow solid29 (4 mg, yield: 45%). MS m/z 711.1 [M+H]⁺. The compound 29 was dissolved in acetonitrile (1 mL). Then NaOH (0.4 mg, 0.01 mmol) in water (0.5 mL) was added. The mixture was freeze dried to give yellow solid disodium salt 29-Na (4 mg, yield: 100%). MS m/z 711.1 [M+H]⁺. Compound 29 (racemate) is separated by chiral column to obtain compounds 29S and 29R

### Example 30: Preparation of Compound 30

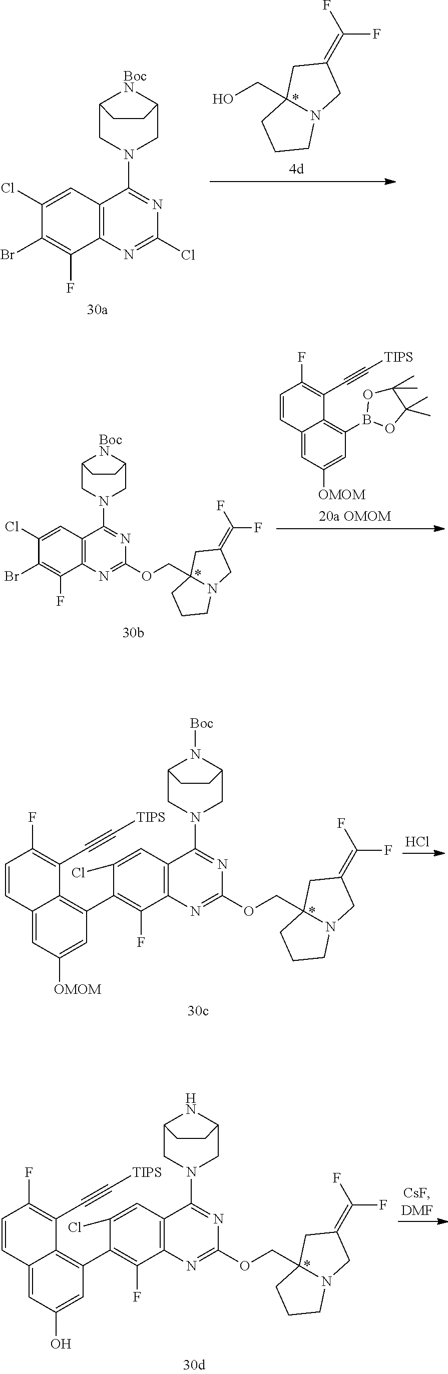

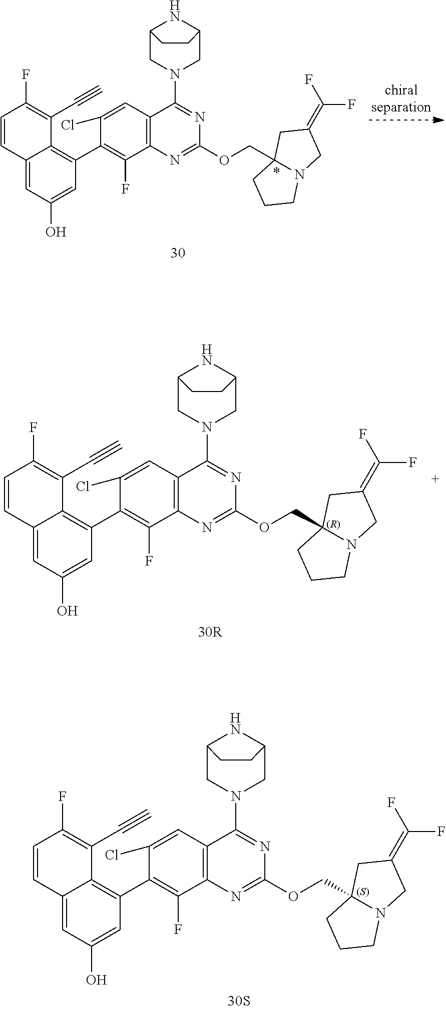

Compound 30a was synthesized according to WO2022002102. Compound 4d (56 mg, 0.30 mmol), cesium carbonate (193 mg, 0.60 mmol) and tri ethyl enedi amine(22 mg, 0.20 mmol) were dissolved in DMF/THF (0.5/0.5 mL). Then the reaction mixture was stirred at RT for 3 h. After the reaction was completed, water was added. Then the mixture was extracted with ethyl acetate (3×10 mL), and the collected organic phase was washed with sat.NaCl, dried with anhydrous sodium sulfate, filterated and concentrated to give the crude product. The crude product was purified by column chromatography (dichloromethane: methanol=30:1) to afford yellow oil 30b (70 mg, yield: 54%). MS m/z 658.0 & 660.0 [M+H]⁺.

Compound 30b (35 mg, 0.05 mmol), potassium phosphate (34 mg, 0.15 mmol), Methylsulfonic acid [n-butyldi (1-adamantinyl) phosphine](2-amino-1,1′-biphenyl-2-yl) palladium (II) (3 mg, 0.005 mmol), 20a (41 mg, 0.08 mmol) were added to the reaction bottle, purged with N₂ for 3 times. Then dioxane/water (0.5 mL/0.1 mL) was added. The reaction mixture was stirred at 100° C. for 2 h under N₂. After the reaction was completed, the mixture was cooled to RT. Water was added and extracted with ethyl acetate (3×10 mL). The collected organic phase was washed with sat.NaCl, dried with anhydrous sodium sulfate filterated and concentrated to give the crude product. The crude product was purified by column chromatography (dichloromethane: methanol=25:1) to afford yellow oil 30c (30 mg, yield: 56%). MS m/z 964.5 & 965.5 [M+H]⁺.

The compound 30c (30 mg, 0.03 mmol) was dissolved in acetonitrile (1 mL). Then 4 M HCl in dioxane (0.1 mL) was added. The reaction mixture was stirred at RT for 1 h. After the reaction was completed, the mixture was concentrated to afford yellow solid 30d (26 mg, yield: 100%). MS m/z 820.3 & 822.3 [M+H]⁺.

The compound 30d(26 mg, 0.03 mmol) was dissolved in DMF (1 mL). Then Cesium Fluoride (45 mg, 0.3 mmol) was added. The mixture was stirred for 12 h at RT. Then the mixture was filterated and concentrated. The crude product was purified by column chromatography (dichloromethane: methanol=25:1) to afford yellow solid30 (4 mg, 20% yield). ¹H NMR (500 MHz, MeOD-d₄) δ 7.84 (dd, J=9.2, 5.9 Hz, 1H), 7.82 (br, J=4.8 Hz, 1H), 7.34-7.27 (m, 2H), 7.03 (d, J=2.1 Hz, 1H), 4.52 (dd, J=34.3, 13.0 Hz, 2H), 4.39-4.32 (m, 1H), 4.30-4.24 (m, 1H), 3.92-3.80 (m, 3H), 3.77-3.63 (m, 2H), 3.47 (d, J=14.1 Hz, 1H), 3.26 (s, 1H), 3.22-3.15 (m, 1H), 2.81 (d, J=15.7 Hz, 1H), 2.78-2.70 (m, 1H), 2.53 (d, J=16.2 Hz, 1H), 2.17-2.10 (m, 1H), 2.05-1.89 (m, 7H). MS m/z 664.1 & 666.1 [M+H]⁺. Compound 30 (racemate) is separated by chiral column to obtain compounds 30S and 30R

### Example 31: 3D cell proliferation inhibition experiment

Compound activity was indicated by AGS (human gastric cancer cell line, KRAS G12D mutation), GP2D (human colon cancer cell line, KRAS G12D mutation), HPAC (human pancreatic cancer cell line, KRAS G12D mutation) and AsPCl (human pancreatic cancer cell line, KRAS G12D mutation) 3D cell proliferation inhibition. The Cell Titer-Glo (CTG) method was adopted. Compound was dissolved with DMSO to 10 mM, and then diluted by medium. The upper medium of cells was removed, washed once with DPBS, added 2 mL of trypsin, and placed at 37° C. for a while. After cells were detached from the petri dish, 5 mL fresh medium was added and the suspension cells were centrifuged at 1000 rpm for 5 minutes at room temperature. Resuspend the cells with 5 mL of fresh culture medium, and use Countess™ to count cells. 200 nL compounds were added to a ultra-low attachment 384-well plate, and 40 μL cells were added to each well. After seven days of culture at 37° C., 5% CO2, CTG was added and the signal was recorded using Envision. The Inhibition rate is calculated using the following formula: Inhibition %=(Ave_H-Sample)/(Ave_H-Ave_L)×100%. Ave_H represents the average reading value of DMSO well, Sample represents the average reading value of compound well, and Ave_L represents the average reading value of 10 μM positive control well. The log value of concentration was taken as the X-axis, and the percentage inhibition rate was taken as the Y-axis. The dose-effect curve was fitted with log(inhibitor) vs. response-Variable slope of the analysis software GraphPad Prism 5. The IC₅₀ value or inhibitory rate of each compound on cell proliferation was obtained. The activities of some representative compounds are shown in Table 1.

TABLE 1
3D cell proliferation inhibition activity

| | AGS | GP2D | HPAC | AsPC1 |
|---|---|---|---|---|
| | inhibition | IC₅₀ | IC₅₀ | IC₅₀ |
| Compounds | (100 nM) | (nM) | (nM) | (nM) |
| 1 | >50% | | | |
| 2 | >50% | | | |
| 3 | >50% | | | |
| 4 | >50% | <0.5 | <15 | |
| 10 | >50% | <0.5 | <10 | <5 |
| 12 | >50% | | <70 | |
| 13 | >50% | | | |
| 14 | >50% | | | |
| 19 | >50% | | | |
| 20 | | | <5 | |
| 20-P1 | | <0.1 | <5 | <1 |
| 20-P2 | | <0.5 | <5 | <5 |
| 21 | | | | <100 |
| 22 | | | | <150 |
| 23 | | | <50 | |
| 24 | | | <70 | |
| 28 | | | <70 | |
| 29 | | | <10 | |
| 30 | | | <25 | |

### Example 32: KRAS G12D&SOS1 Binding Assay

The positive inhibitor and test compounds (10 mM storage solution) were diluted by a 3-fold gradient with 100% DMSO. Transfer 2 μL diluted compound to an 18 μL diluent and mix thoroughly. Take 2 μL of the diluted compound into a 384-well plate and repeated twice for each sample. Centrifuge 384 well plates at 1000 rpm and add 4 μL of KRAS G12D&GTP mixture into each well, followed by 4 μL of SOS1, and incubate for 15 minutes. Then 10 μL of Tag-Tb and Tag-XL665 were added and incubated at 4° C. for 3 hours. The HTRF (665 nM and 615 nM) signals were read using BMG. With the log value of concentration as the X-axis and 665 nM/615 nM as the Y-axis, Y=Bottom+(Top-Bottom)/(1+10∧((LogIC₅₀-X)*HillSlope)) was used to fit the dose-effect curve. The IC₅₀ value of each compound on enzyme activity was obtained. The activities of some representative compounds are shown in Table 2.

TABLE 2
KRAS G12D & SOS1 binding inhibition activity

| Compounds | IC₅₀ (nM) |
|---|---|
| 10 | <15 |
| 20 | <15 |
| 21 | <15 |
| 22 | <15 |
| 28 | <30 |
| 30 | <60 |

All documents referred to in the present invention are incorporated by reference herein as if each document is individually incorporated by reference. Further, it should be understood that upon reading the above teaching of the present invention, various alterations or modifications may be made to the present invention by those skilled in the art, and those equivalents also fall within the scope defined by the appended claims of the present application.

1. A compound of the structure shown in formula(I) below, or an optical isomer, a pharmaceutically acceptable salt, a prodrug, a deuterated derivative, a hydrate, or a solvate thereof: 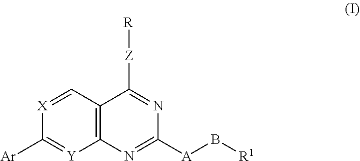
wherein:
Ar is selected from aryl or heteroaryl; the aryl or heteroaryl is optionally substituted by one or more groups selected from the group consisting of: halogen, C₁₋₄ alkyl, C₁₋₄haloalkyl, hydroxyl, C₁₋₄ alkoxy, C₁₋₄ haloalkoxy, C₂₋₄ alkenyl, C₂₋₄ haloalkenyl, C₂₋₄ alkynyl, C₂₋₄ haloalkynyl, C₃₋₆ cycloalkyl, 3- to 6-membered heterocyclyl, C₃₋₆ cycloalkyl-O—, 3- to 6-membered heterocyclyl-O—, C₃₋₆ cycloalkyl C₂₋₄ alkynyl, 3- to 6-membered heterocyclyl C₂₋₄ alkynyl, C₁₋₄ haloalkyl C₂₋₄ alkynyl, NR²R², CN, SR², —OC(O)R^{k}, —O—P(O)(OR^{m})₂, and —O—CH(Rⁿ)—O—P(O)(OR^{m})₂; wherein, each R² is independently hydrogen or C₁₋₄ alkyl; R^{k} is selected from C₁₋₁₂ alkyl, wherein the alkyl is optionally substituted by one or more groups selected from the group consisting of: hydroxyl, C₁₋₄alkoxy, NR²R², C(O)OH, C(O)₀C₁₋₂ alkyl, and CN; R^{m}is selected from hydrogen and C₁₋₄ alkyl; Rⁿ is selected from hydrogen and C₁₋₄ alkyl; the cycloalkyl or heterocyclyl as described above is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄alkynyl, CN, and ═M; wherein M is selected from 0 and CR³R⁴; R³ and R⁴ are independently selected from the group consisting of: hydrogen, fluorine, and C₁₋₄ alkyl;
R is selected from 5 to 12 membered heterocyclyl, including partially unsaturated or saturated monocyclic or polycyclic heterocyclyl, and the polycyclic heterocyclyl includes spiral, fused, or bridged heterocyclyl; the heterocyclyl is optionally substituted by one or more groups selected from the group consisting of: halogen, C₁₋₄ alkyl, ═O, hydroxyl, CN, and —CO(O)—CH(Rⁿ)—O—C(O)—U; wherein, Rⁿ is selected from hydrogen and C₁₋₄ alkyl; U is selected from C₁₋₁₈ alkyl;
Z is selected from chemical bonds, —O—, —S—, —NR⁵—, —C(R^{a})₂—, —C═C—, —CR^{a}═CR^{a}—, —N═, and —CR^{a}═; wherein, R⁵ is selected from hydrogen or C₁₋₄ alkyl; R^{a} is selected from hydrogen, halogen, and C₁₋₄ alkyl;
A is selected from chemical bonds, —O—, —S—, and —NR⁶—; wherein, R⁶ is selected from hydrogen and C₁₋₄ alkyl;
B is selected from —(CR⁷R⁸)ₘ—, —(CR⁷R⁸)ₘ-T —(CR⁷R⁸)ₘ—; wherein, T is selected from —C═C—, —CR^{a}═CR^{a}—, C₃₋₆cycloalkyl, and 3- to 6-membered heterocyclyl; wherein, the cycloalkyl or heterocyclyl is optionally substituted by one or more groups selected from the group consisting of: halogen, C₁₋₄alkyl, C₁₋₄ haloalkyl, hydroxyl, C₁₋₄alkoxy, C₁₋₄haloalkoxy, and CN; R⁷ and R⁸ are independently selected from the group consisting of: hydrogen, halogen, and C₁₋₄ alkyl; or R⁷ and R⁸ together with the C atom to which they attached form a C₃₋₆cycloalkyl; R^{a} is selected from hydrogen, halogen, and C₁₋₄ alkyl; each m is independently selected from 0, 1, 2, and 3;
R¹ is selected from C₃₋₈cycloalkyl or 4- to 12-membered heterocyclyl, wherein the heterocyclyl includes partially unsaturated or saturated monocyclic or polycyclic heterocyclyl, and the polycyclic heterocyclyl includes spiral, fused, or bridged heterocyclyl; the cycloalkyl or heterocyclyl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, —CH₂OC(O)NR⁹R¹⁰, CN, SR², C₂₋₄ alkenyl, C₂₋₄alkynyl, C₃₋₈cycloalkyl, 3 to 8-membered heterocyclyl, NR^{h}R^{h}, —(CR⁷R⁸)ₘ—OR^{h}, —(CR⁷R⁸)ₘ—NR^{h}R^{h}, and ═M; wherein, R⁹ and R¹⁰ are independently selected from hydrogen and C₁₋₄ alkyl, or R⁹ and R¹⁰ together with the N atom to which they attached form a 4-to-8-membered heterocyclyl comprising 1 or 2 N atoms and 0 or 1 heteroatom selected from O and S; each R^{h} is independently hydrogen, C₁₋₄ alkyl, or C₁₋₄haloalkyl; M is selected from O and CR³R⁴; the definitions of R², R³, R⁴, R⁷, R¹, and m are as described above; X and Y are independently selected from N and CR¹¹; wherein, R¹¹ is selected from hydrogen, halogen, C₁₋₄ alkyl, C₁₋₄haloalkyl, C₁₋₄ alkoxy, C₁₋₄haloalkoxy, C₂₋₄alkynyl, C₃₋₆cycloalkyl, and CN;
provided that when R¹ is not substituted by ═M, and M is selected from CR³R⁴, the structural fragment 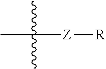
is not 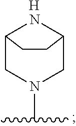
wherein, each of the above-mentioned alkyl, alkenyl, alkynyl, cycloalkyl, heterocyclyl, aryl, and heteroaryl is optionally and independently substituted by 1-3 substituents independently selected from the group consisting of: halogen, C₁₋₄alkyl, C₁₋₄haloalkyl, C₂₋₄ alkenyl, C₂₋₄alkynyl, C₃₋₈cycloalkyl, 3- to 8-membered heterocyclyl, aryl, heteroaryl, CN, NO₂, OR^{h}, SR^{h}, NR^{h}R^{h}, C(O)R^{t}, C(O)OR^{h}, C(O)NR^{h}R^{h}, NR^{h}C(O)R, NR^{h}S(O)₂R and S(O)₂R, provided that the formed chemical structure is stable and meaningful; wherein, R^{t} is C₁₋₄ alkyl, C₂₋₄ alkenyl, C₂₋₄ alkynyl, C₃₋₈cycloalkyl, 4- to 8-membered heterocyclyl, aryl, or heteroaryl; each R^{h} is independently hydrogen, C₁₋₄ alkyl, or C₁₋₄ haloalkyl; or two R^{h} together with the N atom to which they attached form a 3-to-8-membered heterocyclyl comprising 1 or 2 N atoms and 0 or 1 heteroatom selected from O and S; and
unless otherwise specified, the above-mentioned aryl is an aromatic group containing 6-12 carbon atoms; the heteroaryl is a 5- to 15-membered (preferably 5- to 12-membered) heteroaromatic group.

2. The compound according to claim 1, or an optical isomer, a pharmaceutically acceptable salt, a prodrug, a deuterated derivative, a hydrate, or a solvate thereof, wherein, formula (I) is formula (II): 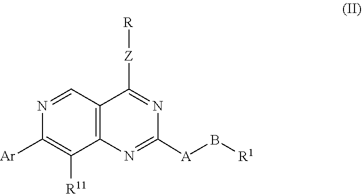
wherein:
Ar is selected from aryl or heteroaryl; the aryl or heteroaryl is optionally substituted by one or more groups selected from the group consisting of: halogen, C₁₋₄ alkyl, C₁₋₄ haloalkyl, hydroxyl, C₁₋₄ alkoxy, C₁₋₄ haloalkoxy, C₂₋₄ alkenyl, C₂₋₄ haloalkenyl, C₂₋₄ alkynyl, C₂₋₄ haloalkynyl, C₃₋₆cycloalkyl, 3- to 6-membered heterocyclyl, C₃₋₆ cycloalkyl-O—, 3- to 6-membered heterocyclyl-O—, C₃₋₆ cycloalkyl C₂₋₄ alkynyl, 3- to 6-membered heterocyclyl C₂₋₄alkynyl, C₁₋₄haloalkyl C₂₋₄alkynyl, NR²R², CN, SR², —OC(O)R^{k}, —O—P(O)(OR^{m})₂, and —O—CH(Rⁿ)—O—P(O)(OR^{m})₂; wherein, each R² is independently hydrogen or C₁₋₄ alkyl; R^{k} is selected from C₁₋₁₂ alkyl, wherein, the alkyl is optionally substituted by one or more groups selected from the group consisting of: hydroxyl, C₁₋₄ alkoxy, NR²R², C(O)OH, C(O)OC₁₋₂ alkyl, and CN; R^{m} is selected from hydrogen, and C₁₋₄ alkyl; Rⁿ is selected from hydrogen and C₁₋₄ alkyl; the cycloalkyl or heterocyclyl as described above is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄alkynyl, CN, and ═M; wherein, M is selected from 0 and CR³R⁴; R³ and R⁴ are independently selected from the group consisting of: hydrogen, fluorine, and C₁₋₄ alkyl;
R is selected from 5 to 12 membered heterocyclyl, including partially unsaturated or saturated monocyclic or polycyclic heterocyclyl, and the polycyclic heterocyclyl includes spiral, fused, or bridged heterocyclyl; the heterocyclyl is optionally substituted by one or more groups selected from the group consisting of: halogen, C₁₋₄ alkyl, ═O, hydroxyl, CN, and —CO(O)—CH(Rⁿ)—O—C(O)—U; wherein, Rⁿ is selected from hydrogen and C₁₋₄ alkyl; U is selected from C₁₋₁₈ alkyl;
Z is selected from chemical bonds, —O—, —S—, —NR⁵—, —C(R^{a})₂—, —C═C—, —CR^{a}═CR^{a}—, —N═, and —CR^{a}═; wherein, R⁵ is selected from hydrogen and C₁₋₄ alkyl; R^{a} is selected from hydrogen, halogen, and C₁₋₄ alkyl;
A is selected from chemical bonds, —O—, —S—, and —NR⁶—; wherein, R⁶ is selected from hydrogen and C₁₋₄ alkyl;
B is selected from —(CR⁷R⁸)ₘ—, and —(CR⁷R⁸)ₘ-T-(CR⁷R⁸)ₘ—; wherein, T is selected from —C═C—, —CR^{a}═CR^{a}—, C₃₋₆cycloalkyl, and 3- to 6-membered heterocyclyl; wherein, the cycloalkyl or heterocyclyl is optionally substituted by one or more groups selected from the group consisting of: halogen, C₁₋₄alkyl, C₁₋₄ haloalkyl, hydroxyl, C₁₋₄alkoxy, C₁₋₄haloalkoxy, and CN; R⁷ and R⁸ are independently selected from the group consisting of: hydrogen, halogen, and C₁₋₄ alkyl; or R⁷ and R⁸ together with the C atom to which they attached form a C₃₋₆cycloalkyl; R^{a} is selected from hydrogen, halogen, and C₁-4 alkyl; each m is independently selected from 0, 1, 2, and 3;
R¹ is selected from C₃₋₈ cycloalkyl or 4- to 12-membered heterocyclyl, wherein the heterocyclyl includes partially unsaturated or saturated monocyclic or polycyclic heterocyclyl, and the polycyclic heterocyclyl includes spiral, fused, or bridged heterocyclyl; the cycloalkyl or heterocyclyl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, —CH₂OC(O)NR⁹R¹⁰, CN, SR², C₂₋₄ alkenyl, C₂₋₄alkynyl, C₃₋₈cycloalkyl, 3 to 8-membered heterocyclyl, NR^{h}R^{h}, —(CR⁷R¹)ₘ—OR^{h}, —(CR⁷R⁸)ₘ—NR^{h}R^{h}, and ═M; wherein, R⁹ and R¹⁰ are each independently selected from hydrogen and C₁₋₄ alkyl, or R⁹ and R¹⁰ together with the N atom to which they attached form a 4-to-8-membered heterocyclyl comprising 1 or 2 N atoms and 0 or 1 heteroatom selected from O and S; each R^{h} is independently hydrogen, C₁₋₄ alkyl, or C₁₋₄haloalkyl; M is selected from 0 and CR³R⁴; the definitions of R², R³, R⁴, R⁷, R¹, and m are as described above; and
R¹¹ is selected from hydrogen, halogen, C₁₋₄ alkyl, C₁₋₄haloalkyl, C₁₋₄ alkoxy, C₁₋₄haloalkoxy, C₂₋₄alkynyl, C₃₋₆cycloalkyl, and CN.

3. The compound according to claim 1 or 2, or an optical isomer, a pharmaceutically acceptable salt, a prodrug, a deuterated derivative, a hydrate, or a solvate thereof, wherein, the structure fragment 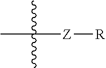
in formula (I) or formula (II) is selected from the group consisting of: 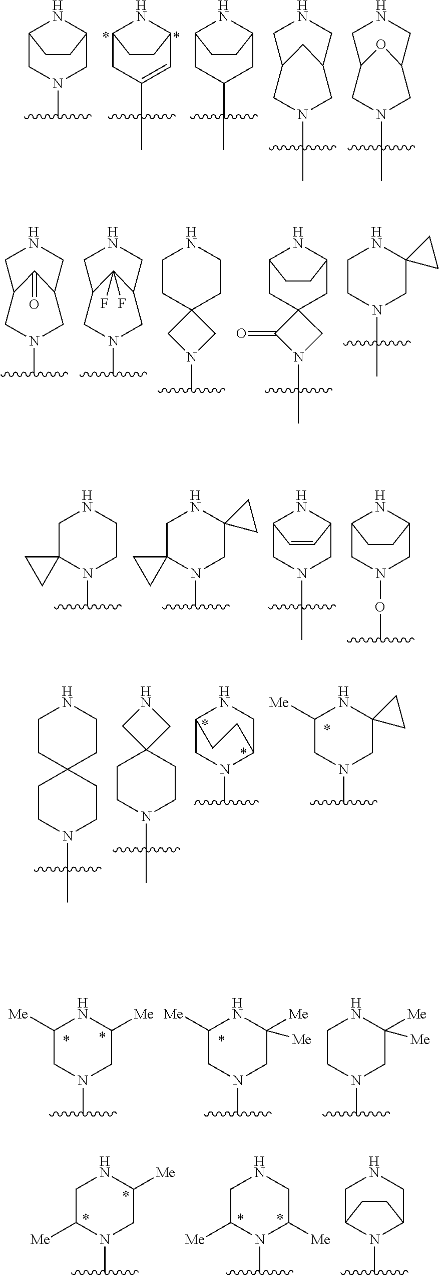 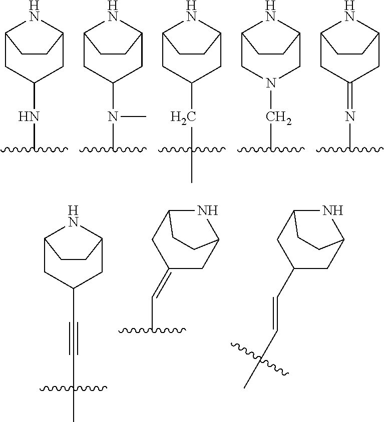
“ [symbol] ” represents the connection site between the above structural fragment and the rest of the structure of formula (I) or formula (II);
“*” represents a chiral center; and
the above-mentioned group is optionally substituted by 0, 1, or 2 R¹³, wherein R¹³ is selected from halogen, C₁₋₄ alkyl, ═O, hydroxyl, and CN.

4. The compound according to any one of claims 1-3, or an optical isomer, a pharmaceutically acceptable salt, a prodrug, a deuterated derivative, a hydrate, or a solvate thereof, wherein, Ar is selected from the group consisting of: 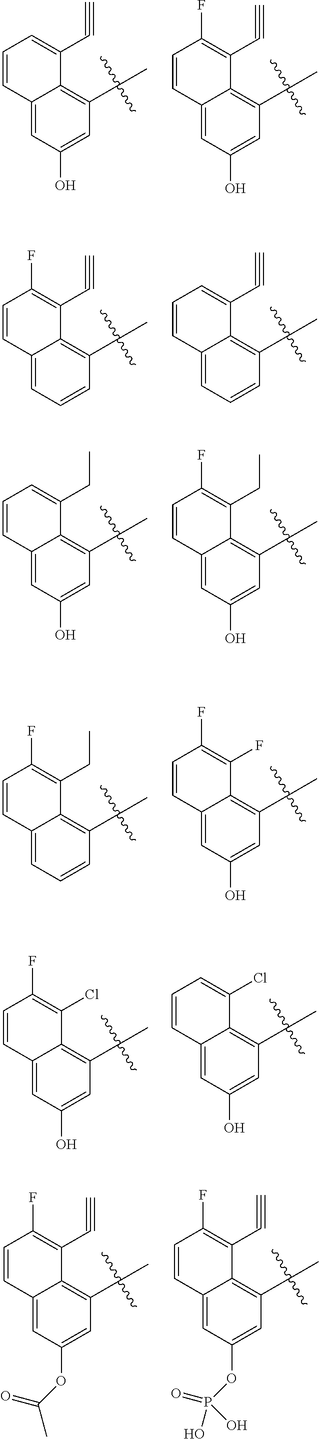 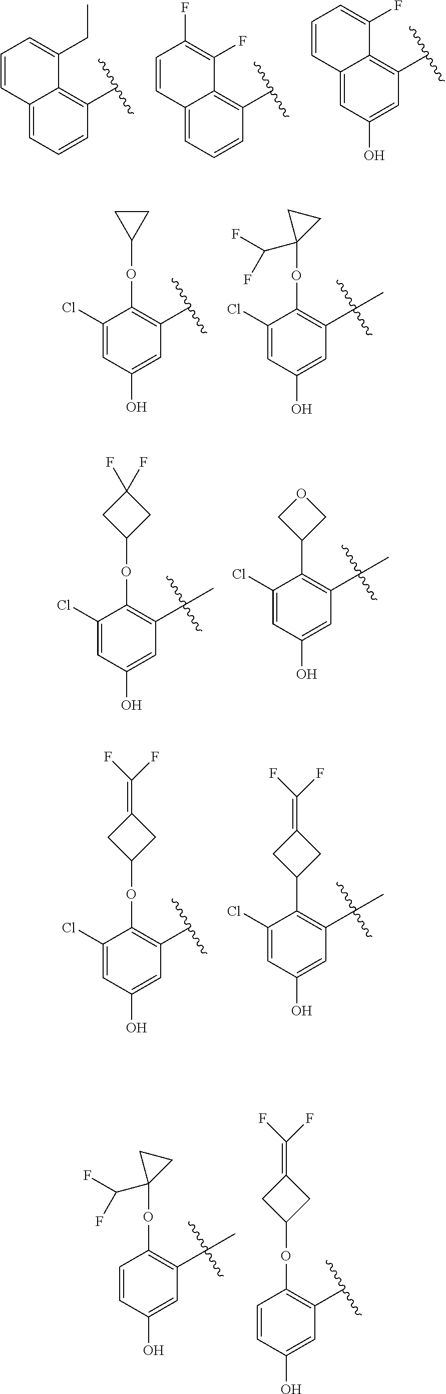 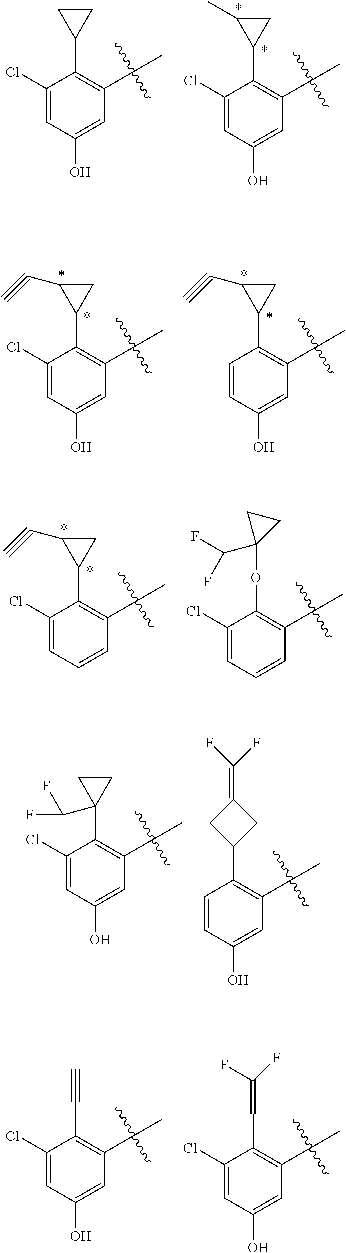 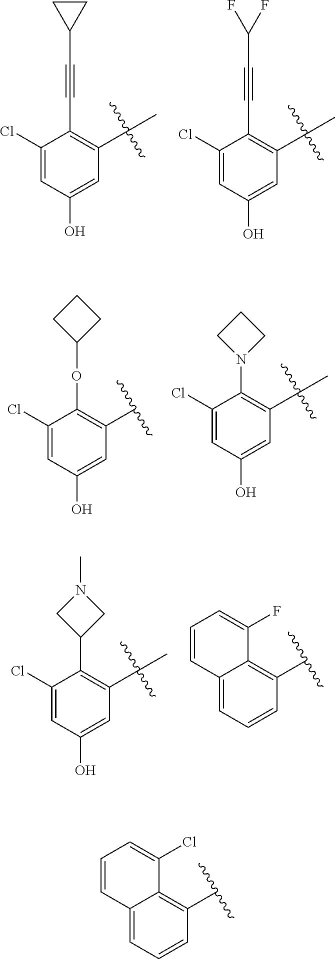
“ [symbol] ” represents the connection site between the above-mentioned structural fragment and the rest of the structure of formula (I) or formula (II); and
“*” represents a chiral center.

5. The compound according to any one of claims 1-4, or an optical isomer, a pharmaceutically acceptable salt, a prodrug, a deuterated derivative, a hydrate, or a solvate thereof, wherein, the structural fragment 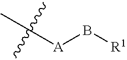
in formula (I) or formula (II) is a group selected from the group consisting of: 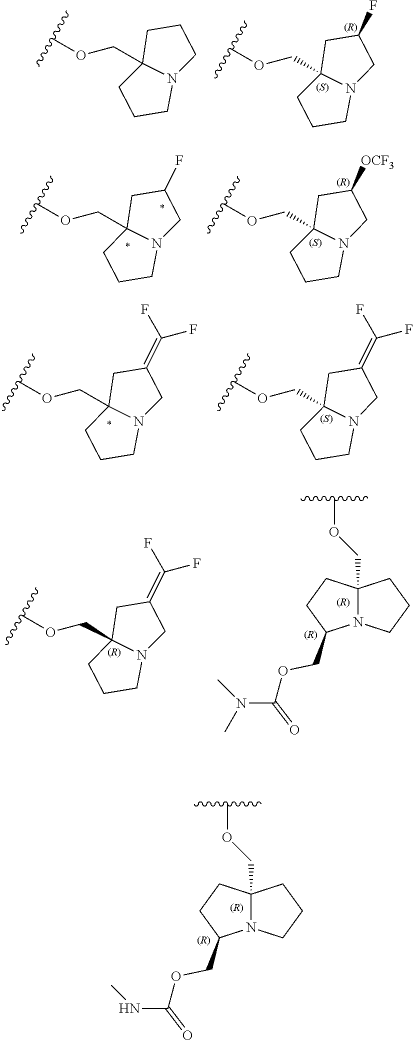
“*” represents a chiral center; and
“ [symbol] ” represents the connection site between the above-mentioned structural fragment another structures in formula (I) or formula (II).

6. The compound according to any one of claims 1-5, or an optical isomer, a pharmaceutically acceptable salt, a prodrug, a deuterated derivative, a hydrate, or a solvate thereof, wherein, formula (I) is formula (IIIa) or formula (IIIb), 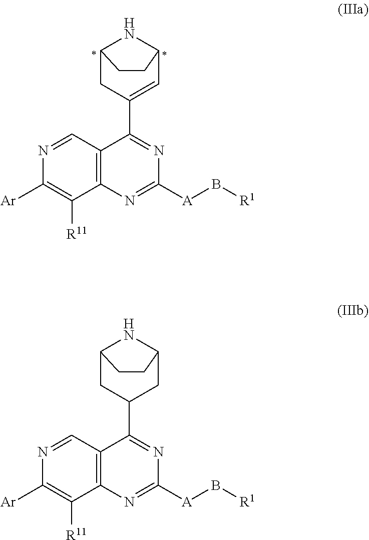
“*” represents a chiral center; and
Ar, A, B, R¹, and R¹¹ are as defined in claim 1.

7. The compound according to claim 1, or an optical isomer, a pharmaceutically acceptable salt, a prodrug, a deuterated derivative, a hydrate, or a solvate thereof, wherein, formula (I) is formula (IV), 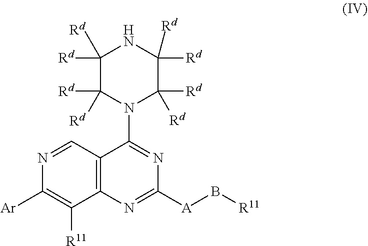
each R^{d} is independently selected from hydrogen, C₁₋₄ alkyl, and C₃₋₆ cycloalkyl; or two R^{d} together with the same C atom they attached form a C₃₋₆ cycloalkyl; and at least two R^{d} together with the same C atom they attached form a C₃₋₆ cycloalkyl; and
Ar, A, B, R¹, and R¹¹ are as defined in claim 1.

8. The compound according to claim 1, or an optical isomer, a pharmaceutically acceptable salt, a prodrug, a deuterated derivative, a hydrate, or a solvate thereof wherein, formula (I) is formula (V), 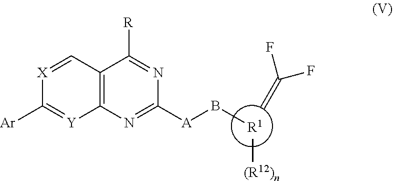
R¹is selected from C₃₋₈cycloalkyl and 4- to 12-membered heterocyclyl;
R¹² is selected from hydrogen, halogen, C₁₋₄ alkyl, C₁₋₄haloalkyl, —CH₂OC(O)NR⁹R¹⁰, C₂₋₄ alkenyl, C₂₋₄ alkynyl, C₃₋₈cycloalkyl, 3 to 8-membered heterocyclyl, CN, OR^{h}, SR^{h}, NR^{h}R^{h}, —(CR⁷R⁸)ₘ—OR^{h}, and —(CR⁷R⁸)ₘ—NR^{h}R^{h};
n is selected from 0, 1, and 2; and
Ar, X, Y, R, A, B, R⁷, R⁸, R⁹, R¹⁰, R^{h}, and m are as defined in claim 1.

9. The compound according to claim 1, or an optical isomer, a pharmaceutically acceptable salt, a prodrug, a deuterated derivative, a hydrate, or a solvate thereof, wherein, formula (I) is formula (VI), 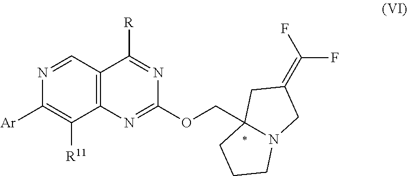
R is a group selected from the group consisting of 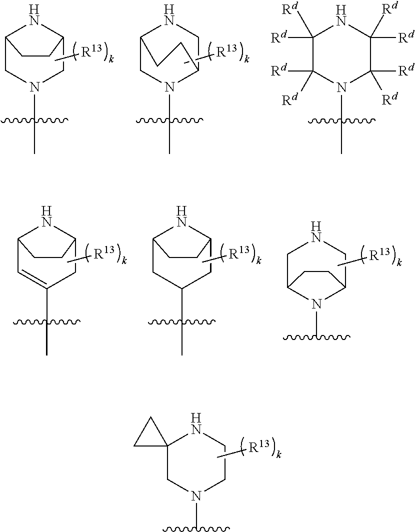
Ar is selected from the group consisting of: 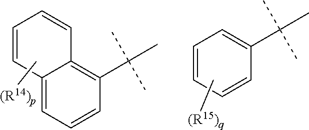
“*” represents a chiral center;
“ [symbol] ” represents the connection site between R and other structures of the compound of formula (VI);
“---” represents the connection site between Ar and other structures of the compound of formula (VI);
R¹³ is selected from halogen, C₁₋₄ alkyl, ═O, hydroxyl, and CN;
R¹⁴ and R¹⁵ are independently selected from halogen, C₁₋₄ alkyl, C₁₋₄haloalkyl, hydroxyl, C₁₋₄alkoxy, C₁₋₄haloalkoxy, C₂₋₄ alkenyl, C₂₋₄ haloalkenyl, C₂₋₄ alkynyl, C₂₋₄haloalkynyl, C₃₋₆ cycloalkyl, 3 to 6-membered heterocyclyl, C₃₋₆cycloalkyl-O—, 3 to 6-membered heterocyclyl-O—, NR²R², CN, SR², —OC(O)R^{k}, —O—P(O)(OR^{m})₂, and —O—CH(Rⁿ)—O—P(O)(OR^{m})₂; wherein, each R² is independently hydrogen or C₁₋₄ alkyl; R^{k} is selected from C₁₋₁₂ alkyl, wherein the alkyl is optionally substituted by one or more groups selected from the group consisting of: hydroxyl, C₁₋₄alkoxy, NR²R², C(O)OH, C(O)OC₁₋₂ alkyl, and CN; R⁸ is selected from hydrogen and C₁₋₄ alkyl; Rⁿ is selected from hydrogen and C₁₋₄ alkyl group; the cycloalkyl or heterocyclyl mentioned above is optionally substituted by one or more groups selected from the group consisting of: halogen, C₁₋₄ alkyl, C₁₋₄ haloalkyl, hydroxyl, C₁₋₄alkoxy, C₁₋₄haloalkoxy, C₂₋₄ alkenyl, C₂₋₄alkynyl, CN, and ═M; wherein, M is selected from 0 and CR³R⁴; R³ and R⁴ are independently selected from the group consisting of: hydrogen, fluorine, and C₁₋₄ alkyl;
k is selected from 0, 1, and 2;
p is selected from 0, 1, 2, 3, 4, and 5;
q is selected from 0, 1, 2, 3 and 4; and
R¹¹ is as defined in claim 1; and R^{d} is as defined in claim 7.

10. The compound according to claim 1, or an optical isomer, a pharmaceutically acceptable salt, a prodrug, a deuterated derivative, a hydrate, or a solvate thereof, wherein, formula (I) is formula (VII), 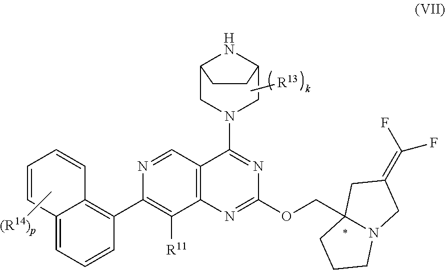
“*” represents a chiral center; and
R¹³, R¹⁴, R¹¹, k, and p are as defined in claim 9.

11. The compound according to claim 1, or an optical isomer, a pharmaceutically acceptable salt, a prodrug, a deuterated derivative, a hydrate, or a solvate thereof, wherein, formula (I) is formula (VIII), 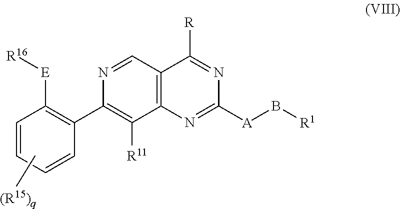
E is selected from chemical bonds, —O—, —S—, —NR—, —C═C—, and —CR^{a}═CR^{a}—;
R¹⁶ is selected from C₃₋₆cycloalkyl and 3- to 6-membered heterocyclyl; the cycloalkyl or heterocyclyl is optionally substituted by one or more groups selected from the group consisting of: halogen, C₁₋₄ alkyl, C₁₋₄haloalkyl, hydroxyl, C₁₋₄alkoxy, C₁₋₄haloalkoxy, C₂₋₄ alkenyl, C₂₋₄alkynyl, CN, and ═M; wherein, M is selected from O and CR³R⁴; R³ and R⁴ are independently selected from the group consisting of: hydrogen, fluorine, and C₁₋₄ alkyl; and
R, A, B, R¹, R¹¹, R⁵, and R^{a} are as defined in claim 1; R¹⁵ and q are as defined in claim 9.

12. The compound according to claim 1, or an optical isomer, a pharmaceutically acceptable salt, a prodrug, a deuterated derivative, a hydrate, or a solvate thereof, wherein, formula (I) is formula (IX), 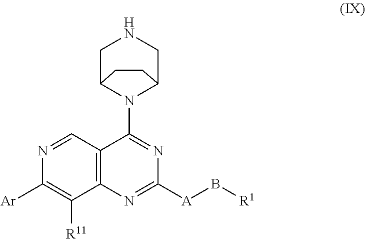
wherein, Ar, A, B, R¹, and R¹¹ are as defined in claim 1.

13. The compound according to claim 1, or an optical isomer, a pharmaceutically acceptable salt, a prodrug, a deuterated derivative, a hydrate, or a solvate thereof, wherein, formula (I) is formula (X), 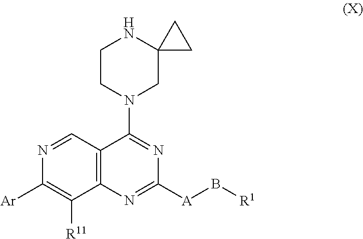
wherein, Ar, A, B, R¹, and R¹¹ are as defined in claim 1.

14. The compound according to claim 12 or 13, or an optical isomer, a pharmaceutically acceptable salt, a prodrug, a deuterated derivative, a hydrate, or a solvate thereof, wherein, in formula (IX) or formula (X),
the structural fragment 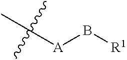
is a group selected form the group consisting of: 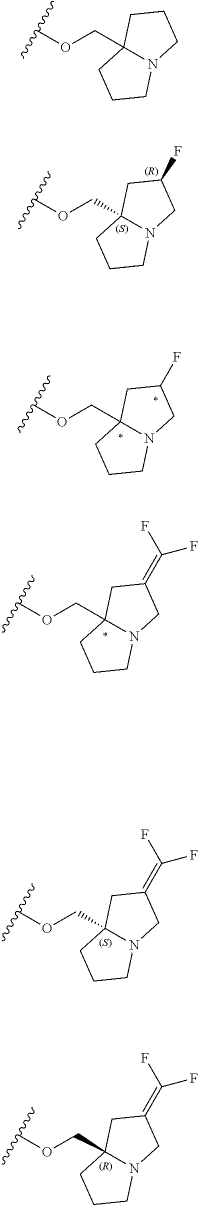
Ar is selected from the group consisting of: 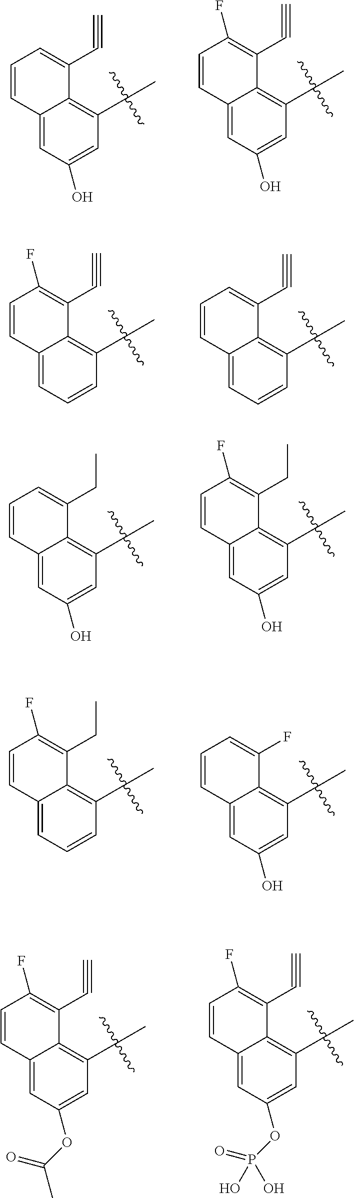
“*” represents a chiral center; and
“ [symbol] ” represents the connection site between the structural fragment 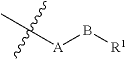
or Ar and other structures in formula (IX) or formula (X).

15. The compound according to claim 1, or an optical isomer, a pharmaceutically acceptable salt, a prodrug, a deuterated derivative, a hydrate, or a solvate thereof, wherein, formula (I) is formula (XI), 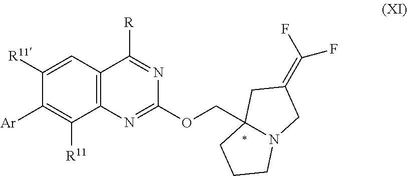
“*” represents a chiral center;
R¹¹ is selected from hydrogen, halogen, and C₁₋₄ alkyl;
R^{11′} is selected from hydrogen, halogen, C₁₋₄ alkyl, C₁₋₄alkoxy, C₁₋₄ haloalkoxy, and CN; and
Ar and R are as defined in claim 9.

16. The compound according to claim 1, or an optical isomer, a pharmaceutically acceptable salt, a prodrug, a deuterated derivative, a hydrate, or a solvate thereof, wherein, formula (I) is formula (XII), 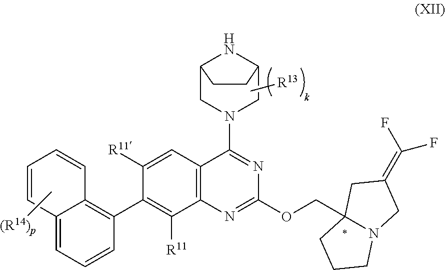
“*” represents a chiral center;
R¹¹ and R^{11′} are as defined in claim 15; and
R¹³, R¹⁴, k, and p are as defined in claim 9.

17. The compound according to claim 1, or an optical isomer, a pharmaceutically acceptable salt, a prodrug, a deuterated derivative, a hydrate, or a solvate thereof, wherein, formula (I) is formula (XIII), 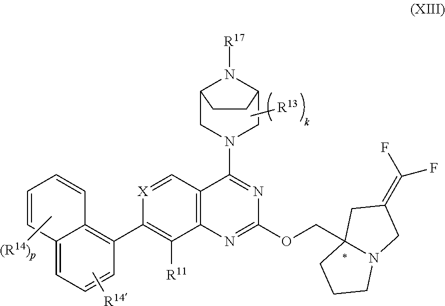
“*” represents a chiral center;
R¹¹ is selected from hydrogen, halogen, and C₁₋₄ alkyl;
X is selected from N and CR^{11′}; wherein, R^{11′} is selected from hydrogen, halogen, C₁₋₄ alkyl, C₁₋₄alkoxy, C₁₋₄ halogenated alkoxy, and CN;
R¹³ is selected from halogen, C₁₋₄ alkyl, ═O, hydroxyl, and CN;
R¹⁴ is selected from halogen, C₁₋₄ alkyl, C₁₋₄haloalkyl, hydroxyl, C₁₋₄alkoxy, C₁₋₄haloalkoxy, C₂₋₄ alkenyl, C₂₋₄haloalkenyl, C₂₋₄alkynyl, C₂₋₄haloalkynyl, C₃₋₆ cycloalkyl, 3 to 6-membered heterocyclyl, C₃₋₆cycloalkyl-O—, 3 to 6-membered heterocyclyl-O—, NR²R², CN, and SR²; wherein, each R² is independently hydrogen or C₁₋₄ alkyl;
R¹⁴ is selected from hydrogen, —OC(O)R^{k}, —O—P(O)(OR^{m})₂, and —O—CH(Rⁿ) —O—P(O)(OR^{m})₂; wherein, R^{k} is selected from C₁₋₁₂ alkyl, wherein the alkyl is optionally substituted by one or more groups selected from the group consisting of: hydroxyl, C₁₋₄alkoxy, NR²R², C(O)OH, C(O)OC₁₋₂ alkyl, and CN; R^{m} is selected from hydrogen and C₁₋₄ alkyl; R¹¹ is selected from hydrogen and C₁₋₄ alkyl;
R¹⁷ is selected from hydrogen and —CO(O)—CH(Rⁿ)—O—C(O)—U; wherein, R¹¹ is selected from hydrogen and C₁₋₄ alkyl; U is selected from C₁₋₁₈ alkyl;
k is selected from 0, 1, and 2; and
p is selected from 0, 1, 2, 3, and 4.

18. A compound of the structure shown in formula (XIV) below, or an optical isomer, a pharmaceutically acceptable salt, a prodrug, a deuterated derivative, a hydrate, or a solvate thereof: 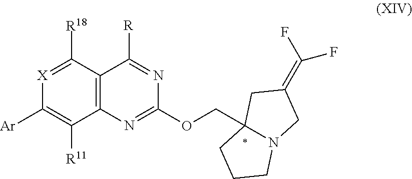
wherein:
“*” represents a chiral center;
R¹¹ is selected from hydrogen, halogen, and C₁₋₄ alkyl;
X is selected from N and CR^{11′}; wherein, R^{11′} is selected from hydrogen, halogen, C₁₋₄ alkyl, C₁₋₄alkoxy, C₁₋₄ haloalkoxy, and CN;
R¹⁸ is selected from halogen, C₁₋₄ alkyl, and C₁₋₄ alkoxy; and
Ar and R are as defined in claim 9.

19. The compound according to claim 1, wherein, the compound of formula (I) is selected form the group consisting of: 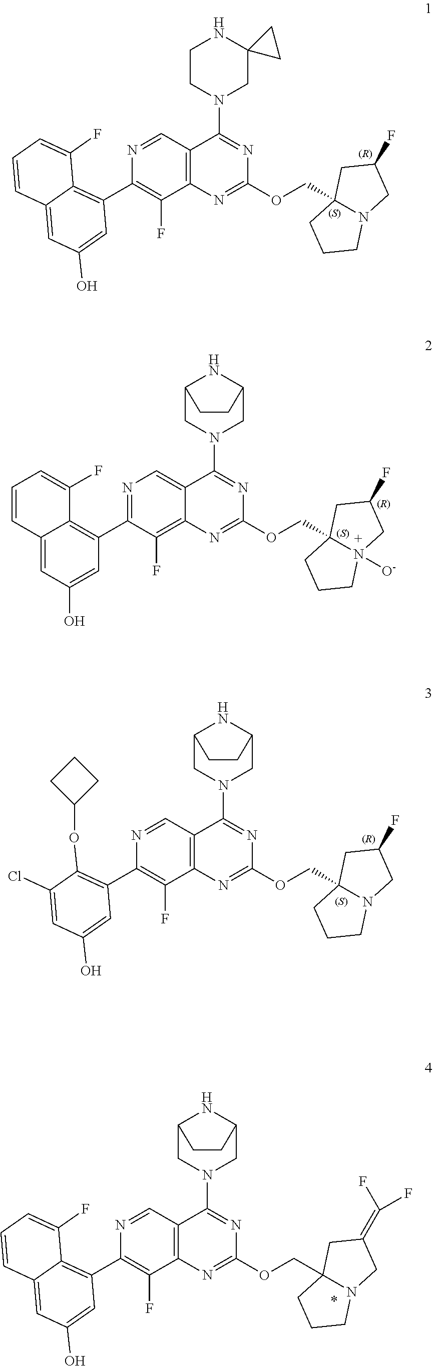 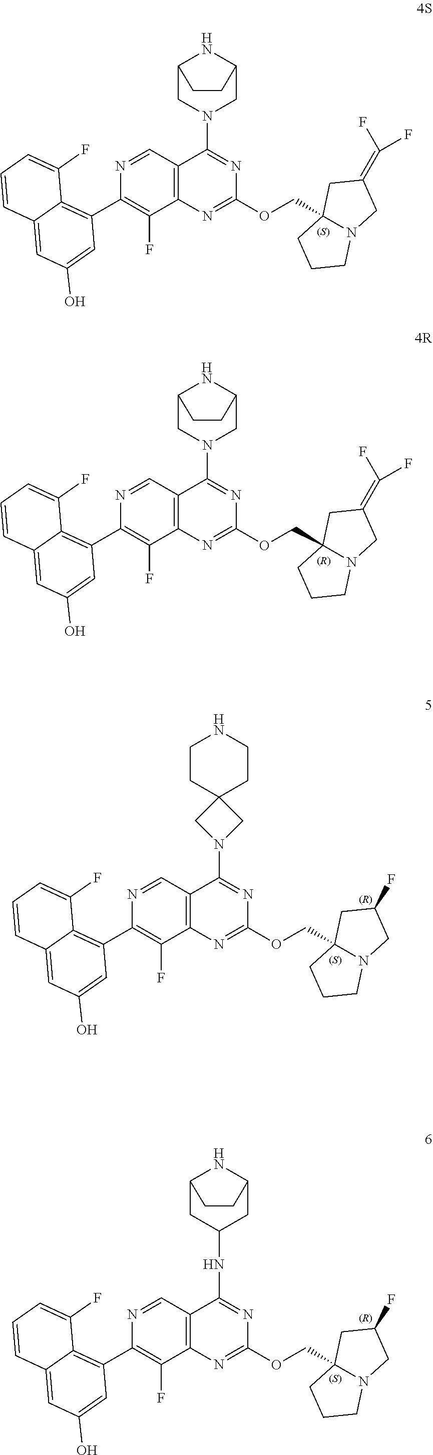 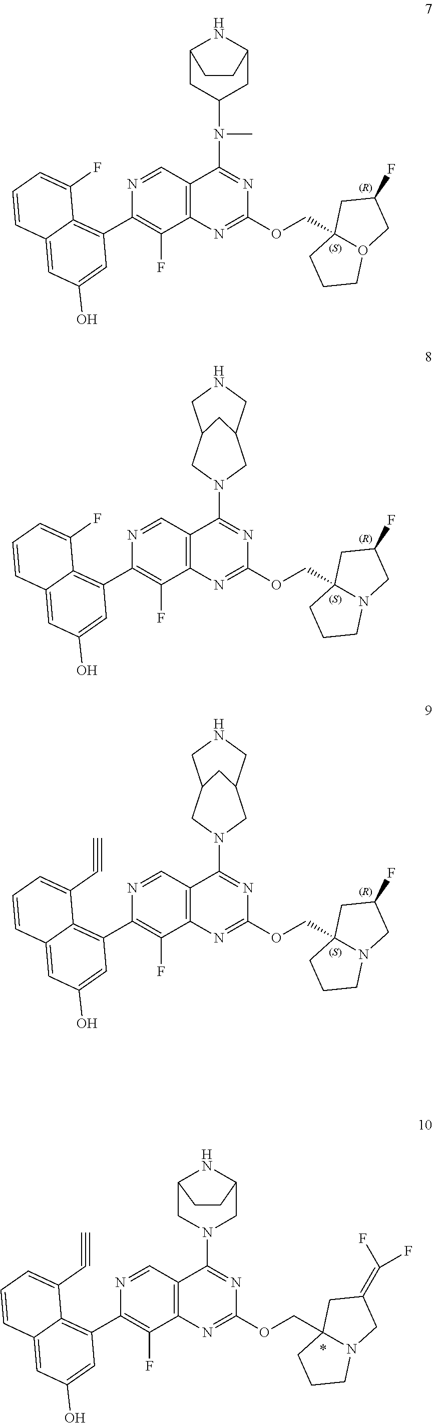 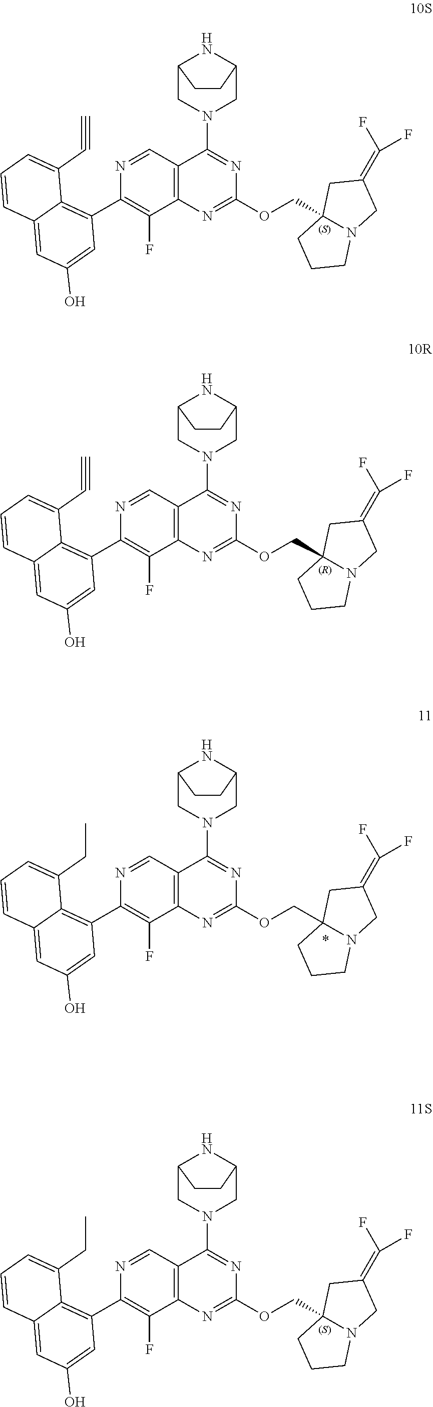 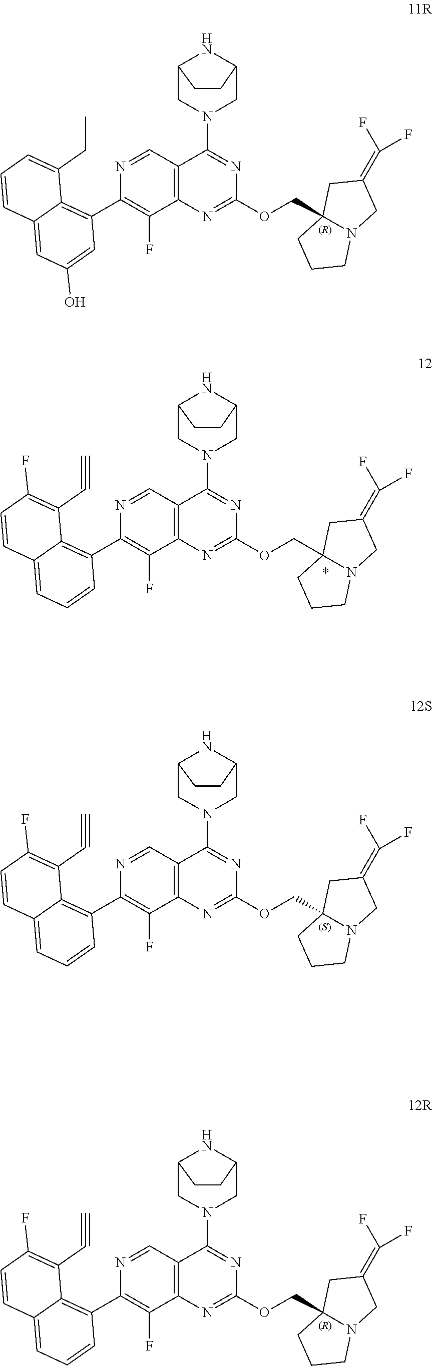 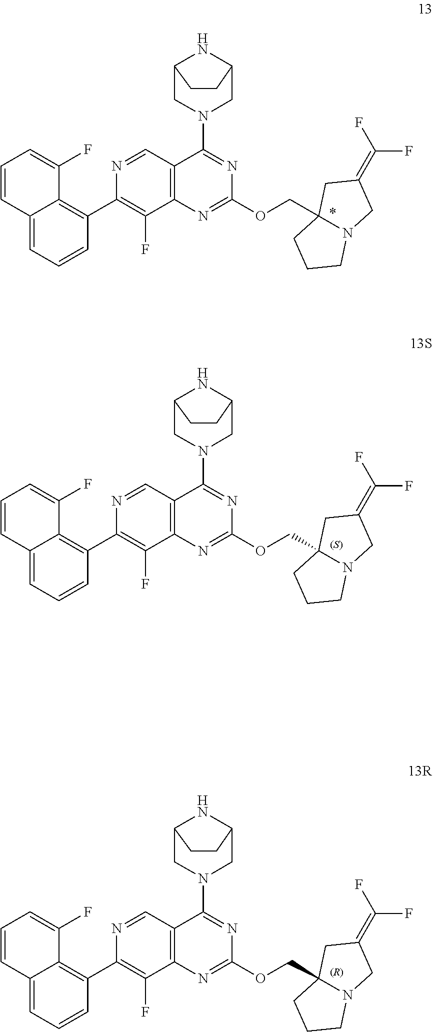 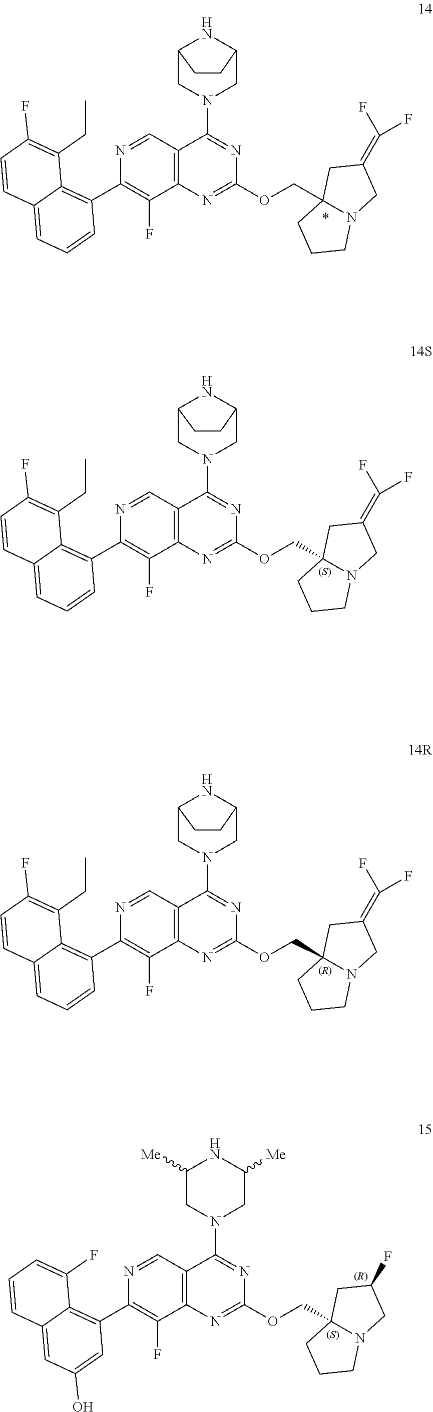 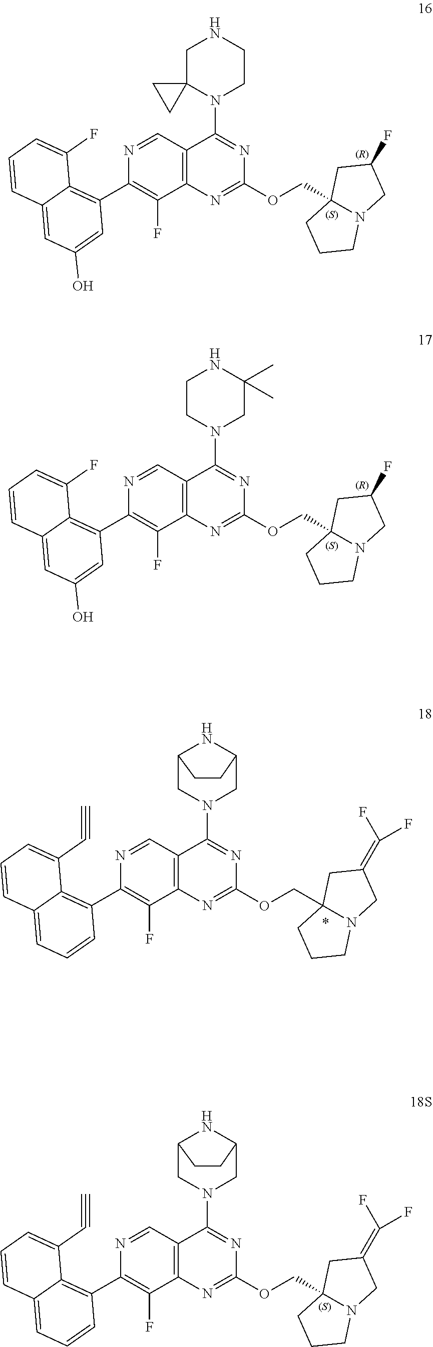 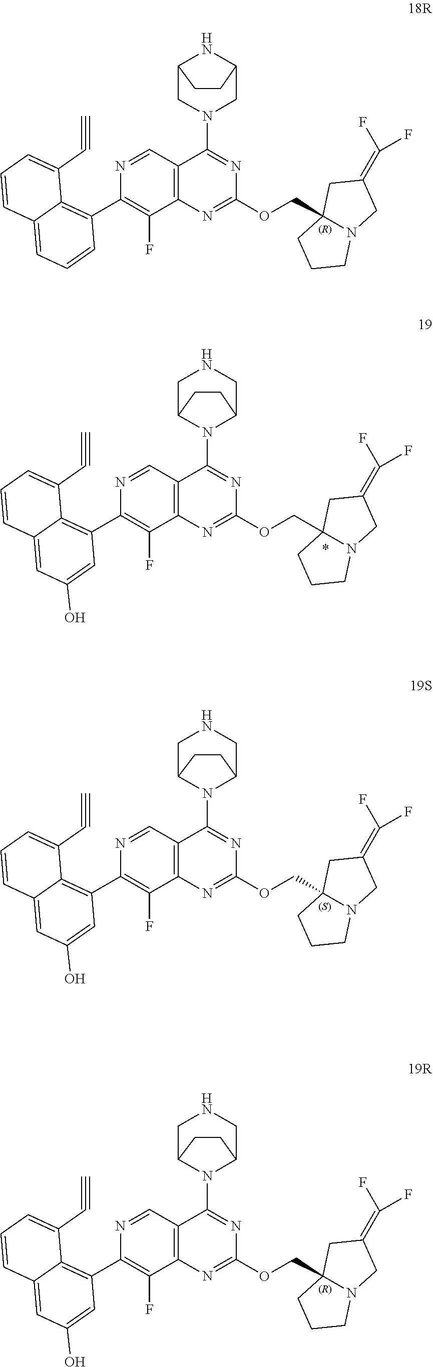 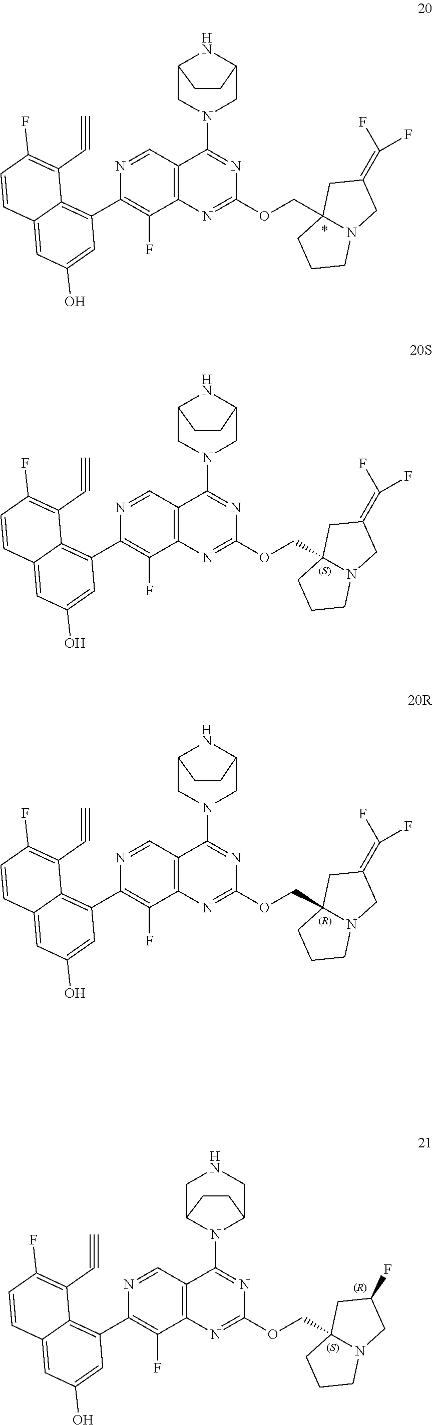 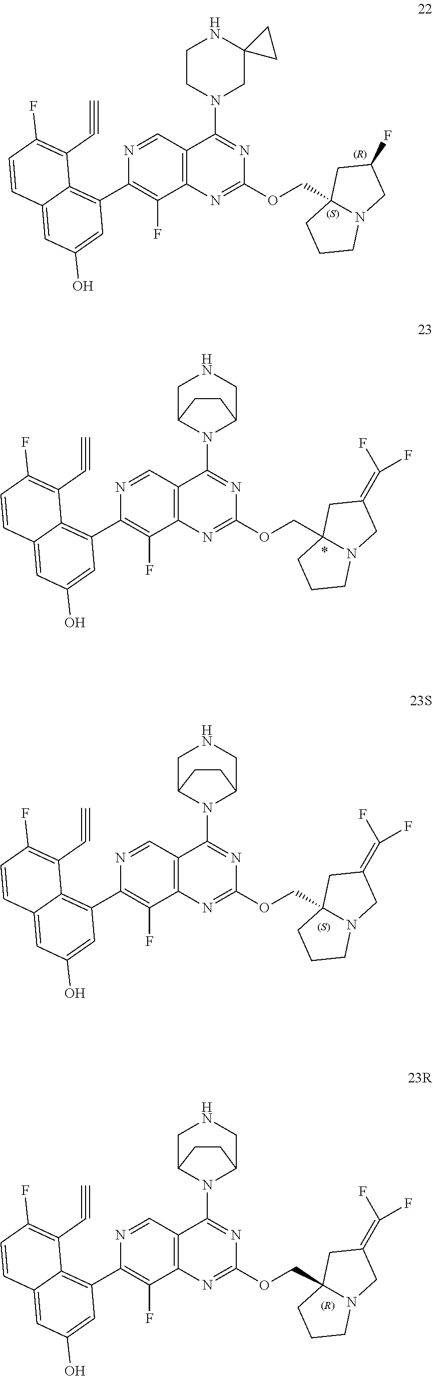 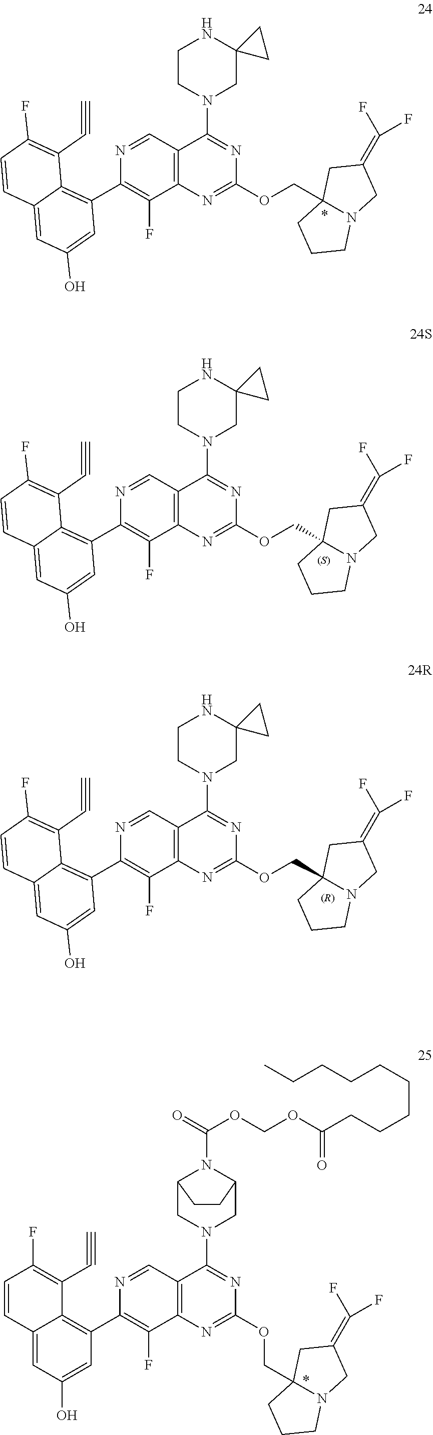 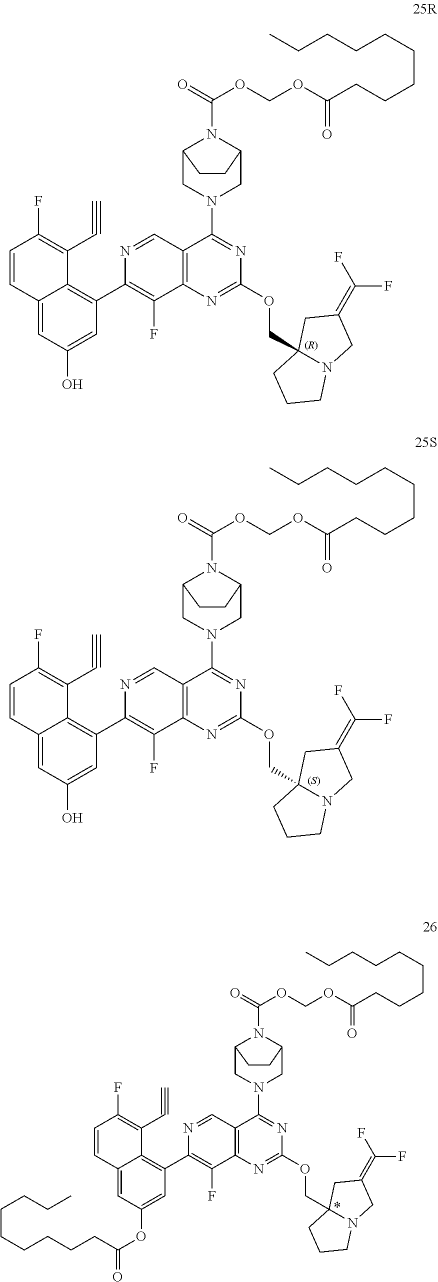 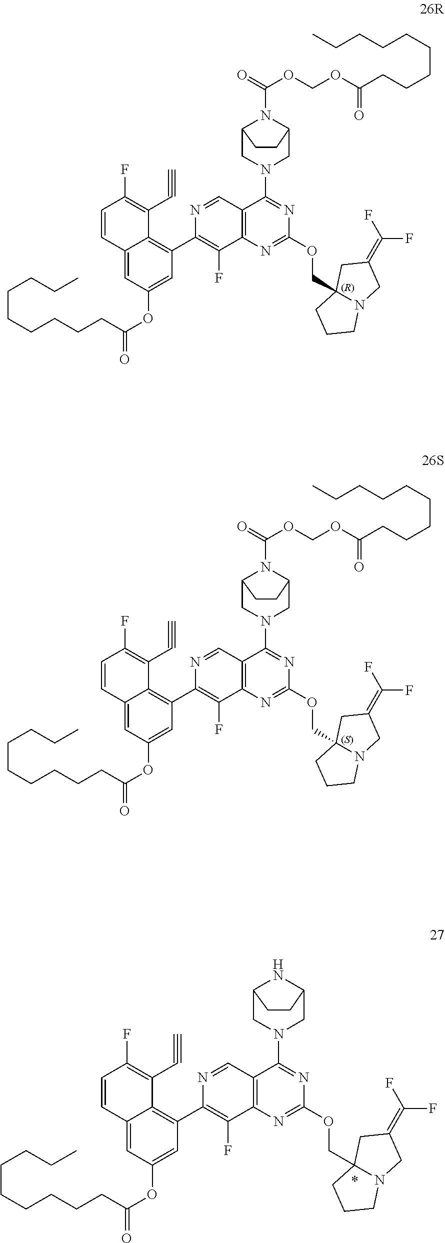 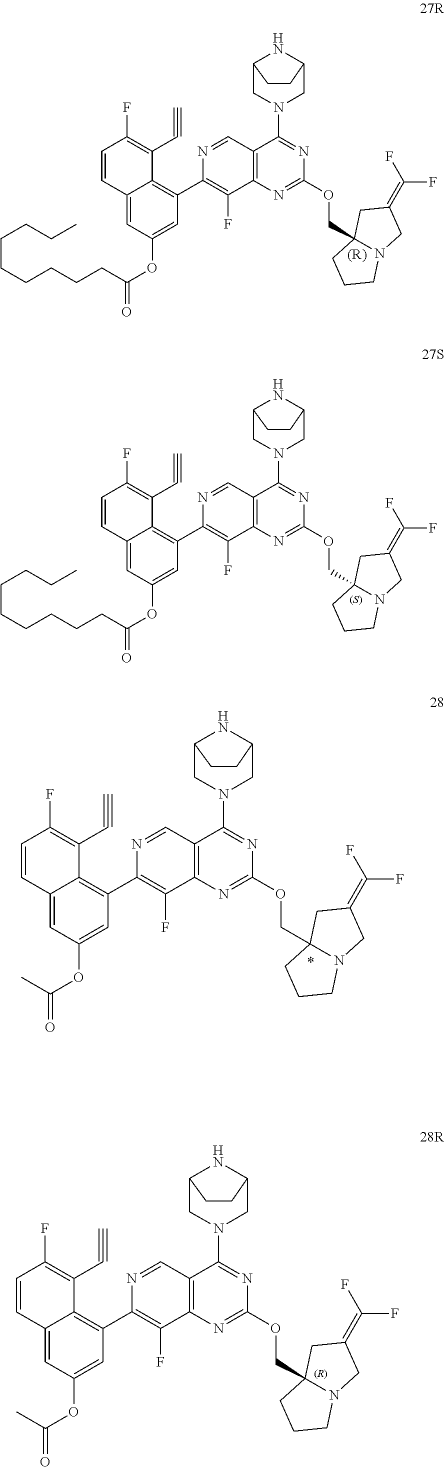 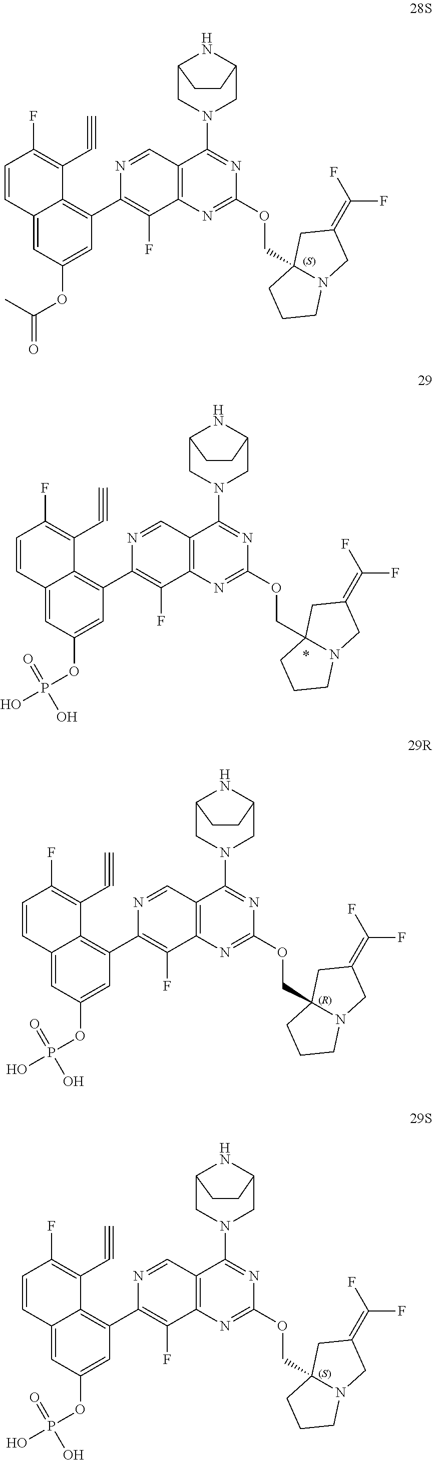 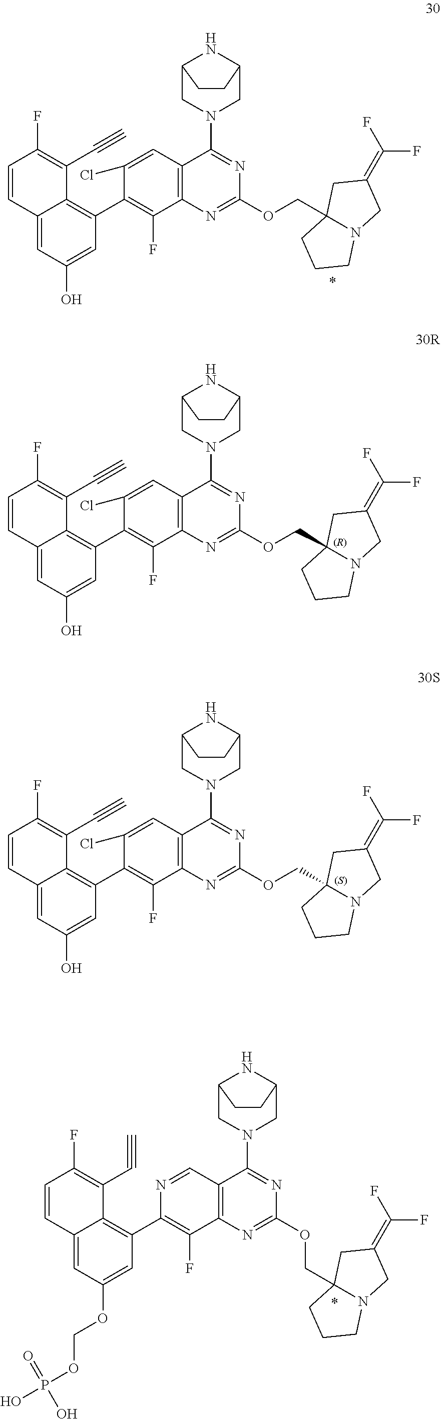 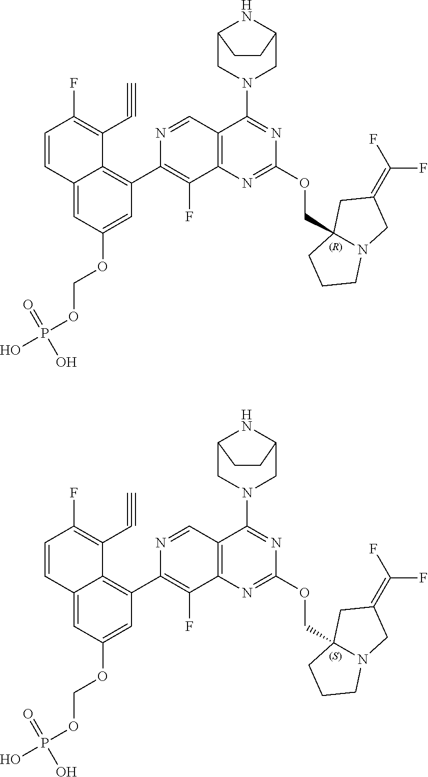 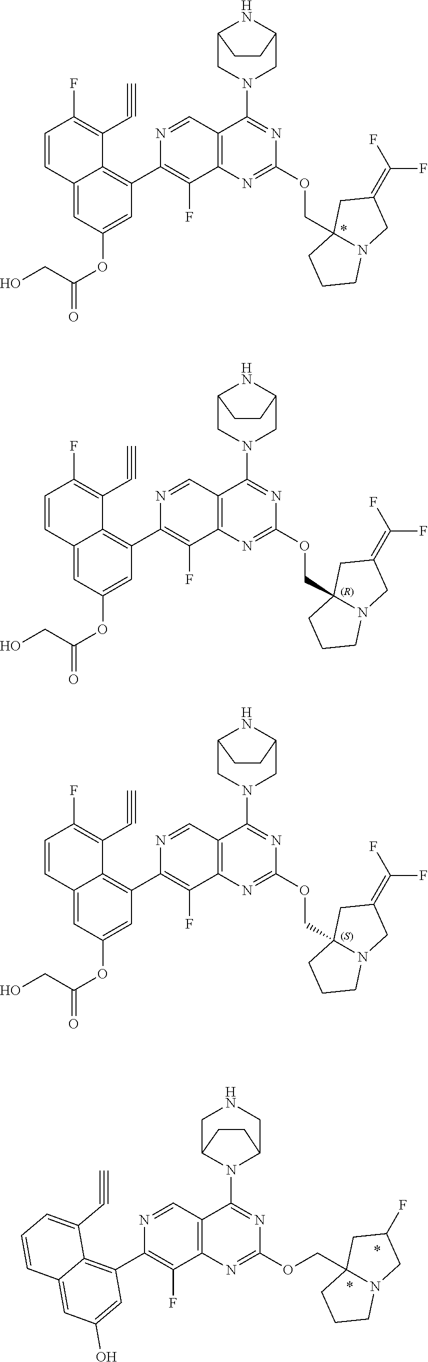 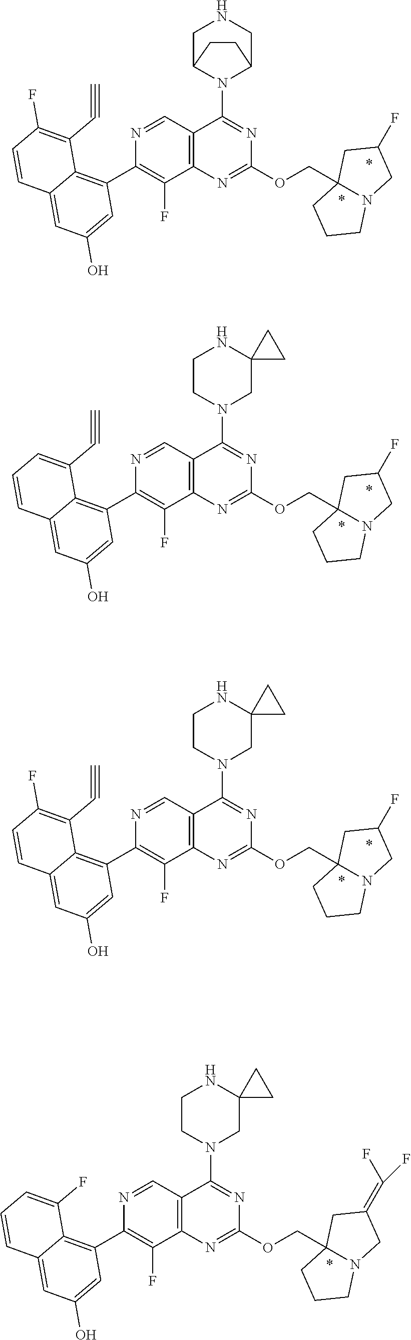 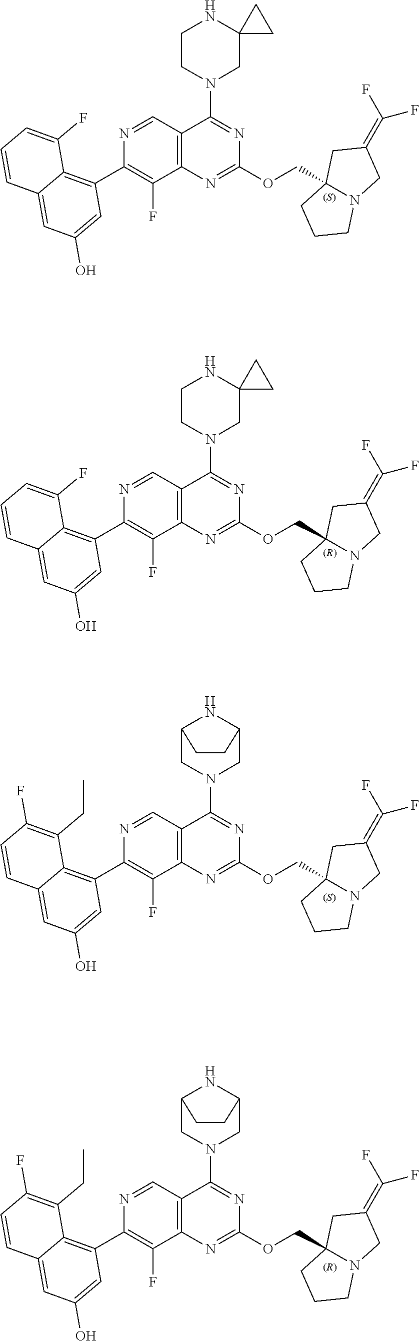 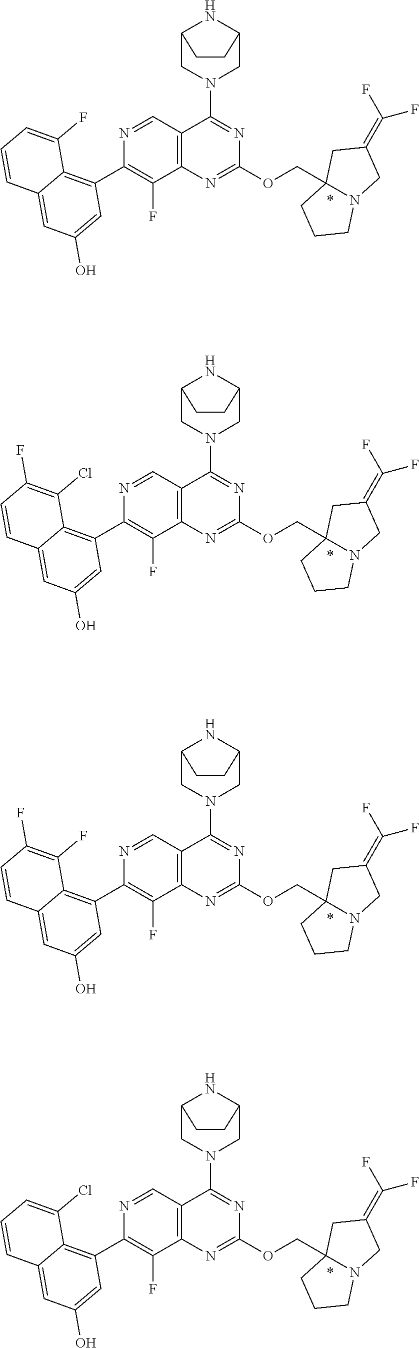 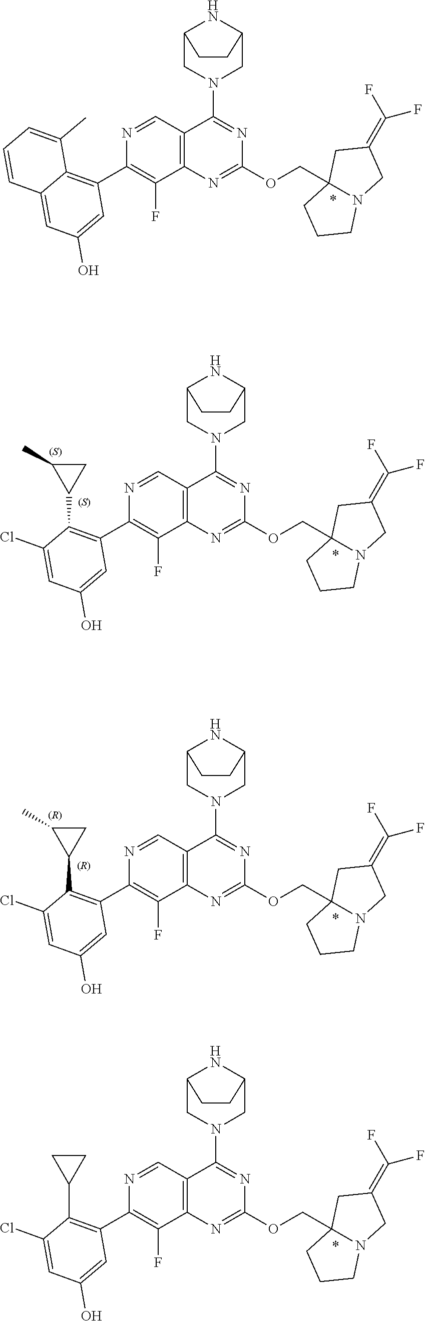 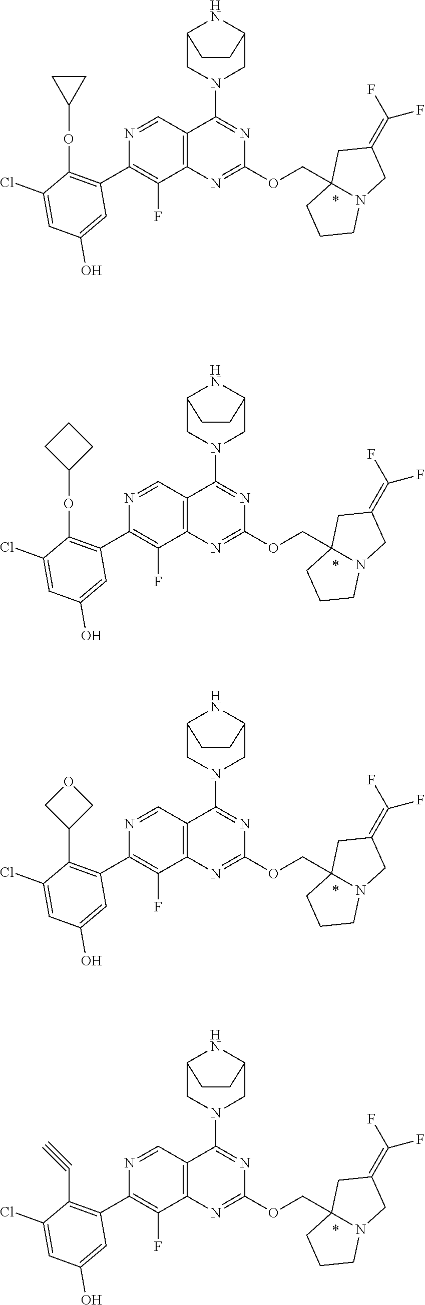 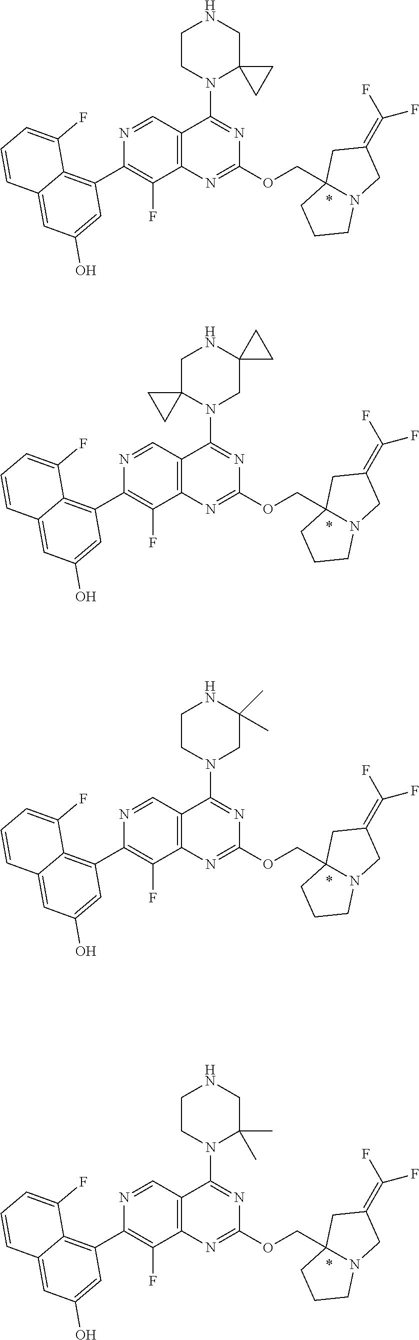 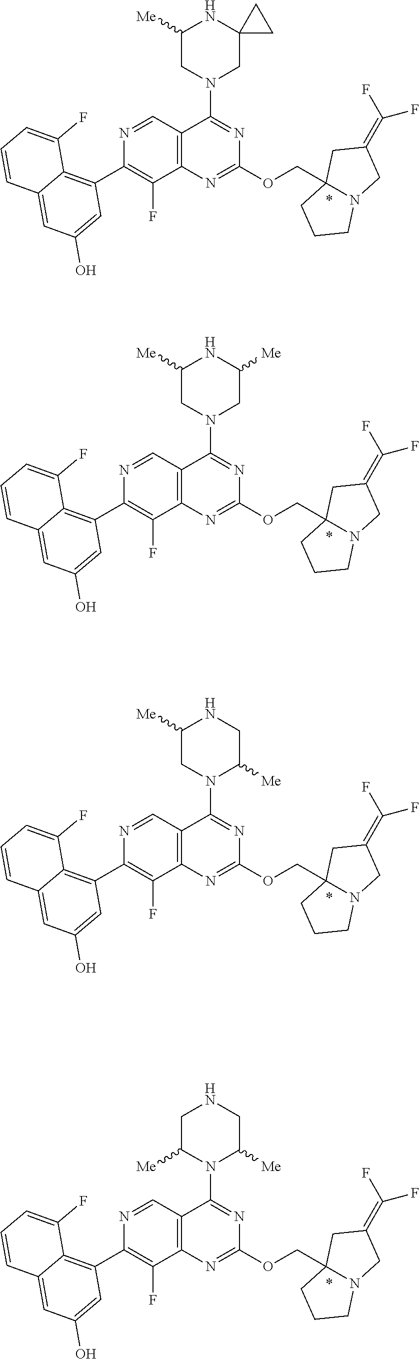 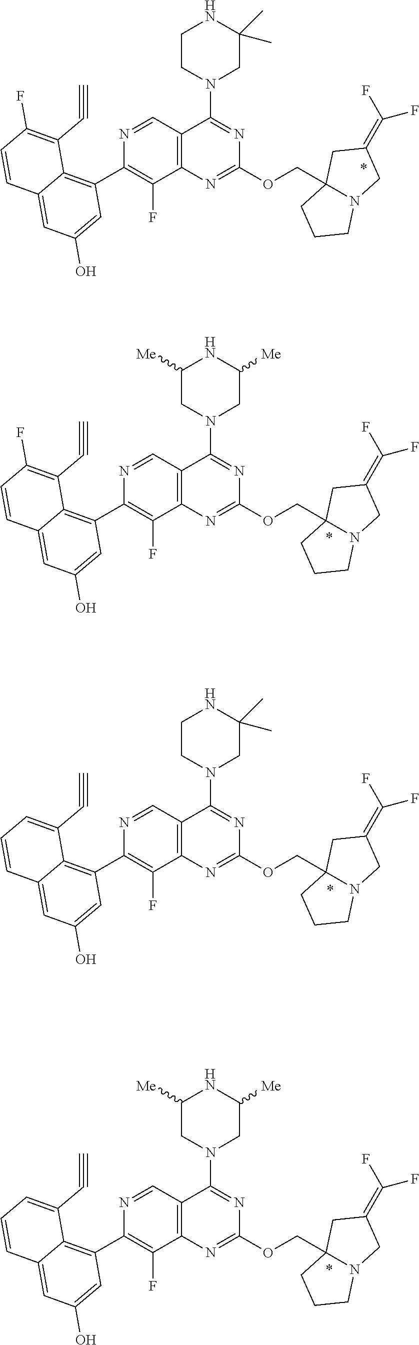 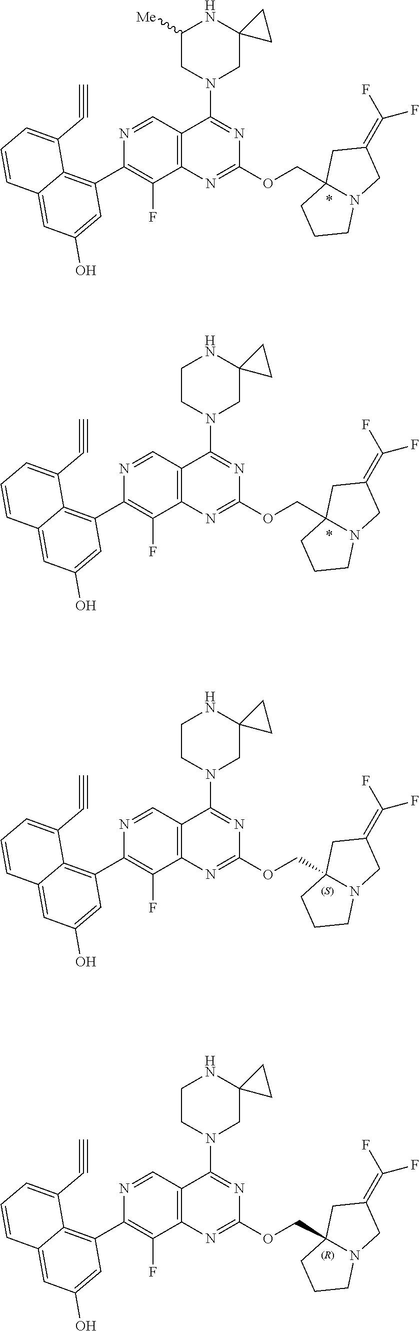 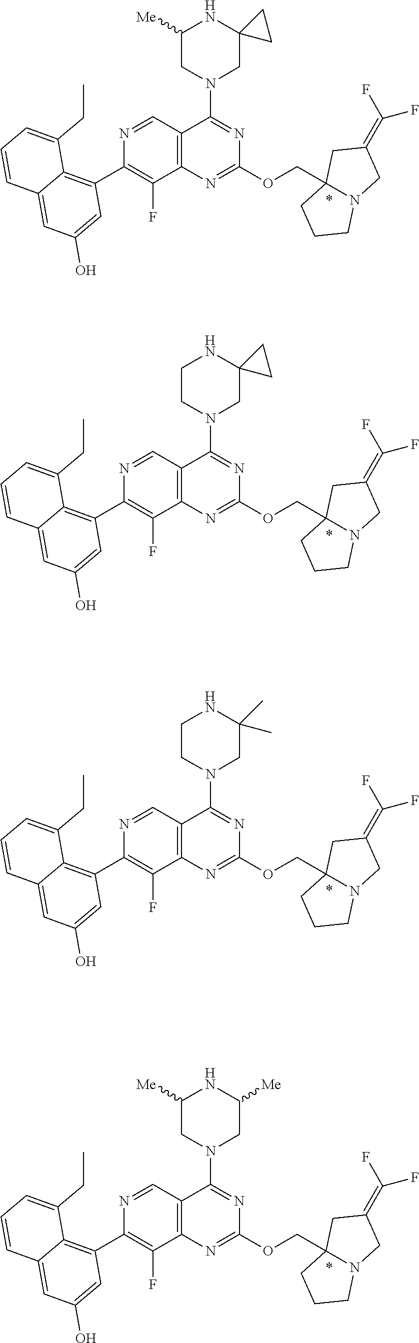 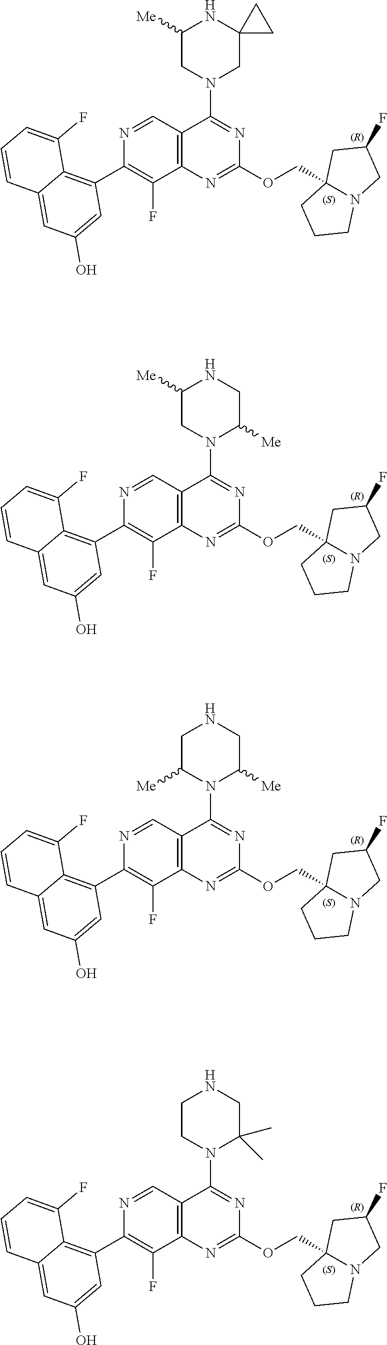 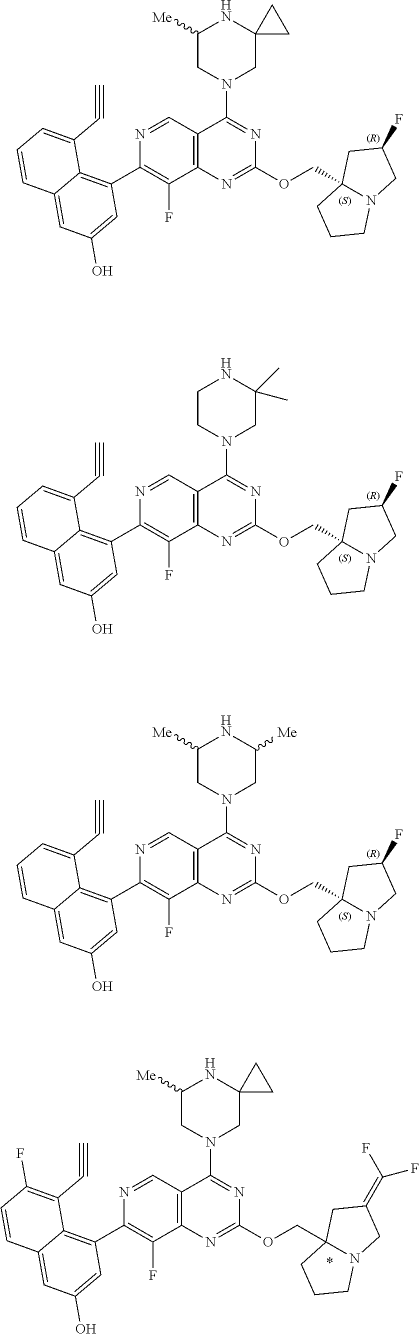 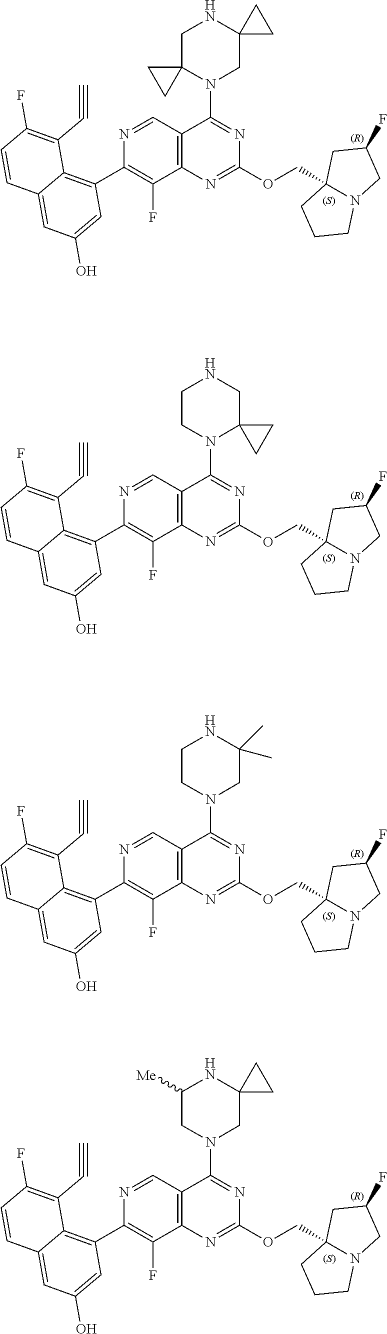 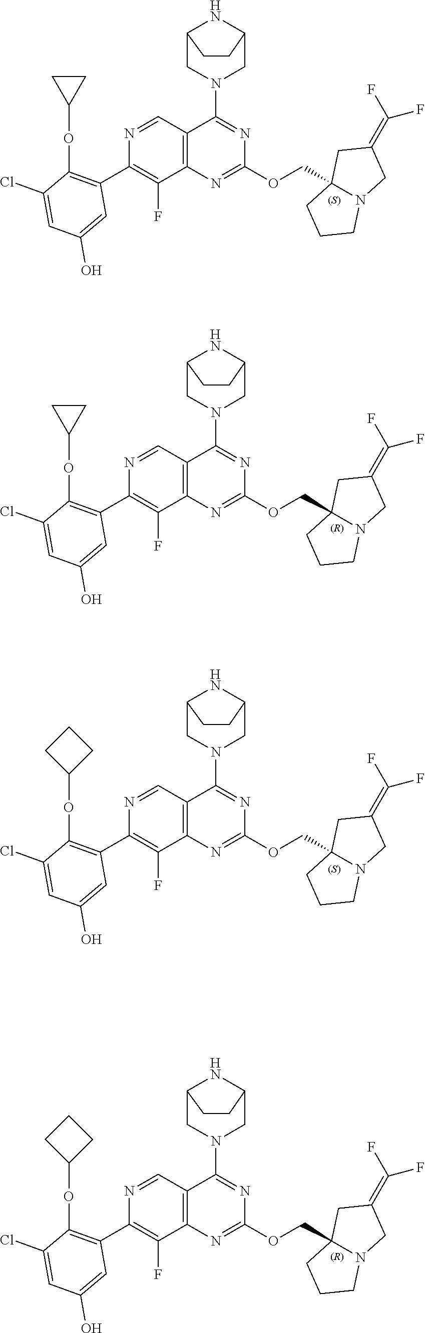 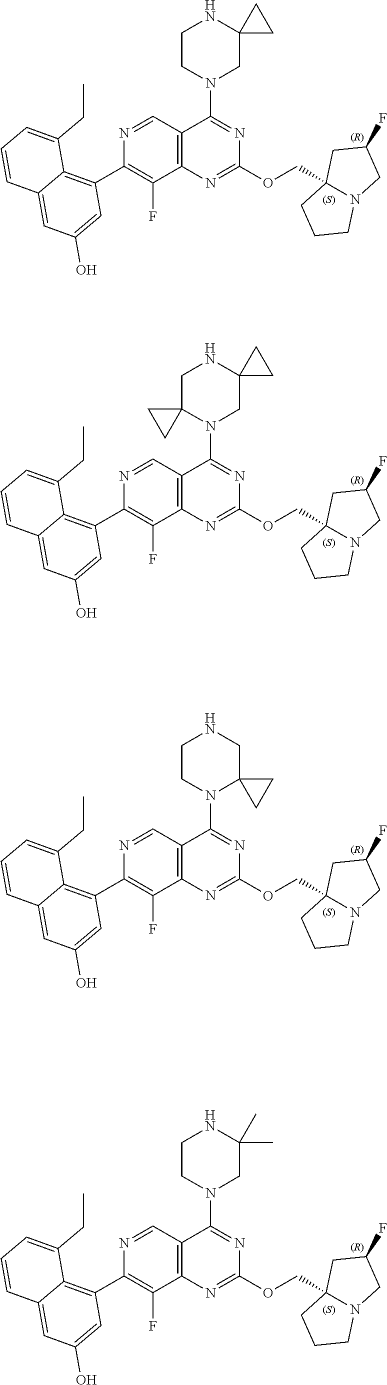 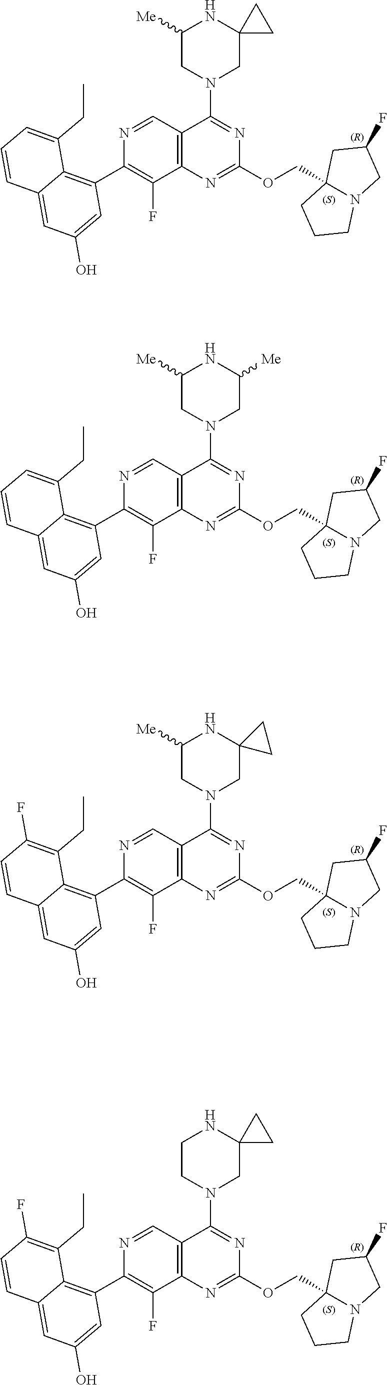 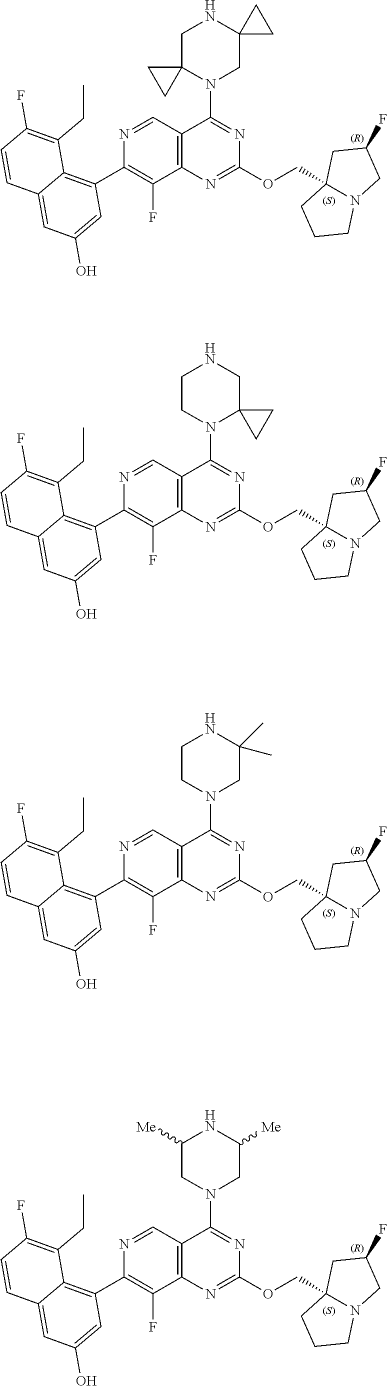 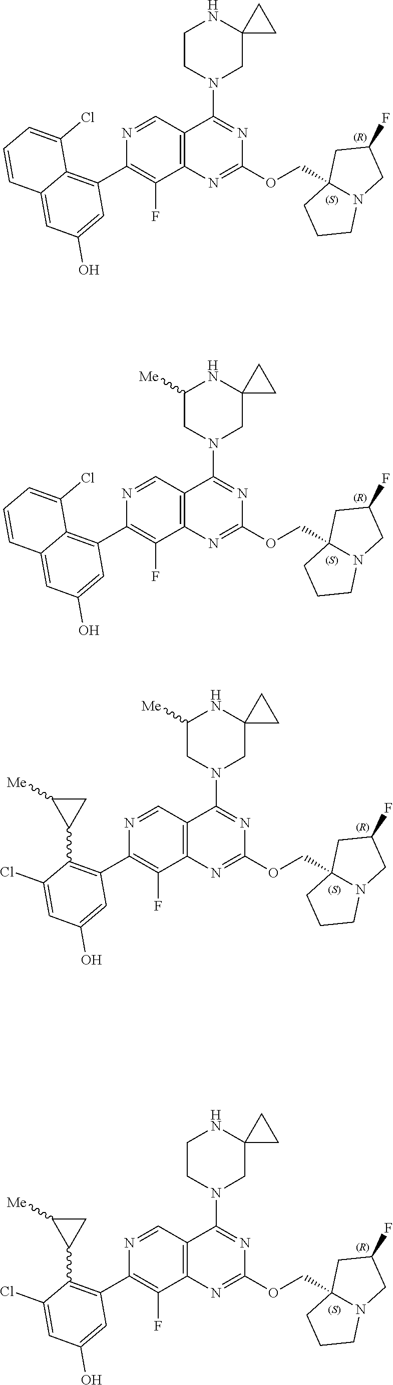 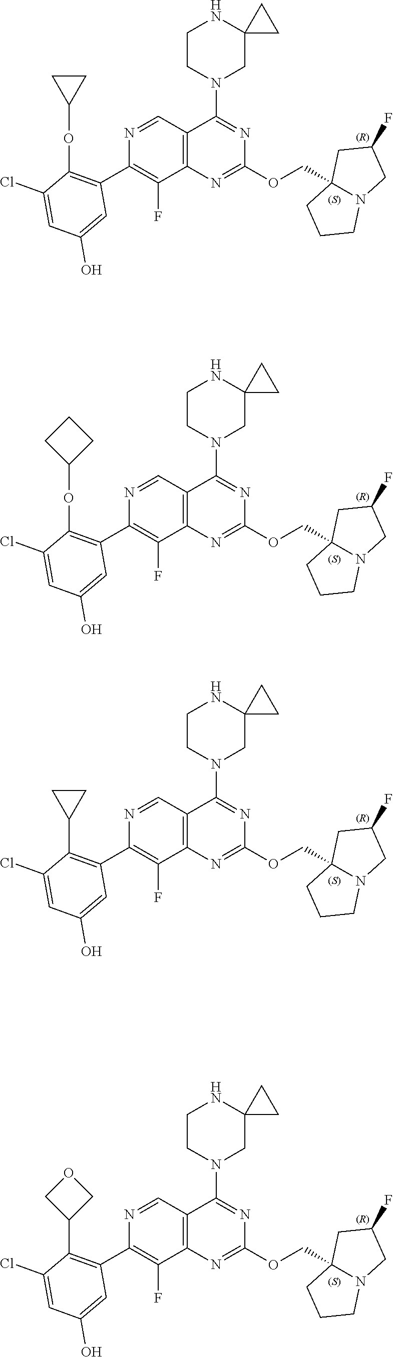 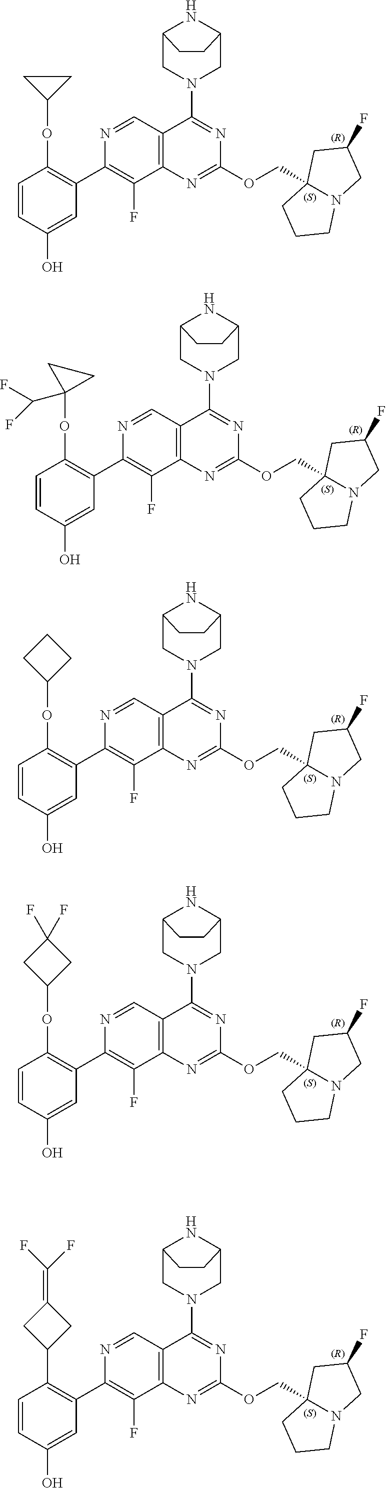 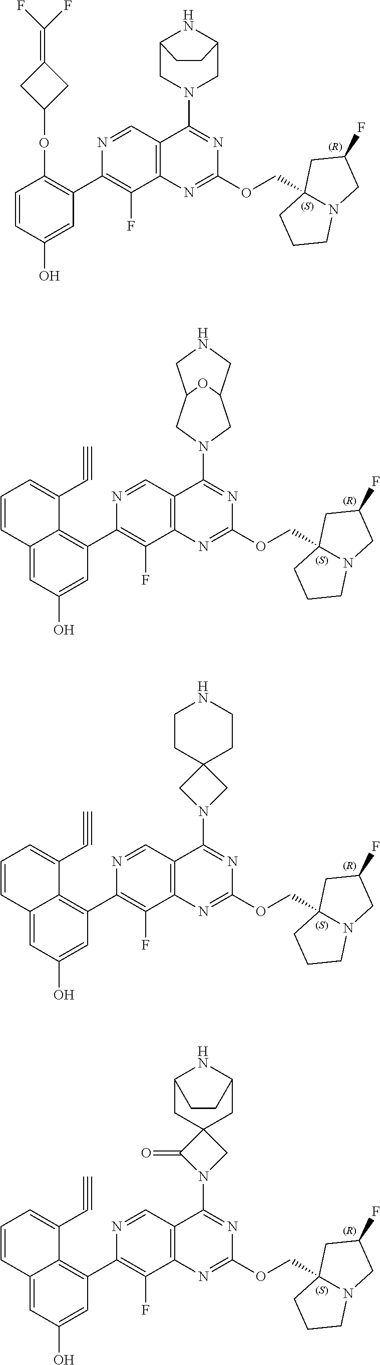 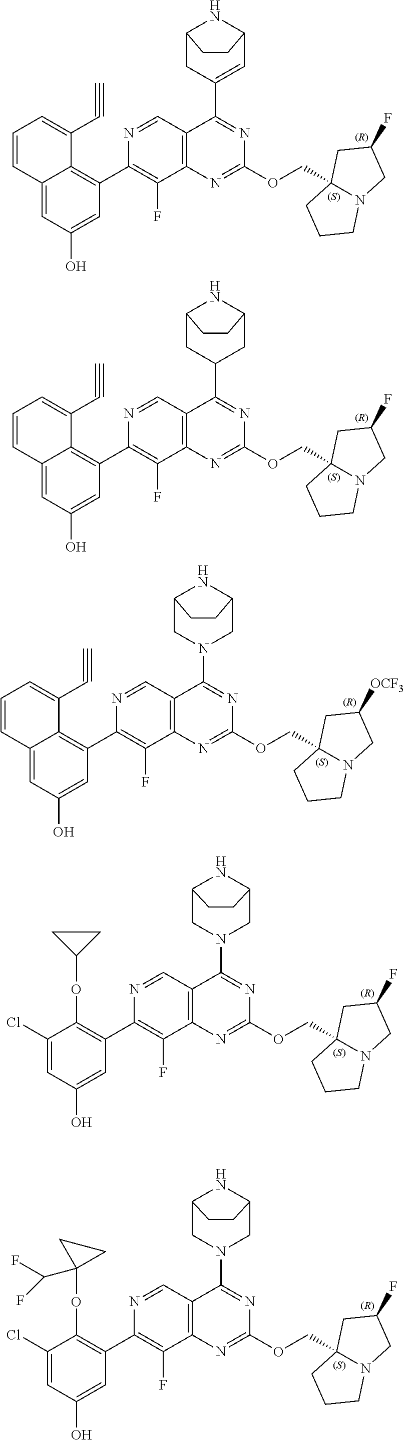 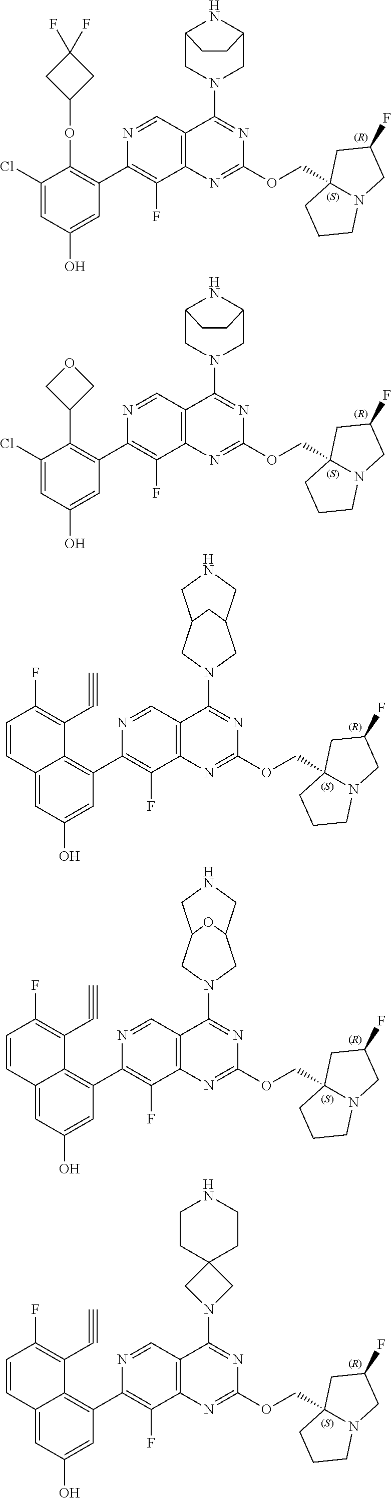 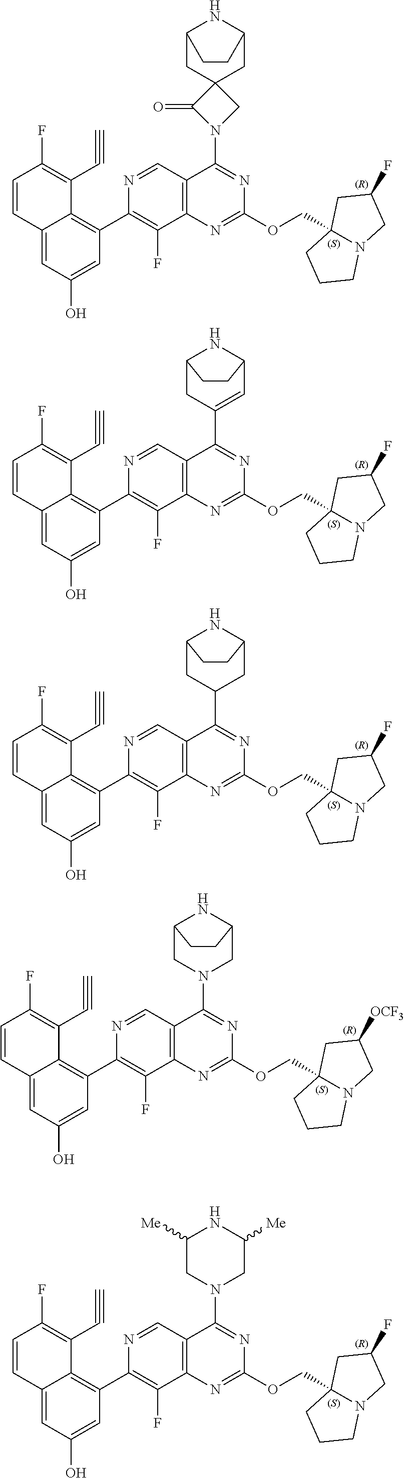 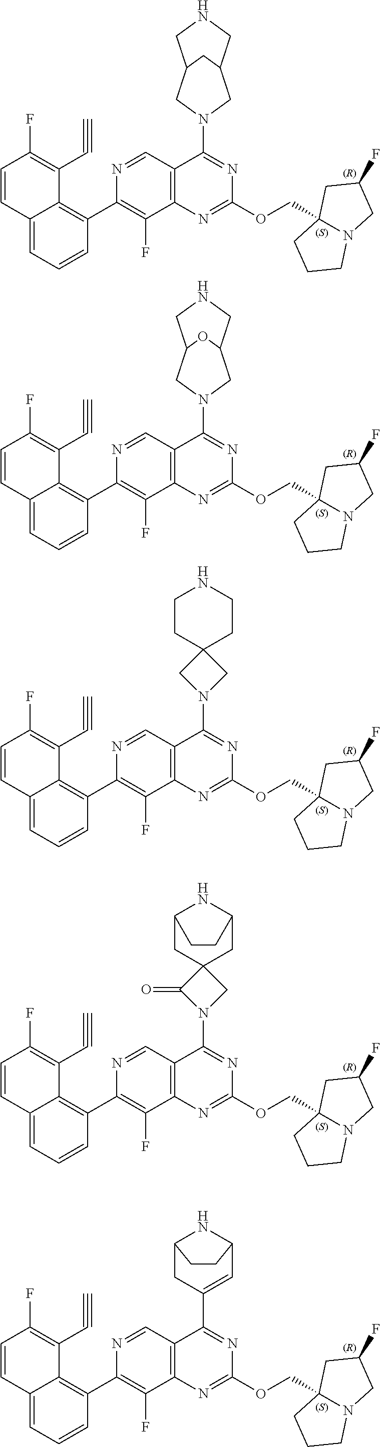 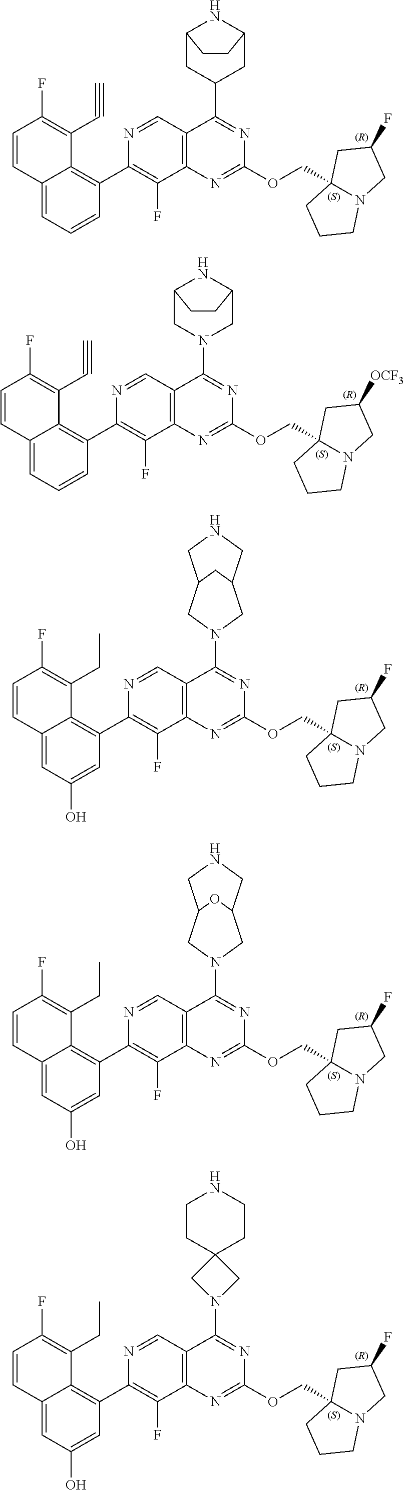 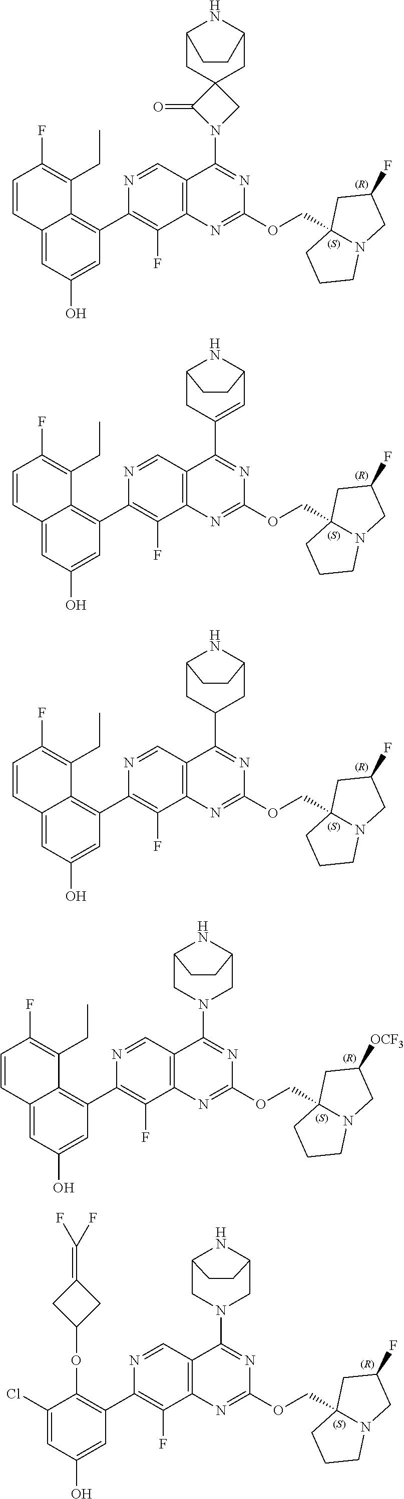 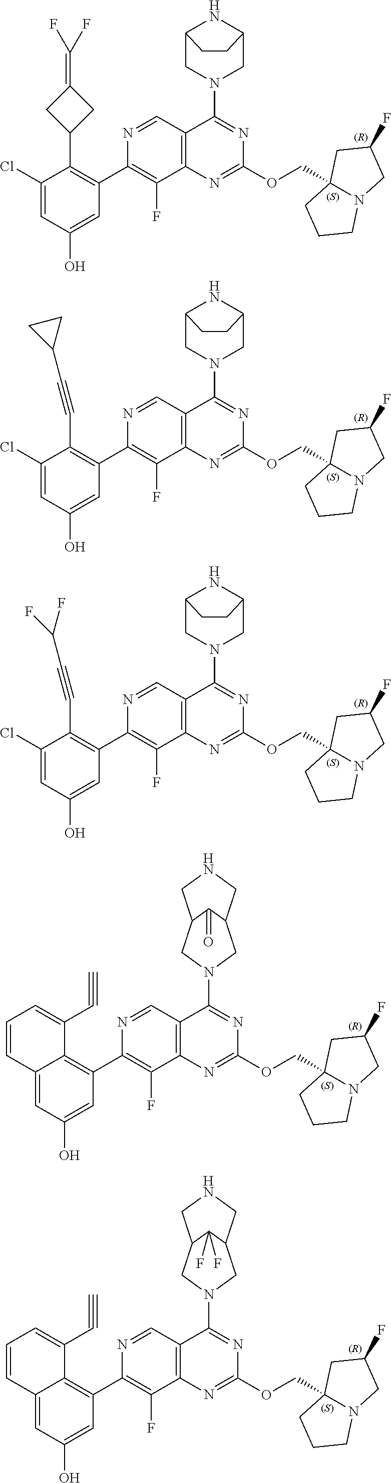 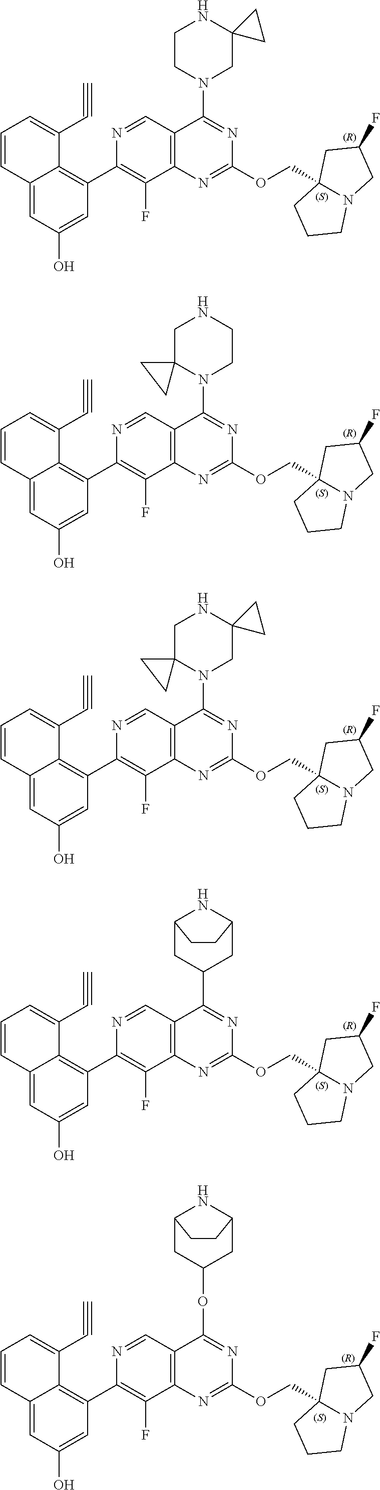 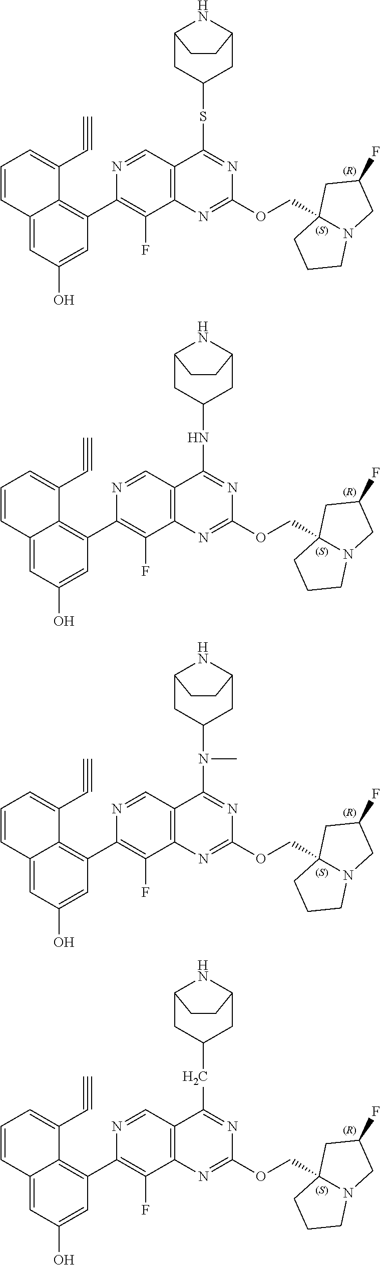 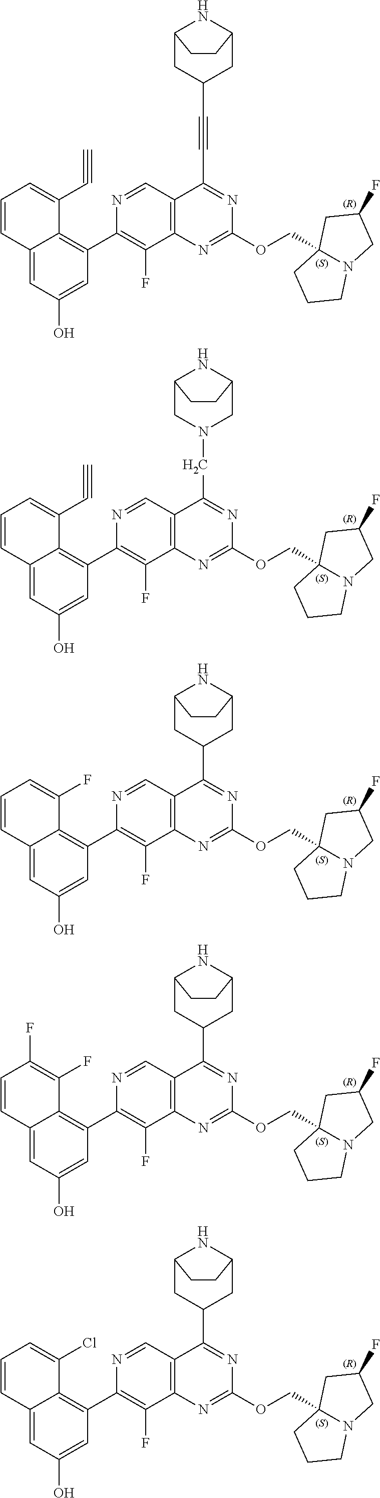 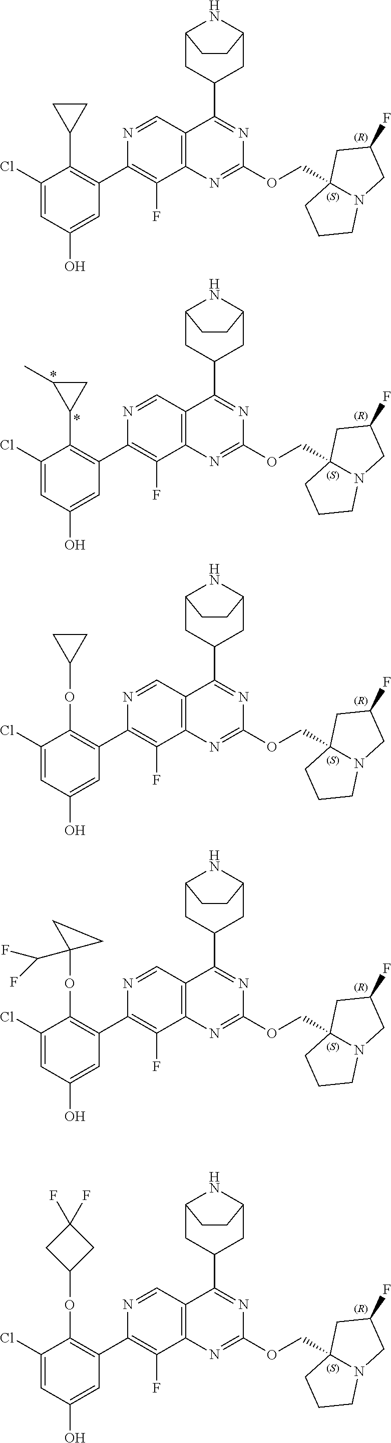 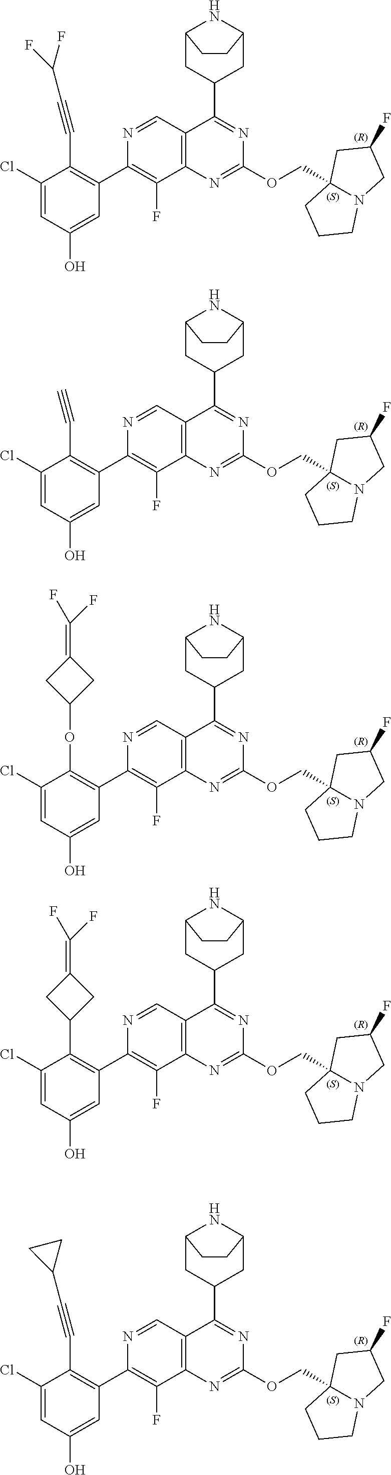 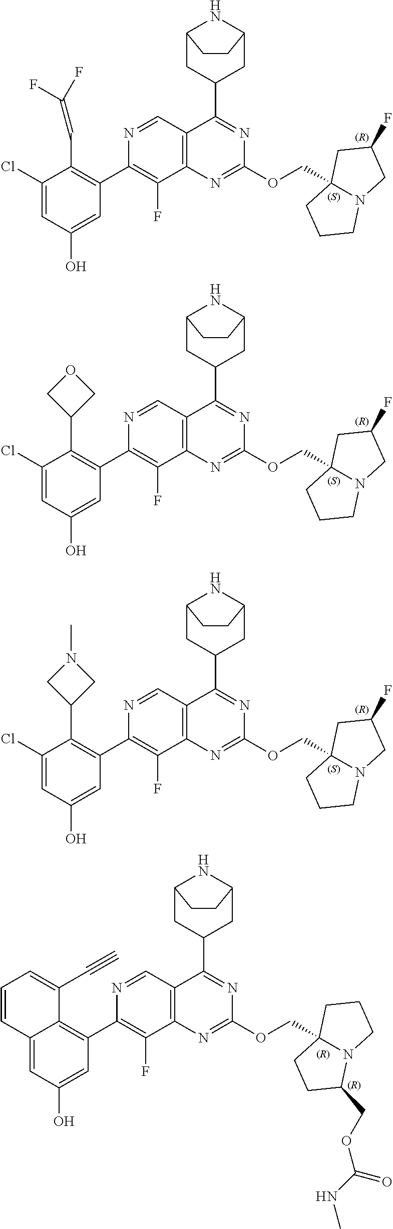 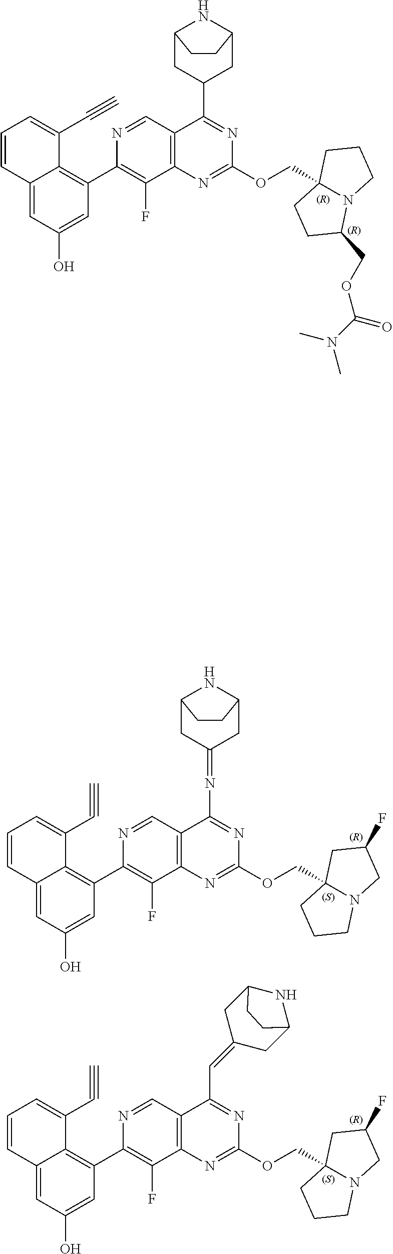 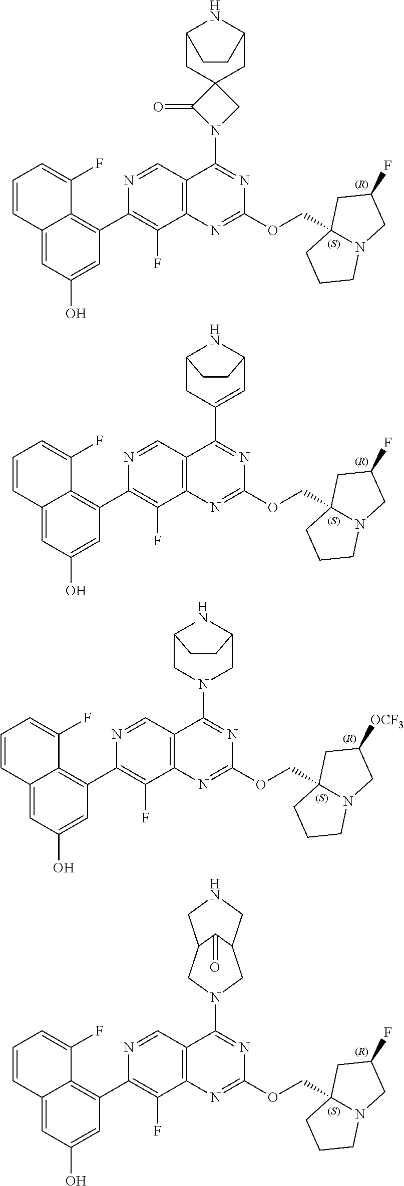 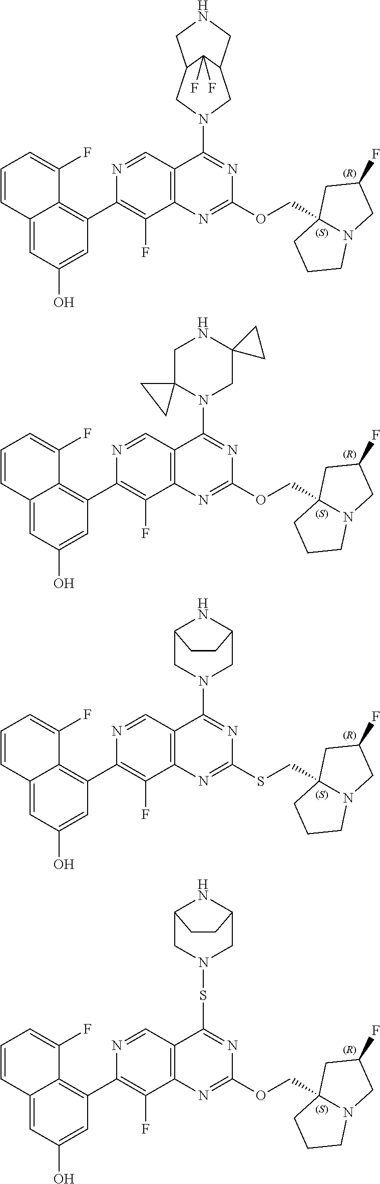 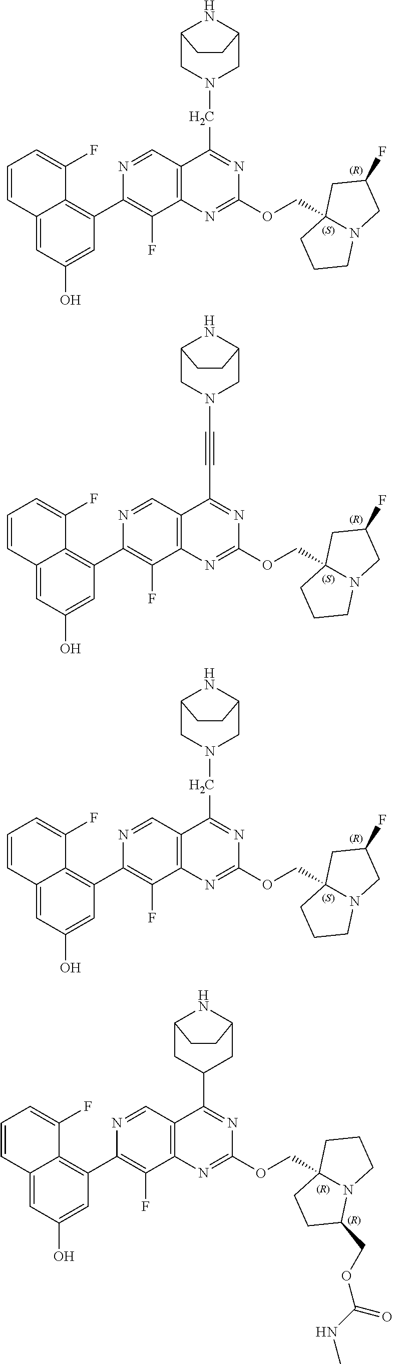 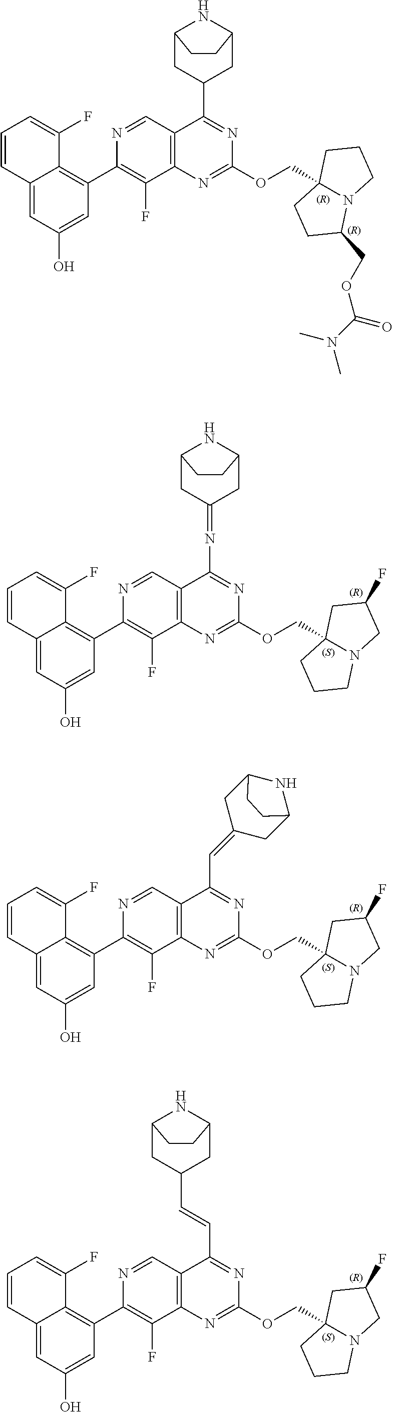 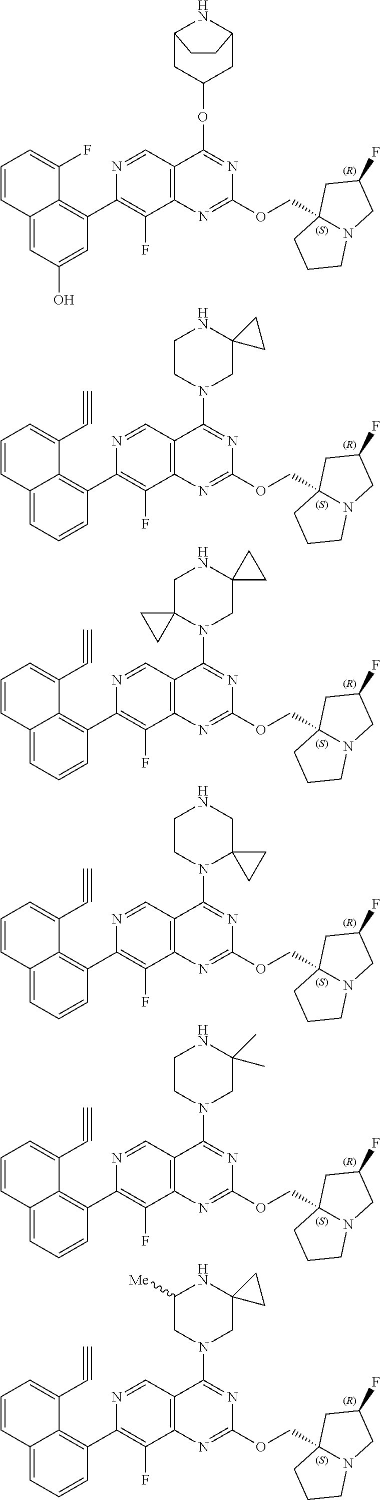 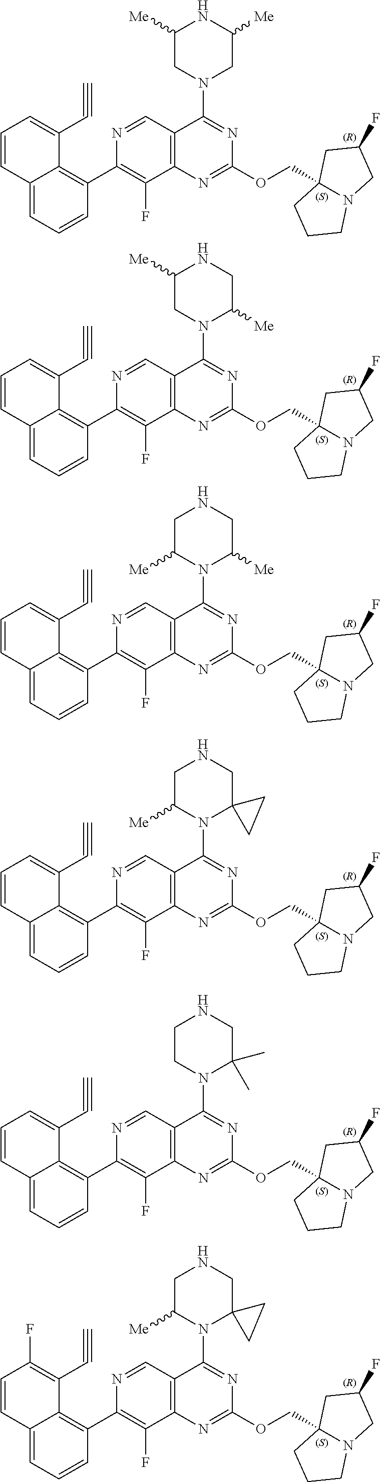 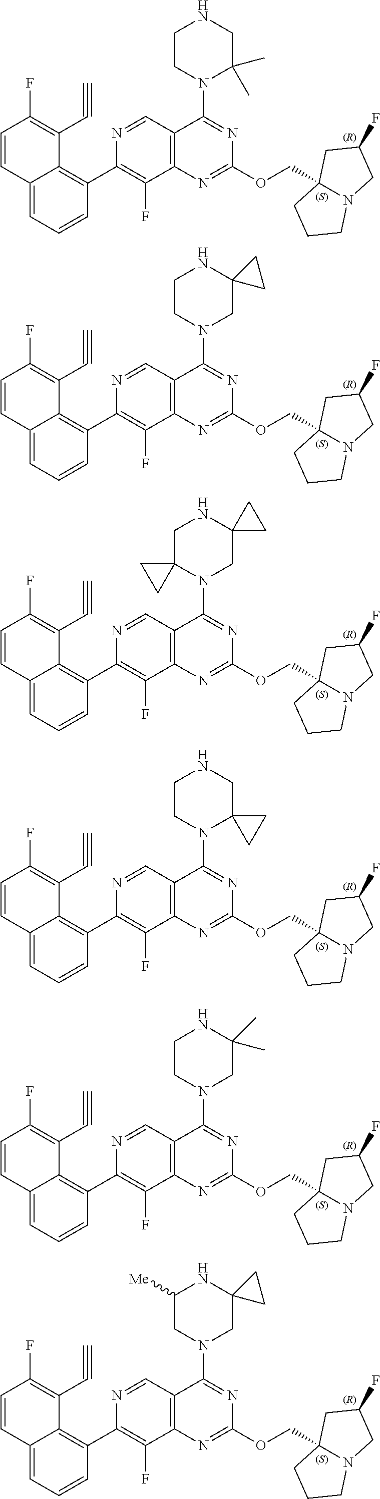 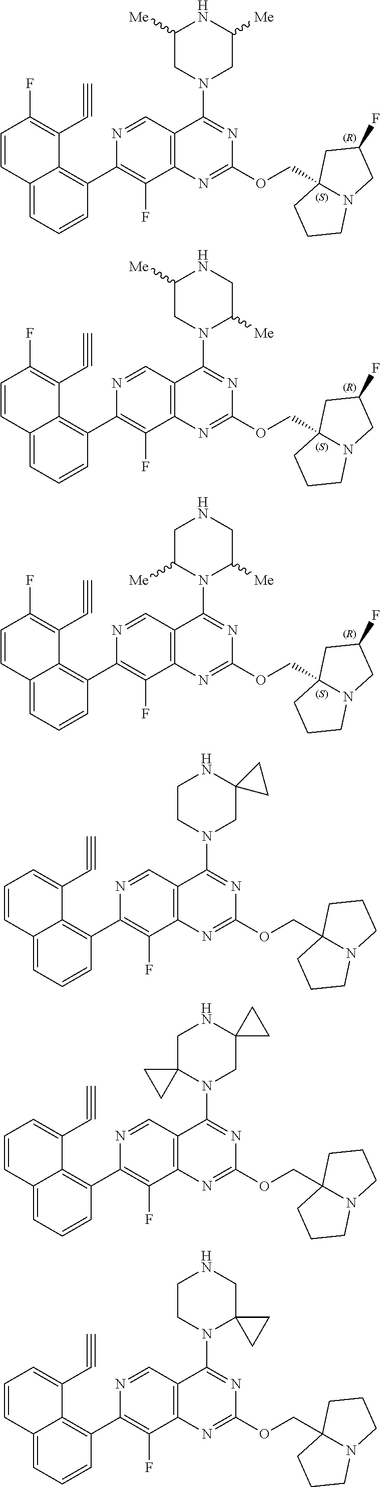 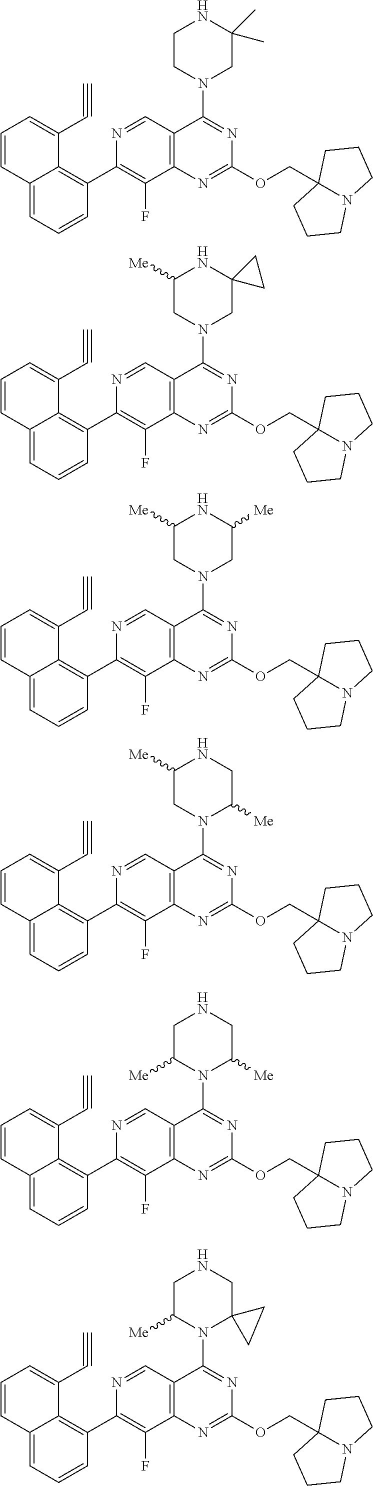 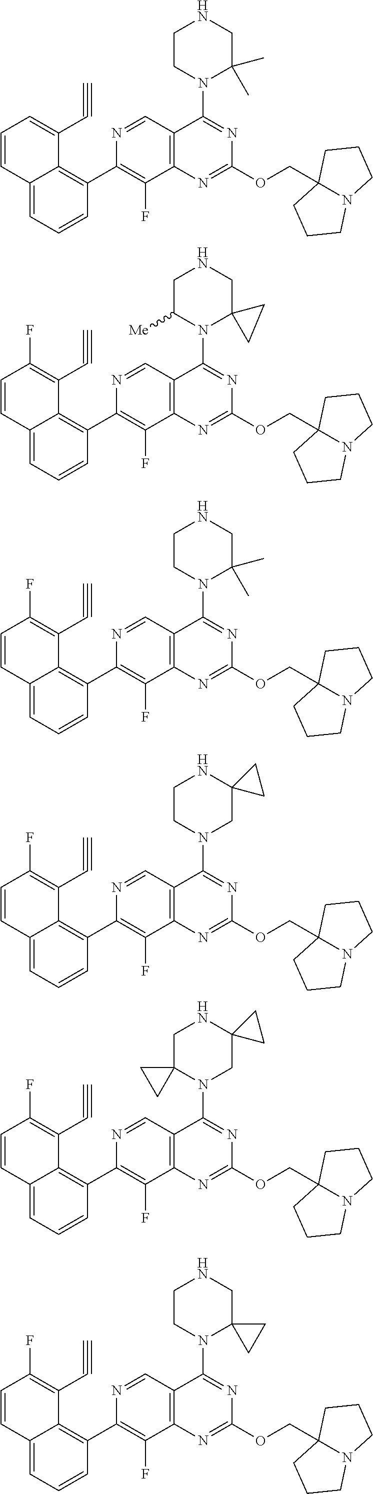 [SMILES: C#Cc1cccc2cccc(-c3ncc4c(N5CCNCC5(C)C)nc(OCC56CCCN5CCC6)nc4c3F)c12 ; C#Cc1c(F)ccc2cccc(-c3ncc4c(N5C(C)CNCC56CC6)nc(OCC56CCCN5CCC6)nc4c3F)c12 ; C#Cc1c(F)ccc2cccc(-c3ncc4c(N5CCNCC5(C)C)nc(OCC56CCCN5CCC6)nc4c3F)c12 ; C#Cc1c(F)ccc2cccc(-c3ncc4c(N5CCNC6(CC6)C5)nc(OCC56CCCN5CCC6)nc4c3F)c12 ; C#Cc1c(F)ccc2cccc(-c3ncc4c(N5CC6(CC6)NCC56CC6)nc(OCC56CCCN5CCC6)nc4c3F)c12 ; C#Cc1c(F)ccc2cccc(-c3ncc4c(N5CCNCC56CC6)nc(OCC56CCCN5CCC6)nc4c3F)c12] 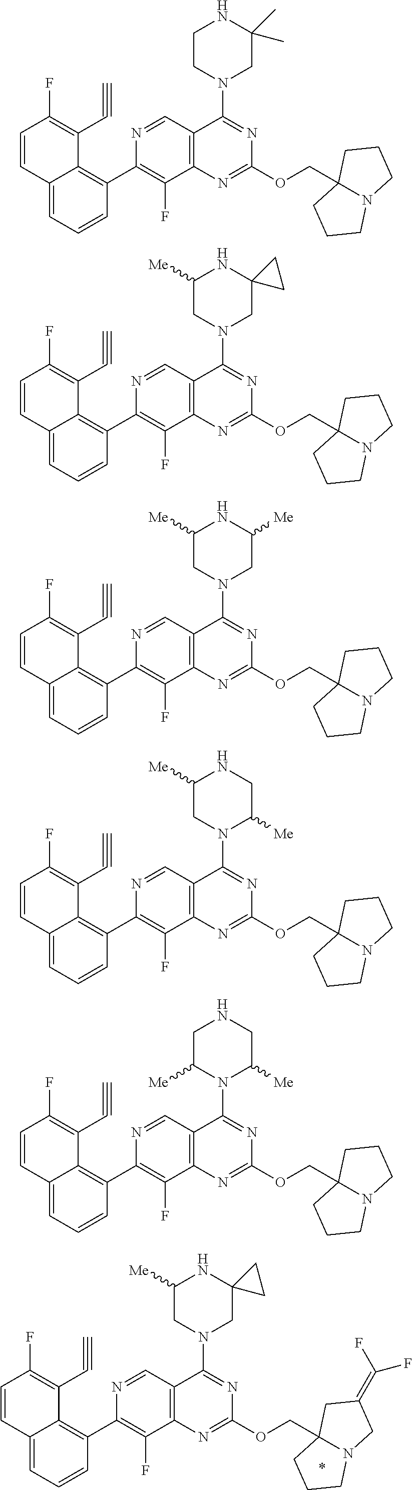 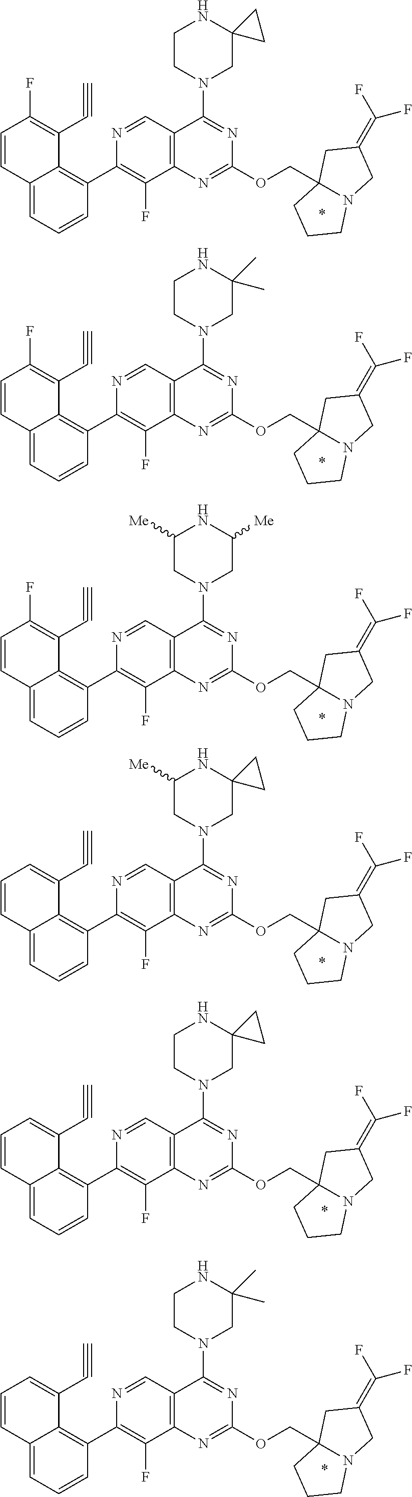 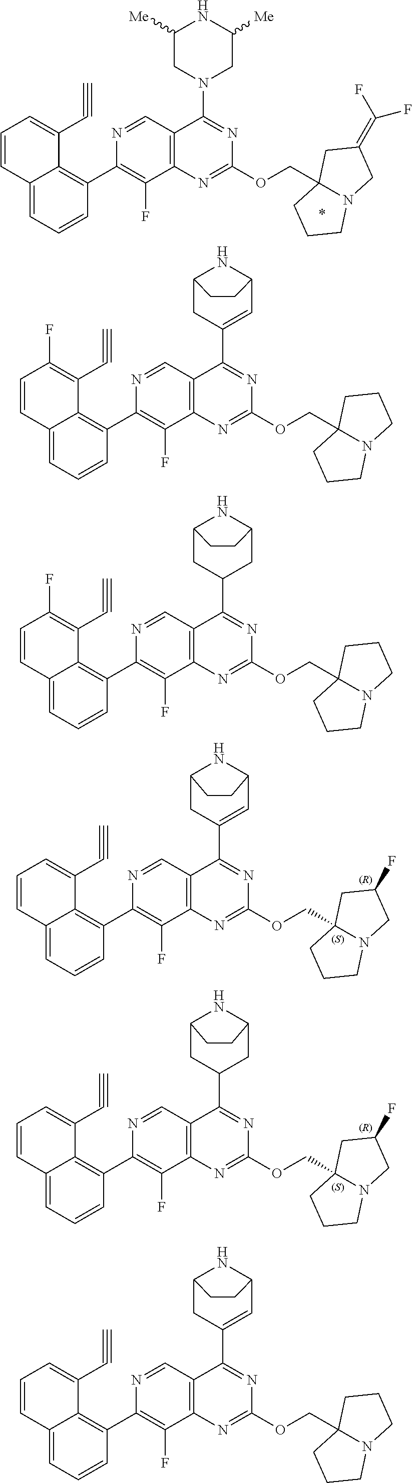 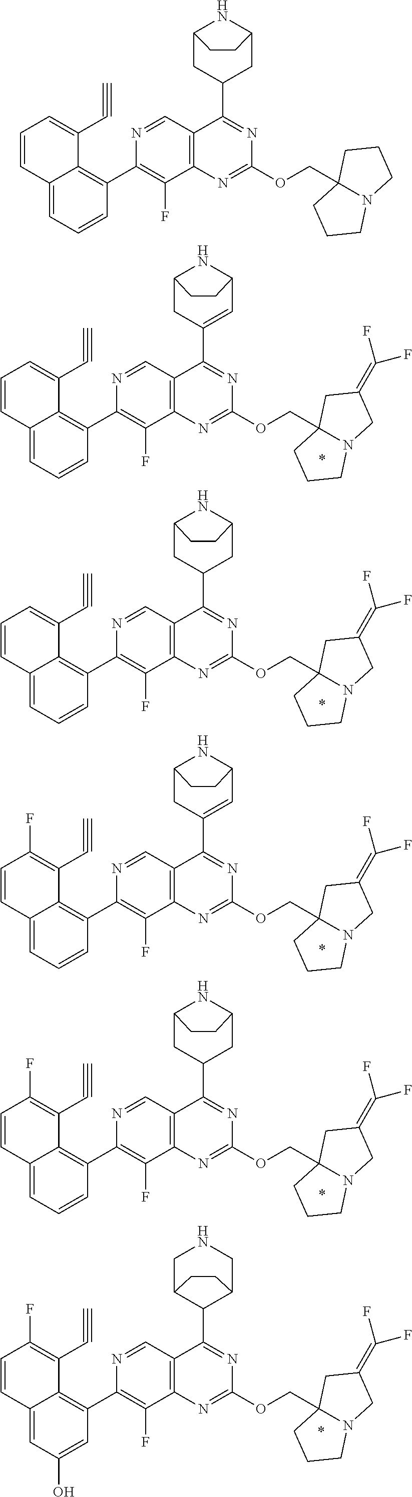 [SMILES: C#Cc1cccc2cccc(-c3ncc4c(C5CC6CCC(C5)N6)nc(OCC56CCCN5CCC6)nc4c3F)c12 ; C#Cc1cccc2cccc(-c3ncc4c(C5=CC6CCC(C5)N6)nc(OCC56CCCN5CC(=C(F)F)C6)nc4c3F)c12 ; C#Cc1cccc2cccc(-c3ncc4c(C5CC6CCC(C5)N6)nc(OCC56CCCN5CC(=C(F)F)C6)nc4c3F)c12 ; C#Cc1c(F)ccc2cccc(-c3ncc4c(C5=CC6CCC(C5)N6)nc(OCC56CCCN5CC(=C(F)F)C6)nc4c3F)c12 ; C#Cc1c(F)ccc2cccc(-c3ncc4c(C5CC6CCC(C5)N6)nc(OCC56CCCN5CC(=C(F)F)C6)nc4c3F)c12 ; C#Cc1c(F)ccc2cc(O)cc(-c3ncc4c(C5C6CCC5CNC6)nc(OCC56CCCN5CC(=C(F)F)C6)nc4c3F)c12] 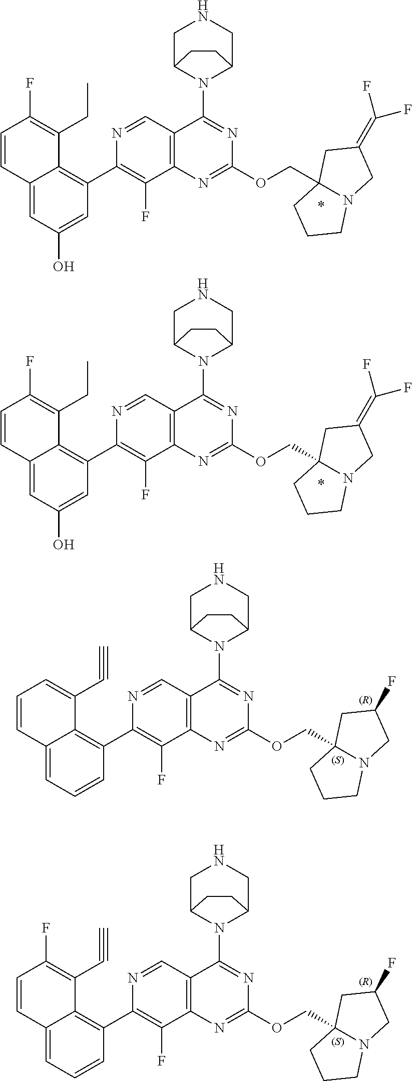 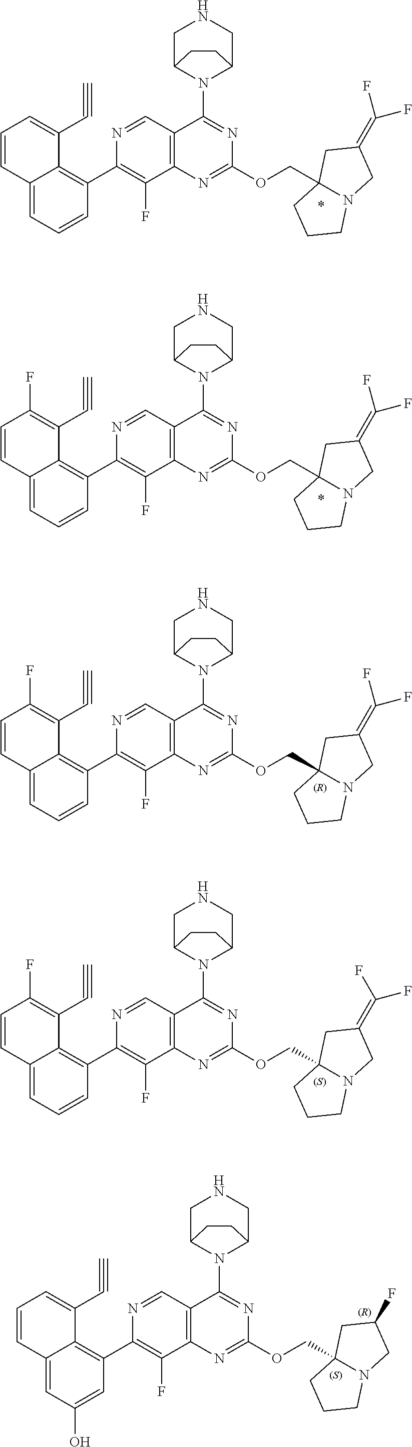 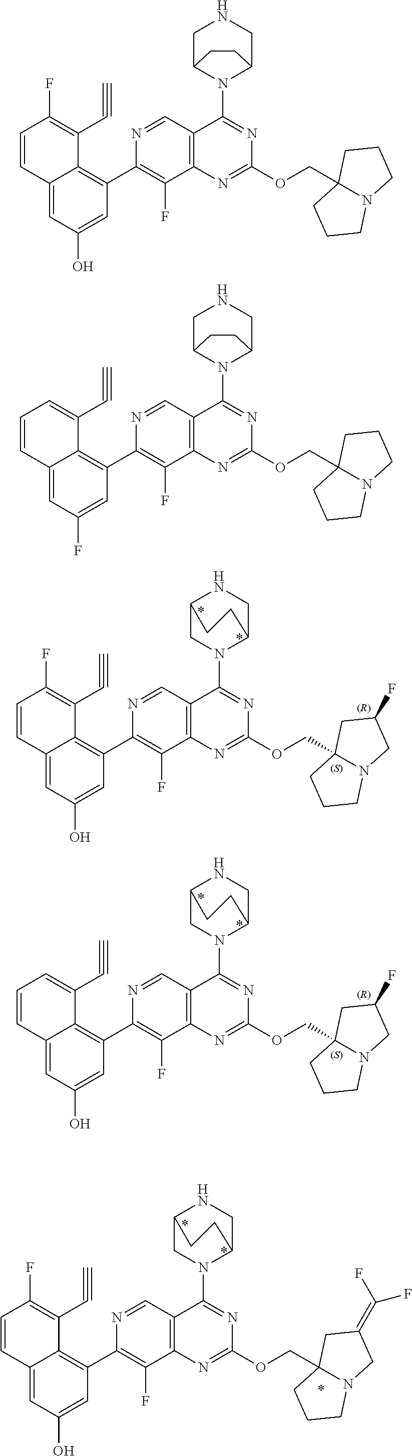 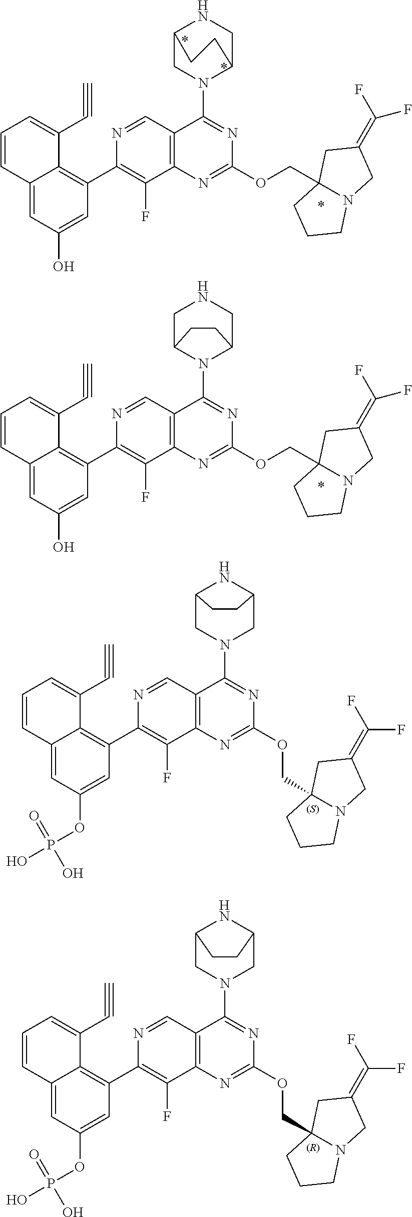 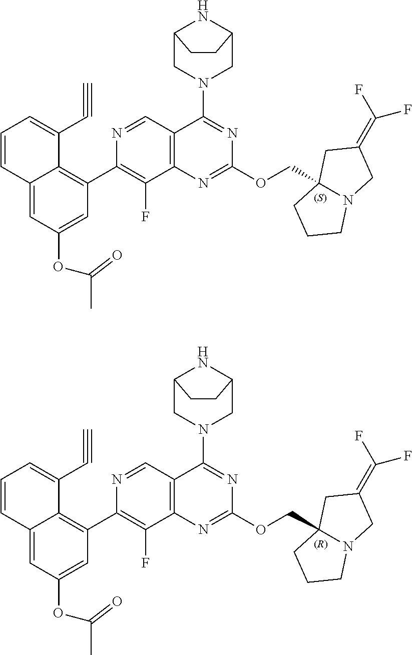
in the above structural formula, “*” represents a chiral center, which can be optionally in the R or S configuration, or a mixture of R and S configurations; the carbon atoms connected by the bond “ [symbol] ” can be optionally in R or S configuration, or optionally in the cis- or trans- configuration.

20. The compound according to any one of claims 1-19, or an optical isomer, a pharmaceutically acceptable salt, a prodrug, a deuterated derivative, a hydrate, or a solvate thereof, wherein, the pharmaceutically acceptable salt is selected from the group consisting of: potassium salts, sodium salts, magnesium salts, calcium salts, sulfate, hydrochloride, phosphate, sulfonate, and carbonate.

21. A pharmaceutical composition, wherein, comprising the compound according to any one of claims 1-20, or an optical isomer, a pharmaceutically acceptable salt, a prodrug, a deuterated derivative, a hydrate, or a solvate thereof, and pharmaceutically acceptable carriers.

22. A use of the compound according to any one of claims 1-20, or an optical isomer, a pharmaceutically acceptable salt, a prodrug, a deuterated derivative, a hydrate, or a solvate thereof, in the preparation of a pharmaceutical composition for treating diseases, disorders or conditions related to KRAS G12D activity or expression level.

23. The use according to claim 22, wherein, the disease, disorder or condition is selected from the various solid tumor and blood tumor consisting of: pancreatic cancer, non-small cell lung cancer, small cell lung cancer, lung adenocarcinoma, lung squamous carcinoma, colon cancer, colorectal cancer, thyroid cancer, embryonal rhabdomyosarcoma, skin granular cell tumor, melanoma, liver cancer, rectal cancer, bladder cancer, throat cancer, breast cancer, prostate cancer, glioma, ovarian cancer, head and neck squamous cell cancer, cervical cancer, esophageal cancer, kidney cancer, skin cancer, lymphoma, stomach cancer, acute myeloid leukemia, myelofibrosis, B-cell lymphoma, monocytic leukemia, polycythemia megalosplenica, eosinophilic leukocytosis syndrome, and myeloma, etc.
